(19) United States
(12) Patent Application Publication
(10) Pub. No.: US 2019/0050534 A1
(43) Pub. Date: Feb. 14, 2019

(54) DISEASE-ASSOCIATED MICROBIOME CHARACTERIZATION PROCESS

(71) Applicant: uBiome, Inc., San Francisco, CA (US)

(72) Inventors: Zachary Apte, San Francisco, CA (US); Jessica Richman, San Francisco, CA (US); Daniel Almonacid, San Francisco, CA (US); Inti Pedroso, Santiago (CL); Victoria Dumas, Buenos Aires (AR); Paz Tapia, Santiago (CL); Rodrigo Ortiz, Santiago (CL); Catalina Valdivia, Santiago (CL); Victor Alegria, Santiago (CL); Elisabeth M. Bik, San Francisco, CA (US); Maureen Hitschfeld, Santiago (CL)

(21) Appl. No.: 16/047,840

(22) Filed: Jul. 27, 2018

Related U.S. Application Data
(60) Provisional application No. 62/658,308, filed on Apr. 16, 2018.
(60) Provisional application No. 62/582,191, filed on Nov. 6, 2017.
(60) Provisional application No. 62/545,039, filed on Aug. 14, 2017.

(51) Int. Cl. G06F 19/24; C12Q 1/6888; G06F 19/12; G16H 70/60

(52) U.S. Cl. CPC G06F 19/24; G16H 70/60; G06F 19/12; C12Q 1/6888

(57) ABSTRACT

Embodiments of a method and/or system for characterizing one or more microorganism-related conditions can include: determining a microorganism dataset associated with a set of subjects; and with a set of microsome characterization modules, applying analytical techniques to perform a characterization process for the one or more microorganism-related conditions based on the microorganism dataset.

### CROSS-REFERENCE TO RELATED APPLICATIONS

This application claims the benefit of U.S. Provisional Application Ser. No. 62/582,191 filed 6 Nov. 2017, U.S. Provisional Application Ser. No. 62/545,039 filed 14 Aug. 2017, and U.S. Provisional Application Ser. No. 62/658,308 filed 16 Apr. 2018, which are each herein incorporated in their entirety by this reference.

### TECHNICAL FIELD

The disclosure generally relates to genomics and microbiology.

### BACKGROUND

A microbiome can include an ecological community of commensal, symbiotic, and pathogenic microorganisms that are associated with an organism. Characterization of the human microbiome is a complex process. The human microbiome includes over 10 times more microbial cells than human cells, but characterization of the human microbiome is still in nascent stages such as due to limitations in sample processing techniques, genetic analysis techniques, and resources for processing large amounts of data. Present knowledge has clearly established the role of microbiome associations with multiple health conditions, and has become an increasingly appreciated mediator of host genetic and environmental factors on human disease development. The microbiome is suspected to play at least a partial role in a number of health/disease-related states (e.g., preparation for childbirth, diabetes, auto-immune disorders, gastrointestinal disorders, rheumatoid disorders, neurological disorders, etc.). Further, the microbiome may mediate effects of environmental factors on human, plant, and/or animal health. Given the profound implications of the microbiome in affecting a subject's health, efforts related to the characterization of the microbiome, the generation of insights from the characterization, and the generation of therapeutics configured to rectify states of dysbiosis should be pursued. Current methods and systems for analyzing the microbiomes of humans and/or providing therapeutic measures based on gained insights have, however, left many questions unanswered.

As such, there is a need in the field of microbiology for a new and useful method and/or system for characterizing, monitoring, diagnosing, and/or intervening in one or more microorganism-related health conditions and/or the associated relationships (e.g., specific features associated with microorganisms and/or conditions, etc.), such as for individualized and/or population-wide use.

### BRIEF DESCRIPTION OF THE FIGURES

FIG. 1 includes a flowchart representation of variations of an embodiment of a method;

FIG. 2 includes a representation of variations of embodiments of a method and system;

FIG. 3 includes a variation of a process for generation of a characterization model in an embodiment of a method;

FIG. 4 includes variations of mechanisms by which probiotic-based therapies operate in an embodiment of a method;

FIG. 5 includes variations of sample processing in an embodiment of a method;

FIG. 6 includes examples of notification provision;

FIG. 7 includes a schematic representation of variations of an embodiment of the method;

FIGS. 8A-8C include variations of performing characterization processes with models;

FIG. 9 includes promoting a therapy in a variation of an embodiment of a method;

FIG. 10 includes a variation of a microbiome characterization module;

FIG. 11 includes a variation of a microbiome characterization module;

FIG. 12 includes a variation of a microbiome characterization module;

FIG. 13 includes a variation of a microbiome characterization module;

FIG. 14 includes a variation of a microbiome characterization module;

FIG. 15 includes a variation of a microbiome characterization module;

FIG. 16 includes a variation of a microbiome characterization module;

FIG. 17 includes a variation of multi-site analyses;

FIG. 18 includes a specific example of a Venn Diagram with comparison of the results from different statistical techniques (e.g., univariate statistical techniques) for sampling site of the gut;

FIG. 19 includes a specific example of a representation of the dimensionality reduction obtained from the application of Analytical Module B, with each Microbiome sub-system detected as represented by a different grey-scale color, and a module of relevance indicated by filled black lines;

FIG. 20 includes a specific example of a representation of interaction between microorganism taxonomies and functions, with functions represented by squares and taxonomies represented by circles;

FIG. 21 includes a specific example of variance explained by microbiome characteristics associated with each condition analyzed, with values corresponding to mean and 32th and 68th percentiles of the variance explained, and with conditions organized on each panel by the main site of manifestation;

FIG. 22 includes a specific example of a representation of clustering analysis using the microbiome-based significance correlations to obtain a data-driven arrangement of the conditions being analyzed;

FIG. 23 includes variations of microbiome characterization modules and associated aspects;

FIG. 24 includes a specific example of a heat map of microbiome-related association amongst microorganism-related conditions; and

FIG. 25 includes a specific example of number of individuals showing intra and inter-cluster comorbidity.

### DESCRIPTION OF THE EMBODIMENTS

The following description of the embodiments is not intended to limit the embodiments, but rather to enable any person skilled in the art to make and use.

### 1. Overview

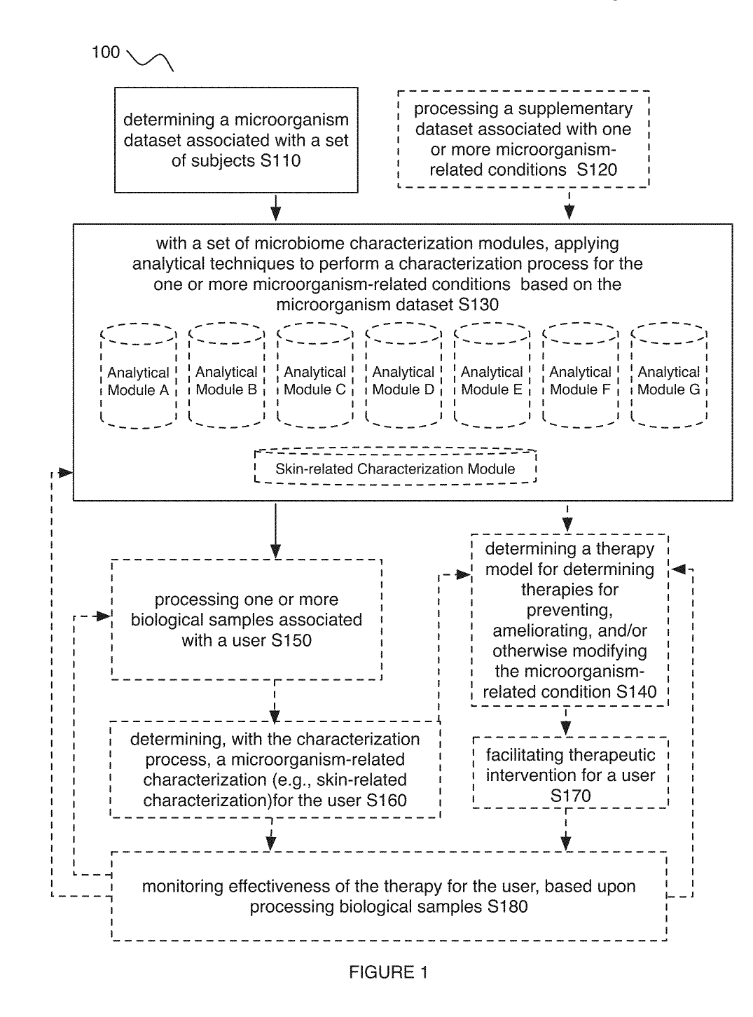

As shown in FIG. 1, embodiments of a method 100 for characterizing one or more microorganism-related conditions (e.g., disease-related conditions, human behavior conditions, etc.) can include: determining a microorganism dataset (e.g., microorganism sequence dataset, microbiome composition diversity dataset such as based upon a microorganism sequence dataset, microbiome functional diversity dataset such as based upon a microorganism sequence dataset, etc.) associated with a set of subjects S110; and with a set of microbiome characterization modules, applying analytical techniques to perform a characterization process (e.g., pre-processing, feature generation, feature processing, multi-site characterization for a plurality of collection sites, cross-condition analysis for a plurality of microorganism-related conditions, model generation, etc.) for the one or more microorganism-related conditions (e.g., human behavior conditions, disease-related conditions, etc.), based on the microorganism dataset (e.g., based on microbiome features derived from the microorganism dataset; etc.) S130.

Embodiments of the method 100 can additionally or alternatively include one or more of: processing a supplementary dataset (e.g., describing one or more characteristics of the user, such as medical condition history, etc.) associated with (e.g., informative of; describing; indicative of; correlated with, etc.) one or more microorganism-related conditions for the set of subjects S120; determining a therapy model for determining therapies for preventing, ameliorating, and/or otherwise modifying one or more microorganism-related conditions S140; processing one or more biological samples associated with a user (e.g., subject, human, animal, patient, etc.) S150; determining, with the characterization process, a microorganism-related characterization (e.g., human behavior characterization, disease-related characterization, etc.) for the user based upon processing a user microorganism dataset (e.g., user microorganism sequence dataset, user microbiome composition dataset, user microbiome function dataset, etc.) derived from the biological sample of the user S160; facilitating therapeutic intervention for the one or more microorganism-related conditions for the user (e.g., based upon the microorganism-related characterization and/or a therapy model; etc.) S170; monitoring effectiveness of the therapy for the user, based upon processing biological samples, to assess microbiome composition and/or functional features associated with the therapy for the user over time S180; and/or any other suitable operations.

Embodiments of the method 100 and/or system 200 can function to apply one or more microbiome characterization modules (e.g., for applying one or more analytical techniques, etc.) to characterize (e.g., assess, evaluate, diagnose, describe, etc.) microorganism-related conditions and/or users in relation to microorganism-related conditions (e.g., human behavior conditions, disease-related conditions, etc.), such as for facilitating therapeutic intervention (e.g., therapy selection; therapy promotion and/or provision; therapy monitoring; therapy evaluation; etc.). In an example, the method 100 can include: determining a microorganism sequence dataset associated with a set of subjects based on microorganism nucleic acids from biological samples associated with the set of subjects, where the microorganism nucleic acids are associated with the microorganism-related condition; with a set of microbiome characterization modules, applying a set of analytical techniques (e.g., at least one of a statistical test such as univariate statistical tests, a dimensionality reduction technique, an artificial intelligence approach, another approach described herein, etc.) to determine a set of microbiome features based on the microorganism sequence dataset; generating a microorganism-related condition model (e.g., for phenotype prediction, such as estimating a propensity-score for a user for the microorganism-related condition, etc.) based on the set of microbiome features (and/or any other suitable data); and determining a characterization of the microorganism-related condition for a user based on the microorganism-related condition model and a sample from the user (e.g., through sample processing and computational processing for generating user microbiome feature values to use with the microorganism-related condition model, etc.).

Additionally or alternatively, embodiments of the method 100 and/or system 200 can function to perform cross-condition analyses (e.g., using one or more microbiome characterization modules, etc.) for a plurality of microorganism-related conditions (e.g., characterization of a plurality of microorganism-related conditions, etc.), such as in the context of characterizing, diagnosing, and/or treating a user. In an example, the method 100 can include determining a microorganism sequence dataset associated with the set of subjects, based on microorganism nucleic acids from biological samples associated with the set of subjects, where the microorganism nucleic acids are associated with the plurality of microorganism-related conditions (e.g., the microorganism nucleic acids are associated with microbiome features correlated with two or more of the plurality of microorganism-related conditions, etc.); with a set of microbiome characterization modules, determining a set of multi-condition microbiome features based on the microorganism sequence dataset, where each multi-condition microbiome feature of the set of multi-condition microbiome features is associated with at least two microorganism-related conditions of the plurality of microorganism-related conditions (e.g., features shared across multiple microorganism-related conditions, in relation to relevance, correlation, covariance, etc.); determining, for a user, a multi-condition characterization of microorganism-related conditions (e.g., a subset, all of, etc.) of the plurality of microorganism-related conditions based on the set of multi-condition microbiome features and a sample from the user; and facilitating therapeutic intervention for the microorganism-related conditions of the plurality of microorganism-related conditions based on the multi-condition characterization.

Additionally or alternatively, embodiments of the method 100 and/or system 200 can identify microbiome features associated with different microorganism-related conditions, such as for use as biomarkers (e.g., for diagnostic processes, for treatment processes, etc.). In examples, microorganism-related characterization can be associated with at least one or more of user microbiome composition (e.g., microbiome composition diversity, etc.), microbiome function (e.g., microbiome functional diversify, etc.), and/or other suitable microbiome-related aspects.

Additionally or alternatively, embodiments can function to facilitate therapeutic intervention for microorganism-related conditions, such as through promotion of associated therapies (e.g., in relation to specific physiological sites gut, skin, nose, mouth, genitals, other suitable physiological sites, other collection sites, etc.). Additionally or alternatively, embodiments can function to generate models (e.g., microbiome characterization modules such as for phenotypic prediction and/or prediction scores, machine learning models such as for feature processing, etc.), such as models that can be used to characterize and/or diagnose users based on their microbiome (e.g., user microbiome features; as a clinical diagnostic; as a companion diagnostic, etc.), and/or that can be used to select and/or provide therapies (e.g., probiotic-based therapeutic measures, phage-based therapeutic measures, small-molecule-based therapeutic measures, clinical measures, etc.) for subjects in relation to one or more microorganism-related conditions. Additionally or alternatively, embodiments can perform any suitable functionality described herein.

As such, data from populations of subjects (e.g., associated with one or more microorganism-related conditions, etc.) can be processed with one or more microbiome characterization modules (e.g., for generating models, etc.) to characterize subsequent users, such as for indicating microorganism-related states of health and/or areas of improvement, and/or to facilitate therapeutic intervention (e.g., promoting one or more therapies; facilitating modulation of the composition and/or functional diversity of a user's microbiome toward one or more of a set of desired equilibrium states, such as states correlated with improved health states associated with one or more microorganism-related conditions; etc.). Variations of the method 100 can further facilitate selection, monitoring (e.g., efficacy monitoring, etc.) and/or adjusting of therapies provided to a user, such as through collection and analysis (e.g., with microbiome characterization modules) of additional samples from a subject over time (e.g., throughout the course of a therapy regimen, through the extent of a user's experiences with microorganism-related conditions; etc.) and/or across collection sites for one or more microorganism-related conditions (e.g., where characterization can include cross-condition characterization for a plurality of conditions, etc.). However, data from populations, subgroups, individuals, and/or other suitable entities can be used by any suitable portions of the method 100 and/or system 200 for any suitable purpose.

Embodiments of the method 100 and/or system 200 can preferably generate and/or promote (e.g., provide; present; notify regarding; etc.) characterizations and/or therapies for one or more microorganism-related conditions, which can include one or more of: diseases, symptoms, causes (e.g., triggers, etc.), disorders, associated risk (e.g., propensity scores, etc.), associated severity, behaviors (e.g., caffeine consumption, habits, diets, etc.), and/or any other suitable aspects associated with microorganism-related conditions. Microorganism-related conditions can include one or more disease-related conditions, which can include any one or more of: skin-related conditions (e.g., acne, dermatomyositis, eczema, rosacea, dry skin, psoriasis, dandruff, photosensitivity, rough skin, itching, flaking, scaling, peeling, fine lines or cracks, gray skin in individuals with dark skin, redness, deep cracks such as cracks that can bleed and lead to infections, itching and scaling of the skin in the scalp, oily skin such as irritated oily skin, skin sensitivity to products such as hair care products, imbalance in scalp microbiome, etc.); gastrointestinal-related conditions (e.g., irritable bowel syndrome, inflammatory bowel disease, ulcerative colitis, celiac disease, Crohn's disease, bloating, hemorrhoidal disease, constipation, reflux, bloody stool, diarrhea, etc.); allergy-related conditions (e.g., allergies and/or intolerance associated with wheat, gluten, dairy, soy, peanut, shellfish, tree nut, egg, etc.); locomotor-related conditions (e.g., gout, rheumatoid arthritis, osteoarthritis, reactive arthritis, multiple sclerosis, Parkinson's disease, etc.); cancer-related conditions (e.g., lymphoma; leukemia; blastoma; germ cell tumor; carcinoma; sarcoma; breast cancer; prostate cancer; basal cell cancer; skin cancer; colon cancer; lung cancer; cancer conditions associated with any suitable physiological region; etc.), cardiovascular-related conditions (e.g., coronary heart disease, inflammatory heart disease, valvular heart disease, obesity, stroke, etc.), anemia conditions (e.g., thalassemia; sickle cell; pernicious; fanconi; haemolyitic; aplastic; iron deficiency; etc.), neurological-related conditions (e.g., ADHD, ADD, anxiety, Asperger's syndrome, autism, chronic fatigue syndrome, depression, etc.), autoimmune-related conditions (e.g., Sprue, AIDS, Sjogren's, Lupus, etc.), endocrine-related conditions (e.g., obesity, Graves' disease, Hashimoto's thyroiditis, metabolic disease, Type I diabetes, Type II diabetes, etc.), Lyme disease conditions, communication-related conditions, sleep-related conditions, metabolic-related conditions, weight-related conditions, pain-related conditions, genetic-related conditions, chronic disease, and/or any other suitable type of disease-related conditions. In variations, portions of embodiments of the method 100 and/or system 200 can be used in promoting (e.g., providing, etc.) one or more targeted therapies to users suffering from one or more microorganism-related conditions (e.g., skin-related conditions, etc.). Additionally or alternatively, microorganism-related conditions can include one or more human behavior conditions which can include any one or more of: caffeine consumption, alcohol consumption, other food item consumption, dietary-supplement consumption, probiotic-related behaviors (e.g., consumption, avoidance, etc.), other dietary behaviors, habituary behaviors (e.g., smoking; exercise conditions such as low, moderate, and/or extreme exercise conditions; etc.), menopause, other biological processes, social behavior, other behaviors, and/or any other suitable human behavior conditions. Conditions can be associated with any suitable phenotypes (e.g., phenotypes measurable for a human, animal, plant, fungi body, etc.).

Embodiments of the method 100 and/or system 200 can be implemented for a single user, such as in relation to applying one or more microbiome characterization modules for processing one or more biological samples (e.g., collected across one or more collection sites) from the user, for microorganism-related characterization, facilitating therapeutic intervention, and/or for any other suitable purpose (e.g., for one or more microorganism-related conditions, etc.). Additionally or alternatively, embodiments can be implemented for a population of subjects (e.g., including the user, excluding the user), where the population of subjects can include subjects similar to and/or dissimilar to any other subjects for any suitable type of characteristics (e.g., in relation to microorganism-related conditions, demographic features behavior, microbiome composition and/or function, etc.); implemented for a subgroup of users (e.g., sharing characteristics, such as characteristics affecting microorganism-related characterization and/or therapy determination; etc.); implemented for plants, animals, microorganisms, and/or any other suitable entities. Thus, information derived from a set of subjects (e.g., population of subjects, set of subjects, subgroup of users, etc.) can be used to provide additional insight for subsequent users. In a variation, an aggregate set of biological samples is preferably associated with and processed for a wide variety of users, such as including users of one or more of; different demographics (e.g., genders, ages, marital statuses, ethnicities, nationalities, socioeconomic statuses, sexual orientations, etc.), different microorganism-related conditions (e.g., health and disease states; different genetic dispositions; etc.), different living situations (e.g., living alone, living with pets, living with a significant other, living with children, etc.), different dietary habits (e.g., omnivorous, vegetarian, vegan, sugar consumption, acid consumption, caffeine consumption, etc.), different behavioral tendencies (e.g., levels of physical activity, drug use, alcohol use, etc.), different levels of mobility (e.g., related to distance traveled within a given time period), and/or any other suitable characteristic (e.g., characteristics influencing, correlated with, and/or otherwise associated with microbiome composition and/or function, etc.). In examples, as the number of users increases, the predictive power of processes implemented in portions of the method 100 can increase, such as in relation to characterizing a variety of users based upon their microbiomes (e.g., in relation to different collection sites for samples for the users, etc.). However, portions of the method 100 and/or system 200 can be performed and/or configured in any suitable manner for any suitable entity or entities.

Data described herein (e.g., microbiome characterization module inputs, microbiome characterization module outputs, microorganism datasets, microbiome features, microorganism-related characterizations, therapy-related data, user data, supplementary data, notifications, etc.) can be associated with any suitable temporal indicators (e.g., seconds, minutes, hours, days, weeks, etc.) including one or more; temporal indicators indicating when the data was collected (e.g., temporal indicators indicating when a sample was collected; etc.), determined, transmitted, received, and/or otherwise processed; temporal indicators providing context to content described by the data (e.g., temporal indicators associated with microorganism-related characterizations, such as where the microorganism-related characterization describes the microorganism-related conditions and/or user microbiome status at a particular time; etc.); changes in temporal indicators (e.g., changes in microorganism-related characterizations over time, such as in response to receiving a therapy; latency between sample collection, sample analysis, provision of a microorganism-related characterization or therapy to a user, and/or other suitable portions of the method 100; etc.); and/or any other suitable indicators related to time.

Additionally or alternatively, parameters, metrics, inputs, outputs, and/or other suitable data can be associated with value types including; scores (e.g., microorganism-related condition propensity scores; feature relevance scores; correlation scores, covariance scores, microbiome diversity scores, severity scores; etc.), individual values (e.g., individual microorganism-related scores, such as condition propensity scores, for different collection sites, etc.), aggregate values, (e.g., overall scores based on individual microorganism-related scores for different collection sites, etc.), binary values (e.g., presence or absence of a microbiome feature; presence or absence of a microorganism-related condition; etc.), relative values (e.g., relative taxonomic group abundance, relative microbiome function abundance, relative feature abundance, etc.), classifications (e.g., microorganism-related condition classifications and/or diagnoses for users; microorganism-related condition cluster classifications for conditions; feature classifications; behavior classifications; demographic classifications; etc.), confidence levels (e.g., associated with microorganism sequence datasets; with microbiome diversity scores; with other microorganism-related characterizations; with other outputs; etc.), identifiers (e.g., identifying the microbiome characterization modules used in processing the data, etc.), values along a spectrum, and/or any other suitable types of values. Any suitable types of data described herein can be used as inputs (e.g., for different modules, models, and/or other suitable components described herein), generated as outputs (e.g., of different models, modules, etc.), and/or manipulated in any suitable manner for any suitable components associated with the method 100 and/or system 200.

One or more instances and/or portions of the method 100 and/or processes described herein can be performed asynchronously (e.g., sequentially), concurrently (e.g., parallel data processing with microbiome characterization modules; concurrent cross-condition analysis; multiplex sample processing, such as multiplex amplification of microorganism nucleic acid fragments corresponding to target sequences associated with microorganism-related conditions; performing sample processing and analysis for substantially concurrently evaluating a panel of microorganism-related conditions; computationally determining microorganism datasets, microbiome features, and/or characterizing microorganism-related conditions in parallel for a plurality of users; such as concurrently on different threads for parallel computing to improve system processing ability; etc.), in temporal relation (e.g., substantially concurrently with, in response to, serially, prior to, subsequent to, etc.) to a trigger event (e.g., performance of a portion of the method 100), and/or in any other suitable order at any suitable time and frequency by and/or using one or more instances of the system 200, components, and/or entities described herein. In an example, the method 100 can include generating a microorganism dataset based on processing microorganism nucleic acids of one or more biological samples with a bridge amplification substrate of a next generation sequencing platform (and/or other suitable sequencing system) of a sample handling system, and determining microsome features and microbiome functional diversity features at computing devices operable to communicate with the next generation sequencing platform. However, the method 100 and/or system 200 can be configured in any suitable manner.

### 2. Benefits.

Microbiome analysis can enable accurate and/or efficient characterization and/or therapy provision (e.g., according to portions of the method 100, etc.) for microorganism-related conditions caused by and/or otherwise associated with microorganisms. Specific examples of the technology can overcome several challenges faced by conventional approaches in characterizing a user condition (e.g., microorganism-related condition) and/or facilitating therapeutic intervention. First, conventional approaches can require patients to visit one or more care providers to receive a characterization and/or a therapy recommendation for a microorganism-related condition (e.g., through diagnostic medical procedures such as blood testing; etc.), which can amount to inefficiencies and/or health-risks associated with the amount of time elapsed before diagnosis and/or treatment, with inconsistency in healthcare quality, and/or with other aspects of care provider visitation. Second, conventional genetic sequencing and analysis technologies for human genome sequencing can be incompatible and/or inefficient when applied to the microbiome (e.g., where the human microbiome can include over 10 times more microbial cells than human cells; where viable analytical techniques and the means of leveraging the analytical techniques can differ; where optimal sample processing techniques can differ, such as for reducing amplification bias; where different approaches to microorganism-related characterizations can be employed; where the types of conditions and correlations can differ; where causes of the associated conditions and/or viable therapies for the associated conditions can differ; where sequence reference databases can differ; where the microbiome can vary across different body regions of the user such as at different collection sites; etc.). Third, the onset of sequencing technologies (e.g., next-generation sequencing, associated technologies, etc.) has given rise to technological issues (e.g., data processing and analysis issues for the plethora of generated sequence data; issues with processing a plurality of biological samples in a multiplex manner; information display issues; therapy prediction issues; therapy provision issues, etc.) that would not exist but for the unprecedented advances in speed and data generation associated with sequencing genetic material. Specific examples of the method 100 and/or system 200 can confer technologically-rooted solutions to at least the challenges described above.

First, specific examples of the technology can transform entities (e.g., users, biological samples, therapy facilitation systems including medical devices, etc.) into different states or things. For example, the technology can transform a biological sample into components able to be sequenced and analyzed to generate microorganism dataset and/or microbiome features usable for characterizing users in relation to one or more microorganism-related conditions (e.g., such as through use of microbiome characterization modules, next-generation sequencing systems, multiplex amplification operations; etc.). In another example, the technology can identify, promote (e.g., present, recommend, etc.), discourage, and/or provide therapies (e.g., personalized therapies based on a microbiome characterization; etc.) and/or otherwise facilitate therapeutic intervention (e.g., facilitating modification of a user's microbiome composition, microbiome functionality, etc.), which can prevent and/or ameliorate one or more microorganism-related conditions, thereby transforming the microbiome and/or health of the patient (e.g., improving a health state associated with a microorganism-related condition; etc.). In another example, the technology can transform microbiome composition and/or function at one or more different physiological sites of a user (e.g., one or more different collection sites, etc.), such as targeting and/or transforming microorganisms associated with a gut, nose, skin, mouth, and/or genitals microbiome. In another example, the technology can control treatment-related systems (e.g., dietary systems; automated medication dispensers; behavior modification systems; diagnostic systems; disease therapy facilitation systems; etc.) to promote therapies (e.g., by generating control instructions for the therapy facilitation system to execute; etc.), thereby-transforming the therapy facilitation system.

Second, specific examples of the technology can confer improvements in computer-related technology (e.g., improving computational efficiency in storing, retrieving, and/or processing microorganism-related data for microorganism-related conditions; computational processing associated with biological sample processing, etc.) such as by facilitating computer performance of functions not previously performable. For example, the technology can leverage a set of microbiome characterization modules to apply a plurality of analytical techniques in a non-generic manner to non-generic microorganism datasets and/or microbiome features (e.g., that are recently able to be generated and/or are viable due to advances in sample processing techniques and/or sequencing technology, etc.) for improving microorganism-related characterizations and/or facilitating therapeutic intervention for microorganism-related conditions.

Third, specific examples of the technology can confer improvements in processing speed, microorganism-related characterization, accuracy, microbiome-related therapy determination and promotion, and/or other suitable aspects in relation to microorganism-related conditions. For example, the technology can leverage a set of a microbiome characterization modules with non-generic microorganism datasets to determine, select, and/or otherwise process microbiome features of particular relevance to one or more microorganism-related conditions (e.g., processed microbiome features associated with relevance scores to a microorganism-related condition; cross-condition microbiome features with relevance to a plurality of microorganism-related conditions, etc.), which can facilitate improvements in accuracy (e.g., by using the most relevant microbiome features; by leveraging tailored analytical techniques; etc.), processing speed (e.g., by selecting a subset of relevant microbiome features; by performing dimensionality reduction techniques; by leveraging tailored analytical techniques; etc.), and/or other computational improvements in relation to phenotypic prediction (e.g., indications of the microorganism-related conditions, etc.), other suitable characterizations, therapeutic intervention facilitation, and/or other suitable purposes. In a specific example, the technology can apply feature-selection rules (e.g., microbiome feature-selection rules for composition, function; for supplemental features extracted from supplementary datasets; etc.) with one or more microbiome characterization modules to select an optimized subset of features (e.g., microbiome functional features relevant to one or more microorganism-related conditions; microbiome composition diversity features such as reference relative abundance features indicative of healthy, presence, absence, and/or other suitable ranges of taxonomic groups associated with microorganism-related conditions; user relative abundance features that can be compared to reference relative abundance features correlated with microorganism-related conditions and/or therapy responses; etc.) out of a vast potential pool of features (e.g., extractable from the plethora of microbiome data such as sequence data; identifiable by statistical tests such as univariate statistical tests; etc.) for generating, applying, and/or otherwise facilitating characterization and/or therapies (e.g., through models, etc.). The potential size of microbiomes (e.g., human microbiomes, animal microbiomes, etc.) can translate into a plethora of data, giving rise to questions of how to process and analyze the vast array of data to generate actionable microbiome insights in relation to microorganism-related conditions. However, the feature-selection rules and/or other suitable computer-implementable rules can enable one or more of: shorter generation and execution times (e.g., for generating and/or applying models; for determining microorganism-related characterizations and/or associated therapies; etc.); optimized sample processing techniques (e.g., improving transformation of microorganism nucleic acids from biological samples through using primer types, other biomolecules, and/or other sample processing components identified through computational analysis of taxonomic groups, sequences, and/or other suitable data associated with microorganism-related conditions, such as while optimizing for improving specificity, reducing amplification bias, and/or other suitable parameters; etc.); model simplification facilitating efficient interpretation of results; reduction in overfitting; network effects associated with generating, storing, and applying microbiome characterizations for a plurality of users over time in relation to microorganism-related conditions (e.g., through collecting and processing an increasing amount of microbiome-related data associated with an increasing number of users to improve predictive power of the microorganism-related characterizations and/or therapy determinations; etc.); improvements in data storage and retrieval (e.g., storing and/or retrieving microbiome characterization modules; storing specific models such as in association with different users and/or sets of users, with different microorganism-related conditions; storing microorganism datasets in association with user accounts; storing therapy monitoring data in association with one or more therapies and/or users receiving the therapies; storing features, microorganism-related characterizations, and/or other suitable data in association with a user, set of users, and/or other entities to improve delivery of personalized characterizations and/or treatments for the microorganism-related conditions, etc.), and/or other suitable improvements to technological areas.

Fourth, specific examples of the technology can amount to an inventive distribution of functionality across a network including a sample handling system, a microorganism-related characterization system (e.g., including a set of microbiome characterization modules, where each module can have differing but complementary functionality, etc.), and a plurality of users, where the sample handling system can handle substantially concurrent processing of biological samples (e.g., in a multiplex manner) from the plurality of users, which can be leveraged by the microorganism-related characterization system in generating personalized characterizations and/or therapies (e.g., customized to the user's microbiome such as in relation to the user's dietary behavior, probiotics-associated behavior, medical history, demographics, other behaviors, preferences, etc.) for microorganism-related conditions.

Fifth, specific examples of the technology can improve the technical fields of at least genomics, microbiology, microbiome-related computation, diagnostics, therapeutics, microbiome-related digital medicine, digital medicine generally, modeling, and/or other relevant fields. In an example, the technology can leverage to a set of microbiome characterization modules to model and/or characterize different microorganism-related conditions, such as through computational identification of relevant microorganism features (e.g., which can act as biomarkers to be used in diagnoses, facilitating therapeutic intervention, etc.) for microorganism-related conditions. In another example, the technology can perform cross-condition analysis to identify and evaluate cross-condition microbiome features associated with (e.g., shared across, correlated across, etc.) a plurality of a microorganism-related conditions (e.g., diseases, phenotypes, etc.). Such identification and characterization of microbiome features can facilitate improved health care practices (e.g., at the population and individual level, such as by facilitating diagnosis and therapeutic intervention, etc.), by reducing risk and prevalence of comorbid and/or multi-morbid microorganism-related conditions (e.g., which can be associated with environmental factors, and thereby associated with the microbiome, etc.).

Sixth, the technology can leverage specialized computing devices (e.g., devices associated with the sample handling system, such as next-generation sequencing systems; microorganism-related characterization systems; therapy facilitation systems; etc.) in performing suitable portions associated with the method 100 and/or system 200.

Specific examples of the technology can, however, provide any other suitable benefits) in the context of using non-generalized computer systems for microorganism-related characterization, microbiome modulation, and/or for performing other suitable portions of the method 100.

### 3. System.

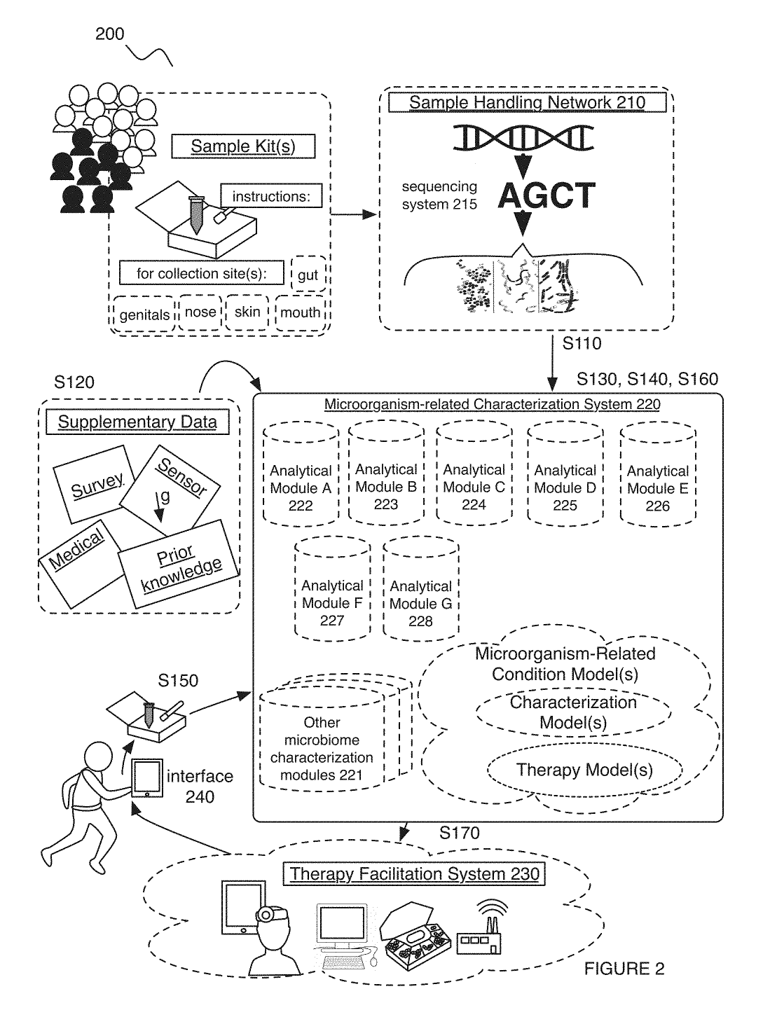

As shown in FIG. 2, embodiments of the system 200 (e.g., for characterizing a microorganism-related condition) can include any one or more of: a handling system (e.g., a sample handling system, etc.) 210 operable to collect and/or process biological samples (e.g., collected by users and included in containers including pre-processing reagents; etc.) from one or more users (e.g., a human subject, patient, animal subject, environmental ecosystem, care provider, etc.) for determining a microorganism dataset (e.g., microorganism genetic sequences; microorganism sequence dataset; etc.); a microorganism-related characterization system 220 operable to determine user microbiome features (e.g., microbiome composition features; microbiome functional features; diversity features; relative abundance ranges; such as based on a microorganism dataset and/or other suitable data; etc.), determine microorganism-related characterizations (e.g., microorganism-related condition characterizations, therapy-related characterizations, characterizations for users, etc.); and/or a therapy facilitation system 230 operable to facilitate therapeutic intervention (e.g., promote a therapy, etc.) for one or more microorganism-related conditions (e.g., based on one or more microorganism-related conditions; etc.).

In a specific example, the system 200 can include a sample handling system including a sequencing system (e.g., a next-generation sequencing system, etc.) operable to determine microorganism genetic sequences based on biological samples associated with a set of subjects, where the biological samples include microorganism nucleic acids associated with the microorganism-related condition; a set of microbiome characterization modules 221 operable to apply a set of analytical techniques including at least two of a statistical test (e.g., univariate statistical test, etc.), a dimensionality reduction technique, an artificial intelligence approach, and/or other suitable approaches described herein, and where the set of microbiome characterization modules 221 includes: a first microbiome characterization module 221′ operable to apply a first analytical technique (e.g., one or more univariate statistical tests and/or suitable statistical tests, etc.), of the set of analytical techniques, to determine a set of microbiome features based on the microorganism genetic sequences, where the set of microbiome features is associated with the microorganism-related condition (e.g., correlated with the microorganism-related condition, etc.); and a second microbiome characterization module 221″ operable to apply a second analytical technique (e.g., a dimensionality reduction technique), of the set of analytical techniques, to determine a processed microbiome feature set (e.g., a feature set of reduced dimensions; a feature set including the most relevant features for one or more microorganism-related conditions; etc.) based on the set of microbiome features (e.g., where the outputs of the first microbiome characterization module 221″ can be used as inputs for the second microbiome characterization module 221″ in a serial, chained, manner, etc.), where the processed microbiome feature set is adapted to improve the characterizing of the microorganism-related condition (e.g., through identifying and leveraging a subset of tailored features from a vast pool of potential features for improving accuracy, processing speed, and thereby improving functionality of the computing system in relation to microorganism-related characterization, therapeutic intervention facilitation, and/or other suitable functionality described herein, etc.); and a microorganism-related condition model generated based on the processed microbiome feature set, where the microorganism-related condition model is operable to determine a characterization of the microorganism-related condition for a user.

The handling system 210 of the system 200 can function to receive and/or process (e.g., fragment, amplify, sequence, generate associated datasets, etc.) biological samples to transform microorganism nucleic acids and/or other components of the biological samples into data (e.g., genetic sequences that can be subsequently aligned and analyzed; microorganism datasets; etc.) for facilitating generation of microorganism-related characterizations and/or therapeutic intervention. The handling system 210 can additionally or alternatively function to provide sample kits 250 (e.g., including sample containers, instructions for collecting samples from one or more collection sites, etc.) to a plurality of users (e.g., in response to a purchase order for a sample kit 250), such as through a mail delivery system. The handling system 210 can include one or more sequencing systems 215 (e.g., a next-generation sequencing systems, sequencing systems for targeted amplicon sequencing, metatranscriptomic sequencing, metagenomic sequencing, sequencing-by-synthesis techniques, capillary sequencing technique, Sanger sequencing, pyrosequencing techniques, nanopore sequencing techniques, etc.) for sequencing one or more biological samples (e.g., sequencing microorganism nucleic acids from the biological samples, etc.), such as in generating microorganism data (e.g., microorganism sequence data, other data for microorganism datasets, etc.). The handling system 210 can additionally or alternatively include a library preparation system operable to automatically prepare biological samples (e.g., fragment and amplify using primers compatible with genetic targets associated with the microorganism-related condition) in a multiplex manner to be sequenced by a sequencing system; and/or any suitable components. The handling system can perform any suitable sample processing techniques described herein. However, the handling system 210 and associated components can be configured in any suitable manner.

The microbiome characterization system 220 of the system 200 can function to determine, analyze, characterize, and/or otherwise process microorganism datasets (e.g., based on processed biological samples leading to microorganism genetic sequences; alignments to reference sequences; etc.), microbiome features (e.g., individual variables; groups of variables; features relevant for phenotypic prediction, for statistical description; variables associated with a sample obtained from an individual; variables associated with microorganism-related conditions; variables describing fully or partially, in relative or absolute quantities the sample's microbiome composition and/or functionality; etc.), models (e.g., microorganism-related condition models, etc.), and/or other suitable data for facilitating microorganism-related characterization and/or therapeutic intervention. In examples, the microbiome characterization system 220 can identify derived from the information of the features that statistically describe the differences between samples associated with one or more microorganism-related conditions (e.g., samples associated with presence, absence, risk of, propensity for, and/or other aspects related to microorganism-related conditions etc.), such as where the differing analyses can provide complementing views into the features differentiating the different samples (e.g., differentiating the subgroups associated with presence or absence of a condition, etc.). In a specific example, individual predictors, a specific biological process, and/or statistically inferred latent variables can provide complementary information at different levels of data complexity to facilitate varied downstream opportunities in relation to characterization, diagnosis, and/or treatment. In a specific example, the microbiome characterization system 220 can generate and/or apply a therapy model (e.g., based on cross-condition analyses, etc.) for identifying and/or characterizing a therapy used to treat one or more microorganism-related conditions. In another specific example, the microbiome characterization system 220 process supplementary data (e.g., prior knowledge to be used in improving application of the microbiome characterization modules 221; such as prior knowledge associated with users, microbiome features, microorganism-related conditions, other components, etc.).

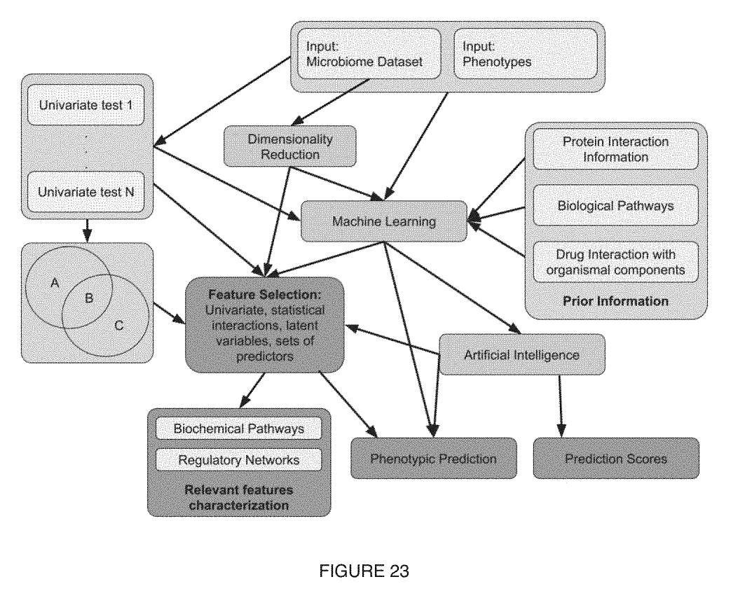

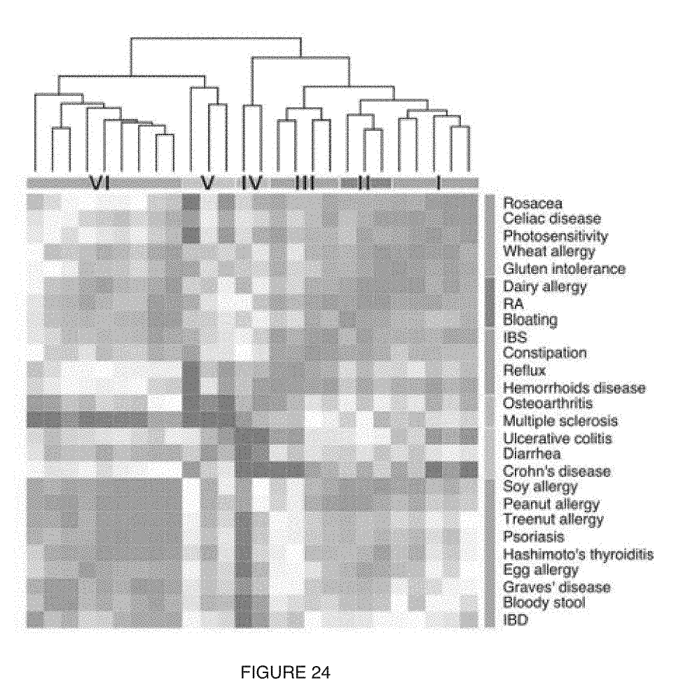

The microbiome characterization system 220 preferably includes one or more microbiome characterization modules 221 (e.g., independent modules, interdependent modules, etc.), which can function to apply one or more analytical techniques in processing microorganism datasets, microbiome features, supplementary data, and/or other suitable data in facilitating microorganism-related characterization and/or therapeutic intervention (e.g., as shown in FIG. 23).

Any suitable microbiome characterization modules 221 (e.g., leveraging any suitable analytical techniques, etc.) can be applied in any suitable combination in a serial (e.g., by chaining microbiome characterization modules 221 in relation to outputs and inputs, etc.), concurrent, repetitive, and/or in any suitable temporal relationship in any suitable manner. For example, an output of a microbiome characterization module 221 can constitute a microorganism-related characterization (e.g., a result of interest by itself, etc.), be treated as an intermediate component (e.g., used as an input for the same or different microbiome characterization module 221, for a model such as a therapy model, etc.), and/or be used for any suitable purpose. In specific examples, a plurality of microbiome characterization modules 221 can be chained (e.g., such as where one or more outputs of a microbiome characterization module 221 can be used as one or more inputs for the same or another microbiome characterization module 221, etc.) and/or otherwise connected (e.g., in relation to data sharing, in relation to contribution to a microorganism-related characterization, in relation to associations with one or more microorganism-related conditions, etc.), which can facilitate one or more feature selection (e.g., selecting a subset of microbiome features for subsequent use, etc.), feature weighting (e.g., for determining different weights for different features, such as up-weighting or down-weighting features, which can be used in any suitable microbiome characterization modules 221, models, and/or other suitable processes, etc.), warm start (e.g., where outputs and/or other processing associated with a first microbiome characterization module 221′ can assist and/or otherwise improve processing associated with a second microbiome characterization module 221″, such as in relation to improving statistical learning and/or inference, which can be in relation to facilitating focus on the most relevant feature, etc.). For example, a first microbiome characterization module 221′ can determine a set of microbiome features (e.g., by applying a first analytical technique); and a second microbiome characterization module 221″ can apply (e.g., be operable to apply) a second analytical technique to perform at least one of feature selection, feature weighting, and warm start, for processing the set of microbiome features into the processed microbiome feature set. However, microbiome characterization modules 221 can be applied at any suitable time and frequency for any number of datasets, users, microorganism-related conditions, therapies, and/or other suitable entities for any suitable purpose.

Different microbiome characterization modules 221 (e.g., different combinations of microbiome characterization modules 221; different modules applying different analytical techniques; different inputs and/or output types; applied in different manners such as in relation to time and/or frequency; etc.) can be applied (e.g., executed, selected, retrieves, stored, etc.) based on one or more of: microorganism-related conditions (e.g., using different combinations microbiome characterization modules 221 depending on the microorganism-related condition or conditions being characterized, such as where different microbiome characterization modules 221 possess differing levels of suitability for processing data in relation to different microorganism-related conditions, etc.), users (e.g., different microbiome characterization modules 221 based on different user data and/or characteristics, such as corresponding sample collection site, demographics, genetics, environmental factors, etc.), microorganism-related characterizations (e.g., different microbiome characterization modules 221 for different types of characterizations, such as a therapy-related characterization versus a diagnosis-related characterization, such as for identifying relevant microbiome composition versus determining a propensity score for a microorganism-related condition; etc.), therapies (e.g., different microbiome characterization modules 221 for monitoring efficacy of different therapies, etc.), and/or any other suitable components. In examples, different microbiome characterization modules 221 can be tailored to different types of inputs, outputs, microorganism-related characterizations, microorganism related conditions (e.g., different phenotypic measures that need to be characterized), and/or any other suitable entities. However, microbiome characterization modules 221 can be tailored and/or used in any suitable manner for facilitating microorganism-related characterization and/or therapeutic intervention.

Microbiome characterization modules 221, models, other components of the system 200, and/or suitable portions of the method 100 (e.g., determining microbiome features, determining microorganism-related characterizations, etc.) can employ analytical techniques including any one or more of: statistical tests (e.g., univariate statistical tests, multivariate statistical tests, etc.) dimensionality reduction techniques, artificial intelligence approaches (e.g., machine learning approaches, etc.), performing pattern recognition on data (e.g., identifying correlations between microorganism-related conditions and microbiome features; etc.), fusing data from multiple sources (e.g., generating characterization models based on microbiome data and/or supplementary data from a plurality of users associated with one or more microorganism-related conditions, such as based on microbiome features extracted from the data; etc.), combination of values (e.g., averaging values, etc.), compression, conversion (e.g., digital-to-analog conversion, analog-to-digital conversion), performing statistical estimation on data (e.g. ordinary least squares regression, non-negative least squares regression, principal components analysis, ridge regression, etc.), wave modulation, normalization, updating (e.g., of characterization models and/or therapy models based on processed biological samples over time; etc.), ranking (e.g., microbiome features; therapies; etc.), weighting (e.g., microbiome features; etc.), validating, filtering (e.g., for baseline correction, data cropping, etc.), noise reduction, smoothing, filling (e.g., gap filling), aligning, model fitting, binning, windowing, clipping, transformations, mathematical operations (e.g., derivatives, moving averages, summing, subtracting, multiplying, dividing, etc.), data association, multiplexing, demultiplexing, interpolating, extrapolating, clustering, image processing techniques, other signal processing operations, other image processing operations, visualizing, and/or any other suitable processing operations. Artificial intelligence approaches can include any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.) reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., näive Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perception method, a back-propagation method, a Hopfield network method, a self-organizing map method, a learning vector quantization method, etc.), an ensemble method (e.g., boosting, boostrapped aggregation, AdaBoost, stacked generalization, gradient boosting machine method, random forest method, etc.), and/or any suitable artificial intelligence approach. However, data processing can be employed in any suitable manner.

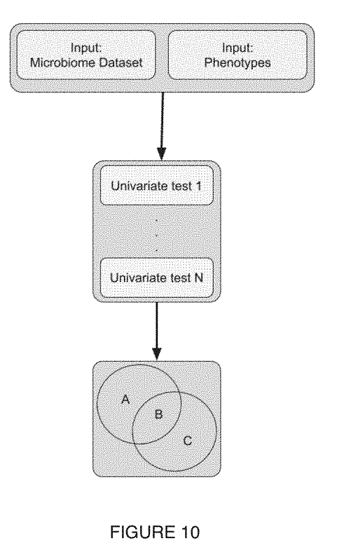

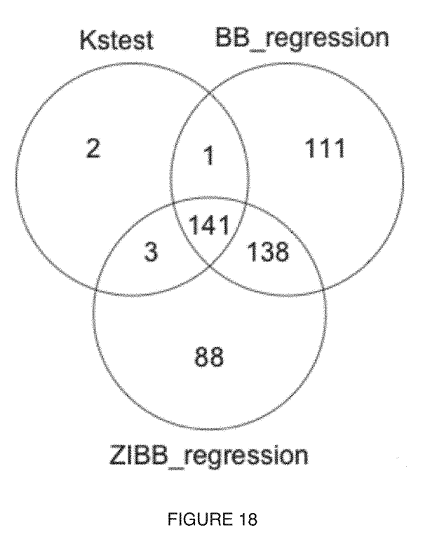

In a first variation, as shown in FIG. 10, a microbiome characterization module 221 (e.g., Analytical Module A 222) can apply one or more statistical tests (e.g., univariate statistical tests, multivariate, etc.), which can include any one or more of a t-test, a Kolmogorov-Smimov test, a regression model, and and/or other suitable techniques related to statistical tests. The microbiome characterization module 221 can apply statistical tests for determining a set of microbiome features (e.g., based on microorganism datasets such as including microorganism genetic sequences, based on prior knowledge such as associations between microbiome features and microorganism-related conditions, supplementary data informative of subjects, users, etc.). The microbiome characterization module 221 can apply a plurality of statistical tests (e.g., univariate statistical tests, multivariate, etc.), which can complement each other by employing different modelling strategies, for instance, for detecting changes in mean and variance or presence and/or absence patterns. In an example, the outputs (e.g., results) of the different types of statistical tests (e.g., univariate statistical tests, multivariate, etc.) (and/or other suitable analytical techniques) can be joined, grouped, and/or otherwise aggregated in order to show associations (e.g., similarities, differences) between the different analytical techniques (e.g., as shown in FIG. 18, indicating different single tests in sections A and C, and a union of outputs from multiple tests in relation to section B, etc.), such as in relation to the microbiome features identified. In a specific example, a first microbiome characterization module 221 can apply (e.g., be operable to apply, etc.) a first statistical test (e.g., univariate statistical test, etc.) to determine first set of microbiome features, and a second microbiome characterization module 221″ (and/or using the same first microbiome characterization module 221′) can apply a second statistical test (e.g., second univariate statistical test, etc.) to determine a second set of microbiome features. The aggregation of outputs from multiple analytical techniques can include the intersection or union among different outputs from different analytical techniques, where leveraging such aggregated outputs can be used for achieving a goal balance of specificity and sensitivity (e.g., higher specificity with lower sensitivity; higher sensitivity with lower specificity; etc.). Any suitable microorganism datasets, inputs and/or outputs of microbiome characterization modules 221, and/or other suitable data can be used as an input or can be an output of the statistical tests, and outputs of the microbiome characterization module 221 can be used as inputs for any other suitable microbiome characterization modules 221. However, the microbiome characterization module 221 (e.g., Analytical Module A 222) can be configured in any suitable manner.

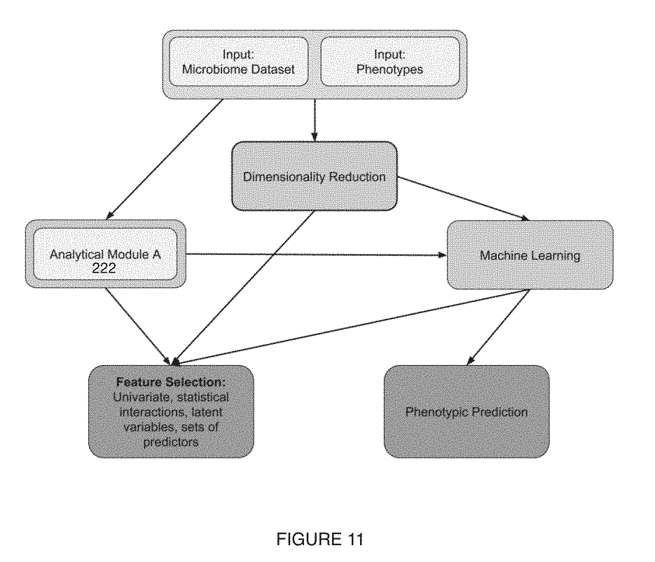

In a second variation, as shown in FIG. 11, a microbiome characterization module 221 (e.g., Analytical Module B 223) can apply one or more dimensionality-reduction techniques including any one or more of: supervised dimensionality reduction techniques; unsupervised dimensionality reduction techniques; missing values ratio; principal component analysis (PGA); probabilistic PGA; matrix factorization techniques; compositional mixtures models such as latent dirichlet allocation or hierarchical dirichlet process; feature embedding techniques as isomap or local linear embedding, partial lest squares regression, Sammon mapping, multidimensional scaling, projection pursuit; and/or any other suitable techniques related to dimensionality reduction. Applying dimensionality reduction techniques can decrease the number of dimensions (e.g., features, samples, etc.) from a dataset. Any suitable microorganism datasets, inputs and/or outputs of microbiome characterization modules 221, and/or other suitable data can be used as an input or can be an output of the dimensionality reduction techniques (e.g., using microbiome features determined by statistical tests as inputs into dimensionality reduction techniques for reducing the number of features; etc.), and outputs of the microbiome characterization module 221 can be used as inputs for any other suitable microbiome characterization modules 221 (e.g., into statistical tests; artificial intelligence approaches such as random forest, kernel machines, support vector machines, regression methods; Analytical Module A 222; Analytical Module C 224, etc.). Applying the microbiome characterization module 221 can facilitate determination of a linear or non-linear association between inferred latent features and phenotypic-related data associated with the one or more microorganism-related conditions. Outputs of the microbiome characterization mexlule 221 can include a microorganism-related characterization (e.g., result of interest by itself), an output for additional analysis (e.g., by providing individual features with predictive value and/or latent features useful for clustering and classifying samples, etc.), and/or be used for any suitable purpose. However, the microbiome characterization module 221 (e.g., Analytical Module B 223) can be configured in any suitable manner.

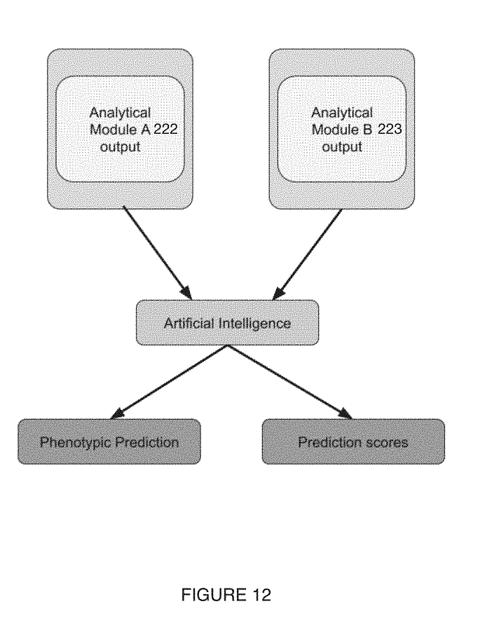

In a third variation, as shown in FIG. 12, a microbiome characterization module 221 (e.g., Analytical Module C 224) can facilitate application of one or more machine learning models (and/or other suitable artificial intelligence approaches). In examples, the microbiome characterization module 221 can function to guide the construction of the architecture and/or parameters estimation of Artificial Intelligence approaches (e.g., Neural Networks, Autoencoder models or Generative Adversarial Networks, etc.), such as through, encoding a non-linear predictive function of the phenotype and/or other microorganism-related condition. Any suitable microorganism datasets, inputs and/or outputs of microbiome characterization modules 221, and/or other suitable data can be used as an input or can be an output (e.g., using outputs of statistical tests, of dimensionality reduction approaches, of Analytical Module A 222, of Analytical Module B 223, and/or using any suitable data as inputs, etc.), and outputs of the microbiome characterization module 221 can be used as inputs for any other suitable microbiome characterization modules 221. Outputs of the microbiome characterization module 221 can include a microorganism-related characterization (e.g., phenotypic predictions such as propensity scores for microorganism-related conditions, etc.), an output for additional analysis (e.g., relevance scores for features describing predictive value, which can be used to identify features most relevant to phenotypic prediction and/or other types of prediction, etc.), and/or can be used for any suitable purpose. However, the microbiome characterization module 221 (e.g., Analytical Module C 224) can be configured in any suitable manner.

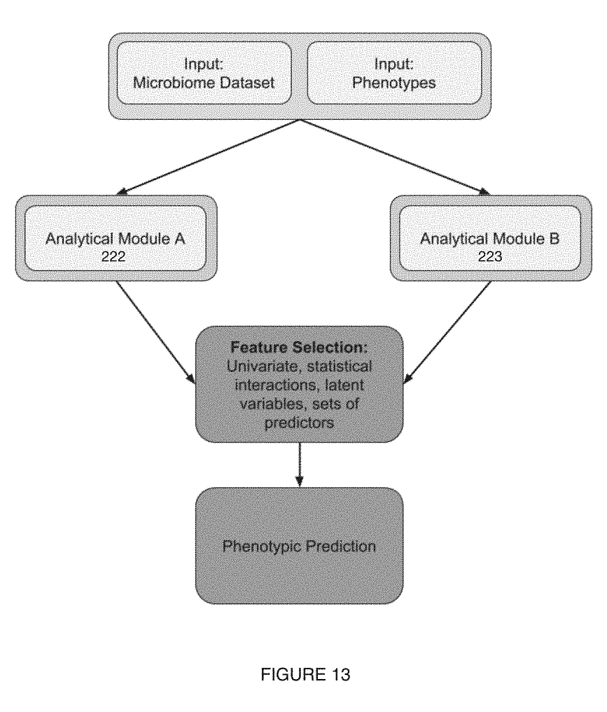

In a fourth variation, as shown in FIG. 13, a microbiome characterization module 221 (e.g., Analytical Module D 225) can apply one or more analytical techniques (e.g., second or higher order testing of interaction via regression and/or equivalent methods; machine learning algorithms such as random forest and/or support vector machines, data compression techniques; kernel machines; etc.) for detection of statistical interactions between microorganism data (e.g., different microbiome composition profiles, etc.), microbiome features, and/or features obtained from their transformations (e.g., ratios, products, features obtained from the application of dimensionality reduction algorithms, etc.). However, the microbiome characterization module 221 (e.g., Analytical Module D 225) can be configured in any suitable manner.

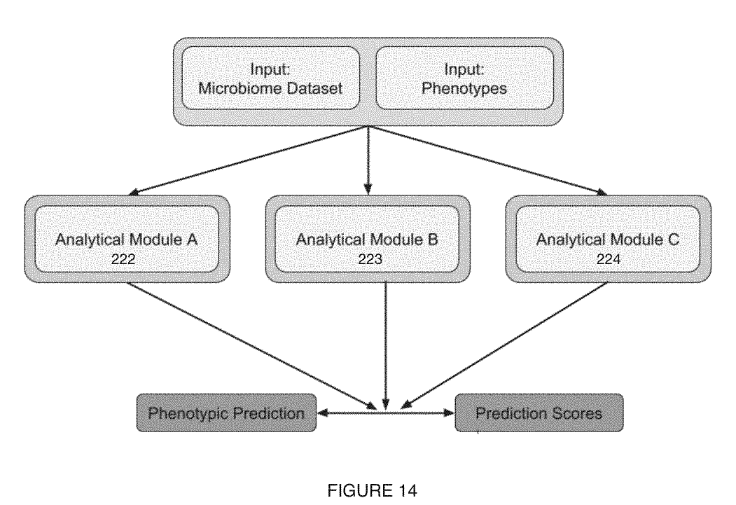

In a fifth variation, as shown in FIG. 14, a microbiome characterization module 221 (e.g., Analytical Module E 226) can determine phenotypic predictions, risk indices, propensity scores, other indices, and/or other suitable metrics associated with microorganism-related conditions (e.g., associated with diagnosing microorganism-related conditions for a user, etc.), such as through applying analytical techniques including at least one or more of: statistical tests (e.g., univariate statistical tests, multivariate statistical tests, etc.), univariate techniques, multivariate techniques, artificial intelligence approaches (e.g., machine learning models, etc.) and/or other suitable techniques (e.g., where outputs can be used as a summary of the microbiome composition, function, and/or other suitable microbiome-related aspects associated with the microorganism-related condition, etc.). The microbiome characterization module 221 can define minimum and/or maximum values for a range of outputs, such as through normalization techniques using empirical analyses. In an example, a score can be calculated for a set of reference samples (e.g., for data corresponding to the reference samples, etc.), where minimum and maximum observed values can be recorded and used to normalize the score of a particular sample (e.g., subsequent sample) according to

$$normalized score = \frac{sample score - minimum score}{maximum score - minimum score} ,$$

which can facilitate a score at the 0 to 1 range. Additionally or alternatively, the microbiome characterization module 221 can determine a calibrated score (e.g., with a recognizable value in characterization, diagnostic, and/or treatment guidance, etc.). In an example, the microbiome characterization module 221 can determine a calibrated score by determining scores (e.g., propensity scores, etc.) for a set of samples (e.g., corresponding to healthy subjects and subjects with one or more microorganism-related conditions of interest, etc.); transforming the propensity scores into calibrated scores (e.g., ranging from 0 to 1) by calculating for each possible value of the propensity score (e.g., 10), the fraction of subjects with the one or more microorganism-related conditions of interest (e.g., # of diseased subjects/(# of diseased subjects+# of healthy subjects)), with score values greater than or equal than it, where

$$calibrated score = \frac{\# cases with score > sample score}{\# cases + controls} ,$$

and where this can be seen as estimating the probability density function of the fraction of diseased individuals as a function of the propensity scores values. However, the microbiome characterization module 221 (e.g., Analytical Module E 226) can be configured in any suitable manner.

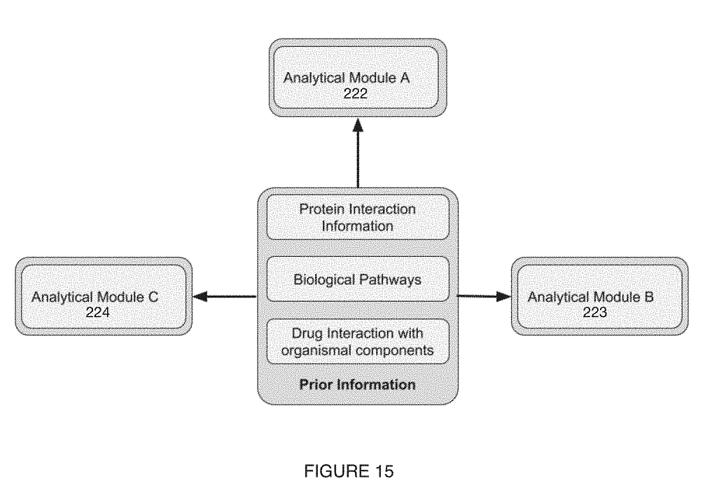

In a sixth variation, as shown in FIG. 15, a microbiome characterization module 221 (e.g., Analytical Module F 226) can apply prior knowledge (e.g., biological data, user data, etc.) of microbiome features (e.g., associations between microbiome features and microorganism-related conditions, associations with user characteristics, etc.), microorganism-related conditions, users, microorganism datasets, and/or other suitable components, for improving processing associated with other microbiome characterization modules 221 (e.g., Analytical Module A 222, Analytical Module B 223, Analytical Module C 224, etc.). In an example, the microbiome characterization module 221 can guide the statistical inference towards improved predictive models with lower error rates, thereby improving functionality of the computing system. In examples, inclusion of such knowledge (e.g., prior information, etc.) can be performed through leveraging hard features, filtering, weighting schemes, including the external variables at the data modelling steps, other analytical techniques, and/or any other suitable processes. However, the microbiome characterization module 221 (e.g., Analytical Module F 226) can be configured in any suitable manner.

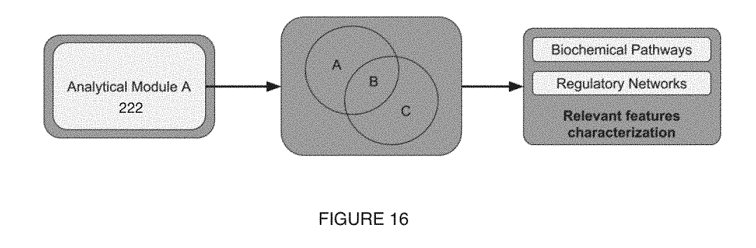

In a seventh variation, as shown in FIG. 16, a microbiome characterization module 221 (e.g., Analytical Module G 227) can process the features identified as statistically associated with one or more microorganism-related conditions to contrast with other features not being associated with the one or more microorganism-related conditions, such as to identify overarching characteristics that are more or less common among those features found to be associated or disassociated with the one or more microorganism-related conditions. The microbiome characterization module 221 can generate and/or leverage mappings (e.g., of the microbiome features, etc.) to biological annotations such as gene-regulatoiy networks or biochemical pathways. However, the microbiome characterization module 221 (e.g., Analytical Module G 227) can be configured in any suitable manner.

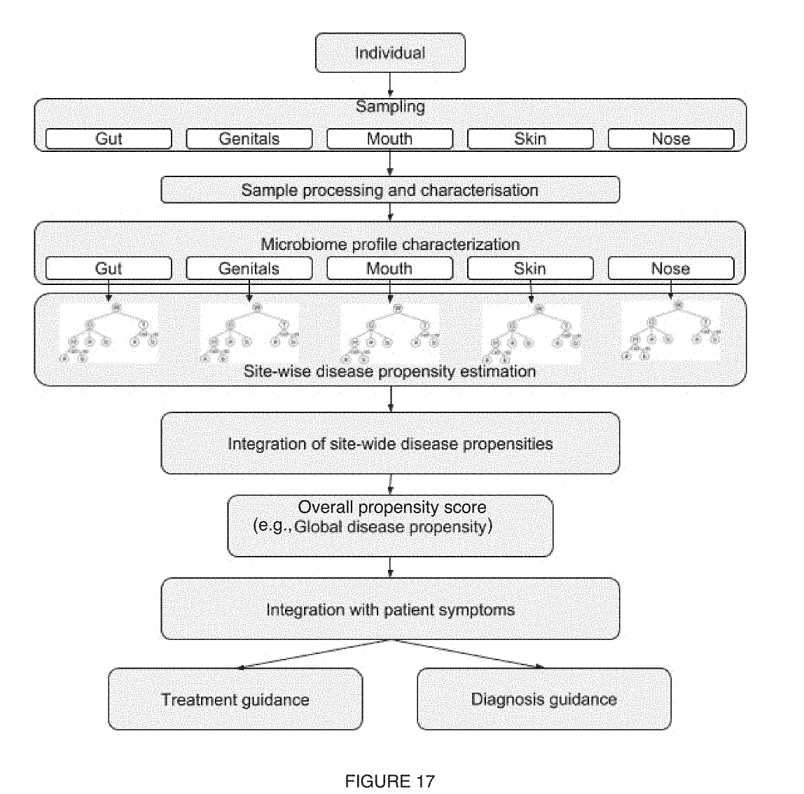

As shown in FIG. 17, the microbiome characterization system 220 can preferably perform multi-site analyses associated samples collected from a plurality of sites (e.g., performing multi-site analyses, with microbiome characterization modules 221, based on multi-site microorganism datasets associated with different collection sites; generating multi-site characterizations based on outputs of microbiome characterization modules 221; etc.). Sites (e.g., collection sites, etc.), can include any one or more regions of: the gut, skin, nose, mouth, genitals, other suitable physiological sites, other sample collection sites, and/or any other suitable sites. Multi-site analyses can be performed at a population level (e.g., in relation to different populations for identifying microbiome features and/or generating associated models, such as different models tailored to analyzing datasets associated with a plurality of collection sites, etc.), an individual level (e.g., for a user), and/or for any suitable entities. Multi-site analyses can be performed with and/or based on (e.g., based on outputs of, etc.) one or more microbiome characterization modules 221 and/or any other suitable components (e.g., remote computing systems, user devices, etc.). For example, the system 200 can include a sample handling network operable to process (e.g., collect, sequence, etc.) biological samples including site-diverse samples collected from a plurality of collection sites including at least two of gut, genitals, mouth, skin, and nose; and a first microbiome characterization module 221 operable to apply a first statistical test (e.g., univariate statistical test, etc.) (and/or other suitable analytical techniques) to determine first subsets of microbiome features of the set microbiome features based on the site-diverse samples, where each subset of microbiome features from the first subsets of microbiome features corresponds to a different collection site from the plurality of collection sites (e.g., different or similar types of microbiome features for different collection sites based on the different microbiome composition and/or function for the different collection sites, etc.). In the example, the system 200 can include a second microbiome characterization module 221 operable to apply an additional statistical test (e.g., univariate statistical test; a different type of statistical test than the first statistical test, such as a different univariate statistical test, etc.) to determine second subsets of microbiome features of the set of microbiome features based on the site-diverse samples (e.g., where the first subsets of microbiome features correspond to the first statistical test and where the different subsets of the first subsets correspond to different collection sites; where the second subsets of microbiome features correspond to the additional statistical test and where the different subsets of the second subsets correspond to different collection sites; etc.), and where the microorganism-related condition model is generated based on the first subsets and the second subsets of microbiome features (e.g., a model for multi-site analysis; where a plurality of microorganism-related condition models can be generated based on the microbiome features, such as different models for different collection sites and/or for different microorganism-related conditions associated with the different collection sites, etc.).

Multi-site analyses can include integration of, combination of, and/or otherwise aggregating site-wise characterizations (e.g., different site-wise individual propensity scores calculated from different microorganism datasets corresponding to samples collected at different collection sites, etc.), site-wise therapeutic intervention facilitation, and/or any other suitable process in the context of multi-site analyses. Multi-site analyses can be performed (e.g., using microbiome characterization modules 221, etc.) by applying at least one or more of: statistical techniques including Bayesian and Frequentist approaches that handle scores or probabilities, and/or other suitable analytical techniques. In a variation, individual metrics (e.g., propensity scores and/or other metrics for one or more microorganism-related conditions), associated with different collection sites (e.g., of a single user, of multiple users, etc.), can be combined to determine an overall metric (e.g., an overall disease propensity score and/or other metrics, etc.) such as through using a mean of the individual metric values. The standard deviation can be calculated using standard formulas to propagate uncertainty from individual site-wise data (e.g., individual propensity scores for an individual, etc.) into the overall metric (e.g., overall disease propensity score, etc.). In examples, the overall metrics (e.g., multi-site characterizations, etc.) can describe additional information relative any single site-wise metric, and where site-wise metrics can provide complementary and non-redundant information. In a specific example, complementarity can indicate that the microbiome-related characterizations (e.g., metrics, etc.) corresponding to different sites are not fully-correlated (e.g., the microbiome composition, function, and/or other suitable characterizations one site cannot be perfectly predicted with that of another site, etc.). Multi-site analyses can account for redundancy of information among sampling sites (e.g., where failing to do so can lead to biased overall metrics, such as by giving exacerbated importance to sites with a strong correlation among them, etc.). In a variation, the microbiome characterization system 220 can use information regarding the covariance/correlation among the sampling (e.g., amongst the microorganism datasets corresponding to the different site-diverse samples, etc.), which can be estimated from the corresponding data, such as to determine an improved overall metric (e.g., with increased accuracy, etc.). In an example, multivariate statistical approaches can be applied (e.g., for estimating covariance and/or correlation, etc.), such as to account for the non-redundant information. In a specific example, mean and standard deviation can be estimated using a specific covariance/correlation pattern among the microbiome characteristics (e.g., microbiome composition, microbiome function, microbiome features, microorganism datasets, other suitable aspects of a microbiome profile, etc.) corresponding to the sites being considered. Mean and variance can be estimated by

$$\frac{1}{S} {\sum }_{i = 1}^{S} {x}_{i}$$

and Σᵢ₌₁^{S}σᵢ+Σᵢ₌₁^{S}Σ_{j<i}^{S}σᵢⱼ with S being the number of sites being considered, xᵢ being a site-wise score, and where σᵢ and σᵢⱼ are the variance of the i-th site-wise score and the covariance parameters between the i-th and j-th site, respectively. Estimation of these covariances and/or correlation can be performed using multivariate statistical methodologies. In a specific example, the microbiome characterization system 220 can, for users with multi-site microorganism data: apply dimensionality reduction techniques to the data of each site separately, such as through using PGA and selecting a subset of the latent variables sufficient to characterize the data; and/or with the latent variables from each site, a covariance/correlation can be estimated using multivariate methods, such as through using canonical correlation analysis, but any suitable analytical techniques and/or microbiome characterization modules 221 can be applied for multi-site analyses.

In a specific example, as shown in FIG. 17, an overall propensity score (e.g., for one or more microorganism-related conditions) can be determined by one or more of: collecting samples from a user from two or more collection sites; determining a multi-site microorganism dataset (e.g., including site-wise microorganism data; through laboratory processing and/or downstream bioinformatics approaches; etc.); determining site-wise propensity scores (e.g., based on site-wise microbiome features determined with microbiome characterization modules 221; through site-wise microorganism-related condition propensity estimation algorithms; through analytical techniques including at least one of machine learning models, regression models, clustering algorithms that score a microbiome profile for propensity to a disease on the basis of parametric or nonparametric functions previously learnt, etc.); and determining an overall propensity-score based on the site-wise propensity scores, the information of the non-obvious correlation pattern of the site-to-site microbiome profile, and/or other suitable data. Multi-site analyses (e.g., combining the complementary information from different sites to generate an overall metric, etc.) can provide a holistic measure of microorganism-related condition propensity, which can, for example, be integrated with patient phenology to guide diagnosis and treatment decisions (e.g., facilitate therapeutic intervention, etc.). However, the microbiome characterization system and/or other suitable components can be configured in any suitable manner to facilitate multi-site analyses (e.g., applying analytical techniques for multi-site analysis purposes; generating multi-site characterizations, etc.).

The microbiome characterization system can preferably perform cross-condition analyses for a plurality of microorganism-related conditions (e.g., using one or more microbiome characterization modules 221; generating multi-condition characterizations based on outputs of microbiome characterization modules 221, such as multi-condition microbiome features; etc.). For example, the microbiome characterization system can characterize relationships between microorganism-related conditions based on microorganism data, microbiome features, and/or other suitable microbiome characteristics of users associated with (e.g., diagnosed with, characterized by, etc.) a plurality of microorganism-related conditions. In a specific example, cross-condition analyses can be performed based on characterizations for individual microorganism-related conditions (e.g., outputs from microbiome characterization modules 221 for individual microorganism-related conditions, etc.). Cross-condition analyses can include identification of condition-specific features (e.g., associated exclusively with a single microorganism-related condition, etc.), multi-condition features (e.g., associated with two or more microorganism-related conditions, etc.), and/or any other suitable types of features. Cross-condition analyses can include determination of parameters informing correlation, concordance, and/or other similar parameters describing relationships between two or more microorganism-related conditions, such as by evaluating different pairs of microorganism-related conditions, where ranked pairs with higher parameter values can be associated with a greater degree of similarity (e.g., sharing) of microbiome features. In an example, cross-condition analyses can include joint analysis of data from a plurality of microorganism-related conditions in relation to associated microbiome characteristics (e.g., microorganism data, microbiome features, etc.). Cross-condition analyses can include application of analytical techniques including any one or more of: multivariate models, canonical correlation models, multi-label artificial intelligence approaches (e.g., multi-label supervised, multi-label unsupervised, multi-label semi-supervised machine learning or artificial intelligence approaches, etc.), and/or any other suitable analytical techniques (e.g., for application of a microbiome characterization module 221 in analyzing individual microorganism-related conditions, and comparing the resulting characterizations, etc.). However, the microbiome characterization system and/or other suitable components can be configured in any suitable manner to facilitate cross-condition analyses (e.g., applying analytical techniques for cross-condition analysis purposes; generating cross-condition characterizations, etc.).

The microbiome characterization system 220 preferably includes a remote computing system (e.g., for applying microbiome characterization modules 221, etc.), but can additionally or alternatively include any suitable computing systems (e.g., local computing systems, user devices, handing system components, etc.). However, the microbiome characterization system 220 can be configured in any suitable manner.

The therapy facilitation system 230 of the system 200 can function to facilitate therapeutic intervention (e.g., promote one or more therapies, etc.) for one or more microorganism-related conditions (e.g., facilitating modulation of a user microbiome composition and functional diversity for improving a state of the user in relation to one or more microorganism-related conditions, etc.). The therapy facilitation system 230 can facilitate therapeutic intervention for any number of microorganism-related conditions associated with any number of collection sites, such as based on multi-site characterizations, multi-condition characterizations, other characterizations, and/or any other suitable data. The therapy facilitation system 230 can include any one or more of: a communications system (e.g., to communicate therapy recommendations, selections, discouragements, and/or other suitable therapy-related information to a user device and/or care provider device; to enable telemedicine between a care provider and a subject in relation to a microorganism-related condition; etc.), an application executable on a user device (e.g., indicating microbiome composition and/or functionality for a user; etc.), a medical device (e.g., a biological sampling device, such as for collecting samples from different collection sites; medication provision devices; surgical systems; etc.), a user device (e.g., biometric sensors), and/or any other suitable component. One or more therapy facilitation systems 230 can be controllable, communicable with, and/or otherwise associated with the microbiome characterization system 220. For example, the microbiome characterization system 220 can generate characterizations of one or more microorganism-related conditions for the therapy facilitation system 230 to present (e.g., transmit, communicate, etc.) to a corresponding user (e.g., at an interface 240, etc.). In another example, the therapy facilitation system 230 can update and/or otherwise modify an application and/or other software of a device (e.g., user smartphone) to promote a therapy (e.g., promoting, at a to-do list application, lifestyle changes for improving a user state associated with one or more microorganism-related conditions, etc.). However, the therapy facilitation system 230 can be configured in any other manner.

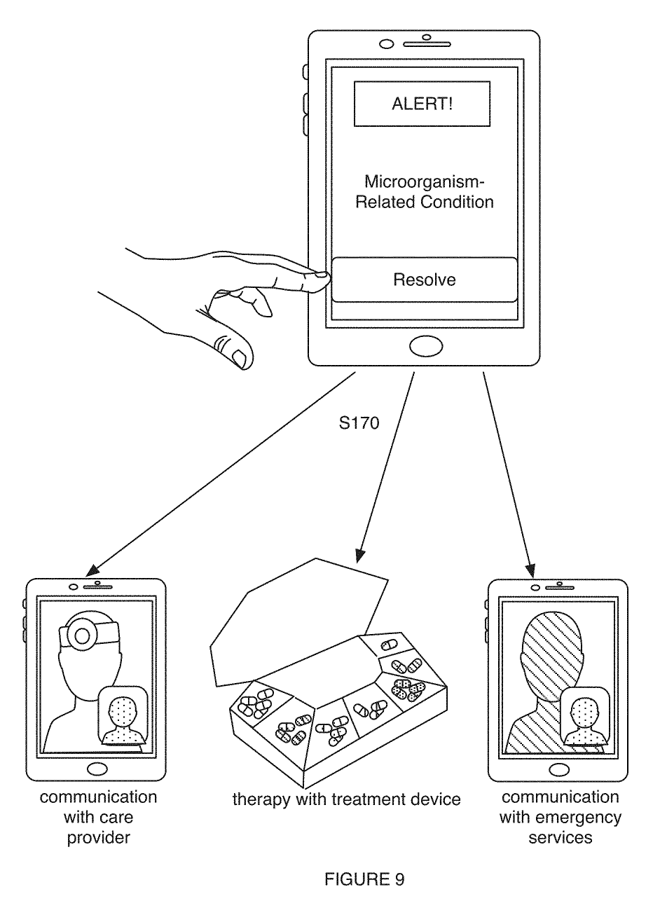

As shown in FIG. 9, the system 200 can additionally or alternatively include an interface 240, which can function to improve presentation of microbiome characteristics, microorganism-related condition information (e.g., propensity metrics; therapy recommendations; comparisons to other users; other characterizations; etc.). In examples, the interface 240 can present microorganism-related condition information including a microbiome composition (e.g., taxonomic groups; relative abundances; etc.), functional diversity (e.g., relative abundance of genes associated with particular functions, and propensity metrics for one or more microorganism-related conditions, such as relative to user groups sharing a demographic characteristic (e.g., smokers, exercisers, users on different dietary regimens, consumers of probiotics, antibiotic users, groups undergoing particular therapies, etc.). However, the interface 240 can be configured in any suitable manner.

While the components of the system 200 are generally described as distinct components, they can be physically and/or logically integrated in any manner. For example, a computing system (e.g., a remote computing system, a user device, etc.) can implement portions and/or all of the microbiome characterization system 220 (e.g., apply a microbiome-related condition model to generate a characterization of microorganism-related conditions for a user, etc.) and the therapy facilitation system 230 (e.g., facilitate therapeutic intervention through presenting insights associated with microbiome composition and/or function; presenting therapy recommendations and/or information; scheduling daily events at a calendar application of the smartphone to notify the user to take probiotic therapies identified based on the characterization, etc.). However, the functionality of the system 200 can be distributed in any suitable manner amongst any suitable system components. Additionally or alternatively, the system 200 and/or method 100 can include any suitable components and/or functions analogous to (e.g., applied in the context of microorganism-related conditions) those described in U.S. application Ser. No. 14/593,424 filed 9 Jan. 2015, which are is hereby incorporated in its entirety by this reference. However, the components of the system 200 can be configured in any suitable manner

### 4.1 Generating a Microorganism Dataset.

Block S110 can include determining a microorganism dataset (e.g., microorganism sequence dataset, microbiome composition diversity dataset such as based upon a microorganism sequence dataset, microbiome functional diversity dataset such as based upon a microorganism sequence dataset, etc.) associated with a set of subjects S110. Block S110 can function to process biological samples (e.g., an aggregate set of biological samples associated with a population of subjects, a subpopulation of subjects, a subgroup of subjects sharing a demographic characteristic and/or other suitable characteristics, etc.), in order to determine compositional, functional, pharmacogenomics, and/or other suitable aspects associated with the corresponding microbiomes, such as in relation to one or more microorganism-related conditions. Compositional and/or functional aspects can include one or more of aspects at the microorganism level (and/or other suitable granularity), including parameters related to distribution of microorganisms across different groups of kingdoms, phyla, classes, orders, families, genera, species, subspecies, strains, and/or any other suitable infraspecies taxon (e.g., as measured in total abundance of each group, relative abundance of each group, total number of groups represented, etc.). Compositional and/or functional aspects can also be represented in terms of operational taxonomic units (OTUs). Compositional and/or functional aspects can additionally or alternatively include compositional aspects at the genetic level (e.g., regions determined by multilocus sequence typing, 16S sequences, 18S sequences, ITS sequences, other genetic markers, other phylogenetic markers, etc.). Compositional and functional aspects can include the presence or absence or the quantity of genes associated with specific functions (e.g. enzyme activities, transport functions, immune activities, etc.). Outputs of Block S110 can thus be used to facilitate determination of microbiome features (e.g., generation of a microorganism sequence dataset usable for identifying microbiome features; etc.) for the characterization process of Block S130 and/or other suitable portions of the method 100 (e.g., where Block S110 can lead to outputs of microbiome composition datasets, microbiome functional datasets, and/or other suitable microorganism datasets from which microbiome features can be extracted, etc.), where the features can be microorganism-based (e.g., presence of a genus of bacteria), genetic-based (e.g., based upon representation of specific genetic regions and/or sequences), functional-based (e.g., presence of a specific catalytic activity), and/or any other suitable microbiome features.

In a variation, Block S110 can include assessment and/or processing based upon phylogenetic markers (e.g., for generating microorganism datasets, etc.) derived from bacteria and/or archaea in relation to gene families associated with one or more of: ribosomal protein S2, ribosomal protein S3, ribosomal protein S5, ribosomal protein S7, ribosomal protein S8, ribosomal protein S9, ribosomal protein S10, ribosomal protein S11, ribosomal protein S12/S23, ribosomal protein S13, ribosomal protein S15P/S13e, ribosomal protein S17, ribosomal protein S19, ribosomal protein L1, ribosomal protein L2, ribosomal protein L3, ribosomal protein L4/Lie, ribosomal protein L5, ribosomal protein L6, ribosomal protein L10, ribosomal protein L11, ribosomal protein L14b/L23e, ribosomal protein L15, ribosomal protein L16/L10E, ribosomal protein L18P/L5E, ribosomal protein L22, ribosomal protein L24, ribosomal protein L25/L23, ribosomal protein L29, translation elongation factor EF-2, translation initiation factor IF-2, metalloendopeptidase, ffh signal recognition particle protein, phenylalanyl-tRNA synthetase beta subunit, phenylalanyl-tRNA synthetase alpha subunit, tRNA pseudouridine synthase B, Porphobilinogen deaminase, ribosomal protein L13, phosphoribosylformylglycinamidine cyclo-ligase, and ribonuclease HII. Additionally or alternatively, markers can include target sequences (e.g., sequences associated with a microorganism taxonomic group; sequences associated with functional aspects; sequences correlated with microorganism-related conditions; sequences indicative of user responsiveness to different therapies; sequences that are invariant across a population and/or any suitable set of subjects, such as to facilitate multiplex amplification using a primer type sharing a primer sequence; conserved sequences; sequences including mutations, polymorphisms; nucleotide sequences; amino acid sequences; etc.), proteins (e.g., serum proteins, antibodies, etc.), peptides, carbohydrates, lipids, other nucleic acids, whole cells, metabolites, natural products, genetic predisposition biomarkers, diagnostic biomarkers, prognostic biomarkers, predictive biomarkers, other molecular biomarkers, gene expression markers, imaging biomarkers, and/or other suitable markers. However, markers can include any other suitable markers) associated with microbiome composition, microbiome functionality, and/or microorganism-related conditions.

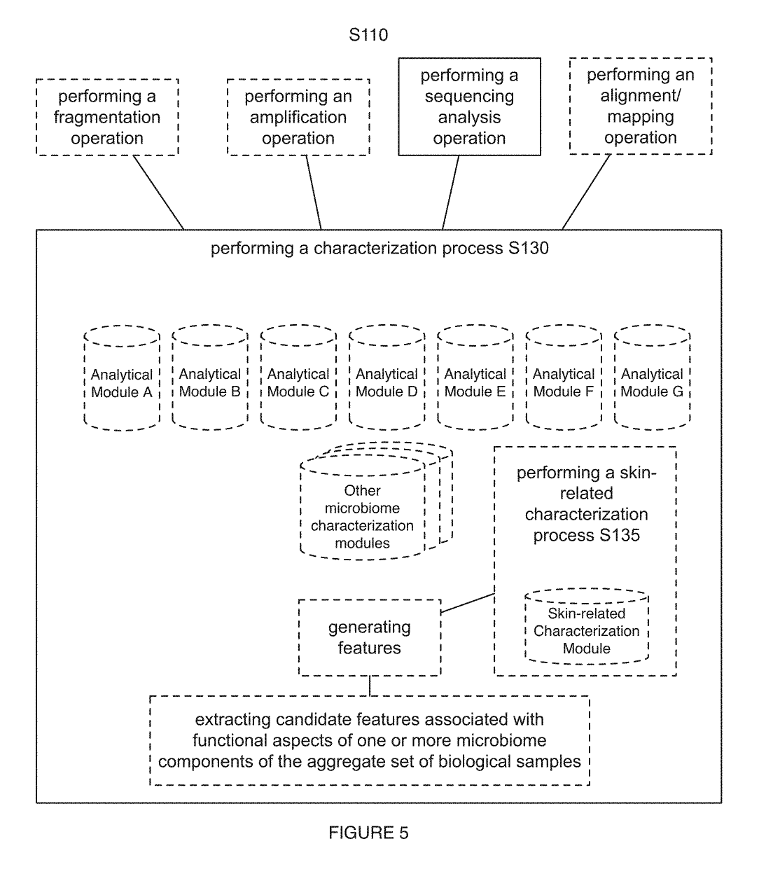

Characterizing the microbiome composition and/or functional aspects for each of the aggregate set of biological samples thus preferably includes a combination of sample processing techniques (e.g., wet laboratory techniques; as shown in FIG. 5), including, but not limited to, amplicon sequencing (e.g., 16S, 18S, ITS), UMIs, 3 step PCR, Crispr, metagenomic approaches, metatranscriptomics, use of random primers, and computational techniques (e.g., utilizing tools of bioinformatics), to quantitatively and/or qualitatively characterize the microbiome and functional aspects associated with each biological sample from a subject or population of subjects.

In variations, sample processing in Block S110 can include any one or more of: lysing a biological sample, disrupting membranes in cells of a biological sample, separation of undesired elements (e.g., RNA, proteins) from the biological sample, purification of nucleic acids (e.g., DNA) in a biological sample, amplification of nucleic acids from the biological sample, further purification of amplified nucleic acids of the biological sample, and sequencing of amplified nucleic acids of the biological sample. In an example, Block S110 can include: collecting biological samples from a set of users (e.g., biological samples collected by the user with a sampling kit including a sample container, etc.), where the biological samples include microorganism nucleic acids associated with the microorganism-related condition (e.g., microorganism nucleic acids including target sequences correlated with a microorganism-related condition; etc.). In another example, Block S110 can include providing a set of sampling kits to a set of users, each sampling kit of the set of sampling kits including a sample container (e.g., including pre-processing reagents, such as lysing reagents; etc.) operable to receive a biological sample from a user of the set of users.

In variations, lysing a biological sample and/or disrupting membranes in cells of a biological sample preferably includes physical methods (e.g., bead beating, nitrogen decompression, homogenization, sonication), which omit certain reagents that produce bias in representation of certain bacterial groups upon sequencing. Additionally or alternatively, lysing or disrupting in Block Suo can involve chemical methods (e.g., using a detergent, using a solvent, using a surfactant, etc.). Additionally or alternatively, lysing or disrupting in Block Suo can involve biological methods. In variations, separation of undesired elements can include removal of RNA using RNases and/or removal of proteins using proteases. In variations, purification of nucleic acids can include one or more of: precipitation of nucleic acids from the biological samples (e.g., using alcohol-based precipitation methods), liquid-liquid based purification techniques (e.g., phenol-chloroform extraction), chromatography-based purification techniques (e.g., column adsorption), purification techniques involving use of binding moiety-bound particles (e.g., magnetic beads, buoyant beads, beads with size distributions, ultrasonically responsive beads, etc.) configured to bind nucleic acids and configured to release nucleic acids in the presence of an elution environment (e.g., having an elution solution, providing a pH shift, providing a temperature shift, etc.), and any other suitable purification techniques.

In variations, amplification of purified nucleic acids can include one or more of: polymerase chain reaction (PCR)-based techniques (e.g., solid-phase PCR, RT-PCR, qPCR, multiplex PCR, touchdown PCR, nanoPCR, nested PCR, hot start PCR, etc.), helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), and any other suitable amplification technique. In amplification of purified nucleic acids, the primers used are preferably selected to prevent or minimize amplification bias, as well as configured to amplify nucleic acid regions/sequences (e.g., of the 16S region, the 18S region, the ITS region, etc.) that are informative taxonomically, phylogenetically, for diagnostics, for formulations (e.g., for probiotic formulations), and/or for any other suitable purpose. Thus, universal primers (e.g., a F27-R338 primer set for 16S RNA, a F515-R806 primer set for 16S RNA, etc.) configured to avoid amplification bias can be used in amplification. Additionally or alternatively include incorporated barcode sequences and/or UMIs specific to biological samples, to users, to microorganism-related conditions, to taxa, to target sequences, and/or to any other suitable components, which can facilitate a post-sequencing identification process (e.g., for mapping sequence reads to microbiome composition and/or microbiome function aspects; etc.). Primers used in variations of Block S110 can additionally or alternatively include adaptor regions configured to cooperate with sequencing techniques involving complementary adaptors (e.g., Illumina Sequencing). Additionally or alternatively, Block S110 can implement any other step configured to facilitate processing (e.g., using a Nextera kit). In a specific example, performing amplification and/or sample processing operations can be in a multiplex manner (e.g., for a single biological sample, for a plurality of biological samples across multiple users; etc.). In another specific example, performing amplification can include normalization steps to balance libraries and detect all amplicons in a mixture independent of the amount of starting material, such as 3 step PCR, bead based normalization, and/or other suitable techniques.

In variations, sequencing of purified nucleic acids can include methods involving targeted amplicon sequencing, metatranscriptomic sequencing, and/or metagenomic sequencing, implementing techniques including one or more of: sequencing-by-synthesis techniques (e.g., Illumina sequencing), capillary sequencing techniques (e.g., Sanger sequencing), pyrosequencing techniques, and nanopore sequencing techniques (e.g., using an Oxford Nanopore technique).

In a specific example, amplification and sequencing of nucleic acids from biological samples of the set of biological samples includes: solid-phase PCR involving bridge amplification of DNA fragments of the biological samples on a substrate with oligo adapters, where amplification involves primers having a forward index sequence (e.g., corresponding to an Illumina forward index for MiSeq/NextSeq/HiSeq platforms), a forward barcode sequence, a transposase sequence (e.g., corresponding to a transposase binding site for MiSeq/NextSeq/HiSeq platforms), a linker (e.g., a zero, one, or two-base fragment configured to reduce homogeneity and improve sequence results), an additional random base, UMIs, a sequence for targeting a specific target region (e.g., 16S region, 18S region, ITS region), a reverse index sequence (e.g., corresponding to an Illumina reverse index for MiSeq/HiSeq platforms), and a reverse barcode sequence. In the specific example, sequencing can include Illumina sequencing (e.g., with a HiSeq platform, with a MiSeq platform, with a NextSeq platform, etc.) using a sequencing-by-synthesis technique. In another specific example, the method 100 can include: identifying one or more primer types compatible with one or more genetic targets associated with one or more microorganism-related conditions (e.g., human behavior conditions, disease-related conditions, etc.); generating a microorganism dataset (e.g., microorganism sequence dataset, etc.) for one or more users (e.g., set of subjects) based on the one or more primer types (e.g., and the microorganism nucleic acids included in collected biological samples, etc.), such as through fragmenting the microorganism nucleic acids, and and/or performing multiplex amplification with the fragmented microorganism nucleic acids based on the one or more identified primer types compatible with the genetic target associated with the human behavior condition; and/or promoting (e.g., providing), based on a microorganism-related characterization derived from the a microorganism dataset a therapy for the user condition (e.g., enabling selective modulation of a microbiome of the user in relation to at least one of a population size of a desired taxon and a desired microbiome function, etc.).

In variations, primers (e.g., of a primer type corresponding to a primer sequence; etc.) used in Block S110 and/or other suitable portions of the method 100 can include primers associated with protein genes (e.g., coding for conserved protein gene sequences across a plurality of taxa, such as to enable multiplex amplification for a plurality of targets and/or taxa; etc.). Primers can additionally or alternatively be associated with microorganism-related conditions (e.g., primers compatible with genetic targets including microorganism sequence biomarkers for microorganisms correlated with microorganism-related conditions such as human behavior conditions and/or disease-related conditions; etc.), microbiome composition features (e.g., identified primers compatible with a genetic target corresponding to microbiome composition features associated with a group of taxa correlated with a microorganism-related condition; genetic sequences from which relative abundance features are derived etc.), functional diversity features, supplementary features, and/or other suitable features and/or data. Primers (and/or other suitable molecules, markers, and/or biological material described herein) can possess any suitable size (e.g., sequence length, number of base pairs, conserved sequence length, variable region length, etc.). Additionally or alternatively, any suitable number of primers can be used in sample processing for performing characterizations (e.g., microorganism-related characterizations; etc.), improving sample processing (e.g., through reducing amplification bias, etc.), and/or for any suitable purposes. The primers can be associated with any suitable number of targets, sequences, taxa, conditions, and/or other suitable aspects. Primers used in Block S110 and/or other suitable portions of the method 100 can be selected through processes described in Block S110 (e.g., primer selection based on parameters used in generating the taxonomic database) and/or any other suitable portions of the method 100. In an example, Block S110 can include: identifying a primer type for a microorganism nucleic acid sequence associated with the microorganism-related condition (e.g., a primer type for a primer operable to amplify microorganism nucleic acid sequences correlated with a microorganism-related condition; etc.); and generating the microorganism sequence dataset based on the primer type and the microorganism nucleic acids (e.g., using primers of the primer type for amplification of microorganism nucleic acids; and sequencing the amplified nucleic acids to generate the microorganism sequence dataset; etc.). In a specific example, Block S110 can include: fragmenting the microorganism nucleic acids; and performing multiplex amplification with the fragmented microorganism nucleic acids based on the fragmented microorganism nucleic acids and the identified primer type associated with the microorganism-related condition. Additionally or alternatively, primers (and/or processes associated with primers) can include and/or be analogous to that described in U.S. application Ser. No. 14/919,614, filed 21 Oct. 2015, which is herein incorporated in its entirety by this reference. However, identification and/or usage of primers can be configured in any suitable manner.

Some variations of sample processing can include further purification of amplified nucleic acids (e.g., PCR products) prior to sequencing, which functions to remove excess amplification elements (e.g., primers, dNTPs, enzymes, salts, etc.). In examples, additional purification can be facilitated using any one or more of: purification kits, buffers, alcohols, pH indicators, chaotropic salts, nucleic acid binding filters, centrifugation, and/or any other suitable purification technique.

In variations, computational processing in Block S110 can include any one or more of: identification of microbiome-derived sequences (e.g., as opposed to subject sequences and contaminants), alignment and mapping of microbiome-derived sequences (e.g., alignment of fragmented sequences using one or more of single-ended alignment, ungapped alignment, gapped alignment, pairing), and generating features associated with (e.g., derived from) compositional and/or functional aspects of the microbiome associated with a biological sample.

Identification of microbiome-derived sequences can include mapping of sequence data from sample processing to a subject reference genome (e.g., provided by the Genome Reference Consortium), in order to remove subject genome-derived sequences. Unidentified sequences remaining after mapping of sequence data to the subject reference genome can then be further clustered into operational taxonomic units (OTUs) based upon sequence similarity and/or reference-based approaches (e.g., using VAMPS, using MG-RAST, using QIIME databases), aligned (e.g., using a genome hashing approach, using a Needleman-Wunsch algorithm, using a Smith-Waterman algorithm), and mapped to reference bacterial genomes (e.g., provided by the National Center for Biotechnology Information), using an alignment algorithm (e.g., Basic Local Alignment Search Tool, FPGA accelerated alignment tool, BWT-indexing with BWA, BWT-indexing with SOAP, BWT-indexing with Bowtie, etc.). Mapping of unidentified sequences can additionally or alternatively include mapping to reference archaeal genomes, viral genomes and/or eukaryotic genomes. Furthermore, mapping of taxons can be performed in relation to existing databases, and/or in relation to custom-generated databases.

Upon identification of represented groups of microorganisms of the microbiome associated with a biological sample, generating features associated with (e.g., derived from) compositional and functional aspects of the microbiome associated with a biological sample can be performed. In a variation, generating features can include generating features based upon multilocus sequence typing (MSLT), in order to identify markers useful for characterization in subsequent blocks of the method 100. Additionally or alternatively, generated features can include generating features that describe the presence or absence of certain taxonomic groups of microorganisms, and/or ratios between exhibited taxonomic groups of microorganisms. Additionally or alternatively, generating features can include generating features describing one or more of: quantities of represented taxonomic groups, networks of represented taxonomic groups, correlations in representation of different taxonomic groups, interactions between different taxonomic groups, products produced by different taxonomic groups, interactions between products produced by different taxonomic groups, ratios between dead and alive microorganisms (e.g., for different represented taxonomic groups, based upon analysis of RNAs), phylogenetic distance (e.g., in terms of Kantorovich-Rubinstein distances, Wasserstein distances etc.), any other suitable taxonomic group-related feature(s), any other suitable genetic or functional aspectfs).

Additionally or alternatively, generating features can include generating features describing relative abundance of different microorganism groups, for instance, using a sparCC approach, using Genome Relative Abundance and Average size (GAAS) approach and/or using a Genome Relative Abundance using Mixture Model theory (GRAMMy) approach that uses sequence-similarity data to perform a maximum likelihood estimation of the relative abundance of one or more groups of microorganisms. Additionally or alternatively, generating features can include generating statistical measures of taxonomic variation, as derived from abundance metrics. Additionally or alternatively, generating features can include generating features associated with (e.g., derived from) relative abundance factors (e.g., in relation to changes in abundance of a taxon, which affects abundance of other taxons). Additionally or alternatively, generating features can include generation of qualitative features describing presence of one or more taxonomic groups, in isolation and/or in combination. Additionally or alternatively, generating features can include generation of features related to genetic markers (e.g., representative 16S, 18S, and/or ITS sequences) characterizing microorganisms of the microbiome associated with a biological sample. Additionally or alternatively, generating features can include generation of features related to functional associations of specific genes and/or organisms having the specific genes. Additionally or alternatively, generating features can include generation of features related to pathogenicity of a taxon and/or products attributed to a taxon. Block S120 can, however, include generation of any other suitable feature(s) derived from sequencing and mapping of nucleic acids of a biological sample. For instance, the feature(s) can be combinatory (e.g. involving pairs, triplets), correlative (e.g., related to correlations between different features), and/or related to changes in features (e.g., temporal changes, changes across sample sites, etc., spatial changes, etc.). However, processing biological samples, generating a microorganism dataset, and/or other aspects associated with Block S110 can be performed in any suitable manner.

### 4.2 Processing a Supplementary Dataset.

The method 100 can additionally or alternatively include Block S120, which can include processing (e.g., receiving, collecting, transforming, etc.) a supplementary dataset associated with (e.g., informative of; describing; indicative of; etc.) one or more microorganism-related conditions (e.g., human behavior condition such as associated with user behavior; disease related condition such as associated medical history, symptoms, medications; etc.) for the set of users. Block S120 can function to acquire data associated with one or more subjects of the set of subjects, which can be used to train, validate, apply, and/or otherwise inform the microorganism-related characterization process (e.g., in Block S130). In Block S120, the supplementary dataset preferably includes survey-derived data, but can additionally or alternatively include any one or more of: site-specific data (e.g., data informative of different collection sites, etc.), microorganism-related condition data (e.g., data information of microorganism-related conditions, etc.), contextual data derived from sensors (e.g., wearable device data, etc.), medical data (e.g., current and historical medical data; medical device-derived data; data associated with medical tests; etc.), social media data, user data (e.g., associated sensor data, demographic data, etc.), mobile phone data (e.g., mobile phone application data, etc.), web application data, prior biological knowledge (e.g., informative of microorganism-related conditions, microbiome characteristics, associations between microbiome characteristics and microorganism-related conditions, etc.), and/or any other suitable type of data. In variations of Block S120 including reception of survey-derived data, the survey-derived data preferably provides physiological, demographic, and behavioral information in association with a subject. Physiological information can include information related to physiological features (e.g., height, weight, body mass index, body fat percent, body hair level, etc.). Demographic information can include information related to demographic features (e.g., gender, age, ethnicity, marital status, number of siblings, socioeconomic status, sexual orientation, etc.). Behavioral information can include information related to one or more erf: health conditions (e.g., health and disease states), living situations (e.g., living alone, living with pets, living with a significant other, living with children, etc.), dietary habits (e.g., alcohol consumption, caffeine consumption, omnivorous, vegetarian, vegan, sugar consumption, acid consumption, consumption of wheat, egg, soy, treenut, peanut, shellfish, and/or other suitable food items, etc.), behavioral tendencies (e.g., levels of physical activity, drug use, alcohol use, habit development, etc.), different levels of mobility (e.g., amount of exercise such as low, moderate, and/or extreme physical exercise activity; related to distance traveled within a given time period; indicated by mobility sensors such as motion and/or location sensors; etc.), different levels of sexual activity (e.g., related to numbers of partners and sexual orientation), and any other suitable behavioral information. Survey-derived data can include quantitative data and/or qualitative data that can be converted to quantitative data (e.g., using scales of severity, mapping of qualitative responses to quantified scores, etc.).

In facilitating reception of survey-derived data, Block S130 can include providing one or more surveys to a subject of the population of subjects, or to an entity-associated with a subject of the population of subjects. Surveys can be provided in person (e.g., in coordination with sample provision and reception from a subject), electronically (e.g., during account setup by a subject, at an application executing at an electronic device of a subject, at a web application accessible through an internet connection, etc.), and/or in any other suitable manner.

Additionally or alternatively, portions of the supplementary dataset can be derived from sensors associated with the subjects) (e.g., sensors of wearable computing devices, sensors of mobile devices, biometric sensors associated with the user, etc.). As such, Block S130 can include receiving one or more of: physical activity- or physical action-related data (e.g., accelerometer and gyroscope data from a mobile device or wearable electronic device of a subject), environmental data (e.g., temperature data, elevation data, climate data, light parameter data, etc.), patient nutrition or diet-related data (e.g., data from food establishment check-ins, data from spectrophotometric analysis, user-inputted data, nutrition data associated with probiotic and/or prebiotic food items, types of food consumed, amount of food consumed, diets, etc.), biometric data (e.g., data recorded through sensors within the patient's mobile computing device, data recorded through a wearable or other peripheral device in communication with the patient's mobile computing device), location data (e.g., using GPS elements), and any other suitable data. In variations, sensor data can include data sampled at one or more: optical sensors (e.g., image sensors, light sensors, etc.), audio sensors, temperature sensors, volatile compound sensors, weight sensors, humidity sensors, depth sensors, location sensors (GPS receivers; etc.), inertial sensors (e.g., accelerators, gyroscope, magnetometer, etc.), biometric sensors (e.g., heart rate sensors, fingerprint sensors, bio-impedance sensors, etc.), pressure sensors, flow sensors, power sensors (e.g., Hall effect sensors), and/or or any other suitable sensor.

Additionally or alternatively, portions of the supplementary dataset can be derived from medical record data and/or clinical data of the subjects). As such, portions of the supplementary dataset can be derived from one or more electronic health records (EHRs) of the subjects).

Additionally or alternatively, the supplementary dataset of Block S120 can include any other suitable diagnostic information (e.g., clinical diagnosis information), which can be combined with analyses derived from features to support characterization of subjects in subsequent blocks of the method 100. For instance, information derived from a colonoscopy, biopsy, blood test, diagnostic imaging, other suitable diagnostic procedures, survey-related information, and/or any other suitable test can be used to supplement (e.g., for any suitable portions of the method 100).

Additionally or alternatively, the supplementary dataset can include therapy-related data including one or more of: therapy regimens, types of therapies, recommended therapies, therapies used by the user, therapy adherence, etc. For example, the supplementary dataset can include user adherence (e.g., medication adherence, probiotic adherence, physical exercise adherence, dietary adherence, etc.) to a recommended therapy. However, processing supplementary datasets can be performed in any suitable manner.

### 4.3 Performing a Characterization Process.

Block S130 can include, with a one or more microbiome characterization modules, applying analytical techniques to perform a characterization process (e.g., pre-processing, feature generation, feature processing, multi-site characterization for a plurality of collection sites, cross-condition analysis for a plurality of microorganism-related conditions, model generation, etc.) for the one or more microorganism-related condition, such as based on a microorganism dataset (e.g., derived in Block Sno, etc.) and/or other suitable data (e.g., supplementary dataset; etc.) S130. Block S130 can function to identify, determine, extract, and/or otherwise process features and/or feature combinations that can be used to determine microorganism-related characterizations for users or and sets of users, based upon their microbiome composition (e.g., microbiome composition diversity features, etc.), function (e.g., microbiome functional diversity-features, etc.), and/or other suitable microbiome features (e.g., such as through the generation and application of a characterization model for determining microorganism-related characterizations, etc.). As such, the characterization process can be used as a diagnostic tool that can characterize a subject (e.g., in terms of behavioral traits, in terms of medical conditions, in terms of demographic traits, etc.) based upon their microbiome composition and/or functional features, in relation to one or more of their health condition states (e.g., microorganism-related condition states), behavioral traits, medical conditions, demographic traits, and/or any other suitable traits. Such characterizations can be used to determine, recommend, and/or provide therapies (e.g., personalized therapies, such as determined by way of a therapy model, etc.), and/or otherwise facilitate therapeutic intervention.

Performing a characterization process S130 can include pre-processing microorganism datasets, microbiome features, and/or other suitable data for facilitation of downstream processing (e.g., determining microorganism-related characterizations, etc.). In an example, performing a characterization process can include, filtering a microorganism dataset (e.g., filtering a microorganism sequence dataset, such as prior to applying a set of analytical techniques to determine the microbiome features, etc.), by at least one of: a) removing first sample data corresponding to first sample outliers of a set of biological samples (e.g., associated with one or more microorganism-related conditions, etc.), such as where the first sample outliers are determined by at least one of principal component analysis, a dimensionality reduction technique, and a multivariate methodology; b) removing second sample data corresponding to second sample outliers of the set of biological samples, where the second sample outliers can determined based on corresponding data quality for the set of microbiome features (e.g., removing samples corresponding to a number of microbiome features with high quality data below a threshold condition, etc.); and c) removing one or more microbiome features from the set of microbiome features based on a sample number for the microbiome feature failing to satisfy a threshold sample number condition, where the sample number corresponds to a number of samples associated with high quality data for the microbiome feature. However, pre-processing can be performed with any suitable analytical techniques in any suitable manner.

In performing the characterization process, Block S130 can use computational methods (e.g., statistical methods, machine learning methods, artificial intelligence methods, bioinformatics methods, etc.) to characterize a subject as exhibiting features associated with one or more microorganism-related conditions (e.g., features characteristic of a set of users with the one or more microorganism-related conditions, etc.).

Block S130 preferably includes applying one or more analytical techniques with one or more microbiome characterization modules (e.g., for determining microbiome features, generating a microorganism-related characterization, etc.). For example, applying a set of analytical techniques to determine a set of microbiome features can include determining an initial set of microbiome features (e.g., based on a microorganism sequence dataset, etc.); and applying, with a first microbiome characterization module (e.g., Analytical Module B, etc.) of a set of microbiome characterization modules, one or more dimensionality reduction techniques on the initial set of microbiome features to determine a set of microbiome features (e.g., where the set of microbiome features includes fewer microbiome features than the initial set of microbiome features, etc.), such as where the dimensionality reduction technique can include at least one of missing values ratio, principal component analysis, probabilistic principal component analysis, matrix factorization techniques, compositional mixture models, and feature embedding techniques. In an example, determining the initial set of microbiome features can include applying, with a second microbiome characterization module (e.g., Analytical Module A, etc.) of the set of microbiome characterization modules, one or more statistical tests (e.g., univariate statistical tests, multivariate, etc.) to determine the initial set of microbiome features (e.g., based on the microorganism sequence dataset, etc.), such as where the statistical test (e.g., univariate statistical test, multivariate, etc.) can include at least one of a t-test, a Kolmogorov-Smirnov test, and a regression model. In an example, the method 100 can include, with a second microbiome characterization module (e.g., Analytical Module C, etc.) of the set of microbiome characterization modules, applying a machine learning approach (and/or other suitable artificial intelligence approach, etc.) to determine relevance scores for the set of microbiome features, where generating the microorganism-related condition model can include generating a microorganism-related condition model (e.g., for determining characterizations of one or more microorganism-related conditions, etc.) based on the set of microbiome features and the relevance scores.

Performing a characterization process (and/or other suitable portions of the method 100 and/or system 200) can be for any suitable type and or number of microorganism-related conditions. In a variation, performing a characterization process can be for one or more skin-related conditions. In an example, for subjects associated with one or more skin-related conditions (e.g., skin photosensitivity; dandruff; dry skin; presence; absence; etc.), the method 100 can include determining microorganism datasets (e.g., microorganism sequence datasets generated from sequencing microorganism nucleic acids from biological samples collected for the subjects, such as at different collection sites, etc.); and with a microbiome characterization module (e.g., Analytical Module A, etc.) of a set of microbiome characterization modules, applying a plurality of statistical tests (e.g., Kolmogorov-Smirnov, beta-binomial regression, and zero-inflated beta-binomial regression tests, univariate statistical tests, multivariate statistical tests, etc.) based on microorganism datasets corresponding to different collections sites of the subjects, for determining microbiome feature subsets, each microbiome feature subset corresponding to a different collection site, a different microorganism-related condition (e.g., different skin-related conditions, etc.), a different statistical test applied (e.g., as shown in Table 1, Table 2, Table 3, Table 4, and Table 5, etc.), different combinations of such entities, and/or any other suitable entities. In the example, performing a characterization process can include, with a second microbiome characterization module (e.g., Analytical Module B, etc.) of the set of microbiome characterization modules, applying a dimensionality reduction technique (e.g., supervised and/or unsupervised dimensionality reduction techniques, etc.) for obtaining a distance matrix calculated from microbiome characteristics (e.g., microbiome features, microorganism datasets, etc.), where such data can be used with a machine learning approach (and/or other suitable artificial intelligence approach) to select a subset of features (e.g., the most relevant features for one or more microorganism-related conditions, etc.). In a specific example, performing a characterization process can include determining feature relevance scores and/or other suitable metrics associated with feature importance (e.g., through applying random forest techniques); and using the feature relevance scores and/or other suitable metrics, along with supplemental data (e.g., prior biological knowledge informative of the microbiome features, such as with a third microbiome characterization modules, Analytical Module F, etc.) to obtain sample level quantification of microbiome functional features (e.g., using any suitable software tools). In another specific example, microbiome features can be integrated into (e.g., assigned to, such as through a soft-assignment, etc.) microbiome-subsystems (e.g., aggregations of microbiome features, groups of microbiome features, etc.), such as based on determination of one or more correlation coefficient between the abundance profiles of the microbiome functional features and the sub-system's principal component on the samples analyzed.

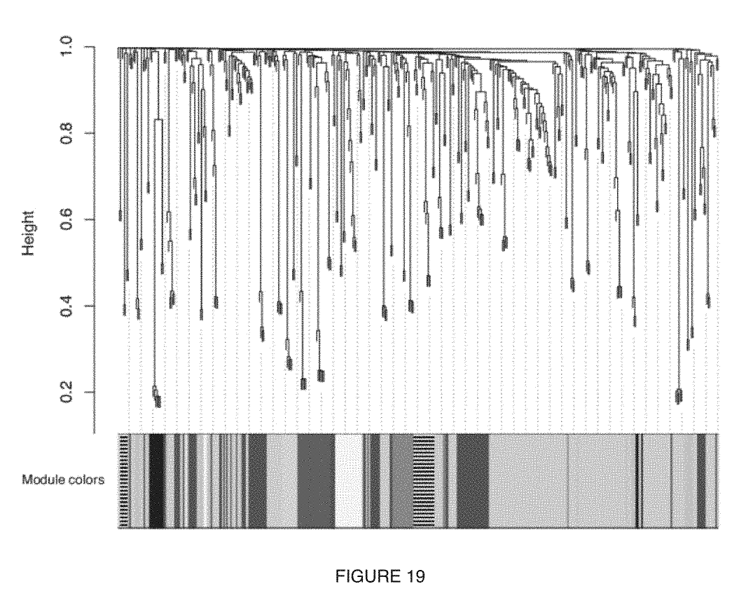

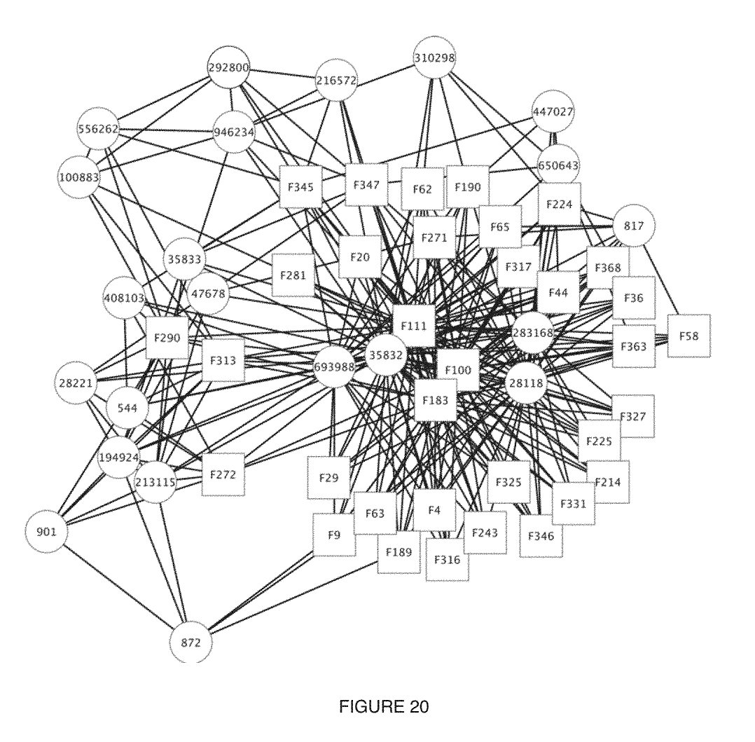

In another variation, performing a characterization process can be for one or more gastrointestinal-related conditions. In an example, for subjects associated with one or more gastrointestinal-related conditions (e.g., inflammatory bowel disease; presence; absence; etc.), the method 100 can include determining microorganism datasets (e.g., corresponding to different collection sites; etc.); and with a microbiome characterization module of a set of microbiome characterization modules, applying a plurality of statistical tests (e.g., Kolmogorov-Smirnov, beta-binomial regression, and zero-inflated beta-binomial regression tests, etc.) based on microorganism datasets corresponding to different collections sites of the subjects, for determining microbiome feature subsets, each microbiome feature subset corresponding to a different collection site, a different microorganism-related condition (e.g., different skin-related conditions, etc.), a different statistical test applied (e.g., as shown in Table 15, Table 16, Table 17, Table 18, and Table 19, etc.), different combinations of such entities, and/or any other suitable entities (e.g., where the different individual results can be compared, such as for identifying the intersection of microbiome features across different applied statistical tests for a given collection site and microorganism-related condition, as shown in FIG. 18, which illustrates the union and intersection of 484 and 141 microbiome features, respectively, etc.). In the example, performing a characterization process can include, with a second microbiome characterization module (e.g., Analytical Module B, etc.) of the set of microbiome characterization modules, applying a dimensionality reduction technique (e.g., supervised and/or unsupervised dimensionality reduction techniques, etc.) for constructing a correlation network among the microbiome features, which can be used in identifying sets of inter-correlated features (e.g., a microbiome sub-system, etc.), such as through suitable software tools and/or packages. In the example, performing a characterization process can include determining a summary variable for each microbiome sub-system (e.g., each set of inter-correlated microbiome features, etc.) such as through applying a PGA approach for obtaining a single number for each sample summarizing microbiome characteristics (e.g., a microbiome profile, etc.) for a subject for the microbiome features included in the microbiome sub-system. In the example, software tools and/or other suitable techniques can be used for network construction and microbiome sub-system detection, such as for empirically determining adequate analyses parameters. In a specific example, for soft-thresholding power, a set of possible values between 1 and 20 can be selected (e.g., choosing a power value of 2 to describe a network keeping high connectivity and relatively clear sub-systems detection, etc.), such as shown in FIG. 19, which describes a representation of the dimensionality reduction obtained from the application of a microbiome characterization module (e.g., Analytical Module B, etc.) on which each microbiome sub-system detected is represented by a different grey-scale color. Applying the dimensionality reduction techniques can result in a low dimensional representation of the original data exemplified by a set of principal components (e.g., one for each microbiome sub-system), where the dimensionality reduction can be by a factor of 47.7× (e.g., approximately two orders of magnitude; by transforming 430 microbiome features initially considered for analyses into 9 variables; etc.); and a direct mapping between each microbiome feature and the microbiome sub-systems identified (e.g., as shown in Table 20, which describes the mapping obtained on which every microbiome feature is assigned to a microbiome sub-system and a soft-assignment is also obtained by means of the correlation between the feature and the sub-system's principal component on the samples analyzed; etc.). In the example, performing the characterization process can include, with a third microbiome characterization module (e.g., Analytical Module F) of the set of microbiome characterization modules, leveraging supplementary data (e.g., prior biological knowledge of the microbiome features, etc.) to obtain sample level quantification of microbiome functional features (e.g., as implemented on a suitable software tool), for integration into microbiome-subsystems for obtaining a soft-assignment of the microbiome functional features to the microbiome sub-systems by means of calculating a correlation coefficient between the abundance profiles of the microbiome functional features and the sub-system's principal component on the samples analyzed (e.g., as shown in Table 21). Outputs of the microbiome characterization module (e.g., outputs of the dimensionality reduction techniques; outputs of Analytical Module B), can be used in generating, executing, and/or otherwise processing one or more machine learning models (e.g., where outputs of Analytical Module B can be used as inputs for Analytical Module C and/or other suitable microbiome characterization modules, etc.). In a specific example, microbiome sub-system principal components can be used as predictors of the inflammatory bowel disease conditions with two labels: cases reporting the conditions and controls not reporting having the conditions, where a machine learning classifier (e.g., random forest classifier) can be generated for determining feature relevance scores and/or other feature importance metric (e.g., for determining the most important microbiome sub-system's principal component predictor, etc.). In the specific example, as shown in Table 22, feature importance metrics identified a ranking of relevance for the different microbiome sub-systems numbered 5, 2, 6, 0, 3, 1, 4, 7, 8, where microbiome sub-system 5 was identified as the most relevant with a feature importance ˜1.5 greater than the second more predictive sub-system and ˜10 times greater than the worst predictive sub-system, where microbiome features associated with sub-system 5 are shown in Table 23 and the microbiome functional features more strongly associated with sub-system 5 are shown in Table 24, and where a graphic representation of interaction between taxonomies and function can be seen in FIG. 20. Supplementary data can be used by a microbiome characterization module (e.g., Analytical Module F), where prior biological knowledge of the relationship between microbiome features and small molecules and drugs metabolization can be used to identify the drugs likely to affect metabolization associated with sub-system 5, other microbiome sub-systems, and/or other suitable microbiome features, where in the specific example, 6 out of 22 microbiome features of sub-system 5 had roles on metabolizing a total of 12 molecules and drugs (e.g., as shown in Table 25), where 4 out of the 12 molecules have roles in inflammation (e.g., associated with inflammatory bowel disease, etc.), and where such processes can identify relevant molecules to determine options for pharmacological treatment, as in the case of Acarbose, and dietary and life-style changes, as in the case of Resveratrol, Taurine and Flavonoids, and/or otherwise facilitate therapeutic intervention. In a specific example, determining a characterization can include determining a drug metabolism characterization associated with one or more microorganism-related conditions, such as based on a microorganism-related condition model, a sample from the user, known associations between the set of microbiome features and drug metabolization, and/or any other suitable data.

In variations, performing a characterization process can include performing one or more multi-site analyses (e.g., with microbiome characterization modules; generating a multi-site characterization, etc.) associated with a plurality of collection sites. For example, determining a microorganism-related characterization (e.g., for one or more microorganism-related conditions, etc.) can include collecting, from a user, a set of site-diverse samples corresponding to a plurality of collection sites including at least two of gut, genitals, mouth, skin, and nose; determining a set of site-wise disease propensity metrics based on the set of site-diverse samples (e.g., using a microorganism-related condition model generated using microbiome characterization modules, etc.), where each site-wise disease propensity metric, of the set of site-wise disease propensity metrics, corresponds to a different collection site of the plurality of collection sites (e.g., and is associated with the one or more microorganism-related conditions, etc.); and determining an overall disease propensity metric for the user based on the set of site-wise disease propensity-metrics (e.g., where the overall disease propensity metric is associated with the one or more microorganism-related conditions. In the example, the method 100 can include determining a microorganism dataset associated with the plurality of collection sites based on the set of site-diverse samples, w-here determining the overall disease propensity metric can include determining at least one of a covariance metric and a correlation metric, based on the microorganism dataset, where the at least one of the covariance metric and the correlation metric is associated with the plurality of collection sites; and determining the overall disease propensity metric for the user based on the set of site-wise disease propensity metrics and the at least one of the covariance metric and the correlation metric. However, multi-site analyses can be performed in any suitable manner.

In variations, performing a characterization process can include performing one or more cross-condition analyses (e.g., using microbiome characterization modules, etc.) for a plurality of microorganism-related conditions. In an example, the method 100 can include analyzing metadata and microbiome characteristics (e.g., microbiome composition, function, etc.) for subjects reporting one or more of 26 (and/or other suitable number of) different microorganism-related conditions including rosacea, celiac disease, photosensibility, wheat allergy, gluten intolerance (e.g., gluten allergy, etc.), dairy allergy, bloating, rheumatoid arthritis, inflammatory bowel syndrome (IBS), hemorrhoidal disease, constipation, reflux, multiple sclerosis, osteoarthritis, ulcerative colitis, Crohn's disease, diarrhea, say allergy, peanut allergy-, tree nut allergy, egg allergy, psoriasis, Hashimoto's thyroiditis, Grave's disease, inflammatory bowel disease, and bloody stool. Microbiome characterization modules (e.g., Analytical Module B and Analytical Module C, etc.) can be applied in constructing predictive models informative of conditions-specific features and multi-condition features (e.g., shared across multiple microorganism-related conditions, etc.), where performing cross-condition analyses can include determining a microbiome correlation parameter that informs the degree to which the microorganism-related condition associations are shared between two conditions, such as based on the multi-condition features. Performing the cross-condition analyses can include applying a dimensionality reduction technique on the distance matrix calculated from the microbiome characteristics (e.g., microbiome features, microorganism datasets, etc.); and using the latent variables with a machine learning model and/or other suitable artificial intelligence approach. In a specific example, performing the cross-condition analyses can include determining a Bray-Curtis dissimilarity between microbiome characteristics (e.g., for the different samples corresponding to the different subjects, etc.); applying the resulting sample dissimilarity matrix as an input into singular value decomposition for deriving principial components and eigenvalues; and performing additional analyses on the principal components explaining more than 1/1000 of the data's total variance. Subsequent cross-condition analyses can be performed, such as including, with a microbiome characterization module (e.g., Analytical Module C), applying a machine learning model and/or other suitable artificial intelligence approach, such as a Bayesian Multi-Kernel Regression for obtaining quantification of the cross-condition correlation explained by the microbiome characteristics. Performing the cross-condition analyses can include quantifying the correlation among conditions explained by the microbiome characteristics using a multivariate variance-component model estimating the variance of each microorganism-related condition (e.g., phenotype) associated with the microbiome and the covariance among the microorganism-related conditions explained by the microbiome characteristics. In a specific example, performing the cross-condition analyses can include fitting a two variance component model of the form y=u+u₀+u₁+∈ where y=(y₁^{T},y₂^{T})^{T}, u₀˜N(0,σᵤ_{₀}²G₀), u₁˜N(0,G₁), ϵ˜N(0,σ_{ϵ}²I), where

$${G}_{0} = [ \begin{matrix}{X}_{1} {X}_{1}^{T} & {X}_{1} {X}_{2}^{T} \\ {X}_{2} {X}_{1}^{T} & {X}_{2} {X}_{2}^{T}\end{matrix} ] \text{/} p and {G}_{1} = [ \begin{matrix}{σ}_{{u}_{1}}^{2} {X}_{1} {X}_{1}^{T} & 0 \\ 0 & {σ}_{{u}_{2}}^{2} {X}_{2} {X}_{2}^{T}\end{matrix} ] \text{/} p ,$$

and where u₀ captures common effects on the two phenotypes which is quantified by σᵤ_{₀}², and u₁ captures phenotypes specific effects quantified by σᵤ_{₁}² and σᵤ_{₂}². In the specific example, the covariance of the phenotypes can be constructed as

$$Var ( y ) = [ \begin{matrix}{σ}_{{u}_{0}}^{2} + {σ}_{{u}_{1}}^{2} & {σ}_{{u}_{0}}^{2} \\ {σ}_{{u}_{0}}^{2} & {σ}_{{u}_{0}}^{2} + {σ}_{{u}_{1}}^{2}\end{matrix} ] + [ \begin{matrix}{σ}_{ϵ 1}^{2} & {σ}_{ϵ 12}^{2} \\ {σ}_{ϵ 12}^{2} & {σ}_{ϵ 2}^{2}\end{matrix} ]$$

leading to a microbiome mediated correlation estimate of r₁₂=σᵤ_{₀}²/(2σᵤ_{₀}²+σᵤ_{₁}²σᵤ_{₂}²), a fraction of the phenotypic variance explained by the microbiome for each trait as R₁²=(σᵤ_{₀}²+σᵤ_{₁}²)/(σᵤ_{₀}²+σᵤ_{₁}²+σ_{ϵ1}²) and R₂²=(σᵤ_{₀}²+σᵤ_{₂}²)/(σᵤ_{₀}²+σᵤ_{₂}²+σ_{ϵ2}²), respectively. In the specific example, the co-correlation can be calculated as co−r₁₂=r₁₂h₁²h₂², analogous to the co-heritability on the quantitative genetics nomenclature, x, for either trait, can correspond to a subset of the principal components obtained from the singular value decomposition of the samples Bray-Curtis similarity matrix. The model can be fitted using a suitable software tool. Gender, age, and/or other suitable user data can be included as fixed-effect covariates on the analyses. In another example, the method 100 can include determining multi-condition microbiome features, where determining the multi-condition microbiome features includes applying, with a first microbiome characterization module (e.g., Analytical Module B, etc.) of the set of microbiome characterization modules, a dimensionality reduction technique to an initial set of microbiome features determined based on the microorganism sequence dataset; determining, with a second microbiome characterization module B, etc) of the set of microbiome characterization modules, a cross-condition correlation metric between different conditions of the plurality of microorganism-related conditions; and determining a multi-condition characterization based on the cross-condition correlation metric, the set of multi-condition microbiome features, and a sample from the user. In the example, determining the multi-condition characterization for the user can include determining a characterization of an additional user condition of the plurality of microorganism-related conditions based on a current user condition of the plurality of microorganism-related conditions (e.g., based on comorbidity between the microorganism-related conditions, based on correlations between the microorganism-related conditions; etc.), the set of multi-condition microbiome features, the sample from the user, and the cross-condition correlation metric. In the example, determining the cross-condition correlation metric with the second microbiome characterization module can include applying at least one of a multivariate model, a canonical correlation model, and a multi-label artificial intelligence approach, for the different conditions of the plurality of microorganism-related conditions. However, determining cross-condition correlation metrics, other suitable metrics associated with cross-condition analyses, and/or performing other suitable cross-condition analyses can be performed in any suitable manner.

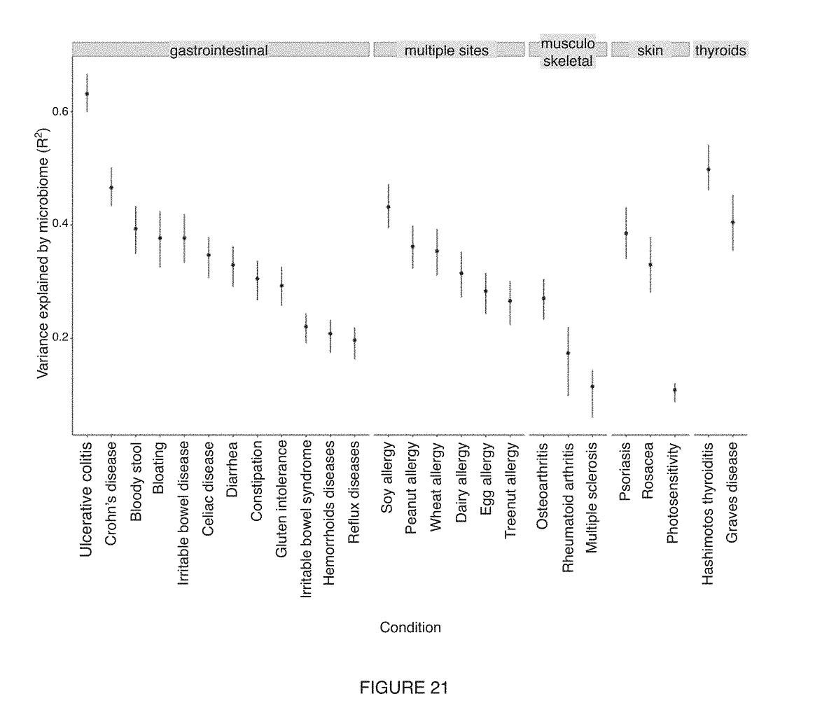

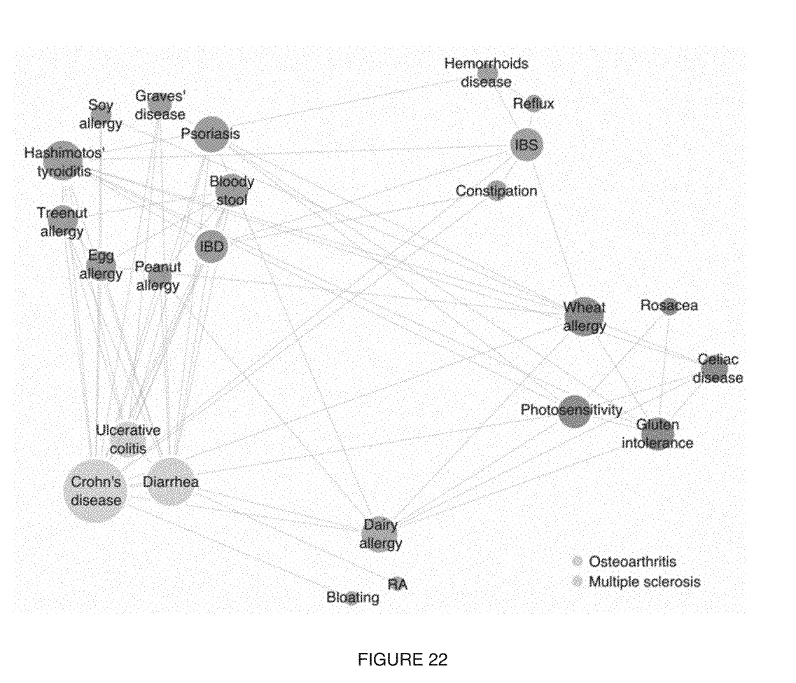

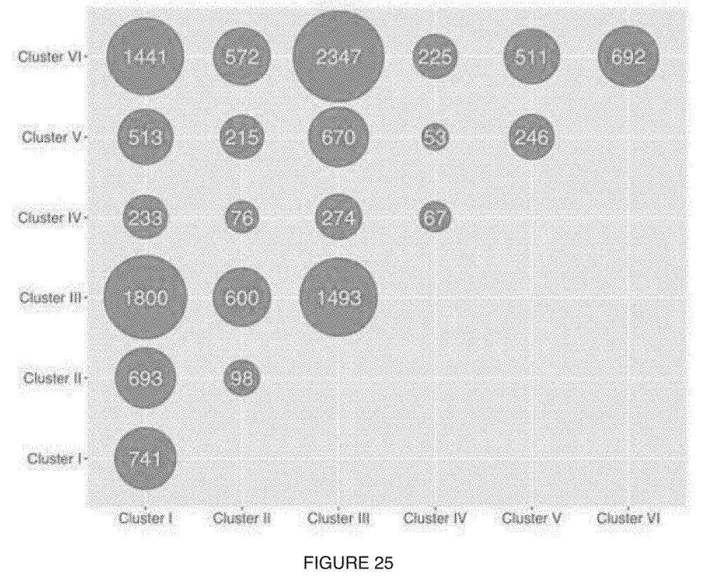

Performing cross-condition analyses can include identifying groups (e.g., clusters) of microorganism-related conditions, such as groups of microorganism-related conditions with similar patterns of shared microbiome characteristics (e.g., shared microbiome-association, etc.). For example, the method 100 can include determining a set of microorganism-related condition groups from the plurality of microorganism-related conditions based on multi-condition microbiome features (e.g., determined using microbiome characterization modules, etc.): and facilitating therapeutic intervention for the microorganism-related conditions based on the set of microorganism-related condition groups (e.g., and a multi-condition characterization, etc.). In an example, identifying groups can include performing unsupervised hierarchical clustering, where inputs can include the matrix of pairwise scaled correlations (co−r₁₂=r₁₂h₁²h₂²); calculating a distance matrix through a Spearman correlation among the rows to estimate their distances; and using the distance matrix as an input for the hierarchical clustering. In the example, Bayesian Multi-Kernel Regression can be used to identify a substantial, but variable, fraction of the phenotypic variance explained by the microorganism data (e.g., microbiome features), where, in a specific example, variance explained (R²) ranged from 63% for ulcerative colitis to 10% for photosensitivity (e.g., as shown in FIG. 21 and Table 26). In the example, application of a multivariate mixed-model can be used to estimate the microbiome-associated correlation (co−r₁₂) between 325 pairs of diseases, where the results can be used for a clustering analysis using the microbiome-based correlations to obtain a data-driven arrangement of the microorganism-related conditions being analyzed (e.g., as shown in FIGS. 22 and 25), and where the hierarchical organization can lead to six microorganism-related condition groups (e.g., clusters; as shown in Table 27; as shown in FIG. 25 where numbers along the diagonal illustrate individuals with comorbidity within a given group such as where they report microorganism-related conditions of the same group, and where numbers that are off-diagonal illustrate individuals with comorbidities across groups such as reporting at least one condition of each group corresponding to the off diagonal point; etc.). Statistically significant pairs of conditions can be identified. In the example, multiple testing correction can lead to identifying 75 out of 325 (23%) as significantly associated correlations (Bonferroni corrected p-value <0.05), which can include 52 out of 75 (69%) inter group associations among 10 of 15 pairs, where Cluster V and Cluster VI had more intercluster significant correlations than expected by chance (binomial p-value=2×10⁻¹⁰; observed=76%, 23 of 30; expected=24%, 79 of 325), and where these clusters are characterized by autoimmune and allergy conditions (e.g., where a summary of correlations can be shown in Table 27, etc.). In examples, cross-condition analyses can indicate disease comorbidity, such as in relation to the human gut microbiome and/or other suitable microbiomes corresponding to other sites, etc.). In examples, derived data supports the association between the human gut microbiome and a plurality of conditions (e.g., comorbid conditions, etc.), such as where derived data can show the microbiome explaining a significant variability of the variance with a plurality of autoimmune diseases (e.g., R²=0.69 for ulcerative colitis; R²=0.49 for Hashimoto's thyroiditis; R²=0.69 for Crohn's disease; etc.).

In the example, cross-condition analyses can lead to the identification of six microorganism-related condition groups: Cluster I (e.g., as shown in Table 28, in relation to co-occurrence, etc.) including wheat and gluten-related disorders, and rosacea and skin photosensitivity; Cluster II including dairy allergy (e.g., as shown in Table 29, etc.), rheumatoid arthritis (RA) and bloating; Cluster III including the irritable bowel syndrome (IBS) (e.g., as shown in Table 30, in relation to co-occurrence with IBD and other microorganism-related conditions, etc.), reflux, constipation and hemorrhoids; Cluster IV including Multiple Sclerosis (MS) and Osteoarthritis (OA); Cluster V including ulcerative colitis and Crohn's disease, the two subtypes of IBD, and the symptom diarrhea, which is prevalent in both conditions; Cluster VI including remaining food allergies (e.g., soy allergy, peanut allergy, tree nut allergy and egg allergy) and autoimmune diseases (e.g., Psoriasis, Hashimoto's thyroiditis, Grave's disease, and IBD). In an example, the set of microorganism-related condition groups can include at least one of a first group including an allergy-related condition, a second group including a locomotor-related condition, and a third group including a gastrointestinal-related condition, and where facilitating therapeutic intervention can include facilitating therapeutic intervention for the microorganism-related conditions based on a multi-condition characterization and the at least one of the first, the second, and the third group (e.g., based on the classifications of the microorganism-related conditions into the clusters, etc.). In an example, a fraction of females and males with different number of comorbidities can be calculated (e.g., as shown in Table 31).

Performing cross-condition analyses can be used in facilitating therapeutic intervention. Performing cross-condition analyses can be used to group microorganism-related conditions to identify biologically relevant condition groups, which can facilitate therapeutic intervention by way of stratifying users on the bases of their microbiome characteristics and risk of comorbid conditions, such as for multilevel therapeutic interventions including primary prevention, early screening, development of personalized therapies, and/or any other suitable therapeutic applications. Microbiome-driven classification (e.g., clustering, etc.) of microorganism-related conditions can enable stratification of users for facilitating prevention, diagnosis, treatment, and/or other suitable therapeutic intervention-related processes, such as for prioritizing therapies and/or improving conditions of the same group and/or discouraging therapies showing opposite results amongst group. For example, facilitating therapeutic intervention can include at least one of: a) promoting a first therapy for a user based on an assignment of the user to at least one microorganism-related condition group of the set of microorganism-related condition groups (e.g., identified using analytical techniques described herein, through one or more microbiome characterization modules; etc.); b) promoting a second therapy for the user based on associations between microorganism-related conditions belonging to a same microorganism-related condition group of the set of microorganism-related condition groups; and c) discouraging a third therapy for the user based on associations between microorganism-related conditions belonging to different microorganism-related condition groups of the set of microorganism-related condition groups. However, cross-condition analyses and/or any other suitable characterization processes can be used to facilitate therapeutic intervention in any suitable manner.

In a variation, characterization can be based upon features associated with (e.g., derived from) a statistical analysis (e.g., an analysis of probability distributions) of similarities and/or differences between a first group of subjects exhibiting a target state (e.g., a microorganism-related condition state) and a second group of subjects not exhibiting the target state (e.g., a “normal” state). In implementing this variation, one or more of a Kolmogorov-Smimov (KS) test, a permutation test, a Cramé-von Mises test, any other statistical test (e.g., t-test, z-test, chi-squared test, test associated with distributions, etc.), and/or other suitable analytical techniques can be used. In particular, one or more such statistical hypothesis tests can be used to assess a set of features having varying degrees of abundance in a first group of subjects exhibiting a target state (e.g., a sick state) and a second group of subjects not exhibiting the target state (e.g., having a normal state). In more detail, the set of features assessed can be constrained based upon percent abundance and/or any other suitable parameter pertaining to diversity in association with the first group of subjects and the second group of subjects, in order to increase or decrease confidence in the characterization. In a specific implementation of this example, a feature can be derived from a taxon of bacteria that is abundant in a certain percentage of subjects of the first group and subjects of the second group, where a relative abundance of the taxon between the first group of subjects and the second group of subjects can be determined from the KS test, with an indication of significance (e.g., in terms of p-value). Thus, an output of Block S130 can include a normalized relative abundance value (e.g., 25% greater abundance of a taxon in subjects with a microorganism-related condition vs. subjects without the microorganism-related condition; in sick subjects vs. healthy subjects) with an indication of significance (e.g., a p-value of 0.0013). Variations of feature generation can additionally or alternatively implement or be derived from functional features or metadata features (e.g., non-bacterial markers). Additionally or alternatively, any suitable microbiome features can be derived based on statistical analyses (e.g., applied to a microorganism sequence dataset and/or other suitable microorganism dataset, etc.) including any one or more of: a prediction analysis, multi hypothesis testing, a random forest test, principal component analysis, and/or other suitable analytical techniques.

In performing the characterization process, Block S130 can additionally or alternatively transform input data from at least one of the microbiome composition diversity dataset and microbiome functional diversity dataset into feature vectors that can be tested for efficacy in predicting characterizations of the population of subjects. Data from the supplementary dataset can be used to provide indication of one or more characterizations of a set of characterizations, where the characterization process is trained with a training dataset of candidate features and candidate classifications to identify features and/or feature combinations that haw high degrees (or low degrees) of predictive power in accurately predicting a classification. As such, refinement of the characterization process with the training dataset identifies feature sets (e.g., of subject features, of combinations of features) having high correlation with specific classifications of subjects.

In variations, feature vectors (and/or any suitable set of features) effective in predicting classifications of the characterization process can include features related to one or more of: microsome diversity metrics (e.g., in relation to distribution across taxonomic groups, in relation to distribution across archaeal, bacterial, viral, and/or eukaryotic groups), presence of taxonomic groups in one's microbiome, representation of specific genetic sequences (e.g., 16S sequences) in one's microbiome, relative abundance of taxonomic groups in one's microbiome, microbiome resilience metrics (e.g., in response to a perturbation determined from the supplementary dataset), abundance of genes that encode proteins or RNAs with given functions (enzymes, transporters, proteins from the immune system, hormones, interference RNAs, etc.) and any other suitable features associated with (e.g., derived from) the microbiome diversity dataset and/or the supplementary dataset. In variations, microbiome features can be associated with (e.g., include, correspond to, typify, etc.) at least one of: presence of a microbiome feature from the microbiome features (e.g., user microbiome features, etc.), absence of the microbiome features from the microbiome features, relative abundance of different taxonomic groups associated with the microorganism-related condition; a ratio between at least two microbiome features associated with the different taxonomic groups, interactions between the different taxonomic groups, and phylogenetic distance between the different taxonomic groups. In a specific example, microbiome features can include one or more relative abundance characteristics associated with at least one of the microbiome composition diversify features (e.g., relative abundance associated with different taxa, etc.) and the microbiome functional diversity features (e.g., relative abundance of sequences corresponding to different functional features; etc.). Relative abundance characteristics and/or other suitable microbiome features (and/or other suitable data described herein) can be extracted and/or otherwise determined based on: a normalization, a feature vector derived from at least one of linear latent variable analysis and non-linear latent variable analysis, linear regression, non-linear regression, a kernel method, a feature embedding method, a machine learning method, a statistical inference method, and/or other suitable analytical techniques. Additionally or alternatively, combinations of features can be used in a feature vector, where features can be grouped and/or weighted in providing a combined feature as part of a feature set. For example, one feature or feature set can include a weighted composite of the number of represented classes of bacteria in one's microbiome, presence of a specific genus of bacteria in one's microbiome, representation of a specific 16S sequence in one's microbiome, and relative abundance of a first phylum over a second phylum of bacteria. However, the feature vectors can additionally or alternatively be determined in any other suitable manner.

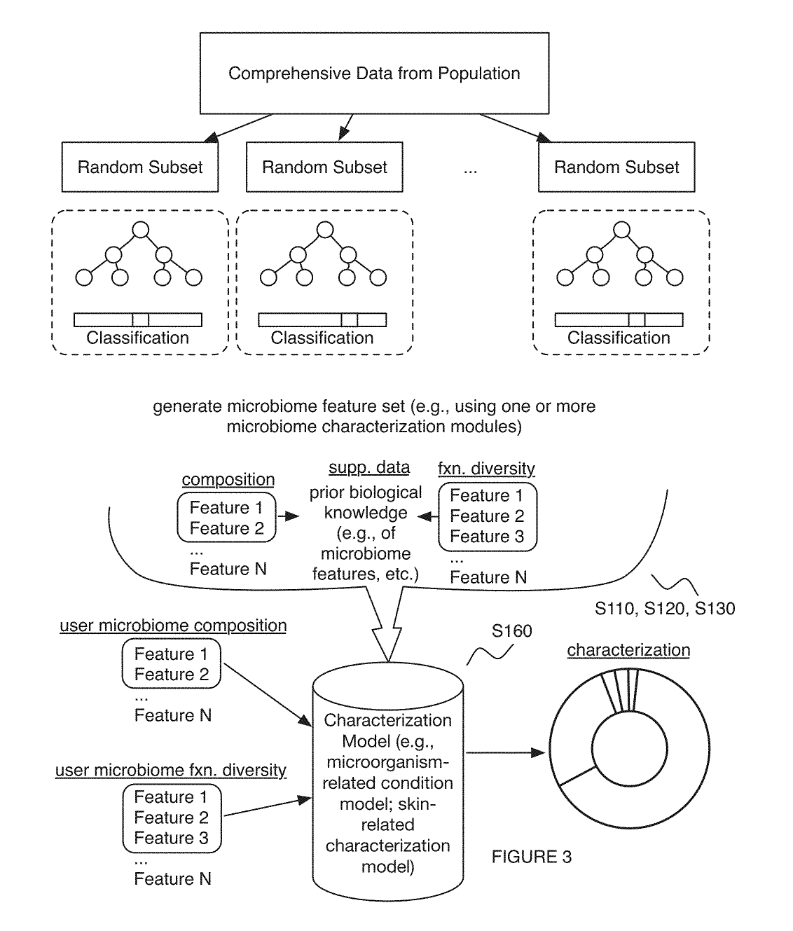

As shown in FIG. 3, in one such alternative variation of Block S130, the characterization process can be generated and trained according to a random forest predictor (RFP) algorithm that combines bagging (e.g., bootstrap aggregation) and selection of random sets of features from a training dataset to construct a set of decision trees, T, associated with the random sets of features. In using a random forest algorithm, N cases from the set of decision trees are sampled at random with replacement to create a subset of decision trees, and for each node, m prediction features are selected from all of the prediction features for assessment. The prediction feature that provides the best split at the node (e.g., according to an objective function) is used to perform the split (e.g., as a bifurcation at the node, as a trifurcation at the node). By sampling many times from a large dataset, the strength of the characterization process, in identifying features that are strong in predicting classifications can be increased substantially. In this variation, measures to prevent bias (e.g., sampling bias) and/or account for an amount of bias can be included during processing, such as to increase robustness of the model.

In a variation, Block S130 and/or other portions of the method 100 can include applying computer-implemented rules (e.g., models, feature selection rules, etc.) to process population-level data, but can additionally or alternatively include applying computer-implemented rules to process microbiome-related data on a demographic-specific basis (e.g., subgroups sharing a demographic feature such as therapy regimens, dietary regimens, physical activity regimens, ethnicity, age, gender, weight, sleeping behaviors, etc.), condition-specific basis (e.g., subgroups exhibiting a specific microorganism-related condition, a combination of microorganism-related conditions, triggers for the microorganism-related conditions, associated symptoms, etc.), a sample type-specific basis (e.g., applying different computer-implemented rules to process microbiome data derived from different collection sites; etc.), a user basis (e.g., different computer-implemented rules for different users; etc.) and/or any other suitable basis. As such, Block S130 can include assigning users from the population of users to one or more subgroups; and applying different computer-implemented rules for determining features (e.g., the set of feature types used; the types of characterization models generated from the features; etc.) for the different subgroups. However, applying computer-implemented rules can be performed in any suitable manner.

In another variation, Block S130 can include processing (e.g., generating, training, updating, executing, storing, etc.) one or more characterization models (e.g., microorganism-related condition characterization models, etc.) for one or more microorganism-related conditions (e.g., for outputting characterizations for users describing user microbiome characteristics in relation to microorganism-related conditions, etc.). The characterization models preferably leverage microbiome features as inputs, and preferably output microorganism-related characterizations and/or any suitable components thereof; but characterization models can use and suitable inputs to generate any suitable outputs. In an example, Block S130 can include transforming the supplementary data, the microbiome composition diversity features, and the microbiome functional diversity features, other microbiome features, outputs of microbiome characterization modules, and/or other suitable data into one or more characterization models (e.g., training a microorganism-related characterization model based on the supplementary data and microbiome features; etc.) for one or more microorganism-related conditions. In another example, the method 100 can include: determining a population microorganism sequence dataset (e.g., including microorganism sequence outputs for different users of the population; etc.) for a population of users associated with one or more microorganism-related conditions, based on a set of samples from the population of users (e.g., and/or based on one or more primer types associated with the microorganism-related condition; etc.); collecting a supplementary dataset associated with diagnosis of the one or more microorganism-related conditions for the population of subjects; and generating the microorganism-related condition characterization model based on the population microorganism sequence dataset and the supplementary dataset.

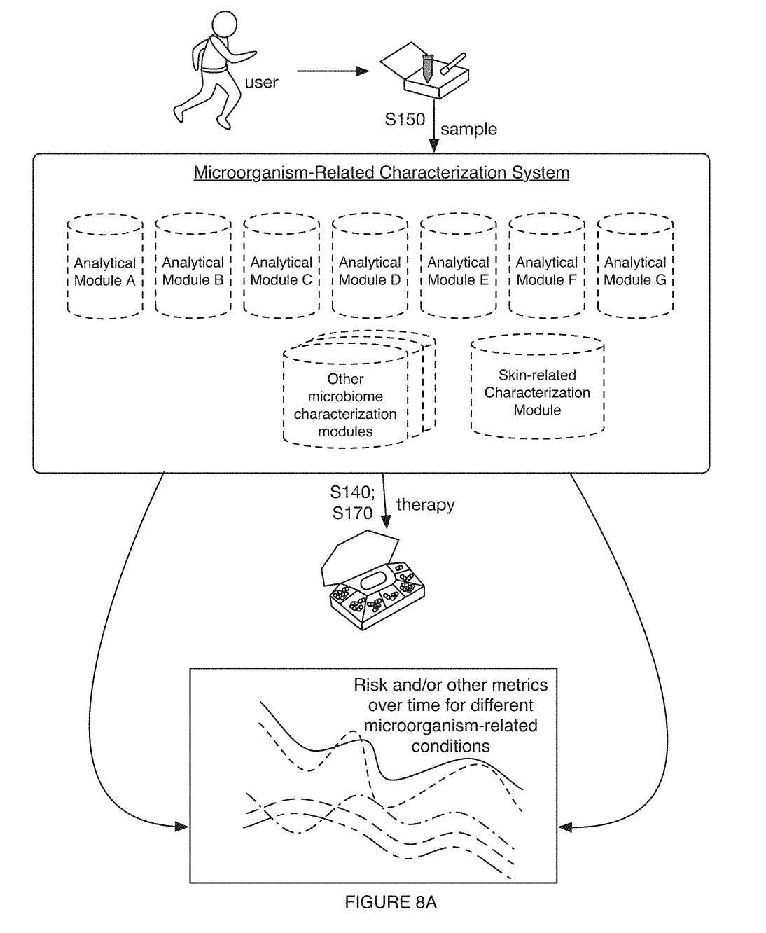

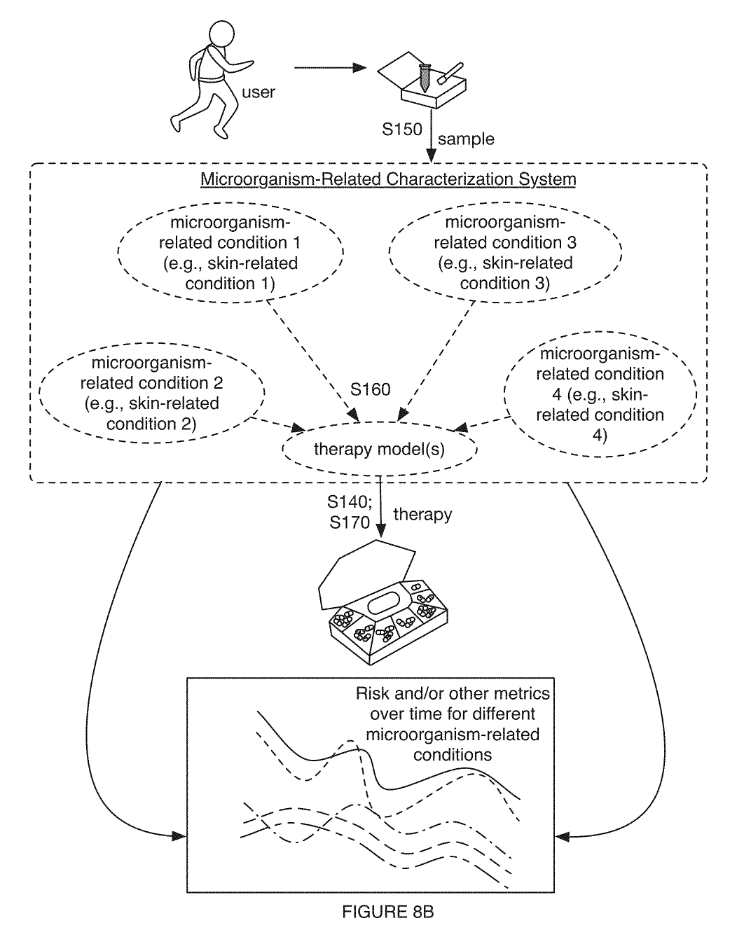

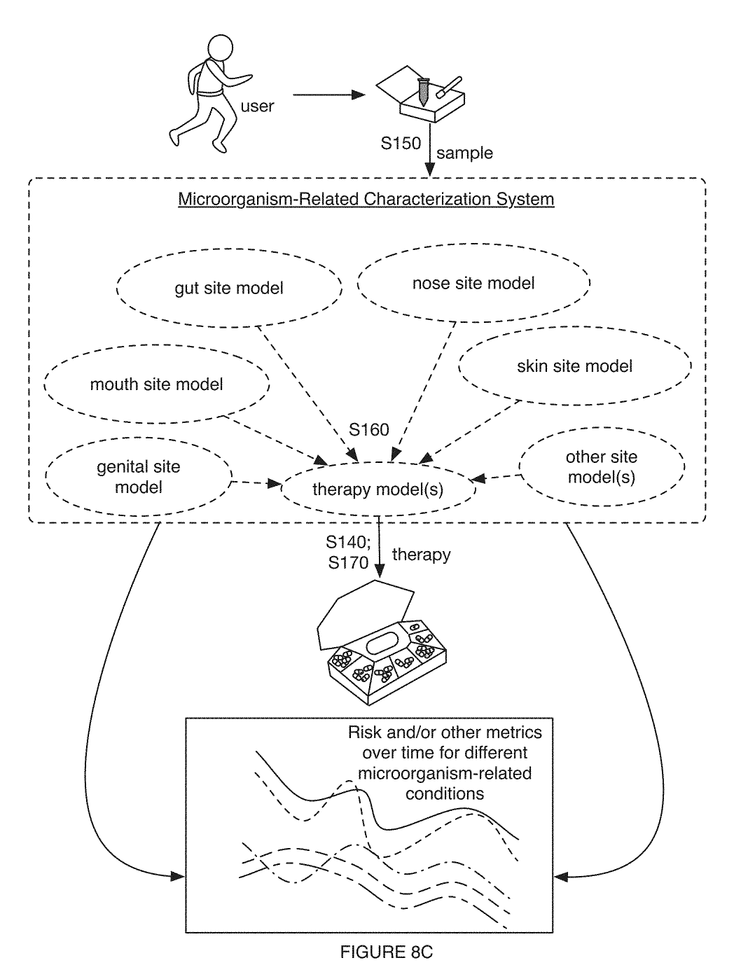

In another variation, as shown in FIGS. 8A-8C, different microorganism-related characterization models and/or other suitable models (e.g., generated with different algorithms, with different sets of features, with different input and/or output types, applied in different manners such as in relation to time, frequency, component applying the model, etc.) can be generated for different microorganism-related conditions, different user demographics (e.g., based on age, gender, weight, height, ethnicity; etc.), different physiological sites (e.g., a gut site model, a nose site model, a skin site model, a mouth site model, a genitals site model, etc.), individual users, supplementary data (e.g., models incorporating prior knowledge of microbiome features, microorganism-related conditions, and/or other suitable components; features associated with biometric sensor data and/or survey response data vs. models independent of supplementary data, etc.), and/or other suitable criteria.

In variations, determining microorganism-related characterizations and/or any other suitable characterizations can include determining microorganism-related characterizations in relation to specific physiological sites (e.g., gut, healthy gut, skin, nose, mouth, genitals, other suitable physiological sites, other sample collection sites, etc.), such as through any one or more of: determining a microorganism-related characterization based on a characterization model derived based on site-specific data (e.g., defining correlations between a microorganism-related condition and microbiome features associated with one or more physiological sites); determining a microorganism-related characterization based on a user biological sample collected at one or more physiological sites, and/or any other suitable site-related processes. In examples, machine learning approaches (e.g., classifiers, deep learning algorithms), parameter optimization approaches (e.g., Bayesian Parameter Optimization), validation approaches (e.g., cross validation approaches), statistical tests (e.g., univariate statistical techniques, multivariate statistical techniques, correlation analysis such as canonical correlation analysis, etc.), dimension reduction techniques, and/or other suitable analytical techniques (e.g., described herein) can be applied in determining site-related (e.g., physiological site-related, etc.) characterizations (e.g., using a one or more approaches for one or more sample collection sites, such as for each type of sample collection site, etc.), other suitable characterizations, therapies, and/or any other suitable outputs. In a specific example, performing a characterization process (e.g., determining a microorganism-related characterization; determining microbiome features; based on a microorganism-related characterization model; etc.) can include applying at least one of: machine learning approaches, parameter optimization approaches, statistical tests, dimension reduction approaches, and/or other suitable approaches (e.g., where microbiome features such as a set of microbiome composition diversity features and/or a set of microbiome functional diversity features can be associated with microorganisms collected at least at one of a gut site, a skin site, a nose site, a mouth site, a genitals site, etc.). In another specific example, characterization processes performed for a plurality of sample collection sites can be used to generate individual characterizations that can be combined to determine an aggregate characterization (e.g., an aggregate microbiome score, such as for one or more conditions described herein, etc.). However, the method 100 can include determining any suitable site-related (e.g., site-specific) outputs, and/or performing any suitable portions of the method 100 (e.g., collecting samples, processing samples, determining therapies) with site-specificity and/or other site-relatedness in any suitable manner.

Characterization of the subject(s) can additionally or alternatively implement use of a high false positive test and/or a high false negative test to further analyze sensitivity of the characterization process in supporting analyses generated according to embodiments of the method 100. However, performing a characterization process S130 can be performed in any suitable manner.

### 4.3.1 Skin-Related Characterization Process

Performing a characterization process S130 can include performing a skin-related characterization process (e.g., determining a characterization for one or more skin-related conditions; determining and/or applying one or more skin-related characterization models such as models applying one or more analytical techniques associated with one or more microbiome characterization modules; applying one or more analytical techniques with one or more microbiome characterization modules to generate a skin-related characterization for one or more skin-related conditions such as comorbid skin-related conditions; determining skin-related characterizations for use in determining and/or promoting one or more therapies for one or more skin-related conditions; etc.) S135, such as for one or more users (e.g., for data corresponding to samples from a set of subjects for generating one or more skin-related characterization models; for a single user for generating a skin-related characterization for the user, such as through using one or more skin-related characterization models; etc.) and/or for one or more skin-related conditions (e.g., using any suitable type and number of microbiome characterization modules, cross-condition analyses, etc.).

In a variation, performing a skin-related characterization process can include determining microbiome features associated with one or more skin-related conditions. In an example, performing a skin-related characterization process can include applying one or more analytical techniques (e.g., statistical analyses) to identify the sets of microbiome features (e.g., microbiome composition features, microbiome composition diversity-features, microbiome functional features, microbiome functional diversity features, etc.) that haw the highest correlations with one or more skin-related conditions (e.g., features associated with a single skin-related condition, cross-condition features associated with multiple skin-related conditions and/or other suitable skin-related conditions, etc.). In a specific example, performing a skin-related characterization process can facilitate therapeutic intervention for one or more skin-related conditions, such as through facilitating intervention associated with therapies having a positive effect on a state of one or more users in relation to the one or more skin-related conditions. In another specific example, performing a skin-related characterization process (e.g., determining features highest correlations to one or more skin-related conditions, etc.) can be based upon a random forest predictor algorithm trained with a training dataset derived from a subset of the population of subjects (e.g., subjects having the one or more skin-related conditions; subjects not having the one or more skin-related conditions; etc.), and validated with a validation dataset derived from a subset of the population of subjects. However, determining microbiome features and/or other suitable aspects associated with one or more skin-related conditions can be performed in any suitable manner.

In variations, performing a skin-related characterization process S135 can include performing a photosensitivity-associated condition characterization process for one or more photosensitivity-associated conditions. In an example, a skin-related characterization process can be based upon statistical analyses for identifying the sets of features that have the highest correlations with photosensitivity-associated conditions for which one or more therapies would have a positive effect, based upon a random forest predictor algorithm trained with a training dataset derived from a subset of the population of subjects, and validated with a validation dataset derived from a subset of the population of subjects. In examples, photosensitivity-associated conditions can include a skin condition characterized by an abnormal reaction of the skin to a component of the electromagnetic spectrum of sunlight. In examples, photosensitivity-associated conditions can be diagnosed by skin examination, phototests and photopatch tests and/or other suitable approaches. Photosensitivity-associated conditions can be associated with specific microbiota diversity and/or health conditions related to relative abundance of gut microorganisms, microorganisms associated with any suitable physiological site, microbiome functional diversity, and/or other suitable microbiome-related aspects.

Microbiome features associated with one or more photosensitivity-associated conditions (and/or other suitable skin-related conditions) (e.g., positively-correlated with; negatively correlated with; useful for diagnosis; etc.) can include features associated with any combination of one or more of the following taxa (e.g., features describing abundance of; features describing relative abundance of; features describing functional aspects associated with; features derived from; features describing presence and/or absence of; etc.): Alloprevotella (genus), Prevotella sp. WAL 2039G (species), Corynebacterium mastitidis (species), Bacteroidaceae (family), Blautia (genus), Bacteroides (genus), Desulfovibrio (genus), Clostridium (genus), Bacteroides vulgatus (species), Faecalibacterium prausnitzii (species), Blautia faecis (species), Alistipes putredinis (species), Bacteroides sp. AR20 (species), Bacteroides sp. AR29 (species), Bacteroides acidifaciens (species), Dielma (genus), Slackia (genus), Eggerthella (genus), Adlercreutzia (genus), Paraprevotella (genus), Alistipes (genus), Holdemania (genus), Eisenbergiella (genus), Enterorhabdus (genus), Adlercreutzia equolifaciens (species), Phascolarctobacterium succinatutens (species), Roseburia inulinivorans (species), Phascolarctobacterium sp. 377 (species), Desulfovibrio piger (species), Eggerthella sp. HGA1 (species), Lactonifactor longoviformis (species), Alistipes sp. HGB5 (species), Holdemania filiformis (species), Collinsella intestinalis (species), Neisseria macacae (species), Clostridiaceae (family), Gemella sanguinis (species), Bacteroides fragilis (species), Enterobacteriaceae (family), Lachnospiraceae (family), Pasteurellaceae (family), Pasteurellales (order), Enterobacteriales (order), Sphingobacteriales (order), Haemophilus (genus), Leuconostoc (genus), Breumdimonas (genus), Prevotella oris (species), Odoribacter (genus), Capnocytophaga (genus), Flavobacterium (genus), Pseudomonas brenneri (species), Flavobacterium ceti (species), Brevundimonas sp. FXJ8.080 (species), Ruminococcaceae (family), Vibrionaceae (family), Flavobacteriaceae (family), Fusobacteriaceae (family), Porphyromonadaceae (family), Brevibacteriaceae (family), Rhodobacteraceae (family), Intrasporangiaceae (family), Bifidobacteriaceae (family), Sphingobacteriaceae (family), Caulobacteraceae (family), Campylobacteraceae (family), Bacteroidia (class), Fusobacteriia (class), Flavobacteriia (class), Bifidobacteriales (order), Neisseriales (order), Bacteroidales (order), Rhodobacterales (order), Flavobacteriales (order), Vibrionales (order), Fusobacteriales (order), Caulobacterales (order), Fusobacteria (phylum), Actinobaculum (genus), Varibaculum (genus), Fusicatenibacter (genus), Brevibacterium (genus), Faecalibacterium (genus), Campylobacter (genus), Actinobacillus (genus), Porphyromonas (genus), Fusobacterium (genus), Chryseobacterium (genus), Megasphaera (genus), Rothia (genus), Neisseria (genus), Lactobacillus sp. BL302 (species), Bacteroides plebeius (species), Corynebacterium ulcerans (species), Varibaculum cambriense (species), Blautia wexlerae (species), Staphylococcus sp. WB18-16 (species), Streptococcus sp. oral taxon G63 (species), Propionibacterium acnes (species), Anaerococcus sp. 9401487 (species), Haemophilus parainfluenzae (species), Staphylococcus epidermidis (species), Campylobacter ureolyticus (species), Janibacter sp. M3-5 (species), Prevotella timonensis (species), Peptoniphilus sp. DNF00840 (species), Finegoldia sp. S8 F7 (species), Prevotella disiens (species), Porphyromonas catoniae (species), Fusobacterium periodonticum (species), and/or other suitable taxa (e.g., described herein); and/or can include functional features associated with any combination of one or more of (e.g., features describing abundance of; features describing relative abundance of; features describing functional aspects associated with; features derived from; features describing presence and/or absence of; etc.): Infectious Diseases (KEGG2), Poorly Characterized (KEGG2), Metabolic Diseases (KEGG2), Immune System Diseases (KEGG2), Cellular Processes and Signaling (KEGG2), Restriction enzyme (KEGG3), Nucleotide excision repair (KEGG3) and/or other suitable functional features (e.g., described herein, etc.). In variations, characterization of a user can include characterization of the user as someone with one or more photosensitivity skin-associated conditions based upon detection of one or more of the above features, in a manner that is an additional or alternative to typical approaches of diagnosis and/or treatment.

In variations, performing a skin-related characterization process S135 can include performing a dry skin-associated condition characterization process for one or more dry skin-associated conditions. In an example, a skin-related characterization process can be based upon statistical analyses for identifying the sets of features that have the highest correlations with dry skin-associated conditions for which one or more therapies would have a positive effect, based upon a random forest predictor algorithm trained with a training dataset derived from a subset of the population of subjects, and validated with a validation dataset derived from a subset of the population of subjects. In examples, dry skin-associated conditions can include one or more of rough skin, itching, flaking, scaling or peeling, fine lines or cracks, gray skin in people with dark skin, redness, deep cracks that can bleed and that can lead to infections, and/or other suitable dry skin-associated conditions. Dry skin-associated conditions can be associated with specific microbiota diversity and/or health conditions related to relative abundance of gut microorganisms, microorganisms associated with any suitable physiological site, microbiome functional diversity, and/or other suitable microbiome-related aspects.

Microbiome features associated with one or more dry skin-associated conditions (and/or other suitable skin-related conditions) (e.g., positively correlated with; negatively correlated with; useful for diagnosis; etc.) can include features associated with any combination of one or more of the following taxa (e.g., features describing abundance of; features describing relative abundance of; features describing functional aspects associated with; features derived from; features describing presence and/or absence of; etc.): Corynebacteriaceae (family), Bacilli (class), Lactobacillales (order), Actinomycetales (order), Firmicutes (phylum), Corynebacterium (genus), Dermabacteraceae (family), Lactobacillaceae (family), Propionibacteriaceae (family), Actinobacteria (class), Dermabacter (genus), Dialister (genus), Facklamia (genus), Lactobacillus (genus), Propionibacterium (genus), Corynebacterium ulcerans (species), Facklamia hominis (species), Corynebacterium sp. (species), Propionibacterium sp. MSP09A (species), Facklamia sp. 1440-97 (species), Staphylococcus sp. C9I2 (species), Anaerococcus sp. 9402080 (species), Corynebacterium glucuronolyticum (species), Dermabacter hominis (species), Enterobacteriaceae (family), Pseudomonadaceae (family), Staphylococcaceae (family), Gammaproteobacteria (class), Bacillales (order), Enterobacteriales (order), Bifidobacterium (genus), Pseudomonas (genus), Anaeroglobus (genus), Kluyvera (genus), Atopobium (genus), Staphylococcus (genus), Lactobacillus sp. BL302 (species), Corynebacterium mastitidis (species), Bifidobacterium longum (species), Anaeroglobus geminatus (species), Anaerococcus sp. S9 PR-16 (species), Prevotella timonensis (species), Kluyvera georgiana (species), Actinobaculum (genus), Finegoldia (genus), Cronobacter (genus), Acinetobacter sp. WB22-23 (species), Anaerococcus octavirus (species), Finegoldia sp. S9 AA1-5 (species), Staphylococcus sp. C-D-MA2 (species), Peptoniphilus sp. 7-2 (species), Cronobacter sakazakii (species), Pasteurellaceae (family), Acidobacteriia (class), Sphingobacteriia (class), Sphingobacteriales (order), Acidobacteria (phylum), Porphyromonas (genus), Haemophilus (genus), Acinetobacter (genus), Anaerococcus sp. 8405254 (species), Sphingomonadaceae (family), Sphingomonadales (order), Kocuria (genus), Gemella (genus), Veillonella sp. CM60 (species), Lactobacillus sp. 7_1_4?FAA (species), Gemella sp. 933-88 (species), Porphyromonas catoniae (species), Haemophilus parainfluenzae (species), Bacteroides sp. AR20 (species), Bacteroides vulgatus (species), Bacteroides sp. D22 (species), Dorea longicatena (species), Parabacteroides merdae (species), Bacteroides sp. AR29 (species), Dorea (genus), Collinsella (genus), Bacteroides (genus), Oscillospiraoeae (family), Ruminococcaoeae (family), Bacteroidaceae (family), Verrucomicrobiaceae (family), Coriobacteriaceae (family), Clostridiales (order), Bacteroidales (order), Verrucomicrobiales (order), Coriobacteriales (order), Thermoanaerobacterales (order), Clostridia (class), Bacteroidia (class), Verrucomicrobiae (class), Verrucomicrobia (phylum), Bacteroidetes (phylum), and/or other suitable taxa (e.g., described herein); and/or can include functional features associated with any combination of one or more of (e.g., features describing abundance of; features describing relative abundance of; features describing functional aspects associated with; features derived from; features describing presence and/or absence of; etc.): Translation (KEGG2), Cellular Processes and Signaling (KEGG2), Amino Acid Metabolism (KEGG2), Cell Growth and Death (KEGG2), Replication and Repair (KEGG2), Metabolism of Other Amino Acids (KEGG2), Neurodegenerative Diseases (KEGG2), Metabolism of Cofactors and Vitamins (KEGG2), Transport and Catabolism (KEGG2), Endocrine System (KEGG2), Immune System Diseases (KEGG2), Excretory System (KEGG2), Enzyme Families (KEGG2), Membrane Transport (KEGG2), Carbohydrate Metabolism (KEGG2), Biosynthesis of Other Secondary Metabolites (KEGG2), Metabolism of Terpenoids and Polyketides (KEGG2), Infectious Diseases (KEGG2), Genetic Information Processing (KEGG2), Nervous System (KEGG2), Environmental Adaptation (KEGG2), Nucleotide Metabolism (KEGG2), Signaling Molecules and Interaction (KEGG2), Signal Transduction (KEGG2), Inorganic ion transport and metabolism (KEGG3), Chromosome (KEGG3), Cell cycle—Caulobacter (KEGG3), Ribosome Biogenesis (KEGG3), DNA replication proteins (KEGG3), Translation factors (KEGG3), Glycine, serine and threonine metabolism (KEGG3), Sulfur metabolism (KEGG3), Other ion-coupled transporters (KEGG3), Valine, leucine and isoleucine biosynthesis (KEGG3), Nitrogen metabolism (KEGG3), Peptidoglycan biosynthesis (KEGG3), Homologous recombination (KEGG3), Peroxisome (KEGG3), Sulfur relay system (KEGG3), Peptidases (KEGG3), Protein kinases (KEGG3), Mismatch repair (KEGG3), Xylene degradation (KEGG3), Ribosome (KEGG3), RNA polymerase (KEGG3), Tryptophan metabolism (KEGG3), Histidine metabolism (KEGG3), Vitamin metabolism (KEGG3), Cell motility and secretion (KEGG3), Pyrimidine metabolism (KEGG3), Cytoskeleton proteins (KEGG3), DNA replication (KEGG3), Amino sugar and nucleotide sugar metabolism (KEGG3), Folate biosynthesis (KEGG3), Carbon fixation in photosynthetic organisms (KEGG3), Phosphatidylinositol signaling system (KEGG3), Lysine degradation (KEGG3), Selenocompound metabolism (KEGG3), Fructose and mannose metabolism (KEGG3), Inositol phosphate metabolism (KEGG3), Protein folding and associated processing (KEGG3), PPAR signaling pathway (KEGG3), Lipid metabolism (KEGG3), Valine, leucine and isoleucine degradation (KEGG3), Glyoxylate and dicarboxylate metabolism (KEGG3), Arginine and proline metabolism (KEGG3), Limonene and pinene degradation (KEGG3), D-Alanine metabolism (KEGG3), Porphyrin and chlorophyll metabolism (KEGG3), C5-Branched dibasic acid metabolism (KEGG3), Chaperones and folding catalysts (KEGG3), Fatty acid metabolism (KEGG3), Glutathione metabolism (KEGG3), Pentose phosphate pathway (KEGG3), Phosphotransferase system (PTS) (KEGG3), Pantothenate and CoA biosynthesis (KEGG3), Proximal tubule bicarbonate reclamation (KEGG3), Galactose metabolism (KEGG3), Starch and sucrose metabolism (KEGG3), Primary immunodeficiency (KEGG3), Cysteine and methionine metabolism (KEGG3), Ubiquinone and other terpenoid-quinone biosynthesis (KEGG3), DNA repair and recombination proteins (KEGG3), Tyrosine metabolism (KEGG3), Phenylalanine, tyrosine and tryptophan biosynthesis (KEGG3), Aminoacyl-tRNA biosynthesis (KEGG3), Alanine, aspartate and glutamate metabolism (KEGG3), Photosynthesis (KEGG3), Other transporters (KEGG3), Butanoate metabolism (KEGG3), Bacterial secretion system (KEGG3), Glycerophospholipid metabolism (KEGG3), Oxidative phosphorylation (KEGG3), Type I diabetes mellitus (KEGG3), Glycolysis/Gluconeogenesis (KEGG3), Photosynthesis proteins (KEGG3), Transporters (KEGG3), Terpenoid backbone biosynthesis (KEGG3), Biosynthesis of unsaturated fatty adds (KEGG3), Signal transduction mechanisms (KEGG3), Synthesis and degradation of ketone bodies (KEGG3), Nucleotide excision repair (KEGG3), Secretion system (KEGG3), Alzheimer's disease (KEGG3), Zeatin biosynthesis (KEGG3), Type II diabetes mellitus (KEGG3), D-Glutamine and D-glutamate metabolism (KEGG3), Taurine and hypotaurine metabolism (KEGG3), Glutamatergic synapse (KEGG3), Plant-pathogen interaction (KEGG3), Vitamin B6 metabolism (KEGG3), Citrate cycle (TCA cycle) (KEGG3), Ethylbenzene degradation (KEGG3), Base extension repair (KEGG3), Replication, recombination and repair proteins (KEGG3), Ribosome biogenesis in eukaryotes (KEGG3), Aminobenzoate degradation (KEGG3), Bacterial motility proteins (KEGG3), Biosynthesis of ansamyrins (KEGG3), Ion channels (KEGG3), Metabolism (KEGG2), Poorly-Characterized (KEGG2), Biosynthesis and biodegradation of secondary metabolites (KEGG3), Lipoic acid metabolism (KEGG3), Amino add related enzymes (KEGG3), Translation proteins (KEGG3), Ascorbate and aldarate metabolism (KEGG3), Thiamine metabolism (KEGG3), Function unknown (KEGG3), Glycosaminoglycan degradation (KEGG3), Others (KEGG3), Pentose and glucuronate interconversions (KEGG3), Biotin metabolism (KEGG3), Phenylalanine metabolism (KEGG3), Glycosphingolipid biosynthesis—ganglio series (KEGG3), Pores ion channels (KEGG3), Membrane and intracellular structural molecules (KEGG3), Purine metabolism (KEGG3), One carbon pool by folate (KEGG3), Phosphonate and phosphinate metabolism (KEGG3), Lysosome (KEGG3), Drug metabolism—other enzymes (KEGG3), Penicillin and cephalosporin biosynthesis (KEGG3), Huntington's disease (KEGG3), Nicotinate and nicotinamide metabolism (KEGG3), Drag metabolism—cytochrome P450 (KEGG3), Lipopolysaccharide biosynthesis proteins (KEGG3), Metabolism of xenobiotics by cytochrome P450 (KEGG3), Tuberculosis (KEGG3), Polycyclic aromatic hydrocarbon degradation (KEGG3) and/or any other suitable functional features (e.g., described herein, etc.). In variations, characterization of a user can include characterization of the user as someone with one or more photosensitivity skin-associated conditions based upon detection of one or more of the above features, in a manner that is an additional or alternative to typical approaches of diagnosis and/or treatment.

In variations, performing a skin-related characterization process S135 can include performing a scalp-related condition characterization process for one or more scalp-related conditions. In an example, a skin-related characterization process can be based upon statistical analyses for identifying the sets of features that have the highest correlations with scalp-related conditions for which one or more therapies would have a positive effect, based upon a random forest predictor algorithm trained with a training dataset derived from a subset of the population of subjects, and validated with a validation dataset derived from a subset of the population of subjects. In examples, scalp-related conditions can include one or more of dandruff (e.g., characterized by flaking, itching, scaling of the skin of the scalp; etc.) and/or other suitable scalp-related conditions, such as caused by dry skin, irritated oily skin, sensitivity to hair care products, other conditions that can lead to imbalance of a scalp microbiome, and/or other suitable scalp-related conditions. Scalp-related conditions can be associated with specific microbiota diversity and/or health conditions related to relative abundance of gut microorganisms, microorganisms associated with any suitable physiological site, microbiome functional diversity, and/or other suitable microbiome-related aspects.

Microbiome features associated with one or more scalp-related conditions (and/or other suitable skin-related conditions) (e.g., positively correlated with; negatively correlated with; useful for diagnosis; etc.) can include features associated with any combination of one or more of the following taxa (e.g., features describing abundance of; features describing relative abundance of; features describing functional aspects associated with; features derived from; features describing presence and/or absence of; etc.): Actinobacteria (class), Lactobacillales (order), Actinomycetales (order), Firmicutes (phylum), Dermabacteraceae (family), Lactobacillaceae (family), Propionibacteriaceae (family), Corynebacteriaceae (family), Lactobacillus (genus), Corynebacterium (genus), Propionibacterium (genus), Dermabacter (genus), Eremococcus (genus), Corynebacterium freiburgense (species), Eremoc(KEGG3)occus coleocola (species), Corynebacterium sp. (species), Staphylococcus sp. C9I2 (species), Anaerococcus sp. 8405254 (species), Corynebacterium glucuronolyticum (species), Dermabacter hominis (species), Coriobacteriaceae (family), Enterobacteriaceae (family), Staphylococcaceae (family), Enterobacteriales (order), Bacillales (order), Bifidobacterium (genus), Staphylococcus (genus), Atopobium (genus), Megasphaera (genus), Corynebacterium mastitidis (species), Streptococcus sp. 68353 (species), Finegoldia magna (species), Staphylococcus aureus (species), Haemophilus influenzae (species), Corynebacterium sp. NML 97-0186 (species), Streptococcus sp. oral taxon G59 (species), Dorea (genus), Roseburia sp. 11SE39 (species), Dorea longicatena (species), Prevotellaceae (family), Veillonellaceae (family), Oscillospiraceae (family), Negativicutes class, Selenomonadales (order), Finegoldia (genus), Oscillospira (genus), Intestinimonas (genus), Flavonifractor (genus), Prevotella (genus), Moryella (genus), Catenibacterium mitsuokai (species), Collinsella aerofaciens (species), Peptoniphilus sp. 2002-2300004 (species), Corynebacterium canis (species), Finegoldia sp. S9 AA1-5 (species), Prevotella buccalis (species), Dialister invisus (species), Moraxella (genus), Neisseria (genus), Neisseria mucosa (species), Rikenellaceae (family), and/or other suitable taxa (e.g., described herein); and/or can include functional features associated with any combination of one or more of (e.g., features describing abundance of; features describing relative abundance of; features describing functional aspects associated with; features derived from; features describing presence and/or absence of; etc.): Metabolism of Cofactors and Vitamins (KEGG2), Enzyme Families (KEGG2), Lipid Metabolism (KEGG2), Immune System Diseases (KEGG2), Glycolysis/Gluconeogenesis (KEGG3), Primary immunodeficiency (KEGG3), Pyruvate metabolism (KEGG3), Transport and Catabolism (KEGG2), Neurodegenerative Diseases (KEGG2), Endocrine System (KEGG2), Amino Acid Metabolism (KEGG2), Cellular Processes and Signaling (KEGG2), Signaling Molecules and Interaction (KEGG2), Metabolism of Other Amino Acids (KEGG2), Replication and Repair (KEGG2), Translation (KEGG2), Cell Growth and Death (KEGG2), Membrane Transport (KEGG2), Biosynthesis of Other Secondary-Metabolites (KEGG2), Metabolism of Terpenoids and Polyketides (KEGG2), Inorganic ion transport and metabolism (KEGG3), Vitamin metabolism (KEGG3), Valine, leucine and isoleucine biosynthesis (KEGG3), Peroxisome (KEGG3), Ribosome Biogenesis (KEGG3), Selenocompound metabolism (KEGG3), Histidine metabolism (KEGG3), Chromosome (KEGG3), Sulfur metabolism (KEGG3), PPAR signaling pathway (KEGG3), Porphyrin and chlorophyll metabolism (KEGG3), Phosphatidylinositol signaling system (KEGG3), Inositol phosphate metabolism (KEGG3), Sulfur relay system (KEGG3), Glycine, serine and threonine metabolism (KEGG3), DNA replication proteins (KEGG3), Pantothenate and CoA biosynthesis (KEGG3), Translation factors (KEGG3), Protein folding and associated processing (KEGG3), Type II diabetes mellitus (KEGG3), Protein kinases (KEGG3), Folate biosynthesis (KEGG3), Lysine degradation (KEGG3), RNA polymerase (KEGG3), D-Alanine metabolism (KEGG3), Carbon fixation in photosynthetic organisms (KEGG3), Nitrogen metabolism (KEGG3), Glycerophospholipid metabolism (KEGG3), Biosynthesis of ansamycins (KEGG3), Valine, leucine and isoleucine degradation (KEGG3), Cytoskeleton proteins (KEGG3), Peptidases (KEGG3), Fatty—acid metabolism (KEGG3), Cell cycle—Caulobacter (KEGG3), Phosphotransferase system (PTS) (KEGG3), Pyrimidine metabolism (KEGG3), Alzheimer's disease (KEGG3), Butanoate metabolism (KEGG3), Tryptophan metabolism (KEGG3), Signal transduction mechanisms (KEGG3), Pentose phosphate pathway (KEGG3), Other ion-coupled transporters (KEGG3), Homologous recombination (KEGG3), Replication, recombination and repair proteins (KEGG3), Xylene degradation (KEGG3), Mismatch repair (KEGG3), Glyoxylate and dicarboxylate metabolism (KEGG3), Arginine and proline metabolism (KEGG3), Peptidoglycan biosynthesis (KEGG3), Chaperones and folding catalysts (KEGG3), Type I diabetes mellitus (KEGG3), DNA replication (KEGG3), Bacterial secretion system (KEGG3), Tyrosine metabolism (KEGG3), Citrate cycle (TCA cycle) (KEGG3), Amino sugar and nucleotide sugar metabolism (KEGG3), Ribosome (KEGG3), Limonene and pinene degradation (KEGG3), Cell motility and secretion (KEGG3), Taurine and hypotaurine metabolism (KEGG3), Oxidative phosphorylation (KEGG3), Fructose and mannose metabolism (KEGG3), Vitamin B6 metabolism (KEGG3), Ion channels (KEGG3), Synthesis and degradation of ketone bodies (KEGG3), Other transporters (KEGG3), Galactose metabolism (KEGG3), Polycyclic aromatic hydrocarbon degradation (KEGG3), Transporters (KEGG3), DNA repair and recombination proteins (KEGG3), Starch and sucrose metabolism (KEGG3), Alanine, aspartate and glutamate metabolism (KEGG3), Ribosome biogenesis in eukaryotes (KEGG3), Secretion system (KEGG3), Biosynthesis of unsaturated fatty acids (KEGG3), Cysteine and methionine metabolism (KEGG3), Base excision repair (KEGG3), Aminobenzoate degradation (KEGG3), Photosynthesis (KEGG3), Photosynthesis proteins (KEGG3), Pores ion channels (KEGG3), Lipid biosynthesis proteins (KEGG3), D-Glutamine and D-glutamate metabolism (KEGG3) and/or any other suitable functional features (e.g., described herein, etc.). In variations, characterization of a user can include characterization of the user as someone with one or more photosensitivity skin-associated conditions based upon detection of one or more of the above features, in a manner that is an additional or alternative to typical approaches of diagnosis and/or treatment.

However, determining one or more skin-related characterizations can be performed in any suitable manner.

### 4.4 Determining a Therapy Model.

The method 100 can additionally or alternatively include Block S140, which can include generating a therapy model configured to modulate microorganism distributions in subjects characterized according to the characterization process. Block S140 can function to identify, rank, prioritize, determine, predict, discourage, and/or otherwise facilitate therapy determination for therapies (e.g., probiotic-based therapies, phage-based therapies, small molecule-based therapies, etc.), such as therapies that can shift a subject's microbiome composition and/or functional features (e.g., for microbiomes at any suitable sites, etc.) toward a desired equilibrium state in promotion of the subject's health, and/or determine therapies for otherwise modifying a state of one or more microorganism-related conditions (e.g., modifying a user behavior associated with a human behavior condition, etc.). Microorganism-related condition models can include one or more therapy models. In Block S140, the therapies can be selected from therapies including one or more of: probiotic therapies, phage-based therapies, small molecule-based therapies, cognitive/behavioral therapies, physical rehabilitation therapies, clinical therapies, medication-based therapies, diet-related therapies, and/or any other suitable therapy designed to operate in any other suitable manner in promoting a user's health. In a specific example of a bacteriophage-based therapy, one or more populations (e.g., in terms of colony forming units) of bacteriophages specific to a certain bacteria (or other microorganism) represented in the subject can be used to down-regulate or otherwise eliminate populations of the certain bacteria. As such, bacteriophage-based therapies can be used to reduce the size(s) of the undesired population(s) of bacteria represented in the subject. Complementarily, bacteriophage-based therapies can be used to increase the relative abundances of bacterial populations not targeted by the bacteriophage(s) used.

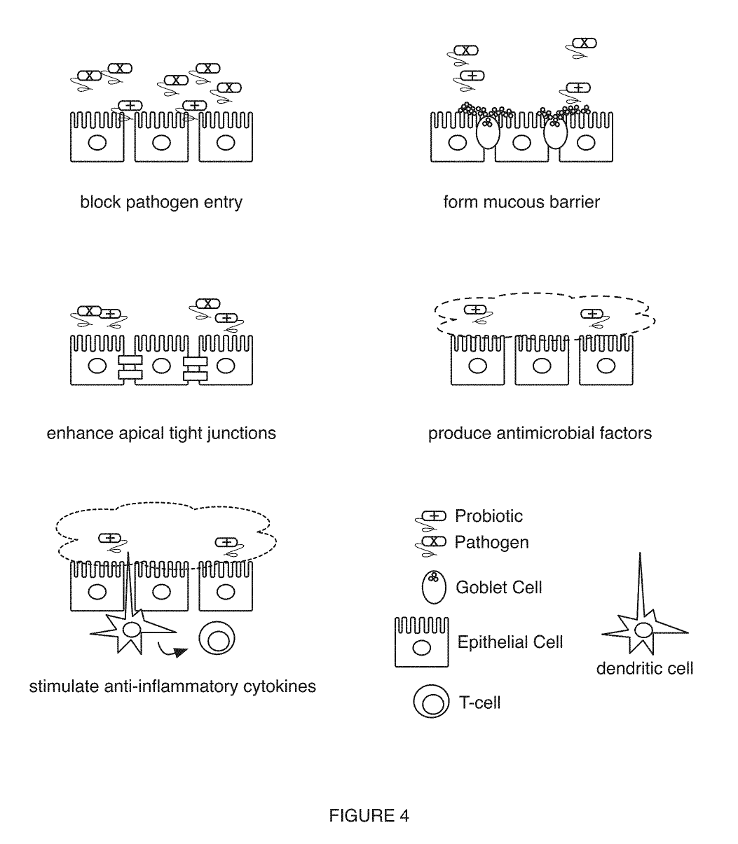

In another specific example of probiotic therapies, as shown in FIG. 4, candidate therapies of the therapy model can perform one or more of: blocking pathogen entry into an epithelial cell by providing a physical barrier (e.g., by way of colonization resistance), inducing formation of a mucous barrier by stimulation of goblet cells, enhance integrity of apical tight junctions between epithelial cells of a subject (e.g., by stimulating up regulation of zona-occludens 1, by preventing tight junction protein redistribution), producing antimicrobial factors, stimulating production of anti-inflammatory cytokines (e.g., by signaling of dendritic cells and induction of regulatory T-cells), triggering an immune response, and performing any other suitable function that adjusts a subject's microbiome away from a state of dysbiosis. In another specific example, therapies can include medical-device based therapies (e.g., associated with human behavior modification, associated with treatment of disease-related conditions, etc.).

In variations, the therapy model is preferably based upon data from a large population of subjects, which can include the population of subjects from which the microbiome diversity datasets are derived in Block S110, where microbiome composition and/or functional features or states of health, prior exposure to and post exposure to a variety of therapeutic measures, are well characterized. Such data can be used to train and validate the therapy provision model, in identifying therapeutic measures that provide desired outcomes for subjects based upon different microorganism-related characterizations. In variations, support vector machines, as a supervised machine learning algorithm, can be used to generate the therapy provision model. However, any other suitable machine learning algorithm described above can facilitate generation of the therapy provision model.

Additionally or alternatively, the therapy model can be derived in relation to identification of a “normal” or baseline microbiome composition and/or functional features, as assessed from subjects of a population of subjects who are identified to be in good health. Upon identification of a subset of subjects of the population of subjects who are characterized to be in good health (e.g., using features of the characterization process), therapies that modulate microbiome compositions and/or functional features toward those of subjects in good health can be generated in Block S140. Block S140 can thus include identification of one or more baseline microbiome compositions and/or functional features (e.g., one baseline microbiome for each of a set of demographics), and potential therapy formulations and therapy regimens that can shift microbiomes of subjects who are in a state of dysbiosis toward one of the identified baseline microbiome compositions and/or functional features. The therapy model can, however, be generated and/or refined in any other suitable manner.

Microorganism compositions associated with probiotic therapies associated with the therapy model preferably include microorganisms that are culturable (e.g., able to be expanded to provide a scalable therapy) and non-lethal (e.g., non-lethal in their desired therapeutic dosages). Furthermore, microorganism compositions can include a single type of microorganism that has an acute or moderated effect upon a subject's microbiome. Additionally or alternatively, microorganism compositions can include balanced combinations of multiple types of microorganisms that are configured to cooperate with each other in driving a subject's microbiome toward a desired state. For instance, a combination of multiple types of bacteria in a probiotic therapy can include a first bacteria type that generates products that are used by a second bacteria type that has a strong effect in positively affecting a subject's microbiome. Additionally or alternatively, a combination of multiple types of bacteria in a probiotic therapy can include several bacteria types that produce proteins with the same functions that positively affect a subject's microbiome.

Probiotic compositions can be naturally or synthetically derived. For instance, in one application, a probiotic composition can be naturally derived from fecal matter or other biological matter (e.g., of one or more subjects having a baseline microbiome composition and/or functional features, as identified using the characterization process and the therapy model). Additionally or alternatively, probiotic compositions can be synthetically derived (e.g., derived using a benchtop method) based upon a baseline microbiome composition and/or functional features, as identified using the characterization process and the therapy model. In variations, microorganism agents that can be used in probiotic therapies can include one or more of: yeast (e.g., Saccharomyces boulardii), gram-negative bacteria (e.g., E. coli Nissle), gram-positive bacteria (e.g., Bifidobacteria bifidum, Bifidobacteria infantis, Lactobacillus rhamnosus, Lactococcus lactis, Lactobacillus plantarum, Lactobacillus acidophilus, Lactobacillus casei, Bacillus polyfermenticus, etc.), and any other suitable type of microorganism agent.

In a variation, a therapy can include a probiotic therapy for one or more skin-related conditions (e.g., for improving a health state associated with the one or more skin-related conditions; etc.), where the probiotic therapy can include a combination of any one or more of: Corynebacterium ulcerans, Facklamia hominis, Corynebacterium sp., Propionibacterium sp. MSP09A, Facklamia sp. 1440-97, Staphylococcus sp. C9I2, Anaeroooccus sp. 9402080, Corynebacterium glucuronolyticum, Dermabacter hominis, Lactobacillus sp. BL302, Corynebacterium mastitidis, Bifidobacterium longum, Anaeroglobus geminatus, Anaerococcus sp. S9 PR-16, Prevotella timonensis, Kluyvera georgiana, Acinetobacter sp. WB22-23, Anaerococcus octavius, Finegoldia sp. S9 AA1-5, Staphylococcus sp. C-D-MA2, Peptoniphilus sp. 7-2, Cronobacter sakazakii, Anaerococcus sp. 8405254, Veillonella sp. CM60, Lactobacillus sp. 7_1_47FAA, Gemella sp. 933-88, Porphyromonas catoniae, Haemophilus parainfluenzae, Bacteroides sp. AR20, Bacteroides vulgatus, Bacteroides sp. D22, Dorea longicatena, Para bacteroides merdae, Bacteroides sp. AR29, Prevotella sp. WAL 2039G, Faecalibacterium prausnitzii, Blautia feeds, Alistipes putredinis, Bacteroides acidifaciens, Adlercreutzia equolifaciens, Phascolarctobacterium succinatutens, Roseburia inulinivorans, Phascolarctobacterium sp. 377, Desulfovibrio piger, Eggerthella sp. HGA1, Lactonifactor longoviformis, Alistipes sp. HGB5, Holdemania filiformis, Collinsella intestinalis, Neisseria macacae, Gemella sanguinis, Bacteroides fragilis, Prevotella oris, Pseudomonas brenneri, Flavobacterium ceti, Brevundimonas sp. FXJ8.080, Bacteroides plebeius, Varibaculum cambriense, Blautia vxexlerae, Staphylococcus sp. WB18-16, Streptococcus sp. oral taxon G63, Propionibacterium acnes, Anaerococcus sp. 9401487, Staphylococcus epidermidis, Campylobacter ureolyticus, Janibacter sp. M3-5, Peptoniphilus sp. DNF00840, Finegoldia sp. S8 F7, Prevotella disiens, Fusobacterium periodonticum, Corynebacterium freiburgense, Eremococcus coleocola, Streptococcus sp. 68353, Finegoldia magna, Staphylococcus aureus, Haemophilus influenzae, Corynebacterium sp. NML 97-0186, Streptococcus sp. oral taxon G59, Roseburia sp. 11SE39, Catenibacterium mitsuokai, Collinsella aerofaciens, Peptoniphilus sp. 2002-2300004, Corynebacterium canis, Prevotella buccalis, Dialister invisus, Neisseria mucosa, and/or any other suitable microorganisms of any suitable taxon (e.g., described herein) and/or phage vector (e.g., bacteriophage, virus, etc.). In a specific example, the probiotic therapy and/or other suitable probiotic therapies can be promoted (e.g., recommended; otherwise provided; etc.) at dosages of 0.1 million to 10 billion CPUs, as determined from a therapy model that predicts positive adjustment of a patient's microbiome in response to the therapy. In examples, a subject can be instructed to ingest capsules comprising the probiotic formulation according to a regimen tailored to one or more of his/her: physiology (e.g., body mass index, weight, height), demographics (e.g., gender, age), severity of dysbiosis, sensitivity to medications, and/or any other suitable factor.

In a variation, for subjects who exhibit on or more skin related-conditions including one or more photosensitivity-associated conditions, dry skin-associated conditions, scalp-related conditions, and/or other suitable skin-related conditions, microorganisms associated with a skin-related condition can provide a dataset based on composition or diversity of recognizable patterns of relative abundance in microorganisms that are present in subject microbiome, and can be used as a diagnostic tool and/or therapeutic tool using bioinformatics pipelines and/or characterizations describe above.

In another variation, microorganism datasets (e.g., based on composition or diversity of recognizable patterns of relative abundance in microorganisms that are present in subject microbiome) can be used as a diagnostic tool using bioinformatics pipelines and characterization describe above. However, probiotic therapies and/or other suitable therapies can include any suitable combination of microorganisms associated with any suitable taxa described herein.

Probiotics and/or other suitable consumables can be provided at dosages of 0.1 million to 10 billion CPUs (and/or other suitable dosages), such as determined from a therapy model that predicts positive adjustment of a patient's microbiome in response to the therapy. In a specific example, a subject can be instructed to ingest capsules including the probiotic formulation according to a regimen tailored to one or more of his/her: physiology (e.g., body mass index, weight, height), demographics (e.g., gender, age), severity of dysbiosis, sensitivity to medications, and any other suitable factor. For subjects who exhibit a microorganism-related condition, associated-microorganisms (e.g., corresponding to correlated microbiome composition features) can provide a dataset based on composition and/or diversity of recognizable patterns of relative abundance in microorganisms that are present in subject microbiome, and can be used as a diagnostic tool using bioinformatics pipelines and characterization describe above.

### 4.5 Processing a User Biological Sample.

The method 100 can additionally or alternatively include Block S150, which can include processing one or more biological samples from a user (e.g., biological samples from different collection sites of the user, etc.). Block S150 can function to facilitate generation of a microorganism dataset for the subject, such as for use in deriving inputs for the characterization process (e.g., for generating a microorganism-related characterization for the user, such as through applying one or more microbiome characterization modules, etc.). As such, Block S150 can include receiving, processing, and/or analyzing one or more biological samples from one or more users (e.g., multiple biological samples for the same user over time, different biological samples for different users, etc.). In Block S150, the biological sample is preferably generated from the subject and/or an environment of the subject in a non-invasive manner. In variations, non-invasive manners of sample reception can use any one or more of: a permeable substrate (e.g., a swab configured to wipe a region of a subject's body, toilet paper, a sponge, etc.), a non-permeable substrate (e.g., a slide, tape, etc.) a container (e.g., vial, tube, bag, etc.) configured to receive a sample from a region of a subject's body, and any other suitable sample-reception element. In a specific example, the biological sample can be collected from one or more of the subject's nose, skin, genitals, mouth, and gut in a non-invasive manner (e.g., using a swab and a vial). However, the biological sample can additionally or alternatively be received in a semi-invasive manner or an invasive manner. In variations, invasive manners of sample reception can use any one or more of: a needle, a syringe, a biopsy element, a lance, and any other suitable instrument for collection of a sample in a semi-invasive or invasive manner. In specific examples, samples can include Mood samples, plasma/serum samples (e.g., to enable extraction of cell-free DNA), and tissue samples.

In the above variations and examples, the biological sample can be taken from the body of the subject without facilitation by another entity (e.g., a caretaker associated with a subject, a health care professional, an automated or semi-automated sample collection apparatus, etc.), or can alternatively be taken from the body of the subject with the assistance of another entity. In one example, where the biological sample is taken from the subject without facilitation by another entity in the sample extraction process, a sample-provision kit can be provided to the subject. In the example, the kit can include one or more swabs for sample acquisition, one or more containers configured to receive the swab(s) for storage, instructions for sample provision and setup of a user account, elements configured to associate the sample(s) with the subject (e.g., barcode identifiers, tags, etc.), and a receptacle that allows the sample(s) from the subject to be delivered to a sample processing operation (e.g., by a mail delivery system). In another example, where the biological sample is extracted from the subject with the help of another entity, one or more samples can be collected in a clinical or research setting from the subject (e.g., during a clinical appointment). The biological sample can, however, be received from the subject in any other suitable manner.

Furthermore, processing and analyzing the biological sample (e.g., to generate a user microorganism dataset; etc.) from the subject is preferably performed in a manner similar to that of one of the embodiments, variations, and/or examples of sample reception described in relation to Block S110 above, and/or any other suitable portions of the method 100. As such, reception and processing of the biological sample in Block S150 can be performed for the subject using similar processes as those for receiving and processing biological samples used to generate the characterization process and/or the therapy model of the method 100, in order to provide consistency of process. However, biological sample reception and processing in Block S150 can alternatively be performed in any other suitable manner.

### 4.6 Determining a Microorganism-Related Characterization.

The method 100 can additionally or alternatively include Block S160, which can include determining, with the characterization process, a microorganism-related characterization for the user, such as based upon processing one or more microorganism dataset (e.g., user microorganism sequence dataset, microbiome composition dataset, microbiome functional diversity dataset; processing of the microorganism dataset to extract microbiome features; etc.) derived from the biological sample of the user. Block S160 can function to characterize one or more microorganism-related conditions for a user, such as through extracting features from microbiome-derived data of the subject, and using the features as inputs into an embodiment, variation, or example of the characterization process described in Block S130 above (e.g., using the user microbiome feature values as inputs into a microbiome-related condition characterization model, etc.). In an example, Block S160 can include generating a microorganism-related characterization for the user based on user microbiome features and a microorganism-related condition characterization model (e.g., generated in Block S130). Microorganism-related characterizations can be for any number and/or combination of microorganism-related conditions (e.g., a combination of microorganism-related conditions, a single microorganism-related condition, and/or other suitable microorganism-related conditions; etc.). Microorganism-related characterizations can include one or more of: diagnoses (e.g., presence or absence of a microorganism-related condition; etc.); risk (e.g., risk scores for developing and/or the presence of a microorganism-related condition; information regarding microorganism-related characterizations (e.g., symptoms, signs, triggers, associated conditions, etc.); comparisons (e.g., comparisons with other subgroups, populations, users, historic health statuses of the user such as historic microbiome compositions and/or functional diversities; comparisons associated with microorganism-related conditions; etc.), and/or any other suitable data.

In another variation, a microorganism-related characterization can include a microbiome diversity score (e.g., in relation to microbiome composition, function, etc.) associated with (e.g., correlated with; negatively correlated with; positively correlated with; etc.) a microbiome diversity score correlated with one or more microorganism-related conditions. In examples, the microorganism-related characterization can include microbiome diversity scores over time (e.g., calculated for a plurality of biological samples of the user collected over time), comparisons to microbiome diversity scores for other users, and/or any other suitable type of microbiome diversity score. However, processing microbiome diversity scores (e.g., determining microbiome diversity scores; using microbiome diversity scores to determine and/or provide therapies; etc.) can be performed in any suitable manner.

Determining a microorganism-related characterization in Block S160 preferably includes identifying features and/or combinations of features associated with the microbiome composition and/or functional features of the subject, inputting the features into the characterization process, and receiving an output that characterizes the subject as belonging to one or more of: a behavioral group, a gender group, a dietary group, a disease-state group, and any other suitable group capable of being identified by the characterization process. Block S160 can additionally or alternatively include generation of and/or output of a confidence metric associated with the characterization of the subject. For instance, a confidence metric can be derived from the number of features used to generate the characterization, relative weights or rankings of features used to generate the characterization, measures of bias in the characterization process, and/or any other suitable parameter associated with aspects of the characterization process. However, leveraging user microbiome features can be performed in any suitable manner to generate any suitable microorganism-related characterizations.

In some variations, features extracted from the microorganism dataset of the subject can be supplemented with supplementary features (e.g., extracted from supplementary data collected for the user; such as survey-derived features, medical history-derived features, sensor data, etc.), where such data, the user microbiome data, and/or other suitable data can be used to further refine the characterization process of Block S130, Block S160, and/or other suitable portions of the method 100.

Determining a microorganism-related characterization preferably includes extracting and applying user microbiome features (e.g., user microbiome composition diversity features; user microbiome functional diversity features; etc.) for the user (e.g., based on a user microorganism dataset), characterization models, and/or other suitable components, such as by employing approaches described in Block S130, and/or by employing any suitable approaches described herein.

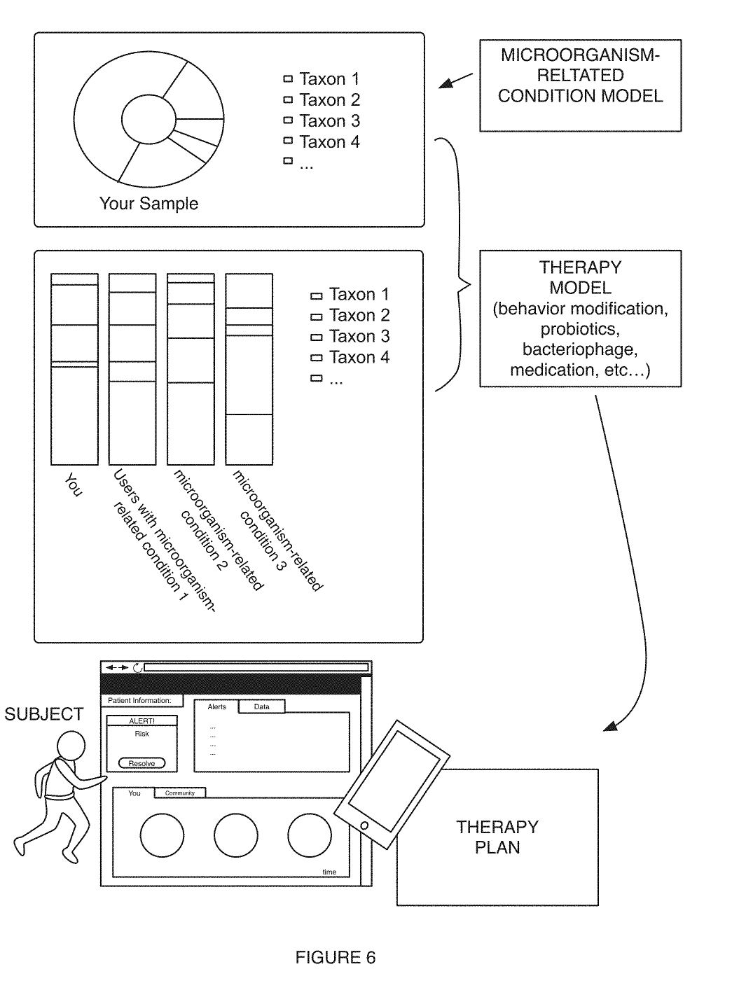

In variations, as shown in FIG. 6, Block S160 can include presenting microorganism-related characterizations (e.g., information extracted from the characterizations, etc.), such as at a web interface, a mobile application, and/or any other suitable interface, but presentation of information can be performed in any suitable manner. However, the microorganism dataset of the subject can additionally or alternatively be used in any other suitable manner to enhance the models of the method 100, and Block S160 can be performed in any suitable manner.

### 4.7 Facilitating Therapeutic Intervention.

As shown in FIG. 9, the method 100 can additionally or alternatively include Block S170, which can include facilitating therapeutic intervention (e.g., promoting therapies, providing therapies, facilitating provision of therapies, etc.) for one or more microorganism-related conditions for one or more users (e.g., based upon a microorganism-related characterization and/or a therapy model). Block S170 can function to recommend, promote, provide, and/or otherwise facilitate therapeutic intervention in relation to one or more therapies for a user, such as to shift the microbiome composition and/or functional diversity of a user toward a desired equilibrium state (and/or otherwise improving a state of the microorganism-related condition, etc.) in relation to one or more microorganism-related conditions. Block S170 can include provision of a customized therapy to the subject according to their microbiome composition and functional features, where the customized therapy can include a formulation of microorganisms configured to correct dysbiosis characteristic of subjects having the identified characterization. As such, outputs of Block S140 can be used to directly promote a customized therapy formulation and regimen (e.g., dosage, usage instructions) to the subject based upon a trained therapy model. Additionally or alternatively, therapy provision can include recommendation of available therapeutic measures configured to shift microbiome composition and/or functional features toward a desired state. In variations, therapies can include any one or more of: consumables, topical therapies (e.g., lotions, ointments, antiseptics, etc.), medication (e.g., medications associated with any suitable medication type and/or dosage, etc.), bacteriophages, environmental treatments, behavioral modification (e.g., diet modification therapies, stress-reduction therapies, physical activity-related therapies, etc.), diagnostic procedures, other medical-related procedures, and/or any other suitable therapies associated with microorganism-related conditions. Consumables can include any-one or more of: food and/or beverage items (e.g., probiotic and/or prebiotic food and/or beverage items, etc.), nutritional supplements (e.g., vitamins, minerals, fiber, fatty acids, amino acids, probiotics, probiotics, etc.), consumable medications, and/or any other suitable therapeutic measure.

For example, a combination of commercially available probiotic supplements can include a suitable probiotic therapy for the subject according to an output of the therapy model. In another example, the method 100 can include determining a microorganism-related condition risk for the user for the microorganism-related condition based on a microorganism-related condition model (e.g., and/or user microbiome features); and promoting a therapy to the user based on the microorganism-related condition risk.

In a variation, promoting a therapy can include promoting a diagnostic procedure (e.g., for facilitating detection of microorganism-related conditions such as human behavior conditions and/or disease-related conditions, which can motivate subsequent promotion of other therapies, such as for modulation of a user microbiome for improving a user health state associated with one or more microorganism-related conditions; etc.). Diagnostic procedures can include any one or more of: medical history analyses, imaging examinations, cell culture tests, antibody tests, skin prick testing, patch testing, blood testing, challenge testing, performing portions of the method 100, and/or any other suitable procedures for facilitating the detecting (e.g., observing, predicting, etc.) of microorganism-related conditions. Additionally or alternatively, diagnostic device-related information and/or other suitable diagnostic information can be processed as part of a supplementary dataset (e.g., in relation to Block S120, where such data can be used in determining and/or applying characterization models, therapy models, and/or other suitable models; etc.), and/or collected, used, and/or otherwise processed in relation to any suitable portions of the method 100 (e.g., administering diagnostic procedures for users for monitoring therapy efficacy in relation to Block S180; etc.)

In another variation, Block S170 can include promoting a bacteriophage-based therapy. In more detail, one or more populations (e.g., in terms of colony forming units) of bacteriophages specific to a certain bacteria (or other microorganism) represented in the subject can be used to down-regulate or otherwise eliminate populations of the certain bacteria. As such, bacteriophage-based therapies can be used to reduce the size(s) of the undesired population(s) of bacteria represented in the subject. Complementarily, bacteriophage-based therapies can be used to increase the relative abundances of bacterial populations not targeted by the bacteriophage(s) used.

In another variation, therapy provision in Block S170 can include provision of notifications to a subject regarding the recommended therapy, other forms of therapy, microorganism-related characterizations, and/or other suitable data. In a specific example, providing a therapy to a user can include providing therapy recommendations (e.g., substantially concurrently with providing information derived from a microorganism-related characterization for a user; etc.) and/or other suitable therapy-related information (e.g., therapy efficacy; comparisons to other individual users, subgroups of users, and/or populations of users; therapy comparisons; historic therapies and/or associated therapy-related information; psychological therapy guides such as for cognitive behavioral therapy; etc.), such as through presenting notifications at a web interface (e.g., through a user account associated with and identifying a user; etc.). Notifications can be provided to a subject by way of an electronic device (e.g., personal computer, mobile device, tablet, wearable, head-mounted wearable computing device, wrist-mounted wearable computing device, etc.) that executes an application, web interface, and/or messaging client configured for notification provision. In one example, a web interface of a personal computer or laptop associated with a subject can provide access, by the subject, to a user account of the subject, where the user account includes information regarding the user's microorganism-related characterization, detailed characterization of aspects of the user's microbiome (e.g., in relation to correlations with microorganism-related conditions; etc.), and/or notifications regarding suggested therapeutic measures (e.g., generated in Blocks S140 and/or S170, etc.). In another example, an application executing at a personal electronic device (e.g., smart phone, smart watch, head-mounted smart device) can be configured to provide notifications (e.g., at a display, haptically, in an auditory manner, etc.) regarding therapy suggestions generated by the therapy model of Block S170. Notifications and/or probiotic therapies can additionally or alternatively be provided directly through an entity associated with a subject (e.g., a caretaker, a spouse, a significant other, a healthcare professional, etc.). In some further variations, notifications can additionally or alternatively be provided to an entity (e.g., healthcare professional) associated with a subject, such as where the entity is able to facilitate provision of the therapy (e.g., by way of prescription, by way of conducting a therapeutic session, through a digital telemedicine session using optical and/or audio sensors of a computing device, etc.). Promoting notifications and/or other suitable therapies can, however, be performed in any suitable manner.

### 4.8 Monitoring Therapy Effectiveness.

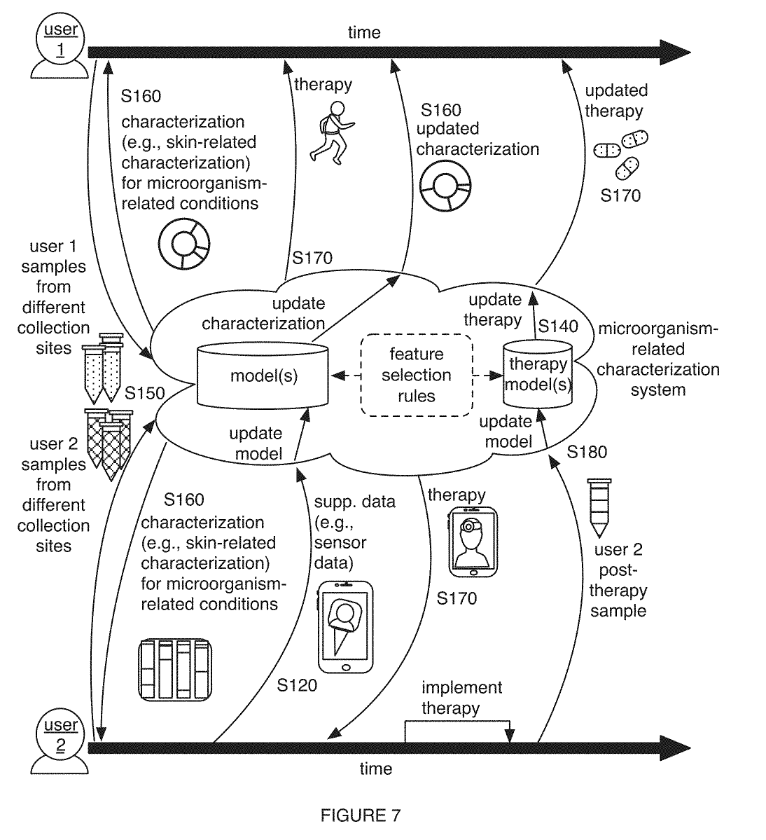

As shown in FIG. 7, the method can additionally or alternatively include Block S180, which recites: monitoring effectiveness of the therapy for the subject, based upon processing biological samples, to assess microbiome composition and/or functional features for the subject at a set of time points associated with the probiotic therapy. Block S180 can function to gather additional data regarding positive effects, negative effects, and/or lack of effectiveness of a probiotic therapy suggested by the therapy model for subjects of a given characterization. Monitoring of a subject during the course of a therapy promoted by the therapy model (e.g., by receiving and analyzing biological samples from the subject throughout therapy, by receiving survey-derived data from the subject throughout therapy) can thus be used to generate a therapy-effectiveness model for each characterization provided by the characterization process of Block S130, and each recommended therapy measure provided in Blocks S140 and S170.

In Block S180, the subject can be prompted to provide additional biological samples at one or more key time points of a therapy regimen that incorporates the therapy, and the additional biological sample(s) can be processed and analyzed (e.g., in a manner similar to that described in relation to Block S120) to generate metrics characterizing modulation of the subject's microbiome composition and/or functional features. For instance, metrics related to one or more of: a change in relative abundance of one or more taxonomic groups represented in the subject's microbiome at an earlier time point, a change in representation of a specific taxonomic group of the subject's microbiome, a ratio between abundance of a first taxonomic group of bacteria and abundance of a second taxonomic group of bacteria of the subject's microbiome, a change in relative abundance of one or more functional families in a subject's microbiome, and any other suitable metrics can be used to assess therapy effectiveness from changes in microbiome composition and/or functional features. Additionally or alternatively, survey-derived data from the subject, pertaining to experiences of the subject while on the therapy (e.g., experienced side effects, personal assessment of improvement, behavioral modifications, symptom improvement, etc.) can be used to determine effectiveness of the therapy in Block S180. For example, the method 100 can include receiving a post-therapy biological sample from the user; collecting a supplementary dataset from the user, where the supplementary dataset describes user adherence to a therapy (e.g., a determined and promoted therapy) and/or other suitable user characteristics (e.g., behaviors, conditions, etc.); generating a post-therapy microorganism-related characterization of the first user in relation to the microorganism-related condition based on the microorganism-related condition characterization model and the post-therapy biological sample; and promoting an updated therapy to the user for the microorganism-related condition based on the post-therapy microorganism-related characterization (e.g., based on a comparison between the post-therapy microorganism-related characterization and a pre-therapy microorganism-related characterization; etc.) and/or the user adherence to the therapy (e.g., modifying the therapy based on positive or negative results for the user microbiome in relation to the microorganism-related condition; etc.). Additionally or alternatively, other suitable data (e.g., supplementary data describing user behavior associated with the human behavior condition; supplementary data describing a disease-related condition such as observed symptoms; etc.) can be used in determining a post-therapy characterization (e.g., degree of change from pre- to post-therapy in relation to the microorganism-related condition; etc.), updated therapies (e.g., determining an updated therapy based on effectiveness and/or adherence to the promoted therapy, etc.). Therapy effectiveness, processing of additional biological samples (e.g., to determine additional microorganism-related characterizations, therapies, etc.), and/or other suitable aspects associated with continued biological sample collection, processing, and analysis in relation to microorganism-related conditions can be performed at any suitable time and frequency for generating, updating, and/or otherwise processing models (e.g., characterization models, therapy models, etc.), and/or for any other suitable purpose (e.g., as inputs associated with other portions of the method 100). However, Block S180 can be performed in any suitable manner.

The method 100 can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microbiome compositions and/or functional features of subjects.

Embodiments of the system and/or method can include every combination and permutation of the various system components and the various method processes, including any variations, examples, and specific examples, where the method and/or processes described herein can be performed asynchronously (e.g., sequentially), concurrently (e.g., in parallel), or in any other suitable order by and/or using one or more instances of the systems, elements, and/or entities described herein.

Any of the variants described herein (e.g., embodiments, variations, examples, specific examples, illustrations, etc.) and/or any portion of the variants described herein can be additionally or alternatively combined, excluded, and/or otherwise applied.

The system and method and embodiments thereof can be embodied and/or implemented at least in part as a machine configured to receive a computer-readable medium storing computer-readable instructions. The instructions are preferably executed by computer-executable components preferably integrated with the system. The computer-readable medium can be stored on any suitable computer-readable media such as RAMs, ROMs, flash memory, EEPROMs, optical derices (CD or DVD), hard drives, floppy drives, or any suitable device. The computer-executable component is preferably a general or application specific processor, but any suitable dedicated hardware or hardware/firmware combination device can alternatively or additionally execute the instructions.

As a person skilled in the art will recognize from the previous detailed description and from the figures and claims, modifications and changes can be made to the embodiments without departing from the scope defined in the following claims.

TABLE 1

| | Technique | | |
|---|---|---|---|
| | | Zero-inflated | |
| | Beta | Beta- | |
| Site: genital | binomial | binomial | Kolmogorov- |
| Taxa name | regression | regression | Smirnov test |
| Bifidobacterium | — | X | — |
| Mobiluncus curtisii | — | X | — |
| Neisseriaceae | — | X | — |
| Neisseriales | — | X | — |
| Prevotella sp. WAL 2039G | — | X | — |
| Propionibacterium sp. | — | X | — |
| MSP09A | | | |
| Pseudomonadaceae | — | X | — |
| Pseudomonas | — | X | — |
| Staphylococcus sp. 334802 | — | X | — |
| Streptococcus sp. oral taxon | — | X | — |
| G59 | | | |

TABLE 2

| | Technique | | |
|---|---|---|---|
| | | Zero-inflated | |
| | Beta | Beta- | |
| Site: gut | binomial | binomial | Kolmogorov- |
| Taxa name | regression | regression | Smirnov test |
| Acidaminococcus | — | X | — |
| intestini | | | |
| Actinobacteria | X | X | X |
| Actinobacteria | X | X | X |
| Actinomycetaceae | — | X | — |
| Aeromonadales | — | X | — |
| Alistipes | X | X | — |
| putredinis | | | |
| Alistipes sp. | X | — | — |
| EBA6-25cl2 | | | |
| Alistipes sp. | X | — | — |
| HGB5 | | | |
| Anaerococcus | — | X | — |
| Anaerosinus | — | X | — |
| Anaerostipes | — | X | — |
| butyraticus | | | |
| Anaerostipes | X | X | X |
| sp. | | | |
| 5_1_63FAA | | | |
| Arcanobacterium | — | X | — |
| Bacillales | X | — | — |
| Bacteroidaceae | X | — | — |
| Bacteroides | X | — | — |
| Bacteroides | X | X | — |
| plebeius | | | |
| Bacteroides | X | — | — |
| sp. AR20 | | | |
| Bacteroides | X | X | — |
| sp. AR29 | | | |
| Bacteroides | X | — | — |
| sp. D22 | | | |
| Bacteroides | X | — | — |
| sp. | | | |
| DJF_B097 | | | |
| Bacteroides | — | X | — |
| vulgatus | | | |
| Betaproteobacteria | X | — | — |
| Bifidobacteriaceae | X | X | X |
| Bifidobacteriales | X | X | X |
| Bifidobacterium | X | X | X |
| Bifidobacterium | X | X | — |
| kashiwanohense | | | |
| Bifidobacterium stercoris | X | X | X |
| Blautia luti | X | X | X |
| Blautia sp. | X | — | — |
| Ser8 | | | |
| Blautia | X | X | X |
| wexlerae | | | |
| Burkholderiales | X | X | — |
| Candidatus | — | X | — |
| Saccharibacteria | | | |
| Candidatus | X | — | — |
| Soleaferrea | | | |
| Cloacibacillus evryensis | — | X | — |
| Clostridium | X | X | — |
| Collinsella | X | X | X |
| Collinsella | X | — | X |
| aerofaciens | | | |
| Coriobacteriaceae | X | X | X |
| Coriobacteriales | X | X | X |
| Dielma | X | X | — |
| Dorea | X | X | X |
| Dorea | X | — | — |
| formicigenerans | | | |
| Dorea | X | X | X |
| longicatena | | | |
| Eisenbergiella | X | X | X |
| Eisenbergiella tayi | X | — | X |
| Enterobacter | — | X | — |
| Enterobacteriaceae | X | X | — |
| Enterobacteriales | X | X | — |
| Erysipelatoclostridium | X | X | X |
| Erysipelatoclostridium | X | — | — |
| ramosum | | | |
| Erysipelotrichaceae | X | X | X |
| Erysipelotrichales | X | X | X |
| Erysipelotrichia | X | X | X |
| Faecalibacterium | X | X | — |
| Faecalibacterium | X | X | X |
| prausnitzii | | | |
| Finegoldia | — | X | — |
| Flavonifractor | X | X | X |
| Flavonifractor plautii | X | X | X |
| Fusicatenibacter | X | X | X |
| Fusicatenibacter | X | X | X |
| saccharivorans | | | |
| Gammaproteobacteria | — | X | — |
| Gardnerella | — | X | — |
| Haemophilus | X | X | — |
| Haemophilus influenzae | — | X | — |
| Haemophilus | X | X | — |
| parainfluenzae | | | |
| Intestinibacter | X | — | — |
| Kluyvera | X | X | — |
| Kluyvera | X | X | — |
| georgiana | | | |
| Lachnospira | X | — | — |
| Lachnospira | X | — | — |
| pectinoschiza | | | |
| Lactobacillaceae | X | X | X |
| Lactobacillus | X | X | — |
| Lactobacillus | — | X | — |
| sp. Aklamro1 | | | |
| Megasphaera | X | X | — |
| Moryella | X | X | — |
| Odoribacter | X | — | — |
| splanchnicus | | | |
| Oscillospira | X | — | X |
| Oscillospiraceae | X | X | — |
| Parabacteroides merdae | X | X | — |
| Pasteureliaceae | X | X | — |
| Pasteurellales | X | X | — |
| Peptococcaceae | X | — | — |
| Peptococcus | X | — | — |
| Peptoniphilus | — | X | — |
| Phascolarctobacterium | X | X | — |
| faecium | | | |
| Porphyromonas | — | X | — |
| Prevotella | — | X | — |
| Roseburia | X | X | — |
| inulinivorans | | | |
| Roseburia | X | X | X |
| sp. 11SE39 | | | |
| Ruminococcaceae | X | X | — |
| Staphylococcaceae | — | X | — |
| Staphylococcus | — | X | — |
| Staphylococcus sp. C9I2 | — | X | — |
| Streptococcus | — | X | — |
| pasteurianus | | | |
| Subdoligranulum | X | X | X |
| Subdoligranulum | X | X | X |
| variabile | | | |
| Succinivibrionaceae | — | X | — |
| Sutterella | X | — | — |
| Sutterellaceae | X | — | — |

TABLE 3

| | Technique | | |
|---|---|---|---|
| | | Zero-inflated | |
| | Beta | Beta- | |
| Site: mouth | binomial | binomial | Kolmogorov- |
| Taxa name | regression | regression | Smirnov test |
| Acinetobacter | — | X | — |
| Bacteroides | — | X | — |
| fragilis | | | |
| Gemella | — | X | — |
| sanguinis | | | |
| Veillonella | — | X | — |
| sp. | | | |
| 2011_Oral_ | | | |
| VSA_D3 | | | |

TABLE 4

| | Technique | | |
|---|---|---|---|
| | | Zero-inflated | |
| | Beta | Beta- | |
| Site: nose | binomial | binomial | Kolmogorov- |
| Taxa name | regression | regression | Smirnov test |
| Bacillus | — | X | — |
| Bacteroides | — | X | — |
| fragilis | | | |
| Corynebacterium | — | X | — |
| atypicum | | | |
| Corynebacterium | — | X | — |
| spheniscorum | | | |
| Enterobacteriaceae | — | X | — |
| Enterobacteriales | — | X | — |
| Fusobacteria | — | X | — |
| Fusobacteriales | — | X | — |
| Fusobacteriia | — | X | — |
| Kluyvera | — | X | — |
| Kluyvera | — | X | — |
| georgiana | | | |
| Lachnospiraceae | — | X | — |
| Lactobacillus | — | X | — |
| sp. | | | |
| 7_1_47FAA | | | |
| Leuconostoc | — | X | — |
| Leuconostocaceae | — | X | — |
| Roseburia | — | X | — |
| faecis | | | |

TABLE 5

| | Technique | | |
|---|---|---|---|
| | | Zero-inflated | |
| | Beta | Beta- | |
| Site: skin | binomial | binomial | Kolmogorov- |
| Taxa name | regression | regression | Smirnov test |
| Aerococcaceae | — | X | — |
| Anaerococcus sp. | — | X | — |
| 9401487 | | | |
| Bacteroidaceae | — | X | — |
| Bacteroides | — | X | — |
| Bifidobacteriaceae | — | X | — |
| Bifidobacteriales | — | X | — |
| Bifidobacterium stercoris | — | X | — |
| Gemella | — | X | — |
| morbillorum | | | |
| Klebsiella | — | X | — |
| Lachnospiraceae | — | X | — |
| Lactobacillus | — | X | — |
| crispatus | | | |
| Massilia | — | X | — |
| Neisseria macacae | — | X | — |
| Peptoniphilus sp. | — | X | — |
| DNF00840 | | | |
| Porphyromonas | — | X | — |
| Porphyromonas bennonis | — | X | — |
| Staphylococcus | — | X | — |
| epidermidis | | | |
| Streptococcus sp. oral | — | X | — |
| taxon G63 | | | |

TABLE 6

| | Technique | | |
|---|---|---|---|
| | | Zero-inflated | |
| | Beta | Beta- | |
| Site: genital | binomial | binomial | Kolmogorov- |
| Taxa name | regression | regression | Smirnov test |
| Anaerococcus sp. S9 PR- | — | X | — |
| 16 | | | |
| Atopobium | — | X | — |
| vaginae | | | |
| Corynebacteriaceae | — | X | — |
| Corynebacterium | — | X | — |
| Corynebacterium | — | X | — |
| freiburgense | | | |
| Corynebacterium | — | X | — |
| glucuronolyticum | | | |
| Corynebacterium sp. | — | X | — |
| Corynebacterium sp.jw37 | — | X | — |
| Facklamia | X | — | — |
| Facklamia | X | X | — |
| hominis | | | |
| Kluyvera | — | X | — |
| Kluyvera | — | X | — |
| georgiana | | | |
| Lactobacillus | — | X | — |
| fornicalis | | | |
| Lactobacillus | — | X | — |
| sp. | | | |
| 7_1_47FAA | | | |
| Neisseria | — | X | — |
| Neisseriaceae | — | X | — |
| Neisseriales | — | X | — |
| Peptoniphilus sp. oral | — | X | — |
| taxon 836 | | | |
| Propionibacteriaceae | — | X | — |
| Propionibacterium | — | X | — |
| Propionibacterium sp. | — | X | — |
| MSP09A | | | |
| Staphylococcus sp. C9I2 | — | X | — |

TABLE 7

| | Technique | | |
|---|---|---|---|
| | | Zero- | |
| | | inflated | |
| | Beta | Beta- | Kolmogorov- |
| Site: gut | binomial | binomial | Smirnov |
| Taxa name | regression | regression | test |
| Actinobacteria | X | X | X |
| Actinobacteria | X | X | X |
| Actinomycetales | X | X | X |
| Alistipes | X | — | — |
| Alistipes finegoldii | X | X | — |
| Alistipes | X | X | — |
| indistinctus | | | |
| Alistipes putredinis | X | X | X |
| Alistipes shahii | X | X | — |
| Alistipes sp. EBA6- | X | X | — |
| 25cl2 | | | |
| Alistipes sp. HGB5 | X | X | — |
| Alistipes sp. RMA | — | X | — |
| 9912 | | | |
| Alloscardovia | X | X | — |
| Alloscardovia | X | X | — |
| omnicolens | | | |
| Alphaproteobacteria | X | X | — |
| Anaerococcus | — | X | — |
| Anaerococcus sp. | X | — | — |
| 8404299 | | | |
| Anaerosporobacter | X | X | — |
| mobilis | | | |
| Anaerostipes | X | — | X |
| Anaerostipes sp. | X | X | — |
| 3_2_56FAA | | | |
| Anaerostipes sp. | X | X | X |
| 5_1_63FAA | | | |
| Anaerotruncus sp. | X | X | — |
| NML 070203 | | | |
| Bacillaceae | — | X | — |
| Bacillales | X | — | — |
| Bacteroidaceae | X | X | X |
| Bacteroides | X | X | X |
| Bacteroides caccae | X | — | — |
| Bacteroides dorei | X | — | — |
| Bacteroides faecis | X | X | — |
| Bacteroides fragilis | X | X | — |
| Bacteroides | — | X | — |
| intestinalis | | | |
| Bacteroides | — | X | — |
| massiliensis | | | |
| Bacteroides | X | X | — |
| plebeius | | | |
| Bacteroides sp. | X | X | — |
| 35A-E37 | | | |
| Bacteroides sp. | X | X | — |
| AR20 | | | |
| Bacteroides sp. | X | X | — |
| AR29 | | | |
| Bacteroides sp. D20 | X | X | — |
| Bacteroides sp. D22 | X | X | — |
| Bacteroides sp. | X | X | — |
| DJW_B097 | | | |
| Bacteroides sp. | X | X | — |
| ERA5-17 | | | |
| Bacteroides sp. | X | X | X |
| SLC1-38 | | | |
| Bacteroides | X | X | X |
| thetaiotaomicron | | | |
| Bacteroides | — | X | — |
| uniformis | | | |
| Bacteroides | X | — | — |
| vulgatus | | | |
| Barnesiella | X | — | — |
| intestinihominis | | | |
| Betaproteobacteria | X | X | — |
| Bifidobacteriaceae | X | — | X |
| Bifidobacteriales | X | — | X |
| Bifidobacterium | X | X | X |
| Bifidobacterium | X | X | — |
| kashiwanohense | | | |
| Bifidobacterium | X | X | — |
| stercoris | | | |
| Bilophila | — | X | — |
| Bilophila sp. | X | — | — |
| 4_1_30 | | | |
| Blautia glucerasea | X | — | — |
| Blautia hansenii | X | X | — |
| Blautia | X | X | — |
| hydrogenotrophica | | | |
| Blautia luti | X | X | X |
| Blautia sp. Ser8 | X | X | — |
| Blautia sp. YHC-4 | X | X | X |
| Blautia stercoris | X | X | — |
| Blautia wexlerae | X | — | — |
| Burkholderiales | X | X | — |
| Butyricimonas | X | X | — |
| Butyricimonas sp. | X | X | — |
| JCM 18677 | | | |
| Campylobacter | X | — | — |
| Campylobacteraceae | X | — | — |
| Campylobacterales | X | — | — |
| Candidatus | X | X | — |
| Soleaferrea | | | |
| Carnobacteriaceae | — | X | — |
| Clostridia | X | X | — |
| Clostridiales | X | X | — |
| Clostridiales Family | — | X | — |
| XI. Incertae Sedis | | | |
| Clostridium | X | — | — |
| Collinsella | X | X | X |
| Collinsella | X | X | X |
| aerofaciens | | | |
| Coriobacteriaceae | X | X | X |
| Coriobacteriales | X | X | X |
| Corynebacteriaceae | X | X | X |
| Corynebacterium | X | X | X |
| Corynebacterium canis | X | X | — |
| Corynebacterium | X | X | — |
| epidermidicanis | | | |
| Corynebacterium | X | X | — |
| sp. | | | |
| Corynebacterium | — | X | — |
| spheniscorum | | | |
| Cyanobacteria | — | X | — |
| Deltaproteobacteria | X | X | — |
| Desulfovibrio | X | X | — |
| desulfuricans | | | |
| Desulfovibrio piger | X | X | — |
| Desulfovibrio naceae | X | X | — |
| Desulfovibrionales | X | X | — |
| Dialister | X | — | — |
| Dialister | X | — | — |
| propionicifaciens | | | |
| Dielma | X | — | — |
| Dorea | X | X | X |
| Dorea | X | X | — |
| formicigenerans | | | |
| Dorea longicatena | X | X | X |
| Eggerthella | X | X | X |
| Eggerthella lenta | X | X | — |
| Eggerthella sp. | X | X | — |
| HGA1 | | | |
| Eisenbergiella | X | X | X |
| Eisenbergiella tayi | X | X | X |
| Enterobacter | X | X | — |
| Enterobacteriaceae | X | X | — |
| Enterobacteriales | X | X | — |
| Enterorhabdus | X | X | — |
| Epsilonproteobacteria | X | — | — |
| Erysipelatoclostridium | X | — | — |
| Erysipelatoclostridium ramosum | X | X | — |
| Erysipelotrichaceae | X | X | — |
| Erysipelotrichales | X | — | — |
| Erysipelotrichla | X | X | — |
| Euryarchaeota | X | — | — |
| Faecalibacterium | X | X | X |
| Faecalibacterium | X | X | X |
| prausnitzii | | | |
| Faecalibacterium | X | — | — |
| sp. canine oral | | | |
| taxon 147 | | | |
| Finegoldia | — | X | — |
| Finegoldia magna | — | X | — |
| Finegoldia sp. S9 | X | — | — |
| AA1-5 | | | |
| Firmicutes | X | X | — |
| Flavobacteriaceae | X | — | — |
| Flavobacteriales | X | — | — |
| Flavobacteriia | X | — | — |
| Flavonifractor | X | X | X |
| Flavonifractor | X | X | X |
| plautii | | | |
| Fusicatenibacter | X | X | X |
| Fusicatenibacter | X | X | X |
| saccharivorans | | | |
| Gammaproteobacteria | — | X | — |
| Gemella | — | X | — |
| Gordonibacter | X | — | — |
| pamelaeae | | | |
| Haemophilus | X | — | — |
| Haemophilus | X | — | — |
| parainfluenzae | | | |
| Holdemania | X | X | — |
| Holdemania | X | X | — |
| filiformis | | | |
| Hydrogenoanaerobacterium | X | X | — |
| Intestinimonas | — | X | — |
| Intestinimonas | X | X | — |
| butyriciproducens | | | |
| Klebsiella sp. | — | X | — |
| SOR89 | | | |
| Kluyvera | X | X | — |
| Kluyvrera georgiana | X | X | — |
| Lachnospira | X | — | — |
| Lachnospira | X | — | — |
| pectinoschiza | | | |
| Lactobacillaceae | X | — | X |
| Lactobacillus | X | X | — |
| Lactobacillus | — | X | — |
| rhamnosus | | | |
| Lactobacillus | — | X | — |
| salivarius | | | |
| Lactobacillus sp. | — | X | — |
| Akhmro1 | | | |
| Lactobacillus sp. | X | X | — |
| TAB-30 | | | |
| Lactonifactor | X | X | — |
| Lactonifactor | X | X | — |
| longoviformis | | | |
| Megasphaera | X | X | — |
| Methanobacteria | X | — | — |
| Methanobacteriaceae | X | — | — |
| Methanobacteriales | X | — | — |
| Methanobrevibacter | X | — | — |
| Methanobrevibacter smithii | X | — | — |
| Mogibacterium | X | — | — |
| Moryella | X | X | X |
| Murdochiella | X | — | — |
| Odoribacter | X | X | — |
| splanchnicus | | | |
| Oscillospira | X | — | X |
| Oscillospiraceae | X | X | X |
| Papillibacter | X | X | — |
| Parabacteroides | X | X | — |
| merdae | | | |
| Parasutterella | — | X | — |
| Parasutterella | — | X | — |
| excrementihominis | | | |
| Pasteurellaceae | X | — | — |
| Pasteurellales | X | — | — |
| Peptoclostridium | X | X | — |
| Peptoniphilus | — | X | — |
| Peptoniphilus sp. | X | — | — |
| 2002-2300004 | | | |
| Peptoniphilus sp. | — | X | — |
| 2002-38328 | | | |
| Peptoniphilus sp. | X | X | — |
| gpac018A | | | |
| Phascolarctobacterium faecium | X | — | — |
| Prevotella | — | X | — |
| Prevotella disiens | — | X | — |
| Prevotella ceae | X | X | X |
| Proteus | — | X | — |
| Proteus mirabilis | X | X | — |
| Pseudomonadales | — | X | — |
| Rhodospirillaceae | X | X | — |
| Rhodospirillales | X | X | — |
| Rikenellaceae | X | X | — |
| Roseburia faecis | X | — | — |
| Roseburia | X | X | — |
| inulinivorans | | | |
| Roseburia sp. | X | X | X |
| 11SE39 | | | |
| Ruminococcaceae | X | X | X |
| Sarcina | X | — | — |
| Shuttleworthia | X | X | — |
| Sporobacter | X | X | — |
| Streptococcus | — | X | — |
| gordonii | | | |
| Streptococcus sp. | X | — | — |
| oral taxon G59 | | | |
| Subdoligranulum | X | X | X |
| Subdoligranulum | X | X | X |
| variabile | | | |
| Sutterella | X | X | X |
| Sutterella | X | X | — |
| wadsworthensis | | | |
| Sutterellaceae | X | X | — |
| Terrisporobacter | X | X | — |
| Thalassospira | X | X | — |
| Weissella | — | X | — |
| Weissella hellenica | — | X | — |
| Xanthomonadales | — | X | — |

TABLE 8

| | Technique | | |
|---|---|---|---|
| | | Zero-inflated | |
| | Beta | Beta- | |
| Site: mouth | binomial | binomial | Kolmogorov- |
| Taxa name | regression | regression | Smirnov test |
| Gammaproteobacteria | — | X | — |
| Haemophilus influenzae | X | — | — |
| Kluyvera | — | X | — |
| Kluyvera | | | |
| georgiana | — | X | — |
| Lactobacillaceae | — | X | — |
| Lactobacillus | — | X | — |
| Veillonella | — | X | — |
| sp. | | | |
| 2011_Oral_ | | | |
| VSA_D3 | | | |

TABLE 9

| | Technique | | |
|---|---|---|---|
| | | Zero-inflated | |
| | Beta | Beta- | |
| Site: nose | binomial | binomial | Kolmogorov- |
| Taxa name | regression | regression | Smirnov test |
| Anaerococcus | X | — | — |
| Anaerococcus | X | X | X |
| octavius | | | |
| Anaerococcus | X | — | — |
| sp. | | | |
| 8405254 | | | |
| Bacillus | — | X | — |
| Clostridia | X | — | — |
| Clostridiales | X | — | X |
| Clostridiales | X | — | — |
| Family XI. | | | |
| Incertae | | | |
| Sedis | | | |
| Corynebacteriaceae | — | X | — |
| Corynebacterium | — | X | — |
| Corynebacterium | X | — | — |
| sp. NML | | | |
| 97-0186 | | | |
| Finegoldia | X | X | — |
| Finegoldia | X | X | — |
| sp. S9 AA1-5 | | | |
| Haemophilus | — | X | — |
| Lachnospiraceae | — | X | — |
| Microbacteriaceae | — | X | — |
| Moraxella | — | X | — |
| Moraxella | — | X | — |
| catarrhalis | | | |
| Neisseria | — | X | — |
| macacae | | | |
| Pasteurellaceae | — | X | — |
| Pasteurellales | — | X | — |
| Peptoniphilus | X | — | X |
| Peptoniphilus | X | X | X |
| sp. 7-2 | | | |
| Porphyromonas | — | X | — |
| Porphyromonas | — | X | — |
| catoniae | | | |
| Sphingobacteriales | — | X | — |
| Sphingobacteriia | — | X | — |

TABLE 10

| | Technique | | |
|---|---|---|---|
| | Beta | Zero-inflated | Kolmogorov- |
| Site: skin | binomial | Beta-binomial | Smirnov |
| Taxa name | regression | regression | test |
| Actinobacteria | X | X | X |
| Actinobacteria | X | X | X |
| Actinomycetales | X | X | X |
| Aerococcaceae | — | X | — |
| Alistipes | X | — | — |
| Alistipes finegoldii | X | X | — |
| Alistipes indistinctus | X | X | — |
| Alistipes putredinis | X | X | X |
| Alistipes shahii | X | X | — |
| Alistipes sp. EBA6-25cl2 | X | X | — |
| Alistipes sp. HGB5 | X | X | — |
| Alistipes sp. RMA 9912 | — | X | — |
| Alloscardovia | X | X | — |
| Alloscardovia omnicolens | X | X | — |
| Alphaproteobacteria | X | X | — |
| Anaerococcus | X | X | — |
| Anaerococcus octavius | X | X | X |
| Anaerococcus sp. | X | — | — |
| 8404299 | | | |
| Anaerococcus sp. | X | — | — |
| 8405254 | | | |
| Anaerococcus sp. S9 PR- | — | X | — |
| 16 | | | |
| Anaerosporobacter | X | X | — |
| mobilis | | | |
| Anaerostipes | X | — | X |
| Anaerostipes sp. | X | X | — |
| 3_2_56FAA | | | |
| Anaerostipes sp. | X | X | X |
| 5_1_63FAA | | | |
| Anaerotruncus sp. NML | X | X | — |
| 070203 | | | |
| Atopobium vaginae | — | X | — |
| Bacillaceae | — | X | — |
| Bacillales | X | — | — |
| Bacillus | — | X | — |
| Bacteroidaceae | X | X | X |
| Bacteroides | X | X | X |
| Bacteroides caccae | X | — | — |
| Bacteroides dorei | X | — | — |
| Bacteroides faecis | X | X | — |
| Bacteroides fragilis | X | X | — |
| Bacteroides intestinalis | — | X | — |
| Bacteroides massiliensis | — | X | — |
| Bacteroides plebeius | X | X | — |
| Bacteroides sp. 35AE37 | X | X | — |
| Bacteroides sp. AR20 | X | X | — |
| Bacteroides sp. AR29 | X | X | — |
| Bacteroides sp. D20 | X | X | — |
| Bacteroides sp. D22 | X | X | — |
| Bacteroides sp. | X | X | — |
| DJF_B097 | | | |
| Bacteroides sp. EBA5-17 | X | X | — |
| Bacteroides sp. SLC1-38 | X | X | X |
| Bacteroides | X | X | X |
| thetaiotaomicron | | | |
| Bacteroides uniformis | — | X | — |
| Bacteroides vulgatus | X | — | — |
| Barnesiella | X | — | — |
| intestinihominis | | | |
| Betaproteobacteria | X | X | — |
| Bifidobacteriaceae | X | — | X |
| Bifidobacteriales | X | — | X |
| Bifidobacterium | X | X | X |
| Bifidobacterium | X | X | — |
| kashiwanohense | | | |
| Bifidobacterium stercoris | X | X | — |
| Bilophila | — | X | — |
| Bilophila sp. 4_1_30 | X | — | — |
| Blautia glucerasea | X | — | — |
| Blautia hansenii | X | X | — |
| Blautia | X | X | — |
| hydrogenotrophica | | | |
| Blautia luti | X | X | X |
| Blautia sp. Ser8 | X | X | — |
| Blautia sp. YHC-4 | X | X | X |
| Blautia stercoris | X | X | — |
| Blautia wexlerae | X | — | — |
| Bradyrhizobiaceae | X | — | — |
| Bradyrhizobium | X | — | — |
| Burkholderiaceae | X | — | — |
| Burkholderiales | X | X | — |
| Butyricimonas | X | X | — |
| Butyricimonas sp. JCM | X | X | — |
| 18677 | | | |
| Campylobacter | X | — | — |
| Campylobacteraceae | X | — | — |
| Campylobacterales | X | — | — |
| Candidatus Soleaferrea | X | X | — |
| Carnobactetiaceae | — | X | — |
| Clostridia | X | X | — |
| Clostridiales | X | X | X |
| Clostridiales Family XI. | X | X | — |
| Incertae Sedis | | | |
| Clostridium | X | — | — |
| Collinsella | X | X | X |
| Collinsella aerofaciens | X | X | X |
| Coriobacteriaceae | X | X | X |
| Coriobacteriales | X | X | X |
| Corynebacteriaceae | X | X | X |
| Corynebacterium | X | X | X |
| Corynebacterium canis | X | X | — |
| Corynebacterium | X | X | — |
| epidermidicanis | | | |
| Corynebacterium | — | X | — |
| freiburgense | | | |
| Corynebacterium | — | X | — |
| glucuronolyticum | | | |
| Corynebacterium sp. | X | X | — |
| Corynebacterium sp. jw37 | — | X | — |
| Corynebacterium sp. | X | — | — |
| NML 97-0186 | | | |
| Corynebacterium | — | X | — |
| spheniscorum | | | |
| Cyanobacteria | — | X | — |
| Deltaproteobacteria | X | X | — |
| Desulfovibrio | X | X | — |
| desulfuricans | | | |
| Desulfovibrio piger | X | X | — |
| Desulfovibrionaceae | X | X | — |
| Desulfovibrionales | X | X | — |
| Dialister | X | — | — |
| Dialister | X | — | — |
| propionicifaciens | | | |
| Dielma | X | — | — |
| Dolosigranulum | — | X | — |
| Dolosigranulum pigrum | — | X | — |
| Dorea | X | X | X |
| Dorea formicigenerans | X | X | — |
| Dorea longicatena | X | X | X |
| Eggerthella | X | X | X |
| Eggerthella lenta | X | X | — |
| Eggerthella sp. HGA1 | X | X | — |
| Eisenbergiella | X | X | X |
| Eisenbergiella tayi | X | X | X |
| Enterobacter | X | X | — |
| Enterobacteriaceae | X | X | — |
| Enterobacteriales | X | X | — |
| Enterorhabdus | X | X | — |
| Epsilonproteobacteria | X | — | — |
| Erysipelatoclostridium | X | — | — |
| Erysipelatoclostridium | X | X | — |
| ramosum | | | |
| Erysipelotrichaceae | X | X | — |
| Erysipelotrichales | X | — | — |
| Erysipelotrichia | X | X | — |
| Euryarchaeota | X | — | — |
| Facklamia | X | — | — |
| Facklamia hominis | X | X | — |
| Faecalibacterium | X | X | X |
| Faecalibacterium | X | X | X |
| prausnitzii | | | |
| Faecalibacterium sp. | X | — | — |
| canine oral taxon 147 | | | |
| Finegoldia | X | X | — |
| Finegoldia magna | — | X | — |
| Finegoldia sp. S9 AA1-5 | X | X | — |
| Firmicutes | X | X | — |
| Flavobacteriaceae | X | — | — |
| Flavobacteriales | X | — | — |
| Flavobacteriia | X | — | — |
| Flavonifractor | X | X | X |
| Flavonifractor plautii | X | X | X |
| Fusicatenibacter | X | X | X |
| Fusicatenibacter | X | X | X |
| saccharivorans | | | |
| Gammaproteobacteria | — | X | — |
| Gemella | — | X | — |
| Gemella sp. 933-88 | — | X | — |
| Gordonibacter pamelaeae | X | — | — |
| Haemophilus | X | X | — |
| Haemophilus influenzae | X | — | — |
| Haemophilus | X | — | — |
| parainfluenzae | | | |
| Holdemania | X | X | — |
| Holdemania filiformis | X | X | — |
| Hydrogenoanaerobacterium | X | X | — |
| Intestinimonas | — | X | — |
| Intestinimonas | X | X | — |
| butyriciproducens | | | |
| Klebsiella sp. SOR89 | — | X | — |
| Kluyvera | X | X | — |
| Kluyvera georgiana | X | X | — |
| Lachnospira | X | — | — |
| Lachnospira | X | — | — |
| pectinoschiza | | | |
| Lachnospiraceae | — | X | — |
| Lactobacillaceae | X | X | X |
| Lactobacillus | X | X | — |
| Lactobacillus fornicalis | — | X | — |
| Lactobacillus rhamnosus | — | X | — |
| Lactobacillus salivarius | — | X | — |
| Lactobacillus sp. | — | X | — |
| 7_1_47FAA | | | |
| Lactobacillus sp. Akhmr01 | — | X | — |
| Lactobacillus sp. TAB-30 | X | X | — |
| Lactonifactor | X | X | — |
| Lactonifactor | X | X | — |
| longoviformis | | | |
| Megasphaera | X | X | — |
| Methanobacteria | X | — | — |
| Methanobacteriaceae | X | — | — |
| Methanobacteriales | X | — | — |
| Methanobrevibacter | X | — | — |
| Methanobrevibacter | X | — | — |
| smithii | | | |
| Microbacteriaceae | — | X | — |
| Mogibacterium | X | — | — |
| Moraxella | — | X | — |
| Moraxella catarrhalis | — | X | — |
| Moryella | X | X | X |
| Murdochiella | X | — | — |
| Neisseria | — | X | — |
| Neisseria macacae | — | X | — |
| Neisseriaceae | — | X | — |
| Neisseriales | — | X | — |
| Odoribacter splanchnicus | X | X | — |
| Oscillospira | X | — | X |
| Oscillospiraceae | X | X | X |
| Papillibacter | X | X | — |
| Parabacteroides merdae | X | X | — |
| Parasutterella | — | X | — |
| Parasutterella | — | X | — |
| excrementihominis | | | |
| Pasteurellaceae | X | X | — |
| Pasteurellales | X | X | — |
| Peptoclostridium | X | X | — |
| Peptoniphilus | X | X | X |
| Peptoniphilus sp. 2002- | X | — | — |
| 2300004 | | | |
| Peptoniphilus sp. 2002- | — | X | — |
| 38328 | | | |
| Peptoniphilus sp. 7-2 | X | X | X |
| Peptoniphilus sp. | X | X | — |
| gpac018A | | | |
| Peptoniphilus sp. oral | — | X | — |
| taxon 836 | | | |
| Phascolarctobacterium | X | — | — |
| faecium | | | |
| Phyllobacteriaceae | — | X | — |
| Phyllobacterium | X | X | — |
| Phyllobacterium sp. T50 | X | X | — |
| Porphyromonas | — | X | — |
| Porphyromonas catoniae | — | X | — |
| Prevotella | — | X | — |
| Prevotella disiens | — | X | — |
| Prevotellaceae | X | X | X |
| Propionibacteriaceae | — | X | — |
| Propionibacterium | — | X | — |
| Propionibacterium acnes | — | X | — |
| Propionibacterium sp. | — | X | — |
| MSP09A | | | |
| Proteus | — | X | — |
| Proteus mirabilis | X | X | — |
| Pseudomonadales | — | X | — |
| Ralstonia sp. S2.MAC.005 | X | — | — |
| Rhodospirillaceae | X | X | — |
| Rhodospirillales | X | X | — |
| Rikenellaceae | X | X | — |
| Roseburia faecis | X | — | — |
| Roseburia inulinivorans | X | X | — |
| Roseburia sp. 11SE39 | X | X | X |
| Ruminococcaceae | X | X | X |
| Sarcina | X | — | — |
| Shuttleworthia | X | X | — |
| Sphingobacteriales | — | X | — |
| Sphingobacteriia | — | X | — |
| Sphingomonadaceae | X | — | — |
| Sphingomonadales | X | — | — |
| Sporobacter | X | X | — |
| Staphylococcus sp. C9I2 | — | X | — |
| Streptococcus gordonii | — | X | — |
| Streptococcus sp. oral | X | — | — |
| taxon G59 | | | |
| Subdoligranulum | X | X | X |
| Subdoligranulum | X | X | X |
| variabile | | | |
| Sutterella | X | X | X |
| Sutterella wadsworthensis | X | X | — |
| Sutterellaceae | X | X | — |
| Terrisporobacter | X | X | — |
| Thalassospira | X | X | — |
| Veillonella sp. | — | X | — |
| 2011_Oral_VSA_D3 | | | |
| Veillonella sp. CM60 | — | X | — |
| Weissella | — | X | — |
| Weissella hellenica | — | X | — |
| Xanthomonadales | — | X | — |

TABLE 11

| | Technique | | |
|---|---|---|---|
| | | Zero- | |
| | | inflated | |
| | Beta | Beta- | |
| Site: genital | binomial | binomial | Kolmogorov- |
| Taxa name | regression | regression | Smirnov test |
| Actinobacteria | X | X | — |
| Actinobacteria | X | X | — |
| Actinomycetales | — | X | — |
| Alistipes | — | X | — |
| Atopobium | — | X | — |
| Bacteroides vulgatus | — | X | — |
| Carnobacteriaceae | — | X | — |
| Corynebacteriaceae | — | X | — |
| Corynebacterium | — | X | — |
| Corynebacterium sp. | — | X | — |
| Corynebacterium sp. NML 97-0186 | — | X | — |
| Dermabacter | X | — | — |
| Eremococcus | — | X | — |
| Firmicutes | X | X | — |
| Hespellia | — | X | — |
| Lactobacillales | X | X | — |
| Megasphaera | — | X | — |
| Propionibacteriaceae | — | X | — |
| Propionibacterium | — | X | — |
| Pseudobutyrivibrio | — | X | — |
| Staphylococcus sp. C9I2 | — | X | — |
| Streptococcus sp. BS35a | — | X | — |
| Subdoligranulum variabile | — | X | — |

TABLE 12

| | Technique | | |
|---|---|---|---|
| | Beta | | Kolmogorov- |
| Site: gut | binomial | Zero-inflated Beta- | Smirnov |
| Taxa name | regression | binomial regression | test |
| Actinobacteria | — | X | — |
| Actinobacteria | — | X | — |
| Actinomycetaceae | — | X | — |
| Actinomycetales | X | X | — |
| Alistipes | X | X | — |
| Alistipes putredinis | X | X | — |
| Alistipes sp. EBA6- | X | — | — |
| 25cl9 | | | |
| Alistipes sp. HGB5 | X | X | — |
| Anaeroglobus | — | X | — |
| Anaerostipes sp. | X | X | X |
| 5_1_63FAA | | | |
| Bacteroides sp. AR29 | X | — | — |
| Bacteroides | — | X | — |
| uniformis | | | |
| Betaproteobacteria | X | — | — |
| Bifidobacteriaceae | X | X | X |
| Bifidobacteriales | X | X | X |
| Bifidobacterium | X | X | X |
| Bifidobacterium | X | — | — |
| kashiwanohense | | | |
| Bifidobacterium | X | — | — |
| stercoris | | | |
| Blautia faecis | X | — | — |
| Blautia luti | X | X | — |
| Blautia wexlerae | X | — | — |
| Burkholderiales | X | — | — |
| Campylobacter | X | X | — |
| hominis | | | |
| Catenibacterium | X | X | — |
| mitsuokai | | | |
| Cloacibacillus | — | X | — |
| evryensis | | | |
| Clostridiaceae | — | X | — |
| Clostridiales Family | X | — | — |
| XI. Incertae Sedis | | | |
| Clostridium | X | X | X |
| Collinsella | X | X | X |
| Collinsella | X | — | — |
| aerofaciens | | | |
| Coriobacteriaceae | X | X | X |
| Coriobacteriales | X | X | X |
| Corynebacteriaceae | X | — | — |
| Corynebacterium | X | — | — |
| Corynebacterium | — | X | — |
| argentoratense | | | |
| Corynebacterium | X | X | — |
| canis | | | |
| Dialister | X | — | — |
| Dorea | X | — | — |
| Dorea | X | — | — |
| formicigenerans | | | |
| Dorea longicatena | X | — | — |
| Eisenbergiella | X | — | — |
| Eisenbergiella tayi | X | — | — |
| Enterobacter | — | X | — |
| Enterobacter sp. | — | X | — |
| BS2-1 | | | |
| Enterobacteriaceae | X | — | — |
| Enterobacteriales | X | — | — |
| Enterococcaceae | — | X | — |
| Enterococcus | — | X | — |
| Enterococcus sp. SI-4 | — | X | — |
| Faecalibacterium | X | X | — |
| Faecalibacterium | X | — | — |
| prausnitzii | | | |
| Finegoldia | X | — | — |
| Finegoldia sp. S9 | X | — | — |
| AA1-5 | | | |
| Flavonifractor | X | X | X |
| Flavonifractor plautii | X | — | — |
| Fusicatenibacter | X | X | X |
| Fusicatenibacter | X | X | X |
| saccharivorans | | | |
| Gammaproteobacteria | — | X | — |
| Gardnerella | — | X | — |
| Haemophilus | X | — | — |
| parainfluenzae | | | |
| Intestinimonas | X | X | — |
| Kluyvera | X | X | — |
| Kluyvera georgiana | X | X | — |
| Lachnospira | X | X | — |
| Lachnospira | X | X | — |
| pectinoschiza | | | |
| Lactobacillus | X | X | — |
| crispatus | | | |
| Lactobacillus | — | X | — |
| fornicalis | | | |
| Lactobacillus | — | X | — |
| salivarius | | | |
| Lactobacillus sp. | — | X | — |
| 7_1_47FAA | | | |
| Megasphaera | X | X | — |
| Moryella | X | — | — |
| Murdochiella | X | — | — |
| Negativicutes | X | X | X |
| Odoribacter | X | — | — |
| splanchnicus | | | |
| Oscillospira | X | X | — |
| Oscillospiraceae | X | X | — |
| Parabacteroides | X | — | — |
| merdae | | | |
| Pasteurellaceae | X | — | — |
| Pasteurellales | X | — | — |
| Peptoniphilus | X | — | — |
| Peptoniphilus sp. | X | — | — |
| 2002-2300004 | | | |
| Phascolarctobacterium | X | X | — |
| faecium | | | |
| Prevotella | X | X | — |
| Prevotella buccalis | X | — | — |
| Prevotellaceae | X | X | — |
| Pseudobutyrivibrio | — | X | — |
| Rhodospirillaceae | X | — | — |
| Rhodospirillales | X | — | — |
| Rikenellaceae | X | X | — |
| Roseburia | X | — | — |
| inulinivorans | | | |
| Roseburia sp. 11SE39 | X | X | — |
| Ruminococcaceae | X | X | — |
| Selenomonadales | X | X | X |
| Streptococcus | — | X | — |
| equinus | | | |
| Subdoligranulum | X | X | — |
| variabile | | | |
| Thalassospira | X | — | — |
| Veillonella | — | X | — |
| Veillonellaceae | X | X | X |

TABLE 13

| | Technique | | |
|---|---|---|---|
| | | Zero-inflated | |
| | Beta | Beta- | |
| Site: nose | binomial | binomial | Kolmogorov- |
| Taxa name | regression | regression | Smirnov test |
| Aggregatibacter | — | X | — |
| Anaerococcus | X | — | — |
| Bacillaceae | — | X | — |
| Clostridiales | X | — | — |
| Family XI. | | | |
| Incertae | | | |
| Sedis | | | |
| Comamonadaceae | — | X | — |
| Haemophilus | — | X | — |
| influenzae | | | |
| Lachnospiraceae | — | X | — |
| Lactococcus | — | X | |
| Peptoniphilus | X | — | — |
| Prevotellaoris | — | X | — |
| Sphingobacterium | — | X | — |

TABLE 14

| | Technique | | |
|---|---|---|---|
| | | Zero-inflated | |
| | Beta | Beta- | |
| Site: skin | binomial | binomial | Kolmogorov- |
| Taxa name | regression | regression | Smirnov test |
| Delftia | — | X | — |
| lacustris | | | |
| Janibacter | — | X | — |
| sp. M3-5 | | | |
| Rhodococcus | — | X | — |
| sp. MARG10 | | | |

TABLE 15

| | Technique | | |
|---|---|---|---|
| | | | Zero-inflated |
| | | Beta | Beta- |
| Site: Genital | Kolmogorov- | binomial | binomial |
| Taxa name | Smirnov test | regression | regression |
| Atopobium | — | — | X |
| Peptoniphilus sp. 2002-38328 | — | X | X |
| Prevotella disiens | — | — | X |
| Delftia lacustris | — | — | X |

TABLE 16

| | Technique | | |
|---|---|---|---|
| | | | Zero-inflated |
| | | Beta | Beta- |
| Site: Gut | Kolmogorov- | binomial | binomial |
| Taxa name | Smirnov test | regression | regression |
| Acetitomaculum | X | X | X |
| Acidaminococcaceae | X | X | X |
| Acidaminococcus | — | — | X |
| Acidaminococcus fermentans | — | X | — |
| Acidaminococcus intestini | — | X | X |
| Acidaminococcus sp. D21 | — | — | X |
| Acidobacteriia | — | X | — |
| Actinomyces | X | X | X |
| Actinomyces neuii | — | X | — |
| Actinomyces odontolyticus | — | — | X |
| Actinomyces sp. ICM54 | — | X | X |
| Actinomyces sp. oral strain | — | — | X |
| Hal-1065 | | | |
| Actinomycetaceae | — | X | X |
| Actinomycetales | — | X | X |
| Adlercreutzia | X | X | X |
| Adlercreutzia equolifaciens | X | X | X |
| Aerococcaceae | — | X | — |
| Aeromonadales | — | X | X |
| Aggregatibacter | — | — | X |
| Aggregatibacter aphrophilus | — | X | X |
| Aggregatibacter segnis | — | X | X |
| Akkermansia | X | X | X |
| Akkermansia muciniphila | X | X | X |
| Alistipes | X | X | X |
| Alistipes finegoldii | — | X | X |
| Alistipes massiliensis | — | X | — |
| Alistipes putredinis | X | X | X |
| Alistipes sp. EBA6-25cl2 | X | X | X |
| Alistipes sp. HGB5 | X | X | X |
| Alistipes sp. NML05A004 | X | X | X |
| Alistipes sp. RMA 9912 | X | X | X |
| Allisonella | — | X | — |
| Allisonella histaminiformans | — | X | — |
| Alphaproteobacteria | X | X | X |
| Anaerobacter | — | — | X |
| Anaerococcus lactolyticus | — | X | — |
| Anaerococcus murdochii | — | X | X |
| Anaerococcus octavius | — | X | — |
| Anaerococcus sp. 8404299 | — | — | X |
| Anaerococcus sp. S8 87-3 | — | — | X |
| Anaerococcus tetradius | — | X | — |
| Anaerococcus vaginalis | — | X | X |
| Anaerofilum | — | X | — |
| Anaerofustis | — | — | X |
| Anaerofustis stercorihominis | — | X | X |
| Anaeroplasmataceae | — | X | — |
| Anaeroplasmatales | — | — | X |
| Anaerosinus | — | — | X |
| Anaerosporobacter mobilis | — | X | X |
| Anaerostipes | X | X | X |
| Anaerostipes butyraticus | — | X | X |
| Anaerostipes caccae | — | — | X |
| Anaerostipes hadrus | — | X | X |
| Anaerostipes sp. 3_2_56FAA | — | X | X |
| Anaerostipes sp. 5_1_63FAA | X | X | X |
| Anaerotruncus | X | X | X |
| Anaerotruncus colihominis | — | X | — |
| Anaerotruncus sp. NML | — | X | — |
| 070203 | | | |
| Anaerovorax | — | X | — |
| Arcanobacterium | — | X | — |
| Asaccharospora | X | — | X |
| Asaccharospora irregularis | X | X | X |
| Atopobium | — | X | — |
| Atopobium sp. F0209 | — | X | — |
| Bacillaceae | — | X | — |
| Bacillales | X | X | X |
| Bacilli | X | X | X |
| Bacillus | — | X | — |
| Bacteroidaceae | — | X | X |
| Bacteroidales | — | — | X |
| Bacteroides | — | X | X |
| Bacteroides acidifaciens | — | X | X |
| Bacteroides caccae | X | X | X |
| Bacteroides clarus | — | X | — |
| Bacteroides coprocola | — | X | X |
| Bacteroides dorei | — | — | X |
| Bacteroides eggerthii | — | X | X |
| Bacteroides faecis | — | X | X |
| Bacteroides fragilis | X | X | X |
| Bacteroides intestinalis | — | — | X |
| Bacteroides massiliensis | — | X | X |
| Bacteroides nordii | — | X | X |
| Bacteroides ovatus | — | X | X |
| Bacteroides plebeius | — | X | X |
| Bacteroides salyersiae | — | X | X |
| Bacteroides sp. 3_1_40A | — | — | X |
| Bacteroides sp. 4072 | — | X | X |
| Bacteroides sp. AR20 | X | X | X |
| Bacteroides sp. AR29 | X | X | X |
| Bacteroides sp. CB57 | — | X | — |
| Bacteroides sp. D20 | — | — | X |
| Bacteroides sp. D22 | X | X | X |
| Bacteroides sp. DJF_B097 | X | X | X |
| Bacteroides sp. J1511 | — | — | X |
| Bacteroides sp. SLC1-38 | X | X | X |
| Bacteroides sp. XB12B | X | X | X |
| Bacteroides sp. XB44A | — | — | X |
| Bacteroides stercorirosoris | — | X | — |
| Bacteroides stercoris | — | X | — |
| Bacteroides uniformis | — | — | X |
| Bacteroides vulgatus | X | X | X |
| Bacteroidetes | — | — | X |
| Bacteroidia | — | — | X |
| Barnesiella | X | X | X |
| Barnesiella intestinihominis | X | X | X |
| Barnesiella sp. 177 | — | — | X |
| Betaproteobacteria | X | X | X |
| Bifidobacteriaceae | X | X | X |
| Bifidobacteriales | X | X | X |
| Bifidobacterium | X | — | X |
| Bifidobacterium adolescentis | — | — | X |
| Bifidobacterium animalis | — | — | X |
| Bifidobacterium biavatii | — | — | X |
| Bifidobacterium bifidum | X | X | X |
| Bifidobacterium choerinum | — | — | X |
| Bifidobacterium dentium | — | — | X |
| Bifidobacterium | — | X | X |
| kashiwanohense | | | |
| Bifidobacterium longum | X | X | X |
| Bifidobacterium | — | — | X |
| pseudocatenulatum | | | |
| Bifidobacterium sp. | — | X | X |
| Bifidobacterium sp. MSX5B | — | — | X |
| Bifidobacterium stercoris | — | X | X |
| Bilophila | X | X | X |
| Bilophila sp. 4_1_30 | X | X | X |
| Blautia | — | X | X |
| Blautia coccoides | — | X | X |
| Blautia faecis | X | X | X |
| Blautia glucerasea | — | X | X |
| Blautia hansenii | — | X | X |
| Blautia hydrogenotrophica | — | X | — |
| Blautia luti | X | X | X |
| Blautia producta | — | X | X |
| Blautia sp. Ser8 | X | X | X |
| Blautia sp. YHC-4 | X | X | X |
| Blautia stercoris | X | X | X |
| Blautia wexlerae | — | X | — |
| Bradyrhizobiaceae | — | X | — |
| Brevibacteriaceae | — | X | — |
| Brevibacterium | — | X | — |
| Burkholderiales | X | X | X |
| Butyricicoccus | — | — | X |
| Butyricicoccus pullicaecorum | — | X | X |
| Butyricimonas | X | X | X |
| Butyricimonas sp. JCM 18676 | — | X | X |
| Butyricimonas sp. JCM 18677 | X | X | X |
| Butyicimonas synergistica | — | X | — |
| Butyicimonas virosa | — | X | X |
| Butyrivibrio | — | X | — |
| Butyrivibrio crossotus | — | X | X |
| Campylobacter hominis | — | — | X |
| Campylobacter ureolyticus | — | X | — |
| Campylobacterales | — | X | — |
| Candidatus Stoquefichus | — | X | X |
| Carnobacteriaceae | — | X | X |
| Catenibacterium | X | X | X |
| Catenibacterium mitsuokai | — | X | — |
| Cellulosityticum | — | X | X |
| Citrobacter | — | X | X |
| Citrobacter sp. BW4 | — | X | X |
| Clostridia | X | X | X |
| Clostridiaceae | X | X | X |
| Clostridiales | X | X | X |
| Clostridiales Family XI. | — | — | X |
| Incertae Sedis | | | |
| Clostridiales Family XIII. | X | X | X |
| Incertae Sedis | | | |
| Clostridium | X | X | — |
| Collinsella | X | X | X |
| Collinsella aerofaciens | X | X | X |
| Collinsella intestinalis | — | X | X |
| Collinsella tanakaei | — | X | — |
| Coprobacillus | — | X | X |
| Coprobacillus sp. D6 | — | X | X |
| Coprobacter | — | X | — |
| Coprobacter fastidiosus | — | X | — |
| Coriobacteriaceae | X | X | X |
| Coriobacteriales | X | X | X |
| Corynebacteriaceae | — | X | — |
| Corynebacterium | — | X | — |
| Corynebacterium | — | X | — |
| epidermidicanis | | | |
| Corynebacterium sp. | — | X | — |
| Corynebacterium sp. | — | — | X |
| 713182/2012 | | | |
| Corynebacterium sp. jw37 | — | — | X |
| Corynebacterium sp. NML 97- | — | X | — |
| 0186 | | | |
| Corynebacterium sp. NML96- | — | X | — |
| 0085 | | | |
| Corynebacterium ulcerans | — | X | — |
| Cronobacter | — | — | X |
| Cronobacter dublinensis | — | — | X |
| Deltaproteobacteria | X | X | X |
| Dermabacter | — | — | X |
| Desulfovibrio | X | X | X |
| Desulfovibrio piger | — | X | X |
| Desulfovibrio sp. | — | X | X |
| 6_1_46AFAA | | | |
| Desulfovibrionaceae | X | X | X |
| Desulfovibrionales | X | X | X |
| Dialister | X | X | X |
| Dialister invisus | X | X | X |
| Dialister pneumosintes | — | — | X |
| Dialister propionicifaciens | — | X | — |
| Dialister sp. E2_20 | — | — | X |
| Dielma | — | X | X |
| Dielma fastidiosa | — | X | — |
| Dorea | X | X | X |
| Dorea formicigenerans | X | X | X |
| Dorea longicatena | X | X | X |
| Dysgonomonas | — | X | X |
| Eggerthella | X | X | X |
| Eggerthella lenta | — | X | X |
| Eggerthella sp. HGA1 | — | X | X |
| Eisenbergiella | X | X | X |
| Eisenbergiella tayi | X | X | X |
| Enterobacter | X | X | X |
| Enterobacter sp. BS2-1 | — | X | X |
| Enterobacteriaceae | X | X | X |
| Enterobacteriales | X | X | X |
| Enterococcaceae | X | X | X |
| Enterococcus | — | X | X |
| Enterococcus raffinosus | — | — | X |
| Enterococcus sp. C6I11 | — | X | X |
| Enterorhabdus | — | X | X |
| Enterorhabdus caecimuris | — | — | X |
| Epsilonproteobacteria | — | X | — |
| Epulopiscium | — | X | X |
| Erysipelatoclostridium | X | X | X |
| ramosum | | | |
| Erysipelotrichaceae | — | — | X |
| Erysipelotrichales | — | — | X |
| Erysipelotrichia | — | — | X |
| Eubacterium limosum | — | — | X |
| Euryarchaeota | X | X | X |
| Facklamia hominis | — | X | — |
| Facklamia languida | — | X | — |
| Faecalibacterium | X | X | X |
| Faecalibacterium prausnitzii | X | X | X |
| Faecalibacterium sp. canine | — | X | X |
| oral taxon 147 | | | |
| Fibrobacterales | — | X | — |
| Finegoldia magna | — | X | X |
| Finegoldia sp. S8 F7 | — | — | X |
| Firmicutes | — | X | — |
| Flavobacteriaceae | X | X | X |
| Flavobacteriales | X | X | X |
| Flavobacteriia | X | X | X |
| Flavobacterium | X | X | X |
| Flavonifractor | X | — | X |
| Flavonifractor plautii | X | X | X |
| Fusicatenibacter | X | X | X |
| Fusicatenibacter | X | X | X |
| saccharivorans | | | |
| Fusobacteria | — | X | X |
| Fusobacteriaceae | — | X | X |
| Fusobacteriales | — | X | X |
| Fusobacteriia | — | X | X |
| Fusobacterium | — | X | X |
| Fusobacterium mortiferum | — | X | — |
| Fusobacterium necrogenes | — | X | X |
| Fusobacterium periodonticum | — | — | X |
| Fusobacterium sp. CM22 | — | — | X |
| Fusobacterium ulcerans | — | X | X |
| Gammaproteobacteria | X | X | X |
| Gardnerella | — | X | — |
| Gardnerella vaginalis | — | X | — |
| Gelria | — | X | X |
| Gemella | — | X | X |
| Gemella morbillorum | — | — | X |
| Gemella sp. 933-88 | — | X | X |
| Granulicatella | — | X | X |
| Granulicatella adiacens | — | X | X |
| Haemophilus | X | X | X |
| Haemophilus influenzae | — | X | X |
| Haemophilus parainfluenzae | X | X | X |
| Herbaspirillum | — | X | X |
| Herbaspirillum seropedicae | — | X | X |
| Hespellia | — | — | X |
| Holdemania | — | X | X |
| Holdemania filiformis | — | X | X |
| Howardella | — | X | — |
| Howardella ureilytica | — | X | — |
| Intestinibacter | — | X | — |
| Intestinimonas | X | X | X |
| Intestinimonas | — | — | X |
| butyriciproducens | | | |
| Klebsiella | — | X | X |
| Klebsiella oxytoca | — | X | X |
| Kluyvera | X | X | X |
| Kluyvera georgiana | X | X | X |
| Lachnospira | X | X | X |
| Lachnospira pectinoschiza | X | X | X |
| Lachnospiraceae | — | X | X |
| Lactobacillaceae | X | X | X |
| Lactobacillales | X | X | X |
| Lactobacillus | X | X | X |
| Lactobacillus crispatus | — | X | X |
| Lactobacillus delbrueckii | — | — | X |
| Lactobacillus gasseri | — | X | X |
| Lactobacillus iners | — | X | — |
| Lactobacillus johnsonii | — | — | X |
| Lactobacillus mucosae | — | — | X |
| Lactobacillus paracasei | — | X | X |
| Lactobacillus plantarum | — | X | X |
| Lactobacillus ruminis | — | X | — |
| Lactobacillus salivarius | — | X | — |
| Lactobacillus sp. 7_1_47FAA | — | — | X |
| Lactobacillus sp. TAB-26 | — | — | X |
| Lactobacillus sp. TAB-30 | — | X | — |
| Lactococcus | — | X | — |
| Lactococcus lactis | — | X | X |
| Lactonifactor | X | X | X |
| Lactonifactor longoviformis | X | X | X |
| Lentisphaerae | X | X | X |
| Lentisphaeria | X | X | X |
| Leptotrichiaceae | — | X | — |
| Leuconostoc | — | X | — |
| Leuconostocaceae | — | X | — |
| Marvinbryantia | X | X | X |
| Megamonas | — | X | X |
| Megamonas funiformis | — | X | X |
| Megasphaera | X | X | X |
| Megasphaera elsdenii | — | X | — |
| Megasphaera genomosp. C1 | — | — | X |
| Megasphaera sp. NP3 | — | — | X |
| Methanobacteria | X | X | X |
| Methanobacteriaceae | X | X | X |
| Methanobacteriales | X | X | X |
| Methanobrevibacter | X | X | X |
| Methanobrevibacter smithii | X | X | X |
| Methanosphaera | — | X | — |
| Methanosphaera stadtmanae | — | X | — |
| Mitsuokella | — | X | — |
| Mobiluncus | — | X | — |
| Mobiluncus mulieris | — | X | — |
| Mollicutes | — | X | — |
| Morganella | — | X | X |
| Morganella morganii | — | X | X |
| Moryella | X | X | X |
| Moryella indoligenes | — | X | — |
| Murdochiella | — | — | X |
| Murdochiella asaccharolytica | — | — | X |
| Negativicutes | X | — | — |
| Neisseria | — | X | — |
| Neisseria mucosa | — | X | X |
| Neisseriaceae | — | X | X |
| Neisseriales | — | — | X |
| Odoribacter | X | X | X |
| Odoribacter splanchnicus | X | X | X |
| Opitutae | — | X | X |
| Oribacterium | — | X | — |
| Oscillospira | — | X | X |
| Oscillospira guilliermondii | — | X | X |
| Oscillospiraceae | X | X | X |
| Oxalobacteraceae | — | X | X |
| Pantoea | — | X | X |
| Pantoea sp. CWB304 | — | — | X |
| Papillibacter | — | X | — |
| Parabacteroides | X | X | X |
| Parabacteroides distasonis | X | X | X |
| Parabacteroides goldsteinii | — | X | X |
| Parabacteroides johnsonii | — | X | X |
| Parabacteroides merdae | X | X | X |
| Parabacteroides sp. dnLKV8 | — | — | X |
| Paraprevotella | — | X | — |
| Paraprevotella clara | — | X | X |
| Parasutterella | X | X | X |
| Parasutterella | — | X | X |
| excrementihominis | | | |
| Parvimonas | — | — | X |
| Parvimonas micra | — | — | X |
| Pasteurella | — | X | — |
| Pasteurella pueumotropica | — | X | — |
| Pasteurellaceae | X | X | X |
| Pasteurellales | X | X | X |
| Pediococcus | — | X | X |
| Pediococcus sp. MFC1 | — | X | X |
| Peptoclostridium | — | X | — |
| Peptoclostridium difficile | X | X | X |
| Peptococcaceae | — | — | X |
| Peptococcus | — | X | X |
| Peptococcus niger | — | X | — |
| Peptoniphilus coxii | — | X | X |
| Peptoniphilus duerdenii | — | X | X |
| Peptoniphilus lacrimalis | — | X | — |
| Peptoniphilus sp. 1-14 | — | X | X |
| Peptoniphilus sp. 2002-38328 | — | — | X |
| Peptoniphilus sp. DNF00840 | — | — | X |
| Peptoniphilus sp. JCM 8143 | — | X | — |
| Peptoniphilus sp. oral taxon | — | X | — |
| 375 | | | |
| Peptoniphilus sp. S9 PR-13 | — | — | X |
| Peptostreptococcaceae | — | X | X |
| Peptostreptococcus | X | X | X |
| Peptostreptococcus | — | X | X |
| anaerobius | | | |
| Peptostreptococcus stomatis | — | — | X |
| Phascolarctobacterium | X | X | X |
| Phascolarctobacterium | X | X | X |
| faecium | | | |
| Phascolarctobacterium sp. 377 | — | X | — |
| Phascolarctobacterium | — | X | X |
| succinatutens | | | |
| Phyllobacteriaceae | — | — | X |
| Phyllobacterium | — | X | X |
| Porphyromonadaceae | X | X | X |
| Porphyromonas bennonis | — | X | — |
| Porphyromonas somerae | — | X | — |
| Porphyromonas sp. 2024b | — | X | — |
| Porphyromonas sp. 2026 | — | — | X |
| Prevotella | — | — | X |
| Prevotella disiens | — | X | — |
| Prevotella timonensis | — | X | X |
| Prevotellaceae | X | X | X |
| Proteiniphilum | — | — | X |
| Proteobacteria | — | — | X |
| Proteus | — | X | X |
| Proteus mirabilis | — | X | X |
| Pseudobutyrivibrio | X | X | X |
| Pseudoclavibacter | — | X | — |
| Pseudoclavibacter sp. Timone | — | X | — |
| Pseudoflavonifractor | — | X | — |
| Pseudoflavonifractor | — | X | — |
| capillosus | | | |
| Pseudomonadaceae | — | X | — |
| Puniceicoccales | — | X | X |
| Rahnella | — | — | X |
| Rhizobiales | — | X | — |
| Rhodocyclaceae | — | X | — |
| Rhodocyclales | — | X | — |
| Rhodospirillaceae | X | X | X |
| Rhodospirillales | X | X | X |
| Rikenellaceae | X | X | X |
| Robinsoniella | — | X | X |
| Romboutsia | — | X | X |
| Roseburia | X | X | X |
| Roseburia cecicola | — | X | — |
| Roseburia faecis | — | X | X |
| Roseburia hominis | — | X | — |
| Roseburia intestinalis | — | — | X |
| Roseburia inulinivorans | — | X | X |
| Roseburia sp. 11SE39 | X | X | X |
| Rothia | — | X | — |
| Ruminococcaceae | X | X | X |
| Sarcina | X | X | X |
| Selenomonadales | X | — | — |
| Slackia | — | X | — |
| Slackia sp. NATTS | — | X | — |
| Staphylococcaceae | — | X | X |
| Staphylococcus | — | X | X |
| Staphylococcus sp. 3348O2 | — | — | X |
| Staphylococcus sp. C9I2 | — | X | X |
| Streptococcaceae | — | X | — |
| Streptococcus | X | X | X |
| Streptococcus agalactiae | — | X | X |
| Streptococcus dysgalactiae | — | X | — |
| Streptococcus equinus | — | — | X |
| Streptococcus parasanguinis | — | X | X |
| Streptococcus pasteurianus | — | — | X |
| Streptococcus sp. 11aTha1 | — | X | X |
| Streptococcus sp. | — | X | X |
| 2011_Oral_MS_A3 | | | |
| Streptococcus sp. oral taxon | — | — | X |
| G59 | | | |
| Streptococcus thermophilus | — | X | — |
| Subdoligranulum | X | X | X |
| Subdoligranulum variabile | X | X | X |
| Succinivibrionaceae | — | X | X |
| Sutterella sp. YIT 12072 | — | X | — |
| Sutterella wadsworthensis | — | X | — |
| Sutterellaceae | — | X | X |
| Synergistaceae | — | X | X |
| Synergistales | — | X | X |
| Synergistetes | — | — | X |
| Synergistia | — | X | X |
| Syntrophococcus | — | X | — |
| Tenericutes | — | X | — |
| Terrisporobacter | X | X | X |
| Terrisporobacter glycolicus | — | X | — |
| Thalassospira | X | X | X |
| Thermoanaerobacteraceae | — | — | X |
| Thermoanaerobacterales | — | X | X |
| Varibaculum | — | X | — |
| Varibaculum cambriense | — | X | X |
| Varibaculum sp. CCUG 45114 | — | — | X |
| Veillonella | X | X | X |
| Veillonella atypica | — | — | X |
| Veillonella parvula | — | X | X |
| Veillonella rogosae | — | X | X |
| Veillonella sp. | — | — | X |
| 2011_Oral_VSA_D3 | | | |
| Veillonella sp. AS16 | — | X | X |
| Veillonella sp. CM60 | — | X | X |
| Veillonella sp. FFA-2014 | — | — | X |
| Veillonella sp. MSA12 | — | X | X |
| Veillonellaceae | X | X | X |
| Verrucomicrobia | X | X | X |
| Verrucomicrobiaceae | X | X | X |
| Verrucomicrobiae | X | X | X |
| Verrucomicrobiales | X | X | X |
| Victivallaceae | X | X | X |
| Victivallis | X | X | X |
| Victivallis vadensis | — | X | X |

TABLE 17

| | Technique | | |
|---|---|---|---|
| | | | Zero-inflated |
| | | Beta | Beta- |
| Site: Mouth | Kolmogorov- | binomial | binomial |
| Taxa name | Smirnov test | regression | regression |
| Enterobacteriaceae | — | — | X |
| Enterobacteriales | — | — | X |
| Gemella sanguinis | — | — | X |
| Leptotrichia hofstadii | — | — | X |

TABLE 18

| | Technique | | |
|---|---|---|---|
| | | | Zero-inflated |
| | | Beta | Beta- |
| Site: Nose | Kolmogorov- | binomial | binomial |
| Taxa name | Smirnov test | regression | regression |
| Haemophilus | — | — | X |
| Haemophilus parainfluenzae | — | — | X |
| Moraxella | — | — | X |

TABLE 19

| | Technique | | |
|---|---|---|---|
| | | | Zero-inflated |
| | | Beta | Beta- |
| Site: Skin | Kolmogorov- | binomial | binomial |
| Taxa name | Smirnov test | regression | regression |
| Acinetobacter | — | — | X |
| Haemophilus | — | — | X |
| Haemophilus parainfluenzae | — | — | X |

TABLE 20

| | | As- | | | | | | | | | |
|---|---|---|---|---|---|---|---|---|---|---|---|
| | | sign- | | | | | | | | | |
| | | ed | | | | | | | | | |
| | | Mi- | | | | | | | | | |
| | | cro- | Micro- | Micro | | Micro- | Micro- | Micro- | Micro- | Micro- | Micro- |
| | Taxon- | biome | biome | biome | Micro- | biome | biome | biome | biome | biome | biome |
| | omy | Sub- | Sub- | Sub- | biome | Sub- | Sub- | Sub- | Sub- | Sub- | Sub |
| | iden- | Sys- | System | System | Sub- | System | System | System | System | System | System |
| Taxa name | tifier | tem | 0 | 1 | System2 | 3 | 4 | 5 | 6 | 7 | 8 |
| Campylobacter | 194 | 1 | 0.0114 | 0.2330 | 0.0426 | 0.0001 | −0.0681 | −0.0745 | 0.0095 | 0.0698 | 0.0070 |
| Flavobacterium | 237 | 1 | −0.0094 | −0.1116 | −0.0179 | 0.0367 | −0.0668 | 0.0352 | 0.0824 | 0.0022 | −0.0442 |
| Enterobacteriaceae | 543 | 1 | 0.0126 | 0.3091 | 0.1084 | 0.0145 | −0.0294 | 0.1360 | 0.0395 | −0.0156 | 0.0495 |
| Citrobacter | 544 | 5 | −0.0737 | 0.0083 | 0.0079 | 0.0137 | 0.0059 | −0.1745 | −0.0642 | −0.0420 | 0.0058 |
| Enterobacter | 547 | 1 | −0.0721 | −0.0478 | 0.0403 | −0.0424 | −0.0122 | −0.0796 | −0.0676 | −0.0386 | 0.0032 |
| Klebsiella | 570 | 1 | −0.0357 | −0.0392 | 0.0423 | −0.0488 | −0.0476 | −0.0460 | −0.0261 | 0.0342 | −0.0173 |
| Kluyvera | 579 | 1 | 0.0063 | 0.2801 | 0.0975 | −0.0056 | −0.0777 | −0.1360 | 0.0462 | 0.0156 | 0.0314 |
| Pasteurellaceae | 712 | 1 | −0.0094 | 0.2112 | 0.1190 | −0.0365 | −0.0454 | −0.0113 | 0.0310 | 0.0026 | 0.0285 |
| Actinobacillus | 713 | 0 | 0.1889 | 0.0039 | −0.0350 | 0.0240 | −0.0711 | −0.0151 | −0.0163 | −0.0067 | −0.0270 |
| Haemophilus | 724 | 1 | −0.0084 | 0.2075 | 0.1204 | −0.0389 | −0.0458 | −0.0150 | 0.0236 | 0.0132 | 0.0198 |
| Haemophilus | 729 | 1 | 0.0139 | 0.2128 | 0.1167 | −0.0273 | −0.0575 | −0.0100 | 0.0180 | 0.0095 | 0.0168 |
| parainfluenzae | | | | | | | | | | | |
| Pasteurella | 745 | 0 | 0.2424 | 0.0270 | −0.0319 | 0.0364 | −0.0583 | −0.0203 | −0.0233 | −0.0001 | −0.0314 |
| Pasteurella | 758 | 0 | 0.2425 | 0.0269 | −0.0319 | 0.0365 | −0.0583 | −0.0202 | −0.0234 | −0.0004 | −0.0314 |
| pneumotropica | | | | | | | | | | | |
| Bacteroidaceae | 815 | 2 | 0.0235 | −0.2905 | −0.7964 | 0.1191 | 0.0956 | 0.0542 | −0.0318 | −0.0425 | −0.0644 |
| Bacteroides | 816 | 2 | 0.0234 | −0.2915 | −0.7968 | 0.1195 | 0.0964 | 0.0540 | −0.0322 | −0.0427 | −0.0649 |
| Bacteroides | 817 | 5 | 0.0564 | 0.0911 | −0.1593 | 0.0728 | −0.0467 | 0.2444 | 0.1178 | −0.0457 | −0.0087 |
| fragilis | | | | | | | | | | | |
| Bacteroides | 818 | 2 | 0.0414 | 0.0534 | 0.0891 | −0.0065 | −0.0096 | 0.0382 | 0.0087 | −0.0327 | −0.0149 |
| thetaiotaomioron | | | | | | | | | | | |
| Bacteroides | 820 | 2 | −0.0130 | 0.0927 | 0.1778 | 0.0330 | −0.0456 | 0.0889 | 0.0878 | −0.0069 | −0.0122 |
| uniformis | | | | | | | | | | | |
| Bacteroides | 821 | 2 | 0.0175 | −0.2557 | −06388 | 0.0212 | 0.0838 | −0.0565 | −0.0541 | −0.0476 | −0.0589 |
| vulgalus | | | | | | | | | | | |
| Parabacteroides | 823 | 2 | 0.0477 | −0.1085 | −0.5243 | 0.0954 | 0.0374 | 0.1520 | 0.0291 | −0.0432 | −0.1006 |
| distasonis | | | | | | | | | | | |
| Campylobacter | 827 | 1 | 0.0387 | 0.1454 | 0.0477 | −0.0261 | −0.0407 | −0.0340 | −0.0207 | 0.0949 | 0.0065 |
| ureolyticus | | | | | | | | | | | |
| Butyrivibrio | 830 | 1 | 0.0487 | 0.0760 | 0.1050 | −0.0743 | −0.0216 | −0.0714 | 0.0014 | −0.0064 | 0.0535 |
| Fibrobacter | 832 | 3 | 0.0233 | 0.0921 | 0.0269 | −0.0151 | −0.0558 | −0.0038 | 0.0878 | −0.0434 | 0.5303 |
| Porphyromonas | 836 | 1 | 0.0489 | 0.2652 | 0.0793 | −0.0009 | −0.0655 | −0.0911 | 0.0022 | 0.0214 | −0.0344 |
| Prevotella | 838 | 1 | −0.0113 | 0.2910 | 0.1210 | −0.0105 | −0.0987 | 0.0562 | 0.0388 | −0.0895 | 0.0372 |
| Roseburia | 841 | 1 | −0.0329 | −0.6363 | −0.1630 | −0.0889 | 0.0200 | −0.2308 | −0.2258 | −0.0447 | −0.0904 |
| Roseburia | 842 | 1 | −0.0155 | 0.0817 | 0.0346 | 0.0173 | 0.0411 | −0.0063 | 0.0303 | 0.0217 | 0.0177 |
| cecicola | | | | | | | | | | | |
| Fusobacterium | 848 | 1 | 0.0307 | 0.1147 | −0.0071 | −0.0125 | −0.0252 | −0.0132 | 0.0445 | 0.0011 | 0.0082 |
| Faecalibacterium | 853 | 1 | −0.0009 | −0.5053 | −0.1565 | −0.0707 | 0.3849 | −0.2373 | −0.2810 | −0.0303 | −0.0914 |
| prausnitzii | | | | | | | | | | | |
| Desulfovibrio | 872 | 5 | −0.0148 | 0.1073 | 0.0312 | −0.1038 | −0.0116 | 0.2882 | 0.1628 | 0.0723 | 0.0065 |
| Desulfovibrio | 901 | 5 | −0.0089 | −0.0375 | −0.0792 | 0.0378 | 0.0954 | 0.2330 | −0.0319 | 0.0442 | −0.0530 |
| piger | | | | | | | | | | | |
| Acidaminococcus | 904 | 2 | 0.0634 | 0.0751 | −0.0728 | −0.0046 | 0.0929 | 0.0459 | 0.0391 | −0.0047 | −0.0366 |
| Megasphaera | 906 | 2 | −0.0140 | 0.0010 | 0.1853 | −0.0720 | 0.0084 | −0.1952 | −0.0970 | 0.0210 | 0.0562 |
| Herbaspirillum | 963 | 1 | 0.0028 | 0.2241 | 0.0709 | −0.1274 | −0.1764 | 0.0140 | 0.1660 | 0.0066 | 0.1032 |
| Herbaspirillum | 964 | 1 | −0.0039 | 0.2134 | 0.0649 | −0.1259 | −0.1834 | 0.0173 | 0.1785 | 0.0250 | 0.0900 |
| seropedicae | | | | | | | | | | | |
| Bacteriodetes | 976 | 2 | 0.0140 | −0.3025 | −0.7737 | 0.1419 | 0.1622 | 0.0569 | −0.1355 | −0.0770 | −0.129l |
| Proteobacteria | 1224 | 4 | −0.0041 | −0.0364 | −0.1500 | 0.0254 | 0.6361 | 0.1315 | −0.1045 | −0.0485 | −0.0398 |
| Gammaproteobacteria | 1236 | 1 | 0.0055 | 0.2838 | 0.1256 | −0.0002 | 0.0276 | 0.1598 | 0.0711 | −0.0355 | 0.0638 |
| Firmicutes | 1239 | 1 | −0.0273 | −0.8076 | −0.2356 | 0.0068 | 0.1440 | −0.1442 | −0.2547 | −0.0601 | −0.2128 |
| Leuconostoc | 1243 | 1 | −0.0142 | 0.1188 | 0.0647 | −0.0016 | −0.0263 | −0.0436 | 0.0037 | −0.0122 | 0.0441 |
| Peptostretococcus | 1257 | 1 | 0.0356 | 0.1558 | 0.0495 | 0.0101 | −0.0283 | 0.0352 | 0.0116 | 0.0397 | −0.0025 |
| Finegoldia magna | 1260 | 2 | −0.0005 | 0.1234 | 0.1084 | −0.0396 | −0.0276 | −0.0629 | −0.0464 | −0.0512 | 0.0120 |
| Peptostreptococcus | 1261 | 1 | 0.0244 | 0.1708 | 0.0495 | 0.0028 | −0.0055 | 0.0341 | 0.0514 | 0.0285 | −0.0005 |
| anaerobius | | | | | | | | | | | |
| Sarcina | 1266 | 1 | −0.0706 | −0.2993 | 0.0130 | 0.1060 | 0.0472 | −0.0660 | −0.3619 | −0.0099 | −0.1998 |
| Micrococcaceae | 1268 | 2 | 0.0849 | −0.0379 | −0.0716 | −0.0286 | 0.0364 | −0.0055 | −0.0636 | 0.0746 | −0.0085 |
| Staphylococcus | 1279 | 1 | 0.0317 | 0.0736 | −0.0184 | −0.0448 | 0.0202 | −0.0664 | −0.0898 | 0.0084 | −0.0072 |
| Streptococcaceae | 1300 | 2 | 0.0241 | −0.0869 | 0.1549 | −0.0213 | −0.0172 | 0.0009 | 0.0107 | −0.0058 | −0.0370 |
| Streptococcus | 1301 | 2 | 0.0362 | −0.0113 | 0.1943 | −0.0337 | −0.0302 | 0.0280 | 0.0600 | 0.0079 | −0.0065 |
| Streptococcus | 1308 | 2 | 0.0127 | 0.0417 | 0.0878 | 0.0428 | −0.0470 | −0.0038 | 0.0722 | 0.0182 | −0.0365 |
| thermophilus | | | | | | | | | | | |
| Streptococcus | 1311 | 1 | −0.0156 | 0.0741 | 0.0428 | 0.0055 | −0.0213 | −0.0320 | −0.0860 | −0.0100 | −0.0042 |
| agalactiae | | | | | | | | | | | |
| Streptococcus | 1318 | 2 | −0.0104 | 0.1134 | 0.1467 | −0.0887 | −0.0207 | −0.1318 | −0.0943 | −0.0149 | 0.0728 |
| parasanguinis | | | | | | | | | | | |
| Enterococcus | 1350 | 0 | −0.5484 | −0.0165 | 0.0243 | −0.0360 | 0.0257 | −0.0614 | −0.0452 | −0.0086 | −0.0066 |
| Lactococcus | 1357 | 1 | 0.0060 | 0.1638 | 0.1032 | −0.0787 | −0.0512 | −0.0378 | −0.0253 | 0.0116 | 0.0564 |
| Gemella | 1378 | 1 | 0.0760 | 0.0158 | −0.0326 | −0.0297 | 0.0197 | −0.0655 | −0.0647 | 0.0333 | −0.0138 |
| Atopobium | 1380 | 7 | 0.0289 | 0.0061 | −0.0373 | 0.0344 | −0.0296 | −0.0203 | −0.0298 | 0.3691 | 0.0289 |
| Bacillales | 1385 | 1 | 0.0161 | 0.1831 | 0.0471 | −0.0960 | −0.0208 | −0.1322 | −0.0132 | −0.0169 | 0.0588 |
| Clostridium | 1485 | 1 | −0.0169 | −0.4907 | −0.1322 | −0.0473 | 0.0354 | −0.1325 | −0.1746 | −0.0015 | −0.0831 |
| Peptoclostridium | 1496 | 2 | 0.0196 | 0.0372 | 0.1218 | −0.0392 | −0.0339 | −0.1015 | −0.0657 | −0.0251 | 0.0457 |
| difficile | | | | | | | | | | | |
| Erysipelatoclostridium | 1547 | 2 | −0.0203 | −0.0242 | 0.2782 | −0.0509 | −0.0013 | −0.1802 | −0.1310 | −0.0097 | 0.0131 |
| ramosum | | | | | | | | | | | |
| Lactobacillus | 1578 | 2 | −0.0582 | 0.1382 | 0.2091 | −0.0946 | −0.0467 | −0.0205 | 0.0761 | −0.0062 | 0.0677 |
| Corynebacteriaceae | 1653 | 1 | −0.0486 | 0.1195 | 0.0949 | −0.0168 | −0.0571 | −0.0661 | 0.0043 | −0.0757 | −0.0266 |
| Actinomyces | 1654 | 1 | 0.0201 | 0.1923 | 0.0735 | −0.0765 | −0.1107 | −0.1455 | −0.0836 | 0.0138 | 0.0723 |
| Bifidobacterium | 1678 | 1 | −0.0767 | −0.1628 | 0.0277 | −0.0365 | −0.0177 | −00174 | 0.0295 | −0.0313 | −0.0318 |
| Bifidobacterium | 1681 | 1 | −0.0370 | −0.0340 | 0.0175 | 0.0388 | 0.0026 | −0.0045 | −0.0436 | −0.0198 | 0.0098 |
| bifidum | | | | | | | | | | | |
| Brevibacterium | 1696 | 0 | 0.2613 | 0.0371 | −0.0358 | 0.0150 | −0.0008 | 0.0632 | 0.0610 | 0.1187 | −0.0228 |
| Corynebacterium | 1716 | 1 | −0.0334 | 0.1310 | 0.0906 | −0.0093 | −0.0605 | −0.0629 | 0.0067 | −0.0744 | −0.0358 |
| Corynebacterium sp. | 1720 | 1 | 0.0197 | 0.1850 | 0.0524 | −0.0290 | −0.0430 | −0.0487 | 0.0172 | 0.0397 | −0.0243 |
| Eubacterium | 1730 | 8 | −0.0417 | 0.0273 | 0.0924 | −0.0582 | 0.0126 | −0.0908 | −0.0093 | −0.0182 | 0.3327 |
| Actinobacteria | 1760 | 1 | −0.0614 | −0.6061 | −0.1443 | −0.0128 | 0.0825 | −0.1270 | −0.1212 | −0.0656 | −0.1324 |
| Actinomycetales | 2037 | 1 | −0.0737 | 0.1405 | 0.1323 | −0.0467 | −0.0909 | −0.1099 | −0.0471 | −0.0795 | 0.0294 |
| Actinomycetaceae | 2049 | 1 | 0.0001 | 0.2967 | 0.1108 | −0.0745 | −0.1291 | −0.1374 | −0.0627 | 0.0439 | 0.0889 |
| Anaeroplasma | 2086 | 0 | −0.1572 | 0.0414 | 0.0218 | −0.0563 | −0.0107 | −0.0084 | −0.0372 | 0.0148 | −0.0306 |
| Asteroleplasma | 2152 | 1 | −0.0060 | 0.0853 | 0.0354 | −0.0448 | −0.0155 | −0.0308 | 0.0022 | −0.0073 | 0.0770 |
| Methanobacteriales | 2158 | 7 | 0.0058 | 0.0096 | 0.0019 | 0.0477 | −0.0685 | 0.0743 | 0.0204 | 0.6737 | −0.0536 |
| Methanobacteriaceae | 2159 | 7 | 0.0058 | 0.0096 | 0.0019 | 0.0477 | −0.0685 | 0.0743 | 0.0204 | 0.6737 | −0.0536 |
| Methanobrevibacter | 2172 | 7 | 0.0161 | 0.0116 | −0.0057 | 0.0329 | −0.0567 | 0.0639 | 0.0341 | 0.6607 | −0.0184 |
| Methanobrevibacter | 2173 | 7 | 0.0165 | 0.0129 | −0.0078 | 0.0391 | −0.0578 | 0.0738 | 0.0284 | 0.6579 | −0.0255 |
| smithii | | | | | | | | | | | |
| Methanosphaera | 2316 | 7 | −0.0059 | 0.0305 | −0.0618 | −0.0037 | −0.0510 | 0.0581 | 0.0603 | 0.2724 | −0.0249 |
| Methanosphaera | 2317 | 7 | −0.0211 | 0.0372 | −0.0504 | 0.0020 | −0.0604 | 0.0728 | 0.0403 | 0.2654 | −0.0159 |
| stadtmanae | | | | | | | | | | | |
| Peptococcus | 2740 | 1 | 0.0106 | 0.2869 | 0.1103 | −0.0318 | −0.0566 | 0.0270 | 0.1498 | 0.0960 | 0.0779 |
| Lachnospira | 28050 | 1 | 0.0071 | −0.4100 | −0.0968 | −0.0419 | 0.0246 | −0.1270 | −0.1167 | −0.0089 | −0.0599 |
| Lachnospira | 28052 | 1 | 0.0176 | −0.2418 | −0.0859 | −0.0610 | 0.0229 | −0.0745 | −0.0356 | −0.0184 | −0.0132 |
| pectonischiza | | | | | | | | | | | |
| Bacteroides | 28111 | 2 | 0.0302 | −0.0519 | −0.1010 | 0.0335 | −0.0181 | −0.0254 | −0.0471 | 0.0279 | 0.0087 |
| egerthii | | | | | | | | | | | |
| Bacteroides ovatus | 28116 | 1 | 0.0076 | 0.0485 | 0.0274 | −0.0327 | −0.0003 | −0.0220 | 0.0341 | −0.0117 | −0.0245 |
| Alistipes putredinis | 28117 | 1 | −0.0281 | −0.1819 | −0.3302 | 0.1624 | 0.0898 | 0.1204 | 0.1094 | −0.0690 | −0.0910 |
| Odoribacter | 28118 | 5 | −0.0120 | 0.1096 | −0.1481 | 0.0969 | −0.0028 | 0.4663 | 0.2309 | −0.0178 | −0.0104 |
| splanchnicus | | | | | | | | | | | |
| Prevotella bivia | 28125 | 1 | 0.0122 | 0.1376 | 0.0313 | 0.0090 | −0.0457 | −0.0677 | −0.0101 | −0.0292 | 0.0237 |
| Prevotella buccalis | 28127 | 1 | 0.0662 | 0.0813 | 0.0373 | 0.0107 | −0.0582 | −0.0499 | 0.0150 | 0.0943 | 0.0063 |
| Prevotella disiens | 28130 | 1 | 0.0383 | 0.1799 | 0.0806 | 0.0068 | −0.0623 | −0.0234 | 0.0536 | 0.0198 | 0.0385 |
| Alphaproteobacteria | 28211 | 2 | 0.0160 | −0.0824 | −0.2191 | 0.0315 | 0.0822 | −0.0277 | −0.1098 | −0.0237 | −0.0785 |
| Betaproteobacteria | 28216 | 4 | 0.0399 | −0.1852 | −0.2019 | −0.0634 | 0.9310 | −0.0367 | −0.1625 | −0.0460 | −0.0431 |
| Deltaproteobacteria | 28221 | 5 | 0.0162 | 0.1041 | −0.0966 | 0.0578 | 0.0208 | 0.8553 | 0.1117 | 0.0432 | −0.0562 |
| Euryarchaeota | 28890 | 7 | 0.0085 | 0.0161 | 0.0065 | 0.0284 | −0.0645 | 0.0789 | 0.0344 | 0.6913 | −0.0318 |
| Asaccharospora | 29359 | 2 | −0.0599 | 0.1007 | 0.1506 | 0.0169 | −0.0559 | 0.1233 | 0.0452 | −0.0143 | −0.0049 |
| irregularis | | | | | | | | | | | |
| Veillonella | 29465 | 1 | −0.0044 | 0.2131 | 0.1848 | −0.0095 | −0.0653 | 0.0335 | 0.0524 | −0.0451 | 0.0673 |
| Epsilonproteobacteria | 29547 | 1 | 0.0118 | 0.2322 | 0.0478 | −0.0062 | −0.0738 | −0.0742 | 0.0179 | 0.0678 | 0.0122 |
| Bifidobacteriaceae | 31953 | 1 | −0.0772 | −0.1618 | 0.0270 | −0.0350 | −0.0169 | −0.0202 | 0.0273 | −0.0292 | −0.0248 |
| Proprionibacteriaceae | 31957 | 1 | 0.0561 | −0.0224 | −0.0317 | −0.0047 | 0.0424 | 0.0363 | −0.0217 | 0.0465 | 0.0595 |
| Mollicutes | 31969 | 1 | −0.0545 | 0.0826 | 0.0006 | −0.0257 | −0.0248 | −0.0248 | 0.0477 | −0.0086 | 0.0168 |
| Veillonellaceae | 31977 | 1 | 0.0876 | 0.0758 | 0.1504 | −0.0287 | 0.0975 | 0.1040 | 0.1383 | −0.0101 | −0.0155 |
| Clostridiaceae | 31979 | 1 | −0.0518 | −0.5309 | −0.1000 | 0.0883 | 0.0489 | −0.1044 | −0.3767 | −0.0044 | −0.2454 |
| Acetitomaculum | 31980 | 1 | 0.0217 | 0.2296 | 0.0007 | −0.0442 | −0.0187 | 0.1026 | 0.2395 | −0.0211 | 0.0916 |
| Fusobacteria | 32066 | 1 | 0.0242 | 0.1251 | −0.0274 | −0.0018 | −0.0328 | −0.0119 | 0.0443 | 0.0002 | 0.0023 |
| Actinomyces neuii | 33007 | 0 | 0.0932 | 0.0276 | 0.0002 | −0.0263 | −0.0278 | −0.0125 | 0.0141 | 0.0381 | 0.0302 |
| Phascolarclobacterium | 33024 | 2 | 0.0084 | −0.1515 | −0.2988 | 0.0518 | 0.1646 | 0.0574 | −0.1155 | −0.0074 | −0.0653 |
| Phascolarclobacterium | 33025 | 2 | −0.0194 | −0.0868 | −0.3119 | 0.0578 | 0.1039 | 0.0765 | −0.0419 | −0.0303 | −0.0436 |
| faecium | | | | | | | | | | | |
| Blautia producta | 33035 | 1 | −0.0656 | −0.0400 | 0.0623 | −0.0537 | −0.0162 | −0.0871 | −0.0747 | −0.0038 | 0.0016 |
| Anaerococcus | 33036 | 1 | 0.0115 | 0.1850 | 0.0406 | −0.0083 | −0.0411 | −0.0524 | 0.0009 | 0.0457 | −0.0280 |
| tetradius | | | | | | | | | | | |
| Anaerococcus | 33037 | 1 | 0.0170 | 0.1665 | 0.0824 | −0.0444 | −0.0471 | −0.0472 | −0.0353 | 0.0106 | −0.0286 |
| vaginalis | | | | | | | | | | | |
| Lactobacillaceae | 33958 | 2 | −0.0340 | 0.1299 | 0.1821 | 0.0122 | −0.0966 | 0.0962 | 0.0603 | −0.0514 | −0.0743 |
| Bilophila | 35832 | 5 | 0.0386 | 0.1697 | −0.0829 | 0.0278 | −0.0636 | 0.7918 | 0.2610 | 0.0085 | −0.0402 |
| Bilophila wadsworthia | 35833 | 5 | 0.0363 | 0.0723 | 0.0433 | −0.0390 | −0.0533 | 0.1423 | 0.0368 | −0.0211 | 0.0203 |
| Terrisporobacter | 36841 | 1 | 0.0427 | 0.1832 | 0.0184 | −0.0185 | −0.0063 | −0.0144 | 0.0071 | −0.0557 | 0.1019 |
| glycolicus | | | | | | | | | | | |
| Dorea | 39486 | 1 | 0.417 | −0.1565 | −0.1002 | −0.0255 | −0.0061 | 0.1121 | 0.0926 | −0.0070 | 0.0095 |
| formicigenerans | | | | | | | | | | | |
| Veillonella atypica | 39777 | 0 | 0.1823 | 0.0365 | −0.0037 | 0.0164 | −0.0275 | 0.0243 | −0.0015 | 0.0268 | −0.0314 |
| Dialister | 39948 | 1 | −0.0213 | −0.0923 | −0.0658 | 0.0188 | 0.1200 | −0.0302 | −0.0241 | 0.0238 | −0.0748 |
| Sutterella | 40544 | 4 | −0.0043 | −0.1306 | −0.0812 | −0.0386 | 0.7703 | −0.0599 | −0.1299 | −0.0326 | −0.0241 |
| Sutterella | 40545 | 4 | 0.0000 | −0.0550 | −0.0947 | −0.0365 | 0.3965 | −0.0286 | 0.0053 | −0.0246 | −0.0241 |
| wadsworthensis | | | | | | | | | | | |
| Bifidobacterium sp. | 41200 | 0 | −0.1346 | 0.0213 | 0.0197 | −0.0033 | −0.0360 | −0.0743 | 0.0197 | −0.0157 | 0.0736 |
| Rhodospirillaceae | 41295 | 2 | −0.0039 | −0.0704 | −0.2230 | 0.0218 | 0.1069 | 0.0137 | −0.0726 | −0.0141 | −0.0728 |
| Sporobacter | 44748 | 3 | −0.0019 | 0.0821 | 0.1139 | −0.1886 | −0.0071 | −0.0708 | −0.0016 | 0.0248 | 0.1170 |
| Butyrivibrio | 45851 | 1 | 0.0443 | 0.0330 | 0.0176 | −0.0458 | 0.0174 | −0.0426 | −0.0637 | 0.0029 | 0.0563 |
| crossotus | | | | | | | | | | | |
| Granulicatella | 46124 | 1 | 0.0010 | 0.0217 | −0.0040 | 0.0251 | −0.0612 | −0.1260 | −0.1067 | 0.0256 | −0.0028 |
| adiacens | | | | | | | | | | | |
| Pseudobutyrivibrio | 46205 | 1 | −0.0306 | −0.5479 | −0.2338 | −0.0195 | 0.0937 | −0.1640 | −0.2637 | 0.0106 | −0.1146 |
| Parabacteroides | 46503 | 2 | 0.0618 | −0.1432 | −0.4291 | 0.1359 | 0.0299 | 0.0759 | −0.0465 | −0.0372 | −0.0461 |
| merdae | | | | | | | | | | | |
| Bacteroides stercoris | 46506 | 1 | −0.0335 | 0.0548 | −0.0172 | −0.0153 | −0.0081 | −0.0063 | −0.0032 | 0.0202 | 0.0110 |
| Parabacteroides | 47678 | 5 | −0.0137 | 0.0232 | −0.1391 | 0.0556 | −0.0018 | 0.1317 | 0.0419 | −0.0204 | −0.0161 |
| caccae | | | | | | | | | | | |
| Lactobacillus | 47715 | 0 | −0.1498 | 0.0380 | −0.0428 | 0.0373 | 0.0093 | 0.0063 | −0.0460 | −0.0136 | 0.0312 |
| rhamnosus | | | | | | | | | | | |
| Lactobacillus | 47770 | 1 | −0.0217 | 0.0724 | 0.0312 | −0.0034 | −0.0271 | 0.0331 | −0.0161 | −0.0095 | 0.0266 |
| crispatus | | | | | | | | | | | |
| Verrucomicobiales | 48461 | 3 | 0.0549 | 0.0570 | −0.0669 | 0.8056 | −0.1168 | 0.0549 | 0.0515 | 0.0236 | −0.0255 |
| Flavobacteriaceae | 49546 | 1 | 0.0354 | −0.2081 | −0.0736 | 0.0251 | −0.0438 | 0.0650 | 0.0571 | 0.0031 | −0.0781 |
| Actinobacillus | 51048 | 0 | 0.1889 | 0.0039 | −0.0350 | 0.0240 | −0.0711 | −0.0151 | −0.0163 | −0.0067 | −0.0270 |
| porcinus | | | | | | | | | | | |
| Blautia | 53443 | 2 | 0.0285 | 0.1182 | 0.2127 | −0.0717 | −0.0071 | −0.0775 | −0.0150 | 0.0477 | 0.0220 |
| hydrogenotrophica | | | | | | | | | | | |
| Anaerococcus | 54007 | 1 | 0.0562 | 0.0184 | −0.0210 | 0.0069 | −0.0269 | −0.0307 | −0.0485 | 0.0401 | 0.0082 |
| octavius | | | | | | | | | | | |
| Holdemania | 61170 | 2 | 0.0150 | 0.2475 | 0.5140 | −0.1295 | −0.0649 | −0.1744 | −0.0393 | −0.0165 | 0.1257 |
| Holdemania filiformis | 61171 | 2 | 0.0059 | 0.2348 | 0.4923 | −0.1384 | −0.0643 | −0.1849 | −0.0474 | −0.0219 | 0.1396 |
| Corynebacterium | 65058 | 1 | 0.0454 | 0.0905 | −0.0013 | −0.0005 | −0.0148 | 0.0194 | 0.0619 | −0.0134 | −0.0136 |
| ulcerans | | | | | | | | | | | |
| Fibrobacteres | 65842 | 8 | 0.0387 | 0.1142 | 0.0445 | −0.0354 | −0.0571 | −0.0043 | 0.0806 | −0.0123 | 0.6197 |
| Facklamia | 66831 | 1 | 0.0718 | 0.0979 | −0.0434 | 0.0162 | −0.0012 | 0.0108 | −0.0071 | 0.0625 | −0.0032 |
| Thermoanaero- | 68295 | 3 | 0.0034 | 0.1574 | 0.1380 | −0.3056 | −0.0463 | −0.0759 | 0.0983 | −0.0296 | 0.1318 |
| bacterales | | | | | | | | | | | |
| Streptococcus peroris | 68891 | 1 | 0.0128 | 0.1002 | 0.0482 | 0.0117 | 0.0616 | −0.0014 | −0.0359 | −0.0102 | 0.0149 |
| Campylobacteraceae | 72294 | 1 | 0.0119 | 0.2322 | 0.0432 | −0.0023 | 0.0711 | −0.0726 | 0.0123 | 0.0737 | 0.0061 |
| Kluyvera georgiana | 73098 | 1 | 0.0023 | 0.2885 | 0.1007 | −0.0034 | −0.0767 | 0.1433 | 0.0519 | 0.0125 | 0.0332 |
| Verrucomicrobia | 74201 | 3 | 0.0548 | 0.0529 | −0.0683 | 0.8059 | −0.1071 | 0.0517 | 0.0598 | 0.0234 | −0.0350 |
| Collinsella | 74426 | 1 | −0.0265 | −0.2860 | −0.1152 | −0.0151 | 0.0597 | 0.0625 | 0.0036 | 0.0192 | −0.0529 |
| aerofaciens | | | | | | | | | | | |
| Oxalobacteraceae | 75682 | 1 | 0.0033 | 0.2224 | 0.0727 | −0.1311 | −0.1763 | 0.0131 | 0.1631 | 0.0062 | 0.1083 |
| Rhodocyclaceae | 75787 | 8 | −0.0185 | 0.0604 | −0.0021 | −0.0408 | −0.0455 | −0.0354 | 0.0725 | −0.0120 | 0.2510 |
| Campylobacter | 76517 | 1 | −0.0046 | 0.1688 | 0.0318 | 0.0082 | −0.0850 | −0.0197 | 0.0636 | 0.0305 | −0.0034 |
| hominis | | | | | | | | | | | |
| Burkholderiales | 80840 | 4 | 0.0420 | −0.1827 | −0.1987 | −0.0619 | 0.9323 | −0.0361 | −0.1624 | −0.0490 | −0.0471 |
| Comamonadaceae | 80864 | 1 | −0.0154 | 0.1139 | −0.0184 | 0.0191 | 0.0065 | 0.0579 | 0.0975 | 0.0601 | 0.0306 |
| Delfia | 80865 | 1 | 0.0530 | 0.0402 | −0.0235 | −0.0143 | 0.0413 | −0.0354 | −0.0011 | 0.0505 | −0.0061 |
| Leuconostocaceae | 81850 | 1 | 0.0004 | 0.1071 | 0.0519 | −0.0195 | −0.0415 | −0.0478 | 0.0024 | −0.0169 | 0.0309 |
| Enterococcaceae | 81852 | 0 | −0.5500 | −0.0203 | 0.0207 | −0.0327 | 0.0239 | −0.0601 | −0.0470 | −0.0039 | −0.0077 |
| Facklamia languida | 82347 | 1 | 0.0686 | 0.0912 | −0.0167 | 0.0341 | −0.0202 | 0.0429 | 0.0275 | 0.0573 | −0.0073 |
| Coriobacteriaceae | 84107 | 1 | −0.0058 | −0.7081 | −0.2455 | 0.0012 | 0.1405 | −0.1957 | −0.2594 | 0.0175 | −0.1801 |
| Slackia | 84108 | 4 | 0.0327 | 0.1853 | 0.0372 | −0.0092 | −0.3233 | 0.0807 | 0.1756 | 0.0131 | 0.0474 |
| Eggerthella | 84111 | 2 | 0.0015 | 0.0892 | 0.4210 | −0.1115 | −0.0331 | −0.1485 | −0.1796 | −0.0091 | 0.0000 |
| Eggerthella lenta | 84112 | 2 | 0.0245 | 0.0789 | 0.2202 | −0.1185 | −0.0399 | −0.0853 | −0.0604 | 0.0065 | −0.0042 |
| Coriobacteriales | 84999 | 1 | −0.0056 | −0.7085 | −0.2458 | 0.0016 | 0.1406 | −0.1957 | −0.2601 | 0.0171 | −0.1801 |
| Bifidobacteriales | 85004 | 1 | −0.0772 | −0.1618 | 0.0270 | −0.0350 | −0.0169 | −0.0202 | 0.0273 | −0.0292 | −0.0248 |
| Brevibacteriaceae | 85019 | 0 | 0.2613 | 0.0371 | −0.0358 | 0.0150 | −0.0008 | 0.0632 | 0.0610 | 0.1187 | −0.0228 |
| Microbacteriacea | 85023 | 7 | 0.1567 | 0.1218 | 0.0321 | −0.0387 | −0.0295 | 0.0464 | 0.0323 | 0.2292 | −0.0103 |
| Bacteroides | 85831 | 2 | 0.0040 | 0.0200 | −0.1414 | 0.0394 | −0.0220 | 0.0728 | 0.0814 | −0.0330 | −0.0161 |
| acidificiens | | | | | | | | | | | |
| Mogibacterium | 86331 | 1 | 0.0593 | 0.2971 | 0.0764 | −0.0718 | −0.0503 | −0.0346 | 0.0592 | 0.0598 | 0.0732 |
| Dorea longicatena | 88431 | 1 | 0.0159 | −0.5107 | −0.1097 | −0.1233 | 0.1059 | −0.1519 | −0.1718 | −0.0137 | −0.0126 |
| Blautia luti | 89014 | 1 | −0.0064 | −0.5176 | −0.0737 | −0.0662 | 0.0327 | −0.1594 | −0.1197 | −0.0151 | −0.0809 |
| Staphylococcaceae | 90964 | 1 | 0.0352 | 0.0732 | −0.0196 | −0.0478 | 0.0226 | −0.0679 | −0.0915 | 0.0122 | −0.0083 |
| Bacilli | 91061 | 2 | 0.0224 | −0.1650 | 0.1070 | 0.0430 | 0.0155 | 0.1117 | 0.0120 | −0.0373 | −0.1475 |
| Enterobacteriales | 91347 | 1 | 0.0126 | 0.3091 | 0.1084 | 0.0145 | −0.0294 | 0.1360 | 0.0395 | −0.0156 | 0.0495 |
| Bacteroides sp. AR20 | 93974 | 2 | 0.0343 | 0.2156 | −0.4650 | 0.0833 | −0.0278 | −0.0812 | −0.1400 | −0.0119 | −0.0975 |
| Bacteroides sp. AR29 | 93975 | 2 | −0.0823 | −0.0299 | −0.3760 | 0.0861 | −0.0424 | 0.1305 | 0.0894 | −0.0275 | −0.0038 |
| Papillibacter | 100175 | 3 | 0.0066 | 0.0615 | 0.1123 | −0.1492 | −0.0084 | −0.0882 | 0.0442 | −0.0296 | 0.0939 |
| Coprobacillus | 100883 | 5 | −0.0231 | 0.0347 | 0.0735 | −0.0100 | −0.0071 | −0.1966 | −0.0528 | 0.0141 | 0.0356 |
| Catenbacterium | 100886 | 6 | −0.0423 | −0.0406 | −0.0102 | −0.0219 | 0.0357 | 0.0174 | 0.0558 | −0.0476 | −0.0333 |
| mitsuokai | | | | | | | | | | | |
| Collinsella | 102106 | 1 | −0.0128 | −0.6749 | −0.1930 | −0.0340 | 0.1116 | −0.2344 | −0.2620 | 0.0130 | −0.1385 |
| Pseudoflavonifractor | 106588 | 2 | −0.0288 | 0.1652 | 0.3520 | −0.0896 | −0.0681 | −0.0983 | −0.0591 | 0.0042 | 0.1106 |
| capillosus | | | | | | | | | | | |
| Granulicatella | 117563 | 1 | −0.0058 | −0.0209 | 0.0011 | 0.0258 | −0.0608 | −0.1265 | −0.1030 | 0.0325 | −0.0025 |
| Flavobacteriia | 117743 | 1 | 0.0354 | −0.2081 | −0.0736 | 0.0251 | −0.0439 | 0.0649 | 0.0571 | 0.0031 | −0.0781 |
| Oscillospira | 119852 | 1 | −0.0123 | 0.2968 | 0.2444 | −0.0435 | −0.1636 | 0.1175 | 0.2275 | 0.0300 | 0.0775 |
| Erysipelotrichaceae | 128827 | 1 | −0.0318 | −0.4252 | −0.1452 | 0.0125 | 0.0823 | 0.0545 | −0.0258 | −0.0432 | −0.0967 |
| Pasteurellales | 135625 | 1 | −0.0094 | 0.2112 | 0.1190 | −0.0365 | −0.0454 | −0.0113 | 0.0310 | 0.0026 | 0.0285 |
| Catenibacterium | 135858 | 6 | −0.0068 | 0.0516 | −0.0153 | −0.0179 | −0.0154 | 0.0162 | 0.2391 | −0.0295 | 0.0530 |
| Collinsella instetinalis | 147207 | 2 | 0.0265 | 0.0504 | 0.0821 | −0.0093 | −0.0427 | 0.0408 | 0.0437 | −0.0264 | −0.0157 |
| Finegoldia | 150022 | 1 | −0.0532 | 0.2340 | 0.1385 | −0.0281 | −0.0833 | −0.0121 | 0.0487 | −0.1004 | −0.0084 |
| Turicibacter sanguinis | 154288 | 1 | 0.0169 | 0.2375 | 0.2037 | −0.0281 | −0.0758 | −0.0227 | 0.0468 | −0.0175 | 0.1203 |
| Megamonas | 158846 | 0 | 0.1764 | −0.0285 | 0.0123 | 0.0181 | 0.0470 | −0.0109 | 0.0033 | −0.0033 | 0.0264 |
| Corynebacterium | 161890 | 1 | 0.0288 | 0.1096 | −0.0009 | 0.0222 | −0.0019 | 0.0185 | 0.0213 | 0.0370 | −0.0027 |
| mastitidis | | | | | | | | | | | |
| Peptoniphilus | 162289 | 1 | −0.0195 | 0.2744 | 0.1462 | −0.0442 | −0.0653 | −0.0183 | 0.0572 | −0.1015 | 0.0092 |
| Gallicola | 162290 | 0 | 0.2476 | 0.0765 | 0.0343 | −0.0318 | 0.0024 | −0.0466 | −0.0140 | 0.0433 | 0.0025 |
| Anaerococcus | 165779 | 1 | −0.0420 | 0.2611 | 0.1147 | −0.0152 | −0.0758 | −0.0532 | 0.0286 | −0.0851 | −0.0035 |
| Roseburia intestinalis | 166486 | 1 | 0.0150 | −0.0856 | −0.0148 | −0.0824 | −0.0223 | −0.0414 | 0.0062 | −0.0169 | 0.0255 |
| Thalassospira | 168934 | 2 | −0.0087 | −0.0630 | −0.2203 | 0.0239 | 0.1067 | 0.0086 | −0.0701 | −0.0078 | −0.0752 |
| Anaerotruncus | 169435 | 2 | −0.0346 | 0.0282 | 0.2317 | −0.1319 | 0.0052 | −0.1669 | −0.0616 | −0.0370 | 0.0887 |
| colihominis | | | | | | | | | | | |
| Brevibacterium | 170994 | 9 | 0.2481 | 0.0621 | −0.0019 | 0.0179 | 0.0075 | 0.0339 | 0.0285 | 0.1254 | −0.0051 |
| paucivorans | | | | | | | | | | | |
| Bacteroidales | 171549 | 2 | 0.0134 | −0.2976 | −0.7766 | 0.1465 | 0.1602 | 0.0628 | −0.1249 | −0.0735 | −0.1243 |
| Rikenellaceae | 171550 | 1 | −0.0599 | −0.3218 | −0.3789 | 0.2417 | 0.0959 | −0.0079 | −0.3619 | −0.0435 | −0.2246 |
| Porphyromonadaceae | 171551 | 2 | 0.0279 | −0.2635 | −0.6158 | 0.2138 | 0.2016 | 0.0223 | −0.2799 | −0.0703 | −0.1958 |
| Prevotellaceae | 171552 | 2 | 0.0314 | 0.0990 | 0.1823 | −0.0293 | 0.1052 | −0.1147 | −0.1270 | −0.0964 | 0.0137 |
| Victivallis | 172900 | 6 | 0.0248 | 0.2028 | 0.0530 | −0.0434 | −0.1336 | 0.0530 | 0.5860 | 0.0062 | 0.0226 |
| Victivallis vadensis | 172901 | 6 | 0.0181 | 0.1051 | −0.0180 | −0.0208 | −0.0094 | −0.0139 | 0.2299 | −0.0532 | 0.0475 |
| Shuttleworthia | 177971 | 8 | −0.0378 | 0.1627 | 0.1852 | −0.1449 | −0.0035 | −0.0378 | 0.0572 | 0.0340 | 0.5236 |
| Methanobacteria | 183925 | 7 | 0.0058 | 0.0096 | 0.0019 | 0.0477 | −0.0685 | 0.0743 | 0.0204 | 0.6737 | −0.0536 |
| Varibaculum | 184869 | 1 | 0.0458 | 0.1427 | 0.0212 | −0.0303 | −0.0118 | −0.0581 | −0.0209 | 0.0695 | 0.0204 |
| Varibaculum | 184870 | 1 | 0.0503 | 0.1365 | 0.0204 | −0.0145 | −0.0253 | −0.0467 | −0.0047 | 0.0787 | −0.0029 |
| cambriense | | | | | | | | | | | |
| Corynebacterium | 185761 | 1 | 0.0856 | 0.0826 | −0.0006 | −0.0064 | −0.0571 | 0.0037 | 0.0212 | 0.0090 | 0.0467 |
| spheniscorum | | | | | | | | | | | |
| Anaeroplasmatales | 186332 | 1 | −0.0770 | 0.1001 | 0.0298 | −0.0557 | −0.0261 | −0.0286 | 0.0129 | −0.0068 | 0.0321 |
| Anaeroplasmataceae | 186333 | 1 | −0.0808 | 0.1011 | 0.0353 | −0.0557 | −0.0244 | −0.0338 | 0.0055 | −0.0002 | 0.0366 |
| Clostridia | 186801 | 1 | −0.0344 | −0.8249 | −0.2672 | 0.0116 | 0.1251 | −0.1675 | −0.2845 | −0.0522 | −0.2077 |
| Clostridiales | 186802 | 1 | −0.0345 | −0.8249 | −0.2675 | 0.0115 | 0.1254 | −0.1677 | −0.2841 | −0.0520 | −0.2076 |
| Lachospiraceae | 186803 | 1 | −0.0135 | −0.7630 | −0.2873 | −0.0198 | −0.0184 | −0.1216 | −0.1769 | −0.0158 | −0.1314 |
| Peptostreptococcaceae | 186804 | 1 | 0.0201 | −0.3608 | −0.0328 | 0.1018 | 0.0547 | 0.0528 | −0.1285 | −0.0489 | −0.1981 |
| Eubacteriaceae | 186806 | 8 | −0.0444 | 0.0200 | 0.1131 | −0.0567 | 0.0060 | −0.1512 | −0.0373 | −0.0333 | 0.3969 |
| Peptococcaceae | 186807 | 1 | −0.0101 | 0.2537 | 0.0781 | −0.0024 | −0.0568 | 0.0570 | 0.1409 | 0.0708 | 0.0637 |
| Thermoanaero- | 186814 | 3 | 0.0071 | 0.1559 | 0.1468 | −0.3122 | −0.0411 | −0.0792 | 0.0736 | −0.0280 | 0.1307 |
| bacteraceae | | | | | | | | | | | |
| Lactobacillales | 186826 | 2 | 0.0348 | −0.1779 | 0.1032 | 0.0365 | 0.0138 | 0.1006 | 0.0110 | −0.0317 | −0.1462 |
| Aerococcaceae | 186827 | 1 | 0.0364 | 0.1103 | −0.0457 | 0.0239 | 0.0049 | −0.0062 | 0.0083 | 0.0522 | 0.0038 |
| Carnobacteriaceae | 186828 | 1 | −0.0168 | 0.0140 | 0.0005 | 0.0373 | −0.0574 | −0.1230 | −0.1027 | 0.0376 | −0.0030 |
| Acidaminococcus | 187327 | 4 | 0.0133 | 0.0361 | −0.0253 | 0.0087 | −0.0710 | −0.0247 | −0.0350 | 0.0471 | −0.0369 |
| instestini | | | | | | | | | | | |
| Gelria | 189326 | 3 | 0.0071 | 0.1559 | 0.1468 | −0.3122 | −0.0411 | −0.0792 | 0.0736 | −0.0280 | 0.1307 |
| Dorea | 189330 | 1 | −0.0063 | −0.5760 | −0.1761 | −0.0908 | 0.1468 | −0.1588 | −0.2054 | −0.0209 | −0.0355 |
| Turicibacter | 191303 | 1 | 0.0150 | 0.2369 | 0.1019 | −0.0350 | −0.0835 | −0.0222 | 0.0507 | −0.0092 | 0.1157 |
| Desulfovibrionaceae | 194924 | 1 | 0.0185 | 0.1056 | −0.0969 | 0.0561 | 0.0218 | 0.8555 | 0.1130 | 0.2410 | −0.587 |
| Bacteroidia | 200643 | 2 | 0.0150 | −0.2967 | −0.7737 | 0.1412 | 0.1622 | 0.0592 | −0.1316 | −0.0768 | −0.1261 |
| Flavobacteriales | 200644 | 1 | 0.0354 | −0.2081 | −0.0736 | 0.0251 | −0.0439 | 0.0649 | 0.0571 | 0.0031 | −0.0781 |
| Actibobacteria | 201174 | 1 | −0.0616 | −0.6061 | −0.1441 | −0.0128 | 0.0826 | −0.1269 | −0.1212 | −0.0656 | −0.1324 |
| Fusobacteriia | 203490 | 1 | 0.0242 | 0.1251 | −0.0274 | −0.0018 | −0.0328 | −0.0119 | 0.0443 | 0.0002 | 0.0023 |
| Fusobacteriales | 203491 | 1 | 0.0242 | 0.1251 | −0.0274 | −0.0018 | −0.0328 | −0.0119 | 0.0443 | 0.0002 | 0.0023 |
| Fusobacteriaceae | 203492 | 1 | 0.0284 | 0.1088 | −0.0139 | −0.0122 | −0.0177 | −0.0095 | 0.0344 | −0.0067 | 0.0035 |
| Verrucomicrobiae | 203494 | 3 | 0.0548 | 0.0571 | −0.0668 | 0.8056 | −0.1169 | 0.0550 | 0.0515 | 0.0237 | −0.0254 |
| Verrucomicrobiaceae | 203557 | 3 | 0.0546 | 0.0589 | −0.0651 | 0.8047 | −0.1182 | 0.0563 | 0.0526 | 0.0263 | −0.0249 |
| Fibrobacteria | 204430 | 8 | 0.0387 | 0.1142 | 0.0445 | −0.0354 | −0.0571 | −0.0043 | 0.0806 | −0.0123 | 0.6197 |
| Fibrobacteraceae | 204431 | 8 | 0.0287 | 0.1134 | 0.0366 | −0.0277 | −0.0594 | −0.0011 | 0.0840 | −0.0244 | 0.6052 |
| Rhodospirillales | 204441 | 2 | −0.0031 | −0.0708 | −0.2125 | 0.0450 | 0.1013 | 0.0087 | −0.0881 | −0.0189 | −0.0723 |
| Bacteroides | 204516 | 1 | −0.0138 | 0.0779 | 0.0508 | 0.0318 | −0.0309 | 0.0230 | 0.0444 | 0.0252 | 0.0216 |
| massiliensis | | | | | | | | | | | |
| Rhodocyclales | 206389 | 8 | −0.0357 | 0.0673 | −0.0128 | −0.0495 | −0.0309 | −0.0196 | 0.0744 | −0.0242 | 0.2666 |
| Anaerostipes | 207244 | 1 | 0.0172 | −0.4938 | −0.1992 | −0.0514 | −0.0457 | −0.0935 | −0.0492 | −0.0218 | −0.0385 |
| Allisonella | 209879 | 4 | −0.0377 | 0.1392 | −0.0042 | 0.0373 | −0.2075 | 0.0252 | 0.0893 | −0.0043 | 0.0239 |
| Allisonella | 209880 | 4 | −0.0377 | 0.1392 | −0.0042 | 0.0373 | −0.2075 | 0.0252 | 0.0893 | −0.0043 | 0.0239 |
| histaminiformans | | | | | | | | | | | |
| Desulfovibrionales | 213115 | 5 | 0.0185 | 0.1056 | −0.0970 | 0.0562 | 0.0217 | 0.8555 | 0.1130 | 0.0409 | −0.0587 |
| Campylobacterales | 213849 | 1 | 0.0150 | 0.2347 | 0.0475 | −0.0084 | −0.0726 | −0.0746 | 0.0197 | 0.0646 | 0.0087 |
| Subdoligranulum | 214851 | 1 | 0.0069 | −0.3132 | −0.0546 | −0.0003 | 0.2863 | −0.1205 | −0.1609 | 0.0037 | −0.0330 |
| variabile | | | | | | | | | | | |
| Alistipes finegoldii | 214856 | 2 | 0.0330 | 0.0276 | −0.0811 | 0.0438 | −0.0315 | 0.0868 | 0.0680 | 0.0099 | −0.0163 |
| Oscillospiraceae | 216572 | 5 | 0.0417 | 0.1778 | −0.0237 | 0.1119 | −0.1134 | 0.3691 | 0.1512 | −0.0415 | −0.0977 |
| Bifidobacterium | 216816 | 2 | −0.0639 | −0.0185 | 0.0634 | −0.0634 | −0.0075 | −0.0086 | 0.0449 | −0.0235 | 0.0335 |
| longum | | | | | | | | | | | |
| Faecalibacterium | 246851 | 1 | −0.0245 | −0.5859 | −0.1805 | −0.0456 | 0.3564 | −0.2518 | −0.2981 | −0.0272 | −0.1255 |
| Dialister invisus | 218358 | 1 | −0.0319 | −0.0968 | −0.0975 | −0.0021 | 0.0856 | 0.0033 | −0.0515 | 0.0240 | −0.0394 |
| Fibrobacterales | 218872 | 8 | 0.0387 | 0.1142 | 0.0445 | −0.0354 | −0.0571 | −0.0043 | 0.0806 | −0.0123 | 0.6197 |
| Peptoniphilus | 226531 | 1 | −0.0092 | 0.1760 | 0.0503 | 0.0060 | −0.0335 | −0.0016 | 0.0126 | −0.0135 | −0.0040 |
| sp. 2002-2300004 | | | | | | | | | | | |
| Sutterella | 234908 | 4 | 0.0042 | −0.0919 | −0.0388 | −0.0048 | 0.4283 | −0.0541 | −0.0688 | −0.0080 | −0.0002 |
| stercoricanis | | | | | | | | | | | |
| Fastidiosipila | 236752 | 1 | 0.0423 | 0.0121 | 0.0204 | −0.0558 | −0.0233 | −0.0111 | −0.0133 | 0.1137 | 0.0505 |
| Alistipes | 239759 | 1 | −0.0573 | −0.3076 | −0.3741 | 0.2459 | 0.0966 | 0.0084 | −0.3529 | −0.0452 | −0.2225 |
| Akkermansia | 239934 | 3 | 0.0543 | 0.0614 | −0.0644 | 0.8030 | −0.1219 | 0.0588 | 0.0546 | 0.0267 | −0.0239 |
| Akkermansia | 239935 | 3 | 0.0316 | 0.0386 | −0.0643 | 0.6195 | −0.0457 | 0.0517 | 0.0246 | 0.0097 | −0.0126 |
| muciniphila | | | | | | | | | | | |
| Hespellia | 241189 | 1 | −0.0168 | 0.3462 | 0.1386 | −0.0465 | −0.1394 | 0.1875 | 0.2547 | 0.0129 | 0.0413 |
| Anaerotruncus | 244127 | 1 | 0.0210 | −0.2390 | −0.0085 | 0.0257 | 0.0137 | −0.0858 | −0.1840 | −0.0155 | −0.0877 |
| Bacteroides | 246789 | 2 | −0.0240 | −0.0102 | −0.1182 | 0.1151 | −0.0340 | 0.0874 | −0.0122 | 0.0100 | −0.0315 |
| sp. 35AE37 | | | | | | | | | | | |
| Marvinbryantia | 248744 | 1 | −0.0106 | 0.2608 | 0.1178 | −0.0191 | −0.1370 | 0.1566 | 0.2108 | −0.0054 | 0.0761 |
| Pseudoclavibacter | 255204 | 7 | 0.1554 | 0.1159 | 0.0300 | −0.0230 | −0.0228 | 0.0423 | 0.0299 | 0.2145 | −0.0116 |
| Victivallaceae | 255528 | 6 | 0.0197 | 0.1995 | 0.0558 | −0.0397 | −0.1275 | 0.0586 | 0.5807 | 0.0142 | 0.0157 |
| Lentisphaerae | 256845 | 6 | 0.0258 | 0.1934 | 0.0580 | −0.0405 | −0.1210 | 0.0639 | 0.5743 | 0.0228 | 0.0081 |
| Alistipes massiliensis | 265312 | 1 | −0.0395 | 0.1529 | 0.0711 | −0.0062 | −0.0784 | 0.0374 | 0.1269 | −0.0111 | 0.0454 |
| Odoribacter | 283168 | 5 | −0.0070 | 0.0844 | −0.1742 | 0.1067 | −0.0053 | 0.4714 | 0.2097 | −0.0037 | −0.0206 |
| Bacteroides salyersiae | 291644 | 2 | −0.0438 | 0.0358 | −0.0443 | 0.0094 | −0.0122 | 0.0588 | −0.0008 | 0.0115 | 0.0411 |
| Bacteroides nordii | 291645 | 1 | 0.0023 | 0.1446 | 0.0883 | 0.0068 | −0.0291 | 0.1053 | 0.1328 | −0.0020 | 0.0402 |
| Subdoligranulum | 292632 | 1 | −0.0339 | −0.5177 | −0.0878 | −0.0070 | 0.2646 | −0.2294 | −0.3156 | 0.0052 | −0.1091 |
| Flavonifractor plautii | 292800 | 5 | 0.0069 | 0.2141 | 0.2304 | −0.0304 | −0.0932 | 0.2089 | 0.1446 | −0.0045 | 0.0310 |
| Roseburia hominis | 301301 | 1 | −0.0131 | 0.0242 | −0.0032 | −0.0233 | −0.0566 | 0.0686 | 0.0877 | −0.0167 | −0.0151 |
| Roseburia faecis | 301302 | 1 | −0.0414 | −0.1246 | −0.0819 | 0.0507 | 0.0478 | 0.0529 | 0.0620 | 0.0235 | −0.0526 |
| Dialister | 308994 | 1 | 0.0341 | 0.0557 | 0.0416 | −0.0253 | 0.0393 | −0.0883 | −0.0483 | 0.0612 | −0.0725 |
| propionicifaciens | | | | | | | | | | | |
| Bacteroides plebeius | 310297 | 2 | −0.0198 | −0.0242 | 0.0176 | −0.0496 | 0.0932 | −0.0818 | −0.0597 | 0.0149 | 0.0280 |
| Bacteroides coprocola | 310298 | 5 | −0.0263 | −0.0088 | 0.0631 | −0.0342 | 0.0419 | −0.0756 | −0.0671 | 0.0173 | 0.0179 |
| Parabacteroides | 328812 | 6 | 0.0031 | 0.1135 | 0.0321 | −0.0259 | −0.0868 | 0.0433 | 0.3042 | −0.0158 | 0.0754 |
| goldsteinii | | | | | | | | | | | |
| Alistipes shahii | 328814 | 2 | 0.0308 | 0.0525 | 0.0813 | −0.0597 | −0.0650 | −0.0039 | 0.0140 | −0.0040 | 0.0336 |
| Bacteroides finegoldii | 338188 | 2 | −0.0051 | 0.0760 | 0.2546 | −0.0091 | −0.0654 | −0.0072 | 0.0512 | 0.0160 | 0.0671 |
| Lactonifactor | 341220 | 2 | −0.0666 | 0.0111 | 0.2842 | −0.0996 | −0.0021 | −0.2106 | −0.1448 | −0.0173 | 0.0435 |
| longoviformis | | | | | | | | | | | |
| Bacteroides dorei | 357276 | 2 | 0.0142 | −0.1000 | 0.1408 | −0.0734 | −0.0688 | −0.0434 | −0.0395 | 0.0343 | 0.0621 |
| Roseburia | 360807 | 1 | 0.0035 | −0.0062 | 0.0047 | −0.1020 | −0.0146 | 0.0161 | 0.0353 | −0.0253 | 0.0157 |
| inulinivorans | | | | | | | | | | | |
| Peptoniphilus sp. | 361493 | 1 | 0.0826 | 0.3393 | 0.0467 | −0.0006 | −0.0893 | −0.0584 | 0.0203 | 0.0183 | −0.0383 |
| gpaco18A | | | | | | | | | | | |
| Bacteroides | 371599 | 2 | 0.0092 | −0.0096 | −0.1227 | 0.0121 | −0.0311 | 0.0855 | 0.0788 | −0.0550 | −0.0425 |
| sp. XB12B | | | | | | | | | | | |
| Bacteroides | 371600 | 2 | 0.0086 | 0.0760 | 0.0898 | −0.0343 | −0.0320 | −0.0036 | 0.0378 | −0.0499 | 0.0853 |
| sp. XB44A | | | | | | | | | | | |
| Parabacteroides | 375288 | 2 | 0.0628 | −0.2481 | −0.6594 | 0.1628 | 0.1494 | 0.0573 | −0.1662 | −0.0518 | −0.1389 |
| Prevotella timonensis | 386414 | 1 | 0.0155 | 0.0575 | 0.0151 | −0.0293 | −0.0403 | −0.0569 | −0.0185 | 0.0464 | 0.0082 |
| Parabacteroides | 387661 | 7 | −0.0102 | 0.0180 | −0.0860 | 0.0411 | −0.0465 | 0.0707 | 0.0595 | −0.4351 | 0.0098 |
| johnsonii | | | | | | | | | | | |
| Barnesiella | 397864 | 3 | −0.0034 | −0.1092 | −0.2579 | 0.3323 | 0.0767 | 0.1080 | −0.1300 | −0.0521 | −0.1151 |
| Howardella | 404402 | 4 | 0.0186 | 0.1110 | −0.0225 | −0.0251 | −0.2385 | 0.0569 | 0.1745 | −0.0428 | 0.0866 |
| Howardella ureilytica | 404403 | 4 | −0.0009 | 0.1284 | −0.0230 | −0.0316 | −0.2430 | 0.0798 | 0.2082 | −0.0414 | 0.0842 |
| Citrobacter sp. BW4 | 408103 | 5 | −0.0512 | 0.0420 | 0.0198 | 0.0347 | 0.0037 | −0.1591 | −0.0539 | −0.0399 | 0.0146 |
| Anaerococcus | 411577 | 1 | 0.0729 | 0.2056 | 0.0841 | −0.0363 | −0.0717 | −0.1230 | −0.0571 | 0.0582 | −0.0032 |
| murdochii | | | | | | | | | | | |
| Opitutae | 414999 | 6 | 0.0243 | 0.0890 | −0.0511 | −0.0084 | −0.0136 | 0.0609 | 0.2088 | −0.0107 | 0.0138 |
| Puniccicoccales | 415001 | 6 | 0.0243 | 0.0889 | −0.0512 | −0.0084 | −0.0136 | 0.0610 | 0.2088 | −0.0106 | 0.0138 |
| Blautia wexlerae | 418240 | 1 | −0.0132 | −0.5053 | −0.2577 | −0.0177 | −0.0246 | −0.0809 | −0.0356 | 0.0245 | −0.0352 |
| Lactonifactor | 420345 | 2 | −0.0482 | 0.1488 | 0.3440 | −0.1139 | −0.0017 | −0.1506 | −0.0893 | −0.0102 | 0.0876 |
| Veillonella rogosae | 423477 | 0 | −0.1526 | 0.0067 | −0.0404 | 0.0684 | −0.0063 | −0.0216 | −0.0493 | −0.0252 | 0.0085 |
| Bacteroides sp. CB57 | 426340 | 6 | 0.0000 | −0.0196 | 0.0300 | −0.0459 | −0.0091 | −0.0560 | −0.1054 | 0.0258 | 0.0120 |
| Moryella | 437755 | 2 | −0.0032 | 0.1093 | 0.1581 | −0.0106 | −0.1131 | 0.0369 | 0.0240 | −0.0065 | −0.0519 |
| Megamonas | 437897 | 0 | 0.1764 | −0.0285 | 0.0124 | 0.0181 | 0.0470 | −0.0109 | 0.0033 | −0.0033 | 0.0264 |
| funiformis | | | | | | | | | | | |
| Adlerereutzia | 446660 | 1 | −0.0039 | 0.3398 | 0.2649 | −0.0489 | −0.1240 | 0.0971 | 0.2036 | −0.0062 | 0.1394 |
| equolifaciens | | | | | | | | | | | |
| Adlerereutzia | 447020 | 1 | −0.0053 | 0.3416 | 0.2619 | −0.0525 | −0.1271 | 0.0955 | 0.2074 | −0.0033 | 0.1370 |
| Alistipes | 447027 | 5 | 0.0019 | 0.1229 | −0.0302 | 0.0495 | −0.0682 | 0.2923 | 0.2268 | 0.0213 | −0.0439 |
| sp. EBA6-25el2 | | | | | | | | | | | |
| Bacteroides | 447029 | 2 | −0.0426 | −0.0360 | −0.1118 | 0.0363 | −0.0720 | 0.0182 | 0.0477 | −0.0274 | −0.0229 |
| sp. EBA5-17 | | | | | | | | | | | |
| Paraprevotella clara | 454154 | 0 | 0.3149 | 0.0105 | −0.0501 | 0.0081 | 0.0719 | 0.0219 | −0.0182 | −0.0197 | −0.0022 |
| Oscillibacter | 459786 | 1 | 0.0828 | 0.2215 | 0.1143 | −0.0278 | −0.1759 | 0.0298 | 0.1726 | 0.0199 | 0.0888 |
| Bacteroides sp. 2_2_4 | 469590 | 1 | 0.0019 | 0.0363 | −0.0587 | −0.0433 | −0.0312 | 0.0307 | 0.0400 | −0.0176 | −0.0094 |
| Gordonibacter | 471189 | 2 | −0.0440 | 0.2492 | 0.3507 | −0.1269 | −0.1597 | −0.1068 | 0.0518 | 0.0111 | 0.1157 |
| pamelaeae | | | | | | | | | | | |
| Alistipes sp. | 478202 | 6 | 0.0051 | 0.2620 | 0.1022 | −0.0250 | −0.1410 | 0.1725 | 0.5424 | 0.0331 | 0.0886 |
| NML05A004 | | | | | | | | | | | |
| Dialister | 487173 | 0 | −0.0589 | 0.0240 | 0.0193 | −0.0041 | 0.0504 | −0.0523 | −0.0470 | 0.0186 | 0.0130 |
| succinatiphilus | | | | | | | | | | | |
| Barnesiella | 487174 | 3 | −0.0026 | −0.0995 | −0.2630 | 0.3023 | 0.0709 | 0.1089 | −0.1020 | −0.0478 | −0.1154 |
| intestinihominis | | | | | | | | | | | |
| Parasutterella | 487175 | 4 | 0.0688 | 0.0756 | −0.0705 | −0.0188 | 0.0807 | 0.0744 | 0.1262 | 0.0346 | |
| excrementihominis | | | | | | | | | | | 0.0136 |
| Porphyromonas | 501496 | 1 | 0.1185 | 0.2470 | 0.0758 | 0.0251 | −0.0550 | −0.0657 | 0.0080 | 0.0485 | −0.0056 |
| bennonis | | | | | | | | | | | |
| Peptoniphilus | 507750 | 1 | 0.0131 | 0.1805 | −0.0008 | 0.0086 | −0.0674 | −0.0172 | 0.0149 | −0.0089 | −0.0192 |
| duerdenii | | | | | | | | | | | |
| Murdochiella | 507844 | 1 | 0.0490 | 0.2551 | 0.0813 | −0.0045 | −0.0312 | −0.0420 | 0.0308 | 0.0620 | −0.0185 |
| asaccharolytica | | | | | | | | | | | |
| Synergistetes | 508458 | 3 | 0.0614 | 0.0499 | 0.0620 | −0.2582 | −0.0179 | −0.0166 | 0.1316 | 0.0232 | 0.0192 |
| Cloacibacillus | 508459 | 3 | 0.0198 | 0.0522 | 0.0441 | −0.2182 | −0.0177 | −0.0522 | 0.1572 | −0.0361 | 0.0424 |
| Erysipelotrichia | 526524 | 1 | −0.0304 | −0.4264 | −0.1460 | 0.0211 | 0.0825 | 0.0507 | −0.0348 | −0.0446 | −0.1057 |
| Erysipelotrichales | 526525 | 1 | −0.0322 | −0.4263 | −0.1463 | 0.0177 | 0.0821 | 0.0508 | −0.0316 | −0.0434 | −0.1010 |
| Blautia glucerasea | 536633 | 2 | −0.0219 | 0.0217 | 0.2349 | −0.0458 | −0.0224 | −0.0751 | −0.0738 | −0.0109 | 0.0517 |
| Bacteroides sp. | 537274 | 2 | 0.0054 | −0.0215 | −0.0875 | 0.0095 | 0.0566 | −0.0246 | −0.0321 | 0.0397 | −0.0063 |
| DJF_B097 | | | | | | | | | | | |
| Ruminococcaceae | 541000 | 1 | −0.0299 | −0.6562 | −0.2133 | −0.0115 | 0.3351 | −0.02631 | −0.3447 | −0.0155 | −0.1651 |
| Clostridiales | 543310 | 1 | −0.0977 | 0.0746 | 0.1177 | −0.0106 | −0.0502 | 0.0186 | 0.0511 | −0.1331 | 0.0189 |
| Family XI. | | | | | | | | | | | |
| Incertae Sedis | | | | | | | | | | | |
| Clostridiales | 543314 | 1 | 0.0309 | 0.4028 | 0.2384 | −0.1135 | −0.1041 | 0.1156 | 0.2585 | 0.0401 | 0.0947 |
| Family XIII. | | | | | | | | | | | |
| Incertae Sedis | | | | | | | | | | | |
| Tenericutes | 544448 | 1 | −0.0532 | 0.0847 | −0.0019 | −0.0203 | −0.0221 | −0.0266 | 0.0552 | −0.0098 | 0.0200 |
| Butyricimonas virosa | 544645 | 6 | 0.0106 | 0.1151 | −0.0008 | −0.0490 | −0.0714 | 0.0631 | 0.3397 | 0.0118 | 0.0589 |
| Anaerotruncus | 545498 | 3 | 0.0131 | 0.0745 | 0.1300 | −0.1521 | 0.0352 | −0.0140 | 0.0786 | −0.0340 | 0.0749 |
| sp. NML 070203 | | | | | | | | | | | |
| Coprobacillus sp. D6 | 556262 | 5 | −0.0240 | 0.0293 | 0.0706 | −0.0059 | −0.0066 | −0.1925 | −0.0588 | 0.0148 | 0.0376 |
| Corynebacterium | 556548 | 1 | 0.0250 | 0.0823 | 0.0221 | −0.0074 | −0.0171 | 0.0008 | 0.0074 | 0.0360 | 0.0158 |
| freiburgense | | | | | | | | | | | |
| Acidaminococcus | 563191 | 2 | 0.0523 | 0.0634 | −0.0665 | −0.0028 | 0.0974 | 0.0515 | 0.0478 | −0.0301 | −0.0277 |
| sp. D21 | | | | | | | | | | | |
| Blautia | 572511 | 1 | −0.0053 | 0.6326 | −0.3135 | 0.0144 | −0.0173 | −0.0471 | −0.0905 | 0.0235 | −0.1003 |
| Butyricimonas | 574697 | 6 | 0.0094 | 0.1264 | −0.0961 | 0.0326 | −0.0754 | 0.2750 | 0.5213 | 0.0361 | 0.0114 |
| Paraprevotella | 577309 | 4 | 0.2438 | −0.0179 | −0.0552 | 0.0031 | 0.1421 | −0.0110 | −0.0704 | −0.0482 | −0.0345 |
| Parasutterella | 577310 | 4 | 0.0630 | 0.0764 | −0.0709 | −0.0297 | 0.0913 | 0.0776 | 0.1262 | 0.0305 | 0.0128 |
| Enterorhabdus | 580024 | 4 | 0.0335 | 0.1562 | −0.0513 | −0.0149 | −0.0760 | 0.0979 | 0.1951 | −0.0037 | 0.0936 |
| Roseburia sp. 11SE39 | 583273 | 1 | 0.0412 | −0.4038 | −0.0038 | −0.1325 | −0.0512 | −0.2411 | −0.1704 | −0.0781 | −0.0267 |
| Bacteroides sp. D22 | 585544 | 2 | 0.0180 | −0.0151 | −0.3155 | 0.0225 | −0.0503 | 0.0702 | 0.1490 | −0.0351 | −0.0319 |
| Robinsoniella | 588605 | 6 | −0.0027 | 0.0726 | −0.0500 | 0.0054 | −0.0681 | 0.0148 | 0.2586 | −0.0220 | 0.0423 |
| Bifidobacterium | 592977 | 1 | 0.0257 | −0.0753 | −0.0043 | −0.0393 | 0.0009 | −0.0253 | −0.0219 | −0.0367 | −0.0579 |
| stercoris | | | | | | | | | | | |
| Hydrogenoanaero- | 596767 | 3 | 0.0477 | 0.1119 | 0.1320 | −0.2836 | 0.0281 | −0.0423 | 0.1057 | −0.0641 | −0.1337 |
| bacterium | | | | | | | | | | | |
| Negativicoccus | 620903 | 1 | 0.0489 | 0.0124 | −0.0308 | 0.0231 | −0.0527 | −0.0134 | −0.0439 | 0.0913 | −0.0137 |
| succinicivornas | | | | | | | | | | | |
| Bacteroides clarus | 626929 | 3 | 0.0030 | 0.0162 | −0.0413 | −0.0885 | 0.0112 | 0.0144 | 0.0689 | −0.0401 | 0.0306 |
| Alistipes indistinetus | 626932 | 3 | 0.0033 | 0.1550 | 0.1599 | −0.1967 | −0.0758 | −0.0829 | 0.1262 | −0.0104 | 0.1146 |
| Odoribacter laneus | 626933 | 7 | 0.0145 | 0.0748 | 0.0255 | −0.0155 | −0.0075 | 0.0537 | 0.0867 | 0.2031 | 0.0171 |
| Slackia piriformis | 626934 | 4 | 0.0123 | 0.1054 | 0.0485 | −0.0414 | −0.1666 | 0.0270 | 0.1084 | −0.0018 | −0.0197 |
| Phascolaretobacterium | 626940 | 2 | −0.0645 | −0.0600 | −0.0168 | −0.0323 | 0.0774 | 0.0013 | −0.0715 | 0.0245 | −0.0239 |
| succinatutens | | | | | | | | | | | |
| Sutterella | 629946 | 4 | −0.0195 | 0.0176 | 0.0091 | 0.0268 | 0.1993 | 0.0057 | −0.0420 | −0.0088 | −0.0059 |
| sp. YIT 12070 | | | | | | | | | | | |
| Bifidobacterium | 630129 | 0 | −0.2118 | −0.0191 | −0.0020 | 0.0036 | −0.0114 | 0.0541 | −0.0360 | 0.0027 | −0.0424 |
| kashiwanohense | | | | | | | | | | | |
| Porphyromonas | 631030 | 1 | 0.0429 | 0.2034 | 0.0285 | 0.0283 | −0.0465 | 0.0162 | 0.0694 | 0.0383 | −0.0148 |
| sp. 2026 | | | | | | | | | | | |
| Gordonibacter | 644652 | 2 | −0.0435 | 0.2382 | 0.3823 | −0.1514 | −0.1662 | −0.1026 | 0.0478 | 0.0194 | 0.1151 |
| Slacka sp. NATTS | 647703 | 4 | −0.0063 | 0.1672 | −0.0327 | 0.0084 | −0.2737 | 0.1010 | 0.1629 | 0.0260 | 0.0112 |
| Anaerostipes hadrus | 649756 | 1 | 0.0063 | 0.0034 | 0.0075 | −0.0134 | 0.0554 | −0.0434 | 0.0277 | −0.0093 | −0.0075 |
| Synergistia | 649775 | 3 | 0.0614 | 0.0494 | 0.0620 | −0.2582 | −0.0179 | −0.0166 | 0.1316 | 0.0232 | 0.0192 |
| Synergistales | 649776 | 3 | 0.0614 | 0.0499 | 0.0620 | −0.2582 | −0.0179 | −0.0166 | 0.1316 | 0.0232 | 0.0192 |
| Synergistaceae | 649777 | 3 | 0.0614 | 0.0499 | 0.0620 | −0.2582 | −0.0179 | −0.0166 | 0.1316 | 0.0232 | 0.0192 |
| Alistipes | 650643 | 5 | −0.0460 | 0.1352 | −0.0605 | 0.0900 | −0.1182 | 0.2521 | 0.2113 | 0.0286 | −0.0324 |
| sp. RMA 9912 | | | | | | | | | | | |
| Anaerosporobacter | 653683 | 3 | −0.0109 | 0.1052 | 0.0911 | −0.2027 | −0.0389 | −0.0227 | 0.1044 | 0.0128 | 0.0977 |
| Anaerostipes sp. | 665937 | 1 | −0.0422 | −0.1107 | 0.0991 | −0.0611 | 0.0171 | −0.1296 | −0.1249 | −0.0148 | 0.0334 |
| 3_2_56FAA | | | | | | | | | | | |
| Desulfovibrio sp. | 665942 | 6 | −0.0162 | 0.1547 | 0.0417 | −0.0520 | −0.0660 | 0.0930 | 0.2025 | 0.0144 | 0.0129 |
| 6_1_46AFAA | | | | | | | | | | | |
| Lactobacillus sp. | 665945 | 1 | −0.0387 | 0.0597 | 0.0360 | −0.0177 | 0.0007 | −0.0187 | 0.0285 | 0.0079 | 0.0093 |
| 7_1_47FAA | | | | | | | | | | | |
| Peptoniphilus sp. oral | 671216 | 1 | 0.0209 | 0.2121 | 0.0292 | 0.0675 | −0.0668 | −0.0422 | 0.0048 | 0.0478 | −0.0283 |
| taxon 836 | | | | | | | | | | | |
| Bacteroides faecis | 674529 | 2 | 0.0302 | 0.0787 | 0.2236 | −0.0674 | −0.0740 | −0.0963 | −0.0597 | 0.0055 | 0.0300 |
| Corynebacterium | 679663 | 1 | −0.0051 | 0.1170 | 0.0132 | 0.0625 | −0.0707 | −0.0184 | 0.0244 | −0.0623 | −0.0433 |
| canis | | | | | | | | | | | |
| Blautia sp. Ser8 | 689777 | 2 | −0.0279 | −0.0431 | 0.2071 | −0.0449 | 0.0217 | −0.0757 | −0.0848 | −0.0018 | 0.0128 |
| Bilophila sp. 4_1_30 | 693988 | 5 | 0.0145 | 0.1233 | −0.0701 | 0.0423 | −0.0484 | 0.7099 | 0.2251 | 0.0110 | −0.0456 |
| Corynebacterium sp. | 702963 | 1 | 0.0235 | 0.0694 | 0.0208 | −0.0190 | −0.0199 | −0.0488 | 0.0035 | 0.0522 | 0.0276 |
| NML96-0085 | | | | | | | | | | | |
| Streptococcus sp. oral | 712699 | 1 | −0.0519 | 0.1728 | 0.0596 | 0.0389 | −0.0646 | −0.0269 | −0.0221 | 0.0467 | 0.0335 |
| taxon G59 | | | | | | | | | | | |
| Caldicoprobacteraceae | 715221 | 3 | 0.0019 | 0.1011 | 0.1072 | −0.1890 | −0.0228 | −0.0453 | 0.0863 | 0.0028 | 0.1265 |
| Lactobacillus | 753938 | 2 | −0.0367 | 0.0874 | 0.0737 | −0.0287 | −0.0148 | 0.0013 | −0.0103 | −0.0642 | 0.0617 |
| sp. TAB-26 | | | | | | | | | | | |
| Peptoniphilus coxii | 755172 | 1 | 0.0437 | 0.1201 | 0.0445 | −0.0337 | −0.0255 | −0.0345 | 0.0052 | 0.0436 | −0.0207 |
| Bacteroides | 871324 | 2 | −0.0399 | 0.0681 | 0.0438 | −0.0053 | 0.0192 | 0.0381 | 0.0682 | −0.0201 | 0.0117 |
| stercorirosoris | | | | | | | | | | | |
| Blautia stercoris | 871664 | 1 | 0.0187 | −0.0779 | −0.0848 | −0.0338 | 0.0572 | 0.0267 | 0.0250 | 0.0440 | −0.0259 |
| Blautia faecis | 871665 | 1 | −0.0266 | −0.4502 | −0.2175 | 0.0852 | −0.0507 | −0.0760 | −0.1639 | −0.0007 | −0.1205 |
| Peptoniphilus sp. 1-14 | 875455 | 1 | −0.0098 | 0.0927 | 0.0551 | −0.0122 | −0.0540 | −0.0284 | −0.0135 | −0.0340 | −0.0192 |
| Peptoniphilus sp. 7-2 | 875465 | 1 | 0.0204 | 0.1791 | 0.0561 | 0.0035 | −0.0031 | −0.0105 | 0.0489 | −0.0474 | −0.0072 |
| Alistipes sp. HGB5 | 908612 | 1 | 0.0349 | 0.2138 | 0.0589 | 0.0432 | −0.0443 | 0.2160 | 0.2705 | 0.0210 | 0.0349 |
| Negativicoccus | 909928 | 1 | 0.0367 | 0.0286 | −0.0325 | −0.0009 | −0.0359 | −0.0304 | −0.0660 | 0.0872 | −0.0049 |
| Selenomonadales | 909929 | 4 | 0.1019 | −0.1116 | −0.1209 | 0.0222 | 0.2957 | 0.1078 | −0.0072 | −0.0386 | −0.0727 |
| Acidaminococcaceae | 909930 | 2 | 0.0311 | −0.1303 | −0.3042 | 0.0391 | 0.2055 | 0.0589 | −0.1176 | −0.0059 | −0.0876 |
| Negativicutes | 909932 | 4 | 0.1019 | −0.1116 | −0.1209 | 0.0222 | 0.2957 | 0.1078 | −0.0072 | −0.0386 | −0.0727 |
| Eggerthella sp. HGA1 | 910311 | 2 | −0.0109 | 0.0553 | 0.3223 | −0.0531 | −0.0118 | −0.1188 | −0.1592 | −0.0106 | −0.0059 |
| Bacteroides | 925962 | 2 | −0.0023 | −0.1582 | −0.1239 | 0.0106 | 0.1331 | −0.1244 | −0.1782 | 0.0283 | 0.0016 |
| sp. SLC1-38 | | | | | | | | | | | |
| Veillonella sp. CM60 | 936384 | 1 | −0.0481 | 0.0290 | 0.0494 | −0.0114 | 0.0002 | −0.0057 | −0.0122 | −0.0061 | 0.0190 |
| Actinomyces | 936549 | 1 | 0.0404 | 0.1352 | 0.0202 | −0.0040 | −0.0876 | −0.1017 | −0.0659 | 0.0024 | 0.0220 |
| sp. ICM54 | | | | | | | | | | | |
| Streptococcus | 936579 | 1 | −0.0330 | 0.0929 | −0.0268 | 0.0272 | −0.0714 | −0.0496 | −0.0287 | −0.0273 | −0.0040 |
| sp. BS35a | | | | | | | | | | | |
| Veillonella | 936592 | 1 | −0.0329 | 0.1233 | 0.0864 | 0.0117 | −0.0688 | 0.0120 | 0.0294 | −0.0031 | 0.0425 |
| sp. MSA12 | | | | | | | | | | | |
| Lactococcus | 937666 | 1 | −0.0118 | 0.1474 | 0.0403 | −0.0272 | −0.0468 | −0.0040 | −0.0058 | 0.0079 | 0.0397 |
| sp. MH5-2 | | | | | | | | | | | |
| Anaerococcus | 938290 | 1 | 0.0123 | 0.2550 | 0.0747 | 0.0184 | −0.0395 | −0.0247 | 0.0333 | −0.0596 | 0.0152 |
| sp. 8404299 | | | | | | | | | | | |
| Anaerococcus | 938292 | 1 | 0.0646 | 0.0695 | 0.0426 | 0.0035 | −0.0039 | 0.0136 | −0.0322 | 0.0687 | 0.0304 |
| sp. 9401487 | | | | | | | | | | | |
| Anaerococcus | 938292 | 1 | 0.1151 | 0.1189 | 0.0082 | 0.0194 | −0.0485 | 0.0251 | −0.0503 | 0.1099 | −0.0417 |
| sp. 9402080 | | | | | | | | | | | |
| Enterococcus sp. SI-4 | 946073 | 0 | −0.4580 | 0.0074 | 0.0451 | −0.0192 | 0.0088 | 0.0062 | −0.0010 | −0.0048 | −0.0128 |
| flavonifractor | 946234 | 5 | 0.0527 | 0.0858 | −0.0185 | 0.1690 | −0.0756 | 0.3538 | 0.0934 | −0.0001 | −0.1495 |
| Staphylococcus | 990512 | 1 | 0.0364 | 0.0497 | −0.0431 | −0.0139 | −0.0026 | −0.0490 | −0.0812 | 0.0067 | −0.0110 |
| sp. C9I2 | | | | | | | | | | | |
| Enterobacter | 994321 | 1 | −0.0596 | 0.0418 | 0.0559 | −0.0377 | 0.0012 | −0.0048 | −0.0025 | −0.0684 | 0.0258 |
| sp. BS2-1 | | | | | | | | | | | |
| Sutterellaceae | 995019 | 4 | 0.0375 | −0.1778 | −0.1860 | −0.0570 | 0.9281 | −0.0285 | −0.1571 | −0.0455 | −0.0438 |
| Anaerostipes sp. | 999930 | 1 | −0.0059 | −0.5150 | −0.1939 | −0.0770 | −0.0366 | −0.1361 | −0.0825 | −0.0157 | −0.0218 |
| 5_1_63FAA | | | | | | | | | | | |
| Pseuodoflavonifractor | 1017280 | 2 | −0.0283 | 0.1662 | 0.3548 | −0.0914 | −0.0705 | −0.0997 | −0.0609 | 0.0038 | 0.1093 |
| Dielma fastidiosa | 1034346 | 2 | −0.0272 | 0.0060 | 0.2509 | −0.0952 | −0.0236 | −0.1225 | −0.0696 | −0.0389 | 0.0503 |
| Corynebacterium | 1050174 | 1 | 0.0723 | 0.0923 | −0.0043 | 0.0206 | −0.0327 | −0.0021 | 0.0045 | 0.0092 | −0.0018 |
| epidermidicanis | | | | | | | | | | | |
| Coprobacter | 1099853 | 1 | −0.0267 | 0.1545 | 0.0484 | −0.0316 | −0.0515 | 0.1607 | 0.2508 | −0.0148 | 0.0410 |
| fastidiosus | | | | | | | | | | | |
| Fusicatenibacter | 1150298 | 1 | 0.0218 | −0.5978 | −0.1332 | −0.0666 | 0.0006 | −0.2077 | −0.1819 | 0.0060 | −0.0567 |
| saccharivorans | | | | | | | | | | | |
| Faecalibacterium sp. | 1151492 | 1 | −0.0291 | −0.1265 | −0.0836 | 0.0262 | 0.1957 | 0.0008 | 0.0140 | 0.0575 | −0.0128 |
| canine oral taxon 147 | | | | | | | | | | | |
| Blautia sp. YHC-4 | 1157314 | 2 | −0.0153 | 0.1091 | 0.1981 | −0.0003 | −0.0460 | 0.0142 | −0.0202 | −0.0105 | −0.0140 |
| Murdochiella | 1161127 | 1 | 0.0533 | 0.2798 | 0.0662 | 0.0193 | −0.0271 | −0.0640 | 0.0384 | 0.0599 | 0.0061 |
| Phascolaretobacterium | 1217279 | 0 | 0.1750 | 0.0423 | −0.0507 | 0.0284 | 0.0417 | −0.0104 | −0.0366 | −0.0188 | −0.0579 |
| sp. 377 | | | | | | | | | | | |
| Parabacteroides | 1217282 | 1 | 0.0205 | 0.0416 | 0.0706 | −0.0546 | −0.0442 | 0.0197 | 0.0460 | 0.0338 | 0.0343 |
| sp. 157 | | | | | | | | | | | |
| Corynebacterium | 1234514 | 1 | 0.0184 | 0.0919 | 0.0501 | −0.0432 | −0.0690 | −0.0870 | −0.0568 | 0.0035 | 0.0875 |
| sp. jw37 | | | | | | | | | | | |
| Streptococcus sp. | 1244424 | 2 | 0.0300 | 0.0421 | 0.1425 | −0.0873 | −0.0290 | −0.0808 | −0.0680 | 0.0041 | 0.0791 |
| 2011_Oral_MS_A3 | | | | | | | | | | | |
| Sutterella sp. 252 | 1248467 | 4 | −0.0658 | 0.0009 | −0.0356 | 0.0306 | 0.0822 | 0.0002 | −0.0104 | −0.0508 | −0.0085 |
| Alloprevotella | 1283313 | 4 | 0.0151 | −0.0532 | 0.0099 | 0.0430 | 0.1994 | −0.0589 | −0.1209 | −0.0147 | −0.0051 |
| Megasphaera | 1287023 | 1 | 0.0502 | 0.0564 | −0.0428 | 0.0289 | 0.0563 | 0.0373 | 0.0438 | −0.0054 | 0.0024 |
| sp. S6-MB2 | | | | | | | | | | | |
| Intestinimonas | 1297617 | 3 | −0.0171 | −0.0127 | 0.2060 | −0.1851 | 0.0071 | −0.0663 | −0.0142 | 0.0208 | 0.1068 |
| butyriciproducens | | | | | | | | | | | |
| Lentisphaeria | 1313211 | 6 | 0.0197 | 0.1995 | 0.0558 | −0.0397 | −0.1275 | 0.0586 | 0.5807 | 0.0142 | 0.0157 |
| Finegoldia | 1344331 | 1 | −0.0543 | 0.1991 | 0.0972 | −0.0032 | −0.0642 | 0.0129 | 0.0849 | −0.0835 | −0.0157 |
| sp. S9 AA1-5 | | | | | | | | | | | |
| Coprobacter | 1348911 | 1 | −0.0246 | 0.1533 | 0.0462 | −0.0330 | −0.0495 | 0.1626 | 0.2525 | −0.0115 | 0.0439 |
| Bacteroides sp. J1511 | 1365140 | 2 | −0.0148 | −0.0546 | −0.1091 | 0.0372 | −0.0413 | 0.0294 | 0.0552 | 0.0078 | −0.0195 |
| Intestinimonas | 1392389 | 1 | 0.0165 | 0.2903 | 0.1869 | 0.0022 | −0.1052 | 0.2005 | 0.2018 | −0.0081 | 0.0231 |
| Fusicatenibacter | 1407607 | 1 | 0.0111 | −0.6172 | −0.2280 | −0.0206 | −0.0057 | −0.1851 | −0.1592 | 0.0294 | −0.0689 |
| Eisenbergiella | 1432051 | 2 | 0.0332 | 0.1040 | 0.4055 | −0.1618 | −0.0395 | −0.1117 | −0.0144 | −0.0181 | 0.0725 |
| Eisenbergiella tayi | 1432052 | 2 | 0.0409 | 0.1251 | 0.4151 | −0.1511 | −0.0476 | −0.1138 | −0.0038 | −0.0232 | 0.0657 |
| Candidatus | 1470349 | 1 | −0.0164 | 0.0821 | 0.1535 | −0.0393 | −0.0214 | −0.0545 | −0.0451 | −0.0333 | 0.0332 |
| Stoquefielius | | | | | | | | | | | |
| Candidatus | 1470353 | 2 | −0.0224 | 0.1377 | 0.2756 | −0.1516 | 0.0034 | −0.1773 | −0.0742 | 0.0510 | 0.1540 |
| Soleaferrea | | | | | | | | | | | |
| Butyricimonas | 1472416 | 6 | 0.0151 | 0.1418 | 0.0203 | 0.0382 | −0.1379 | 0.1095 | 0.3761 | −0.0056 | 0.0256 |
| sp. JCM 18676 | | | | | | | | | | | |
| Butyricimonas | 1472417 | 6 | 0.0043 | 0.1400 | 0.0260 | −0.0269 | −0.1166 | 0.1627 | 0.4750 | 0.0039 | 0.0013 |
| sp. JCM 18677 | | | | | | | | | | | |
| Dielma | 1472649 | 2 | −0.0155 | 0.0643 | 0.2815 | −0.0896 | −0.0313 | −0.0990 | −0.0447 | 0.0076 | 0.0682 |
| Senegalimassilia | 1473205 | 8 | −0.0304 | 0.0815 | 0.0230 | −0.0232 | −0.0690 | −0.0111 | 0.0619 | −0.0259 | 0.3387 |
| Peptoclostridium | 1481960 | 1 | 0.0349 | 0.1704 | 0.1471 | −0.0767 | −0.1155 | −0.0806 | 0.0615 | 0.0228 | 0.1008 |
| Romboutsia | 1501226 | 2 | −0.0465 | −0.0068 | 0.0822 | −0.0245 | −0.0337 | −0.0673 | −0.0485 | −0.0220 | −0.0250 |
| Alistepes sp. 627 | 1501391 | 6 | 0.0456 | 0.0301 | −0.0199 | −0.0320 | −0.0380 | 0.1201 | 0.2422 | 0.0431 | 0.0168 |
| Barnesiella sp. 177 | 1501392 | 1 | −0.0440 | 0.1419 | 0.0909 | −0.0149 | −0.1549 | 0.0373 | 0.1497 | −0.0118 | 0.0388 |
| Terrisporobacter | 1502652 | 1 | 0.0190 | 0.1595 | −0.0083 | 0.0150 | −0.0419 | 0.1674 | 0.1384 | −0.0207 | −0.0001 |
| Intestinibacter | 1505657 | 1 | 0.0134 | −0.1365 | 0.0955 | −0.0425 | −0.0334 | −0.0237 | −0.0602 | 0.0279 | −0.0472 |
| Asaccharospora | 1505660 | 2 | −0.0599 | 0.1007 | 0.1506 | 0.0169 | −0.0559 | 0.1233 | 0.0452 | −0.0143 | −0.0049 |
| Erysipelatoclostridium | 1505663 | 1 | −0.0317 | −0.2956 | −0.0668 | −0.0424 | 0.0455 | 0.0170 | 0.0177 | 0.0015 | 0.0347 |

TABLE 21

| | | | Micro- | Micro- | Micro- | Micro- | Micro- | Micro- | Micro- | Micro- | Micro- |
|---|---|---|---|---|---|---|---|---|---|---|---|
| | | | biome | biome | biome | biome | biome | biome | biome | biome | biome |
| | | | Sub- | Sub- | Sub- | Sub- | Sub- | Sub- | Sub- | Sub- | Sub- |
| Function | Function | | System | System | System | System | System | System | System | System | System |
| ID | name | Level | 0 | 1 | 2 | 3 | 4 | 5 | 6 | 7 | 8 |
| F1 | Nucleotide | 2 | −0.0709 | −0.4758 | 0.0567 | −0.1000 | 0.1812 | −0.1892 | −0.3273 | −0.1140 | −0.1173 |
| | Metabolism | | | | | | | | | | |
| F2 | Biosynthesis of Other | 2 | −0.0438 | −0.5078 | −0.1933 | −0.0327 | 0.1326 | −0.1510 | −0.300 | −0.0881 | −0.1084 |
| | Secondary | | | | | | | | | | |
| | Metabolites | | | | | | | | | | |
| F3 | Excretory System | 2 | −0.0105 | −0.0033 | −0.3775 | 0.2207 | 0.0563 | 0.0793 | −0.1137 | −0.0788 | −0.0845 |
| F4 | Cell Growth and | 2 | −0.0574 | −0.5020 | 0.0241 | −0.0802 | 0.1825 | −0.2090 | −0.3559 | −0.0942 | −0.1244 |
| | Death | | | | | | | | | | |
| F5 | Enzyme Families | 2 | −0.0702 | −0.5103 | 0.328 | −0.0978 | 0.1674 | −0.1766 | −0.3113 | −0.1069 | −0.1142 |
| F6 | Membrane Transport | 2 | −0.0702 | −0.4831 | 0.1116 | −0.0917 | 0.0268 | −0.1190 | −0.1774 | −0.0906 | −0.0923 |
| F7 | Immune System | 2 | −0.0224 | −0.2930 | 0.0774 | −0.1041 | 0.0918 | −0.0884 | −0.1691 | −0.1315 | −0.0386 |
| | Diseases | | | | | | | | | | |
| F8 | Neirodegenerative | 2 | −0.0423 | −0.0980 | −0.0878 | 0.0686 | 0.2648 | 0.0441 | −0.2132 | −0.1225 | −0.0876 |
| | Diseases | | | | | | | | | | |
| F9 | Immune System | 2 | −0.0452 | −0.5156 | −0.0872 | −0.0674 | 0.2097 | −0.2071 | −0.3898 | −0.0870 | −0.1309 |
| F10 | Infectious Diseases | 2 | −0.0811 | −0.3668 | 0.0725 | −0.0559 | 0.1534 | −0.0929 | −0.2710 | −0.1414 | −0.0941 |
| F11 | Cell Motility | 2 | −0.0374 | −0.3173 | 0.1554 | −0.0540 | −0.0303 | −0.1483 | −0.2329 | −0.0507 | −0.0775 |
| F12 | Metabolism | 2 | −0.0604 | −0.4245 | −0.0600 | −0.0208 | 0.1389 | −0.0531 | −0.2420 | −0.1242 | −0.1153 |
| F13 | Signaling Molecules | 2 | −0.0263 | −0.1840 | −0.1781 | 0.0095 | 0.2219 | 0.0263 | −0.1777 | −0.1359 | −0.0769 |
| | and Interaction | | | | | | | | | | |
| F14 | Glycan Biosynthesis | 2 | −0.0169 | −0.1622 | −0.2548 | 0.0333 | 0.2841 | −0.0334 | −0.2758 | −0.1087 | −0.0751 |
| | and Metabolism | | | | | | | | | | |
| F16 | Signal Transduction | 2 | −0.0583 | −0.3710 | 0.0085 | −0.0299 | 0.1350 | −0.0601 | −0.2122 | −0.1051 | −0.0948 |
| F17 | Metabolism of | 2 | −0.0549 | −0.5129 | −0.0018 | −0.0955 | 0.1601 | −0.1814 | −0.3099 | −0.1053 | −0.1095 |
| | Cofactors and | | | | | | | | | | |
| | Vitamins | | | | | | | | | | |
| F18 | Cardiovascular | 2 | 0.0201 | 0.1743 | 0.0173 | −0.0264 | −0.1250 | 0.1271 | 0.2657 | 0.0114 | 0.0624 |
| | Diseases | | | | | | | | | | |
| F19 | Transcription | 2 | −0.0663 | −0.5503 | 0.0280 | −0.0729 | 0.0605 | −0.1402 | −0.2581 | −0.2581 | −0.1190 |
| F20 | Translation | 2 | −0.0753 | −0.5137 | 0.0625 | −0.0907 | 0.1680 | −0.2232 | −0.3545 | −0.1015 | −0.1312 |
| F21 | Digestive System | 2 | −0.0285 | 0.0569 | −0.0814 | −0.0043 | 0.2124 | −0.0166 | −0.2004 | −0.0904 | −0.0434 |
| F22 | Metabolism of Other | 2 | −0.0613 | −0.4439 | −0.0309 | −0.0805 | 0.1716 | −0.1320 | −0.2784 | −0.1318 | −0.0945 |
| | Amino Acids | | | | | | | | | | |
| F23 | Poorly Characterized | 2 | −0.0664 | −0.4905 | −0.0182 | −0.0592 | 0.1482 | −0.1372 | −0.2927 | −0.1093 | −0.1166 |
| F24 | Lipid Metabolism | 2 | −0.0478 | −0.5257 | −0.1166 | −0.0199 | 0.1143 | −0.1341 | −0.2946 | −0.1016 | −0.1373 |
| F25 | Metabolic Diseases | 2 | −0.0556 | −0.4542 | −0.0453 | −0.0493 | 0.1611 | −0.1567 | −0.3300 | −0.1186 | −0.1291 |
| F26 | Xenobiotics | 2 | −0.0679 | −0.4566 | −0.0070 | −0.0046 | 0.1434 | −0.0923 | −0.2638 | −0.1176 | −0.1286 |
| | Biodegradation and | | | | | | | | | | |
| | Metabolism | | | | | | | | | | |
| F27 | Cancers | 2 | −0.0466 | −0.3110 | −0.0577 | 0.0069 | 0.2664 | −0.1240 | −0.3633 | −0.1023 | −0.1261 |
| F28 | Transport and | 2 | 0.0061 | −0.1334 | −0.4701 | 0.0886 | 0.2334 | 0.0197 | −0.2292 | −0.0913 | −0.0682 |
| | Catabolism | | | | | | | | | | |
| F29 | Replication and | 2 | −0.0691 | −0.5022 | 0.0372 | −0.0860 | 0.1783 | −0.2016 | −0.3490 | −0.1081 | −0.1287 |
| | Repair | | | | | | | | | | |
| F30 | Metabolism of | 2 | −0.0645 | −0.4884 | 0.0003 | −0.0625 | 0.1755 | −0.1773 | −0.3321 | −0.1105 | −0.1232 |
| | Terpenoids and | | | | | | | | | | |
| | Polyketides | | | | | | | | | | |
| F31 | Cellular Processes | 2 | −0.0476 | −0.4801 | −0.0693 | −0.0609 | 0.1631 | −0.1012 | −0.2621 | −0.1149 | −0.1020 |
| | and Signaling | | | | | | | | | | |
| F32 | Endocrine System | 2 | −0.0510 | −0.4341 | −0.2168 | 0.0034 | 0.2395 | −0.1680 | −0.3741 | −0.0917 | −0.1370 |
| F33 | Carbohydrate | 2 | −0.0555 | −0.5389 | −0.1193 | −0.0534 | 0.1060 | −0.1285 | −0.2639 | −0.0988 | −0.1185 |
| | Metabolism | | | | | | | | | | |
| F34 | Amino Acid | 2 | −0.0533 | −0.5656 | −0.0680 | −0.0613 | 0.1312 | −0.1766 | −0.3137 | −0.0934 | −0.1301 |
| | Metabolism | | | | | | | | | | |
| F35 | Genetic Information | 2 | −0.0722 | −0.4890 | 0.0453 | −0.0519 | 0.1504 | −0.1495 | −0.3055 | −0.1159 | −0.1310 |
| | Processing | | | | | | | | | | |
| F36 | Environmental | 2 | −0.0384 | −0.5552 | 0.0284 | −0.0895 | 0.0889 | −0.2162 | −0.3111 | −0.0842 | −0.1316 |
| | Adaptation | | | | | | | | | | |
| F38 | Folding, Sorting and | 2 | −0.0644 | −0.4804 | −0.0330 | −0.0686 | 0.2144 | −0.1793 | −0.3489 | −0.1128 | −0.1254 |
| | Degradation | | | | | | | | | | |
| F39 | Nervous System | 2 | −0.0525 | −0.5443 | −0.2329 | −0.0372 | 0.1390 | −0.1755 | −0.3303 | −0.0779 | −0.1218 |
| F40 | Circulatory System | 2 | 0.0659 | 0.2116 | −0.0056 | −0.0338 | −0.1216 | 0.0126 | 0.1125 | 0.0367 | 0.0279 |
| F41 | Energy Metabolism | 2 | −0.0558 | −0.5527 | −0.0585 | −0.0807 | 0.1704 | −0.1819 | −0.3166 | −0.0888 | −0.1092 |
| F42 | Bacterial chemotaxis | 3 | −0.0317 | −0.3620 | 0.1462 | −0.0871 | −0.0706 | −0.1718 | −0.2139 | −0.0353 | −0.0683 |
| F44 | Cell cycle- | 3 | −0.0574 | −0.5097 | 0.0323 | −0.0912 | 0.1753 | −0.2123 | −0.3501 | −0.0945 | −0.1204 |
| | Caulobacter | | | | | | | | | | |
| F45 | Membrane and | 3 | −0.0200 | −0.0388 | −0.2353 | 0.0331 | 0.3144 | 0.0316 | −0.2162 | −0.0996 | −0.0325 |
| | intracellular | | | | | | | | | | |
| | structural molecules | | | | | | | | | | |
| F46 | Chloroalkane and | 3 | −0.0515 | −0.5008 | −0.0824 | −0.0131 | −0.0143 | −0.0899 | −0.2022 | −0.0545 | −0.1299 |
| | chloroalkene | | | | | | | | | | |
| | degradation | | | | | | | | | | |
| F47 | Pentose and | 3 | −0.0297 | −0.4052 | −0.2792 | −0.0195 | 0.0825 | −0.0547 | −0.1690 | −0.0778 | −0.0794 |
| | glucoronate | | | | | | | | | | |
| | interconverions | | | | | | | | | | |
| F48 | Cell division | 3 | −0.0374 | −0.4351 | −0.0910 | −0.0987 | 0.1653 | −0.1496 | −0.2530 | −0.0752 | −0.0603 |
| F49 | RNA polymerase | 3 | −0.0755 | −0.4931 | 0.0837 | −0.0905 | 0.1506 | −0.1941 | −0.3193 | −0.0959 | −0.1282 |
| F50 | Energy metabolism | 3 | −0.0570 | −0.4729 | −0.0447 | −0.0478 | 0.2195 | −0.1475 | −0.3428 | −0.1129 | −0.1403 |
| F51 | Antigen processing | 3 | −0.0509 | −0.4782 | −0.0345 | −0.0573 | 0.2017 | −0.1975 | −0.4005 | −0.0749 | −0.1323 |
| | and presentation | | | | | | | | | | |
| F52 | N-Glycan | 3 | −0.0501 | −0.1460 | −0.1148 | 0.0817 | 0.1257 | −0.0544 | −0.2125 | −0.0464 | −0.0535 |
| | biosynthesis | | | | | | | | | | |
| F53 | Synthesis and | 3 | −0.0541 | −0.3138 | 0.1411 | −0.0672 | 0.0397 | −0.1061 | −0.1630 | −0.0959 | −0.0922 |
| | degradation of ketone | | | | | | | | | | |
| | bodies | | | | | | | | | | |
| F54 | Biosynthesis of | 3 | −0.0736 | −0.3512 | 0.1252 | 0.0689 | 0.0845 | −0.0741 | −0.2776 | −0.1067 | −0.1072 |
| | unsaturated fatty | | | | | | | | | | |
| | acids | | | | | | | | | | |
| F57 | Sulfur metabolism | 3 | −0.0272 | −0.4910 | −0.1762 | −0.0063 | 0.1235 | −0.1261 | −0.2693 | −0.0993 | −0.0947 |
| F58 | Nucleotide excision | 3 | −0.0651 | −0.4778 | 0.0669 | −0.0808 | 0.1711 | −0.2025 | −0.3473 | −0.1009 | −0.1322 |
| | repair | | | | | | | | | | |
| F59 | C5-Branched dibasic | 3 | −0.0440 | −0.5669 | −0.0801 | −0.0423 | 0.0317 | −0.1727 | −0.2674 | −0.0637 | −0.1128 |
| | acid metabolism | | | | | | | | | | |
| F60 | Biotin metabolism | 3 | −0.0278 | −0.3790 | −0.2015 | −0.0374 | 0.0805 | −0.0843 | −0.2093 | −0.0915 | −0.0729 |
| F61 | Vibrio cholerae | 3 | 0.0118 | 0.1596 | −0.0383 | −0.0772 | −0.1008 | 0.0151 | 0.2889 | 0.1813 | 0.1589 |
| | infection | | | | | | | | | | |
| F62 | One carbon pool by | 3 | −0.0578 | −0.4669 | 0.0324 | −0.0851 | 0.2008 | −0.2107 | −0.3628 | −0.1105 | −0.1221 |
| | folate | | | | | | | | | | |
| F63 | Peptidoglycan | 3 | −0.0781 | −0.5150 | 0.0882 | −0.0989 | 0.1596 | −0.2214 | −0.3468 | −0.1028 | −0.1219 |
| | biosynthesis | | | | | | | | | | |
| F64 | Xylene degradation | 3 | −0.0475 | −0.4383 | 0.0262 | −0.0554 | −0.0789 | −0.1123 | −0.1607 | −0.0473 | −0.0824 |
| F65 | Aminoacyl-tRNA | 3 | −0.0752 | −0.5218 | 0.0626 | −0.0823 | 0.1478 | −0.2241 | −0.3452 | −0.0989 | −0.1339 |
| | biosynthesis | | | | | | | | | | |
| F66 | Ascorbate and | 3 | −0.0231 | −0.1533 | −0.0044 | 0.0051 | 0.0443 | 0.1123 | −0.0166 | −0.0994 | −0.0201 |
| | aldarate metabolism | | | | | | | | | | |
| F67 | Huntington's diseases | 3 | −0.0150 | 0.1323 | −0.2472 | 0.1754 | 0.2261 | 0.1573 | −0.0907 | −0.1098 | −0.0657 |
| F68 | Colorectal cancer | 3 | 0.0221 | 0.1799 | 0.0204 | −0.0247 | −0.1277 | 0.1296 | 0.2684 | 0.0131 | 0.0665 |
| F70 | Apoptosis | 3 | −0.0211 | 0.0278 | −0.0770 | 0.0868 | 0.0414 | 0.0217 | −0.1490 | −0.0524 | −0.1046 |
| F71 | RNA degradation | 3 | −0.0602 | −0.4702 | −0.0689 | −0.0541 | 0.2189 | −0.1717 | −0.3440 | −0.1164 | −0.1298 |
| F72 | Primary bile acid | 3 | −0.0127 | −0.3966 | −0.3493 | −0.0219 | −0.0680 | −0.1092 | −0.1248 | −0.0597 | −0.0368 |
| | biosynthesis | | | | | | | | | | |
| F73 | MAPK signaling | 3 | 0.0270 | −0.3334 | −0.3817 | 0.0500 | 0.0742 | −0.0094 | −0.1580 | −0.0800 | −0.0976 |
| | pathway-yeast | | | | | | | | | | |
| F74 | Inositol phosphate | 3 | −0.0663 | −0.3541 | −0.2183 | 0.0266 | 0.1252 | −0.0412 | −0.1984 | −0.1171 | −0.0725 |
| | metabolism | | | | | | | | | | |
| F76 | Amino acid | 3 | −0.0524 | −0.2757 | −0.0794 | 0.0173 | 0.0197 | 0.0543 | −0.1205 | −0.0979 | −0.0704 |
| | metabolism | | | | | | | | | | |
| F77 | DNA replication | 3 | −0.0683 | −0.4876 | 0.0189 | −0.0799 | 0.1929 | −0.1928 | −0.3582 | −0.1077 | −0.1322 |
| | proteins | | | | | | | | | | |
| F78 | Cell cycle | 3 | 0.0042 | 0.1957 | −0.0251 | −0.0790 | −0.0990 | 0.0282 | 0.3080 | 0.1800 | 0.1665 |
| F79 | Glycosyltransferases | 3 | −0.0573 | −0.2609 | −0.0001 | −0.0271 | 0.1936 | −0.0759 | −0.2532 | −0.1431 | −0.0664 |
| F80 | Insulin signaling | 3 | −0.0312 | −0.5144 | −0.1213 | 0.0045 | 0.0310 | −0.1594 | −0.2825 | −0.0676 | −0.1376 |
| | pathway | | | | | | | | | | |
| F81 | Others | 3 | −0.0521 | −0.4157 | −0.0664 | −0.0284 | 0.0706 | −0.0365 | −0.1878 | −0.1268 | −0.1059 |
| F82 | Propanoate | 3 | −0.0598 | −0.4970 | 0.0048 | −0.0501 | 0.0812 | −0.1063 | −0.2411 | −0.1165 | −0.1106 |
| | metabolism | | | | | | | | | | |
| F83 | Lipopolysaccharide | 3 | −0.0049 | 0.1631 | −0.1937 | 0.1076 | 0.3331 | 0.1027 | −0.1414 | −0.0798 | −0.0197 |
| | biosynthesis proteins | | | | | | | | | | |
| F84 | Phosphatidylinositol | 3 | −0.0522 | −0.3153 | −0.2789 | 0.0504 | 0.2107 | −0.0229 | −0.2485 | −0.1316 | −0.1346 |
| | signaling system | | | | | | | | | | |
| F85 | Pores ion channels | 3 | −0.0040 | 0.0268 | −0.2584 | 0.0603 | 0.2727 | 0.0749 | −0.1654 | −0.1107 | −0.0373 |
| F87 | Flavonoid | 3 | −0.0577 | −0.1540 | −0.0099 | 0.1716 | 0.0285 | −0.0810 | −0.2642 | −0.0318 | −0.1508 |
| | biosynthesis | | | | | | | | | | |
| F88 | Betalain biosynthesis | 3 | 0.0228 | 0.1212 | 0.0932 | −0.0223 | −0.1054 | −0.0499 | 0.1280 | −0.0478 | 0.0370 |
| F89 | Biosynthesis and | 3 | −0.0180 | −0.2749 | −0.1625 | 0.0601 | 0.0839 | 0.0332 | −0.1287 | −0.0887 | −0.0504 |
| | biodegradation of | | | | | | | | | | |
| | secondary | | | | | | | | | | |
| | metabolites | | | | | | | | | | |
| F90 | Starch and sucrose | 3 | −0.0570 | −0.5457 | −0.1119 | −0.0716 | 0.0223 | −0.1746 | −0.2614 | −0.0803 | −0.0947 |
| | metabolism | | | | | | | | | | |
| F91 | Zeatin biosynthesis | 3 | −0.0587 | −0.3705 | −0.0560 | −0.0735 | 0.2550 | −0.1973 | −0.3568 | −0.1029 | −0.0997 |
| F92 | Various types of N- | 3 | 0.0328 | 0.2576 | 0.0985 | −0.1028 | −0.1639 | 0.0262 | 0.2483 | 0.1700 | 0.0882 |
| | glycan biosynthesis | | | | | | | | | | |
| F93 | Phosphonate and | 3 | −0.0183 | −0.2181 | −0.3093 | 0.0604 | 0.1241 | 0.0325 | −0.1204 | −0.1004 | −0.0667 |
| | phosphinate | | | | | | | | | | |
| | metabolism | | | | | | | | | | |
| F94 | Arginine and proline | 3 | −0.0438 | −0.5744 | −0.1556 | −0.0328 | 0.0938 | −0.1574 | −0.2897 | −0.0801 | −0.1377 |
| | metabolism | | | | | | | | | | |
| F96 | Caprolactam | 3 | −0.0091 | 0.0262 | 0.0788 | 0.1819 | 0.0713 | 0.1968 | 0.0192 | −0.1267 | −0.0423 |
| | degradation | | | | | | | | | | |
| F97 | Tetracycline | 3 | −0.0266 | −0.5452 | −0.1040 | −0.0364 | −0.0226 | −0.1327 | −0.1911 | −0.0647 | −0.0963 |
| | biosynthesis | | | | | | | | | | |
| F98 | Dioxin degradation | 3 | −0.0534 | −0.3992 | 0.0507 | −0.0554 | −0.0817 | −0.0902 | −0.1428 | −0.0530 | −0.0723 |
| F100 | Ribosome Biogenesis | 3 | −0.0783 | −0.5265 | 0.0783 | −0.0997 | 0.1535 | −0.2183 | −0.3367 | −0.1059 | −0.1240 |
| F101 | Benzoate degradation | 3 | −0.0621 | −0.4604 | 0.0787 | −0.0574 | 0.0591 | −0.1290 | −0.2316 | −0.1113 | −0.1057 |
| F102 | Bacterial invasions of | 3 | −0.0115 | 0.3544 | 0.2704 | −0.0303 | −0.0672 | 0.1515 | 0.1067 | −0.0038 | 0.0406 |
| | epithelial cells | | | | | | | | | | |
| F103 | Translation proteins | 3 | −0.0722 | −0.5057 | 0.0762 | −0.0773 | 0.1683 | −0.1955 | −0.3437 | −0.1070 | −0.1383 |
| F104 | Cell motility and | 3 | −0.0131 | −0.0638 | −0.1200 | 0.1295 | 0.3288 | 0.0462 | −0.2299 | −0.1033 | −0.0947 |
| | secretion | | | | | | | | | | |
| F105 | Other ion-coupled | 3 | −0.0444 | −0.3643 | −0.0512 | −0.0988 | 0.2278 | −0.0426 | −0.2014 | −0.1533 | −0.0589 |
| | transporters | | | | | | | | | | |
| F106 | Histidine metabolism | 3 | −0.0372 | −0.5659 | −0.2010 | −0.0262 | 0.0934 | −0.1618 | −0.2975 | −0.0839 | −0.1386 |
| F107 | Protein folding and | 3 | −0.0700 | −0.3712 | −0.0101 | −0.0064 | 0.2162 | −0.0820 | −0.2801 | −0.1145 | −0.1181 |
| | associated processing | | | | | | | | | | |
| F110 | Lipid biosynthesis | 3 | −0.0572 | −0.5052 | −0.1106 | −0.0239 | 0.1875 | −0.1742 | −0.3677 | −0.0989 | −0.1547 |
| | proteins | | | | | | | | | | |
| F111 | Photosynthesis | 3 | −0.0630 | −0.5623 | 0.0336 | −0.1416 | 0.1166 | −0.2429 | −0.2694 | −0.0758 | −0.0522 |
| F113 | D-Alanine | 3 | −0.0745 | −0.4344 | 0.1537 | −0.0954 | 0.1679 | −0.1828 | −0.3202 | −0.1318 | −0.1230 |
| | metabolism | | | | | | | | | | |
| F114 | Bisphenol | 3 | −0.0101 | −0.4221 | −0.2597 | −0.0088 | 0.0591 | −0.0595 | −0.1521 | −0.0725 | −0.0946 |
| | degradation | | | | | | | | | | |
| F115 | Glycosphingolipid | 3 | 0.0126 | −0.0759 | −0.4033 | 0.0573 | 0.1466 | 0.0388 | −0.1778 | −0.0589 | −0.0310 |
| | biosynthesis-globo | | | | | | | | | | |
| | series | | | | | | | | | | |
| F116 | Alanine, aspartate | 3 | −0.0458 | −0.5433 | −0.1485 | −0.0722 | 0.1864 | −0.1813 | −0.3305 | −0.0985 | −0.1227 |
| | and glutamate | | | | | | | | | | |
| | metabolism | | | | | | | | | | |
| F117 | Glycine, serine and | 3 | −0.0609 | −0.5207 | −0.0277 | −0.0717 | 0.1608 | −0.1730 | −0.3175 | −0.1062 | −0.1152 |
| | threonine metabolism | | | | | | | | | | |
| F118 | P53 signaling | 3 | 0.0250 | 0.1812 | 0.0175 | −0.0344 | −0.1323 | 0.1239 | 0.2723 | 0.0112 | 0.0645 |
| | pathway | | | | | | | | | | |
| F119 | Phagosome | 3 | 0.0042 | 0.1957 | −0.0251 | −0.0790 | −0.0990 | 0.0282 | 0.3080 | 0.1800 | 0.1665 |
| F121 | Circadian rhythm- | 3 | 0.0563 | 0.2087 | 0.1219 | 0.0448 | −0.0632 | 0.1011 | 0.1930 | 0.0893 | 0.0788 |
| | plant | | | | | | | | | | |
| F122 | Vitamin B6 | 3 | −0.0533 | −0.4292 | −0.0351 | −0.0536 | 0.2411 | −0.1715 | −0.3658 | −0.1118 | −0.1224 |
| | metabolism | | | | | | | | | | |
| F123 | Valine, leucine and | 3 | −0.0551 | −0.1954 | −0.0081 | 0.0129 | 0.2201 | −0.0116 | −0.2157 | −0.1592 | −0.0785 |
| | isoleucine | | | | | | | | | | |
| | degradation | | | | | | | | | | |
| F124 | Butirosin and | 3 | −0.0498 | −0.4926 | −0.1045 | 0.0080 | 0.0716 | −0.1843 | −0.3329 | −0.0731 | −0.1400 |
| | neomycin | | | | | | | | | | |
| | biosynthesis | | | | | | | | | | |
| F125 | Lysosome | 3 | 0.0290 | 0.0032 | −0.5292 | 0.1364 | 0.1647 | 0.0951 | −0.1181 | −0.0596 | −0.0377 |
| F127 | Basal transcription | 3 | 0.0437 | 0.1221 | 0.0660 | −0.0593 | 0.0516 | 0.0129 | 0.0181 | 0.0714 | 0.0157 |
| | factors | | | | | | | | | | |
| F128 | Transcription | 3 | −0.0561 | −0.5310 | −0.1410 | −0.0206 | 0.1479 | −0.1622 | −0.3427 | −0.0895 | −0.1409 |
| | machinery | | | | | | | | | | |
| F129 | Oxidative | 3 | −0.0520 | −0.4805 | −0.0752 | −0.0610 | 0.2291 | −0.1689 | −0.3334 | −0.0969 | −0.0993 |
| | phosphorylation | | | | | | | | | | |
| F130 | Proximal tubule | 3 | −0.0105 | −0.0033 | −0.3775 | 0.2207 | 0.0563 | 0.0793 | −0.1137 | −0.0788 | −0.0845 |
| | bicarbonate | | | | | | | | | | |
| | reclamation | | | | | | | | | | |
| F131 | Stilbenoid, | 3 | −0.0814 | 0.0860 | 0.0744 | 0.0213 | −0.0046 | 0.0125 | −0.1107 | −0.0508 | −0.0402 |
| | diarylheptanoid and | | | | | | | | | | |
| | gingerol biosynthesis | | | | | | | | | | |
| F132 | Purine metabolism | 3 | −0.0702 | −0.4672 | 0.0541 | −0.0958 | 0.1752 | −0.1742 | −0.3121 | −0.1175 | −0.1128 |
| F133 | Fatty acid | 3 | −0.0890 | −0.3889 | 0.0248 | −0.0086 | 0.1235 | −0.0741 | −0.2362 | −0.1426 | −0.1047 |
| | metabolism | | | | | | | | | | |
| F134 | Ether lipid | 3 | −0.1041 | −0.2089 | 0.0413 | 0.0109 | 0.0792 | −0.0486 | −0.0855 | −0.0596 | −0.0605 |
| | metabolism | | | | | | | | | | |
| F135 | Inorganic ion | 3 | −0.0322 | −0.0776 | −0.1335 | 0.1029 | 0.1081 | 0.1472 | −0.0849 | −0.1203 | −0.0515 |
| | transport and | | | | | | | | | | |
| | metabolism | | | | | | | | | | |
| F136 | Caffeine metabolism | 3 | 0.0268 | 0.1926 | 0.1266 | 0.0205 | −0.0798 | 0.0930 | 0.2074 | 0.0582 | 0.0978 |
| F138 | Influenza A | 3 | 0.0178 | 0.2238 | 0.0229 | −0.0324 | −0.1523 | 0.1333 | 0.3212 | 0.0717 | 0.1144 |
| F140 | Tryptophan | 3 | −0.0800 | −0.2514 | 0.0752 | 0.0562 | 0.1242 | −0.0050 | −0.1895 | −0.1498 | −0.0884 |
| | metabolism | | | | | | | | | | |
| F141 | Linoleic acid | 3 | −0.0161 | −0.4476 | −0.2948 | −0.0246 | 0.0616 | −0.0953 | −0.1713 | −0.0698 | −0.0914 |
| | metabolism | | | | | | | | | | |
| F142 | Secretion system | 3 | −0.0669 | −0.3273 | 0.1480 | −0.0025 | 0.1314 | −0.0896 | −0.2758 | −0.1167 | −0.1150 |
| F143 | Spliceosome | 3 | −0.0449 | 0.0616 | 0.0574 | −0.0238 | −0.0914 | 0.0176 | 0.0264 | 0.0370 | 0.0533 |
| F146 | Ethylbenzene | 3 | −0.0430 | −0.1203 | 0.0159 | 0.0765 | 0.1590 | −0.0305 | −0.2932 | −0.0916 | −0.1078 |
| | degradation | | | | | | | | | | |
| F147 | Riboflavin | 3 | −0.0635 | −0.3497 | 0.0578 | −0.1101 | 0.2532 | −0.1774 | −0.2991 | −0.1151 | −0.0713 |
| | metabolism | | | | | | | | | | |
| F148 | Phosphotransferase | 3 | −0.0189 | −0.2741 | 0.1664 | −0.1190 | −0.0507 | 0.0515 | 0.0438 | −0.0573 | −0.0259 |
| | system (PTS) | | | | | | | | | | |
| F149 | Methane metabolism | 3 | −0.0570 | −0.5769 | −0.0590 | −0.0721 | 0.0339 | −0.1656 | −0.2676 | −0.0692 | −0.1196 |
| F150 | Alzheimer's disease | 3 | −0.0738 | −0.4100 | 0.0010 | −0.0261 | 0.2016 | −0.1893 | −0.3620 | −0.1018 | −0.1374 |
| F151 | African | 3 | −0.0445 | 0.0625 | 0.1122 | 0.1945 | 0.0095 | 0.1300 | 0.0070 | −0.0912 | −0.0562 |
| | trypanosomiasis | | | | | | | | | | |
| F152 | Drug metabolism- | 3 | −0.0532 | −0.0224 | −0.1067 | 0.1798 | 0.0881 | 0.0316 | −0.1921 | −0.0698 | −0.1049 |
| | cytochrome P450 | | | | | | | | | | |
| F153 | Pentose phosphate | 3 | −0.0505 | −0.5619 | −0.0883 | −0.0572 | 0.0607 | −0.1412 | −0.2466 | −0.0906 | −0.1252 |
| | pathway | | | | | | | | | | |
| F154 | Fatty acid elongation | 3 | 0.0027 | 0.2087 | 0.1253 | 0.0245 | −0.0635 | 0.0866 | 0.2480 | 0.0051 | 0.1101 |
| | in mitochondria | | | | | | | | | | |
| F155 | Other glycan | 3 | 0.0151 | −0.1312 | −0.5331 | 0.0192 | 0.1120 | 0.0279 | −0.1062 | −0.0754 | −0.0110 |
| | degradation | | | | | | | | | | |
| F157 | Peptidases | 3 | −0.0664 | −0.5012 | 0.0278 | −0.1004 | 0.1871 | −0.1815 | −0.3220 | −0.1083 | −0.1166 |
| F160 | Sulfur relay system | 3 | −0.0521 | −0.4432 | 0.0585 | −0.0603 | 0.0376 | −0.0886 | −0.1637 | −0.1070 | −0.0874 |
| F161 | Carotenoid | 3 | −0.0740 | −0.0943 | 0.0462 | 0.1685 | −0.0152 | 0.0033 | −0.1660 | −0.0649 | −0.1246 |
| | biosynthesis | | | | | | | | | | |
| F162 | Bacterial secretion | 3 | −0.0627 | −0.3821 | 0.0044 | 0.0191 | 0.2431 | −0.1252 | −0.3549 | −0.1204 | −0.1427 |
| | system | | | | | | | | | | |
| F163 | Renin-angiotensin | 3 | 0.0453 | −0.0445 | −0.0599 | −0.0272 | −0.0348 | −0.0344 | −0.0138 | 0.0260 | 0.0026 |
| | system | | | | | | | | | | |
| F165 | G protein-coupled | 3 | 0.0211 | 0.2569 | 0.0562 | 0.0109 | −0.1006 | 0.0046 | 0.0521 | 0.0182 | 0.1315 |
| | receptors | | | | | | | | | | |
| F166 | Germination | 3 | −0.0415 | −0.5451 | 0.0466 | −0.0899 | −0.0920 | −0.1937 | −0.2066 | −0.0287 | −0.0842 |
| F168 | Type I diabetes | 3 | −0.0522 | −0.4242 | −0.1786 | −0.0190 | 0.2065 | −0.1346 | −0.3357 | −0.1275 | −0.1309 |
| | mellitus | | | | | | | | | | |
| F170 | Phenylalanine | 3 | −0.0288 | −0.3517 | −0.1709 | 0.0197 | 0.2335 | −0.0293 | −0.2573 | −0.1010 | −0.1087 |
| | metabolism | | | | | | | | | | |
| F171 | Porphyrin and | 3 | −0.0207 | −0.5334 | −0.0531 | −0.1148 | 0.0478 | −0.1417 | −0.1939 | −0.0648 | −0.0941 |
| | chlorophyll | | | | | | | | | | |
| | metabolism | | | | | | | | | | |
| F172 | Vibrio cholerae | 3 | −0.0457 | −0.2510 | 0.2199 | −0.1399 | 0.1843 | −0.0656 | −0.2210 | −0.1381 | −0.0411 |
| | pathogenic cycle | | | | | | | | | | |
| F173 | Fatty acid | 3 | −0.0401 | −0.5705 | −0.1127 | −0.0385 | 0.1203 | −0.1669 | −0.3137 | −0.0892 | −0.1405 |
| | biosynthesis | | | | | | | | | | |
| F175 | Adipocytokine | 3 | −0.0439 | −0.2055 | −0.2749 | 0.0345 | 0.3243 | −0.1022 | −0.3350 | −0.0872 | −0.1010 |
| | signaling pathway | | | | | | | | | | |
| F176 | Selenocompound | 3 | −0.0620 | −0.5139 | 0.0137 | −0.0845 | 0.1414 | −0.1504 | −0.2749 | −0.1151 | −0.1144 |
| | metabolism | | | | | | | | | | |
| F177 | RIG-I-like receptor | 3 | −0.0141 | −0.3576 | 0.0308 | −0.0870 | −0.0354 | −0.1345 | −0.0612 | −0.0440 | 0.0107 |
| | signaling pathway | | | | | | | | | | |
| F178 | Lysine | 3 | −0.0639 | −0.2147 | 0.0389 | 0.0484 | 0.1293 | −0.0008 | −0.1930 | −0.1647 | −0.0798 |
| | degradation | | | | | | | | | | |
| F179 | Glycosaminoglycan | 3 | 0.0352 | 0.0682 | −0.5134 | 0.1435 | 0.1722 | 0.1204 | −0.0896 | −0.0568 | −0.0295 |
| | degradation | | | | | | | | | | |
| F180 | Secondary bile acid | 3 | −0.0141 | −0.4017 | −0.3525 | −0.0202 | −0.0642 | −0.1102 | −0.1287 | −0.0606 | −0.0406 |
| | biosynthesis | | | | | | | | | | |
| F181 | Indole alkaloid | 3 | 0.0226 | 0.1211 | 0.0932 | −0.0222 | −0.1054 | −0.0498 | 0.1278 | −0.0477 | 0.0371 |
| | biosynthesis | | | | | | | | | | |
| F182 | Fructose and | 3 | −0.0523 | −0.5391 | −0.1104 | −0.0688 | 0.0421 | −0.1069 | −0.2173 | −0.0841 | −0.1065 |
| | mannose metabolism | | | | | | | | | | |
| F183 | Terpenoid backbone | 3 | −0.0672 | −0.4982 | 0.0681 | −0.0975 | 0.1757 | −0.2217 | −0.3459 | −0.1068 | −0.1216 |
| | biosynthesis | | | | | | | | | | |
| F184 | Phenylpropanoid | 3 | −0.0261 | −0.3772 | −0.2495 | −0.0524 | −0.0233 | −0.1413 | −0.1704 | −0.0661 | −0.0299 |
| | biosynthesis | | | | | | | | | | |
| F185 | Penicillin and | 3 | −0.0127 | −0.1044 | −0.4574 | 0.1400 | 0.0884 | 0.0642 | −0.0388 | −0.0511 | −0.0518 |
| | cephalosporin | | | | | | | | | | |
| | biosynthesis | | | | | | | | | | |
| F186 | mTOR signaling | 3 | 0.0042 | 0.1957 | −0.0251 | −0.0790 | −0.0990 | 0.0282 | 0.3080 | 0.1800 | 0.1665 |
| | pathway | | | | | | | | | | |
| F187 | Carbohydrate | 3 | −0.0272 | −0.3914 | −0.1770 | −0.0285 | −0.0414 | −0.0609 | −0.1414 | −0.0696 | −0.0575 |
| | metabolism | | | | | | | | | | |
| F189 | Polycyclic aromatic | 3 | −0.0665 | −0.5541 | −0.0278 | −0.0856 | 0.1130 | −0.2463 | −0.3267 | −0.0944 | −0.1065 |
| | hydrocarbon | | | | | | | | | | |
| | degradation | | | | | | | | | | |
| F190 | Ribosome | 3 | −0.0747 | −0.4876 | 0.0605 | −0.0896 | 0.1872 | −0.2242 | −0.3655 | −0.1028 | −0.1310 |
| F192 | Glycan biosynthesis | 3 | −0.0255 | 0.0527 | 0.0212 | 0.0440 | 0.3846 | 0.0447 | −0.2315 | −0.0757 | −0.0586 |
| | and metabolism | | | | | | | | | | |
| F193 | Aminobenzoate | 3 | −0.0521 | −0.2772 | −0.0641 | 0.0954 | 0.2046 | −0.0274 | −0.2577 | −0.1373 | −0.1180 |
| | degradation | | | | | | | | | | |
| F195 | Cardiac muscle | 3 | 0.0659 | 0.2116 | −0.0056 | −0.0338 | −0.1216 | 0.0126 | 0.1125 | 0.0367 | 0.0279 |
| | contraction | | | | | | | | | | |
| F196 | Vitamin metabolism | 3 | −0.0776 | −0.2526 | 0.0943 | 0.0061 | 0.1658 | 0.0066 | −0.1398 | −0.0892 | −0.0536 |
| F197 | Renal cell carcinoma | 3 | −0.0489 | 0.1221 | −0.0202 | 0.1449 | 0.3018 | 0.1065 | −0.1153 | −0.1016 | −0.0690 |
| F198 | Butanoate | 3 | −0.0550 | −0.4472 | 0.0041 | −0.0516 | 0.2184 | −0.1099 | −0.2522 | −0.1192 | −0.1125 |
| | metabolism | | | | | | | | | | |
| F199 | Other transporters | 3 | −0.0373 | −0.4852 | −0.1421 | −0.0340 | 0.1583 | −0.0885 | −0.2723 | −0.1098 | −0.1393 |
| F200 | Glyoxylate and | 3 | −0.0496 | −0.4942 | −0.1401 | −0.0412 | 0.1292 | −0.0945 | −0.2273 | −0.0974 | −0.1028 |
| | dicarboxylate | | | | | | | | | | |
| | metabolism | | | | | | | | | | |
| F201 | Carbon fixation | 3 | −0.0473 | −0.4451 | −0.0791 | −0.0385 | 0.2821 | −0.1533 | −0.3493 | −0.1062 | −0.1291 |
| | pathways in | | | | | | | | | | |
| | prokaryotes | | | | | | | | | | |
| F202 | Lysine biosynthesis | 3 | −0.0484 | −0.5967 | −0.0595 | −0.0756 | 0.073 | −0.1838 | −0.2892 | −0.0771 | −0.1228 |
| F203 | Mismatch repair | 3 | −0.0622 | −0.4916 | 0.0486 | −0.0883 | 0.1487 | −0.1946 | −0.3411 | −0.1103 | −0.1247 |
| F206 | Ion channels | 3 | 0.0177 | 0.0025 | −0.0532 | 0.1380 | −0.0192 | 0.1322 | 0.0458 | −0.0838 | −0.0043 |
| F207 | General function | 3 | −0.0631 | −0.5272 | −0.0298 | −0.0704 | 0.1523 | −0.1635 | −0.3129 | −0.1005 | −0.1223 |
| F210 | Citrate cycle (TCA | 3 | −0.0366 | −0.2575 | −0.1167 | 0.0219 | 0.3859 | −0.0855 | −0.3473 | −0.1087 | −0.1168 |
| | cycle) | | | | | | | | | | |
| F212 | Lipopolysaccharide | 3 | 0.0002 | 0.1865 | −0.1565 | 0.1266 | 0.3398 | 0.0815 | −0.1581 | −0.0651 | −0.0230 |
| | biosynthesis | | | | | | | | | | |
| F213 | Geraniol degradation | 3 | −0.0381 | 0.1671 | −0.1062 | 0.1637 | 0.2044 | 0.1407 | −0.0911 | −0.1253 | −0.0183 |
| F214 | Cytoskeleton proteins | 3 | −0.0563 | −0.6043 | −0.0217 | −0.0878 | 0.0780 | −0.2175 | −0.3120 | −0.0739 | −0.1248 |
| F215 | Polyketide sugar unit | 3 | −0.0473 | −0.4733 | −0.1759 | −0.0568 | 0.1389 | −0.1312 | −0.2784 | −0.0687 | −0.0894 |
| | biosynthesis | | | | | | | | | | |
| F217 | Prenyltransferases | 3 | −0.0606 | −0.3809 | 0.0010 | −0.0308 | 0.2374 | −0.1880 | −0.3665 | −0.1145 | −0.1197 |
| F218 | Bladder cancer | 3 | 0.0054 | 0.2846 | 0.1792 | −0.0584 | −0.1206 | −0.1221 | −0.0313 | 0.0106 | 0.0539 |
| F219 | Cellular antigens | 3 | −0.0031 | 0.0902 | −0.2067 | 0.0149 | 0.3029 | 0.0253 | −0.1462 | −0.0767 | −0.0170 |
| F220 | Carbon fixation in | 3 | −0.0571 | −0.5443 | −0.0479 | −0.0853 | 0.1599 | −0.1916 | −0.3292 | −0.0988 | −0.1281 |
| | photosynthetic | | | | | | | | | | |
| | organisms | | | | | | | | | | |
| F221 | Folate biosynthesis | 3 | −0.0528 | −0.4067 | 0.0190 | −0.0632 | 0.1881 | −0.1524 | −0.2912 | −0.1163 | −0.0810 |
| F224 | Ribosome biogenesis | 3 | −0.0757 | −0.4548 | 0.1012 | −0.0922 | 0.2063 | −0.2014 | −0.3386 | −0.0976 | −0.1173 |
| | in eukaryotes | | | | | | | | | | |
| F225 | Epithelial cell | 3 | −0.0543 | −0.5392 | 0.0461 | −0.0847 | 0.0745 | −0.2074 | −0.3031 | −0.0716 | −0.1052 |
| | signaling in | | | | | | | | | | |
| | Helicobacter pylori | | | | | | | | | | |
| | infection | | | | | | | | | | |
| F226 | Protein digestion and | 3 | 0.0019 | 0.0829 | −0.2495 | −0.0061 | 0.2304 | 0.0232 | −0.1184 | −0.0571 | −0.0057 |
| | absorption | | | | | | | | | | |
| F227 | Peroxisome | 3 | −0.0448 | −0.3295 | −0.2817 | −0.0040 | 0.2775 | −0.1099 | −0.3399 | −0.1193 | −0.0998 |
| F230 | beta-Alanine | 3 | −0.0518 | −0.3620 | 0.1108 | −0.1024 | 0.0869 | −0.1032 | −0.2042 | −0.1356 | −0.0359 |
| | metabolism | | | | | | | | | | |
| F232 | Carbohydrate | 3 | −0.0443 | 0.0126 | 0.2251 | −0.0259 | 0.0927 | −0.0660 | −0.1991 | −0.0910 | −0.0329 |
| | digestion and | | | | | | | | | | |
| | absorption | | | | | | | | | | |
| F233 | RNA transport | 3 | −0.0374 | −0.5550 | −0.0286 | −0.0859 | −0.0292 | −0.1762 | −0.2305 | −0.0450 | −0.1016 |
| F234 | Biosynthesis of | 3 | −0.0211 | 0.2176 | 0.0957 | 0.0237 | 0.2125 | 0.1433 | −0.0236 | −0.1072 | 0.0021 |
| | siderophore group | | | | | | | | | | |
| | nonribosomal | | | | | | | | | | |
| | peptides | | | | | | | | | | |
| F235 | Lipoic acid | 3 | −0.0146 | 0.1732 | −0.4141 | 0.1967 | 0.0882 | 0.1665 | −0.0544 | −0.1043 | −0.0411 |
| | metabolism | | | | | | | | | | |
| F236 | Valine, leucine and | 3 | −0.0473 | −0.5830 | −0.0961 | −0.0347 | 0.0446 | −0.1876 | −0.2858 | −0.0694 | −0.1272 |
| | isoleucine | | | | | | | | | | |
| | biosynthesis | | | | | | | | | | |
| F238 | Flavone and flavonol | 3 | −0.0381 | −0.1084 | −0.2224 | 0.0067 | 0.1066 | 0.0238 | −0.0418 | −0.0500 | 0.0062 |
| | biosynthesis | | | | | | | | | | |
| F239 | Naphthalene | 3 | −0.0525 | −0.3204 | −0.0574 | 0.0769 | 0.0934 | −0.0491 | −0.2883 | −0.1009 | −0.1472 |
| | degradation | | | | | | | | | | |
| F240 | D-Arginine and D- | 3 | −0.0097 | 0.1023 | 0.1343 | −0.0436 | 0.1061 | 0.0894 | 0.0654 | −0.0395 | 0.0669 |
| | ornithine metabolism | | | | | | | | | | |
| F241 | Measles | 3 | 0.0042 | 0.1957 | −0.0251 | −0.0790 | −0.0990 | 0.0282 | 0.3080 | 0.1800 | 0.1665 |
| F242 | Novobiocin | 3 | −0.0371 | −0.5406 | −0.0051 | −0.0880 | 0.1874 | −0.1703 | −0.3120 | −0.0837 | −0.1242 |
| | biosynthesis | | | | | | | | | | |
| F243 | Translation factors | 3 | −0.0705 | −0.4889 | 0.0358 | −0.0801 | 0.1991 | −0.2107 | −0.3618 | −0.1042 | −0.1374 |
| F244 | Glutamatergic | 3 | −0.0525 | −0.5443 | −0.2329 | −0.0372 | 0.1390 | −0.1755 | −0.3303 | −0.0779 | −0.1218 |
| | synapse | | | | | | | | | | |
| F245 | Atrazine degradation | 3 | −0.0522 | −0.2737 | −0.0684 | 0.1678 | −0.0533 | −0.0118 | −0.0928 | −0.0163 | −0.0626 |
| F246 | Chlorocyclohexane | 3 | −0.0564 | −0.2046 | −0.0238 | 0.1020 | −0.0332 | 0.0427 | 0.0045 | −0.0405 | −0.0141 |
| | and chlorobenzene | | | | | | | | | | |
| | degradation | | | | | | | | | | |
| F248 | Arachidonic acid | 3 | −0.0325 | 0.1235 | 0.1605 | 0.0655 | 0.2458 | 0.0567 | −0.1424 | −0.0808 | −0.0664 |
| | metabolism | | | | | | | | | | |
| F249 | Glutathione | 3 | −0.0485 | −0.0903 | 0.0464 | −0.0315 | 0.2517 | −0.0080 | −0.2087 | −0.1512 | −0.0433 |
| | metabolism | | | | | | | | | | |
| F250 | Sphingolipid | 3 | 0.117 | −0.1920 | −0.4763 | 0.0401 | 0.0783 | 0.0229 | −0.1094 | −0.0736 | −0.0299 |
| | metabolism | | | | | | | | | | |
| F252 | Amino sugan nd | 3 | −0.0519 | −0.4909 | −0.1278 | −0.0690 | 0.1384 | −0.1239 | −0.2759 | −0.0969 | −0.1133 |
| | nucleotide sugar | | | | | | | | | | |
| | metabolism | | | | | | | | | | |
| F253 | Steroid biosynthesis | 3 | 0.0805 | 0.2259 | 0.0979 | 0.1075 | −0.0876 | 0.0477 | 0.1414 | 0.1090 | 0.0461 |
| F254 | Bile secretion | 3 | 0.0915 | 0.2275 | −0.0378 | −0.0262 | −0.0750 | 0.0069 | 0.1521 | 0.1904 | 0.0797 |
| F255 | Thiamine metabolism | 3 | −0.0494 | −0.5722 | 0.0040 | −0.1040 | 0.0807 | −0.1978 | −0.2744 | −0.1002 | −0.1120 |
| F256 | Signal transduction | 3 | −0.0636 | −0.5412 | 0.0772 | −0.0671 | 0.0313 | −0.1634 | −0.2369 | −0.0940 | −0.1070 |
| | mechanisms | | | | | | | | | | |
| F258 | D-Glutamine and D- | 3 | −0.0641 | −0.4641 | −0.0366 | −0.0840 | 0.2022 | −0.1755 | −0.3260 | −0.1206 | −0.1261 |
| | glutamate | | | | | | | | | | |
| | metabolism | | | | | | | | | | |
| F259 | Bacterial toxins | 3 | −0.0479 | −0.3624 | −0.1851 | −0.0082 | 0.0580 | 0.0014 | −0.1310 | −0.1187 | −0.1072 |
| F260 | beta-Lactam | 3 | −0.0248 | −0.3349 | −0.2145 | −0.0217 | −0.0417 | −0.0729 | −0.0562 | −0.0291 | −0.0295 |
| | resistance | | | | | | | | | | |
| F261 | Limonene and pinene | 3 | −0.0581 | −0.1920 | 0.0863 | 0.0868 | 0.1220 | 0.0346 | −0.1794 | −0.1446 | −0.1028 |
| | degradation | | | | | | | | | | |
| F262 | Type II diabetes | 3 | −0.0565 | −0.4430 | 0.1005 | −0.0795 | 0.0949 | −0.1636 | −0.3023 | −0.1054 | −0.1138 |
| | mellitus | | | | | | | | | | |
| F264 | Steroid hormone | 3 | 0.0250 | 0.0810 | −0.4341 | 0.2170 | 0.1472 | 0.1196 | −0.0796 | −0.0211 | −0.0305 |
| | biosynthesis | | | | | | | | | | |
| F265 | Pyruvate metabolism | 3 | −0.0551 | −0.5484 | −0.1124 | −0.0338 | 0.0653 | −0.1370 | −0.2709 | −0.0979 | −0.1298 |
| F266 | Prostate cancer | 3 | −0.0425 | −0.4278 | −0.1071 | −0.0230 | 0.2010 | −0.1448 | −0.3866 | −0.0772 | −0.1307 |
| F267 | Pathways in cancer | 3 | −0.0501 | −0.3548 | −0.0366 | −0.0080 | 0.2658 | −0.1555 | −0.3785 | −0.1004 | −0.1338 |
| F268 | Glycerophospholipid | 3 | −0.0596 | −0.5354 | 0.0075 | −0.0579 | 0.0791 | −0.1693 | −0.2846 | −0.0978 | −0.1304 |
| | metabolism | | | | | | | | | | |
| F269 | Transcription factors | 3 | −0.0711 | −0.4912 | 0.1279 | −0.0934 | 0.0036 | −0.1084 | −0.1847 | −0.0921 | −0.0886 |
| F271 | Pertussis | 3 | 0.0136 | 0.2268 | −0.3985 | 0.0864 | 0.1022 | 0.2002 | 0.0543 | −0.0765 | 0.0306 |
| F272 | Transcription related | 3 | −0.0062 | −0.0057 | 0.0499 | 0.1012 | −0.0294 | 0.2502 | 0.0446 | −0.0177 | −0.0225 |
| | proteins | | | | | | | | | | |
| F273 | Tropane, piperidine | 3 | −0.0312 | −0.4329 | −0.0293 | −0.0543 | 0.2340 | −0.1347 | −0.3230 | −0.1110 | −0.1250 |
| | and pyridine alkaloid | | | | | | | | | | |
| | biosynthesis | | | | | | | | | | |
| F274 | Sporulation | 3 | −0.0328 | −0.5560 | 0.0642 | −0.1003 | −0.0845 | −0.1940 | −0.2000 | −0.0402 | −0.0891 |
| F276 | mRNA surveillance | 3 | 0.0180 | 0.1884 | −0.0042 | −0.0885 | −0.0987 | 0.0287 | 0.2989 | 0.2031 | 0.1816 |
| | pathway | | | | | | | | | | |
| F277 | Lipid metabolism | 3 | −0.0673 | −0.5136 | 0.0560 | −0.0635 | 0.0532 | −0.1572 | −0.2668 | −0.0798 | −0.1208 |
| F280 | Meiosis-yeast | 3 | −0.0429 | 0.0623 | −0.0581 | 0.2212 | 0.2813 | 0.0861 | −0.1533 | −0.0791 | −0.0646 |
| F281 | Amino acid related | 3 | −0.0652 | −0.5436 | 0.0066 | −0.0855 | 0.1700 | −0.2138 | −0.3461 | −0.0960 | −0.1280 |
| | enzymes | | | | | | | | | | |
| F282 | Streptomycin | 3 | −0.0540 | −0.4844 | −0.1924 | −0.0277 | 0.1453 | −0.1392 | −0.3028 | −0.0805 | −0.1075 |
| | biosynthesis | | | | | | | | | | |
| F283 | Transporters | 3 | −0.0676 | −0.5114 | 0.0851 | −0.0999 | 0.0022 | −0.1371 | −0.1761 | −0.0816 | −0.0882 |
| F285 | Staphylococcus | 3 | 0.0034 | −0.1109 | 0.1611 | −0.0557 | 0.0248 | 0.1189 | 0.0657 | −0.0669 | −0.0548 |
| | aureus infection | | | | | | | | | | |
| F286 | Cysteine and | 3 | −0.0577 | −0.5264 | 0.0546 | −0.0969 | 0.1656 | −0.1713 | −0.3131 | −0.1035 | −0.1197 |
| | methionine | | | | | | | | | | |
| | metabolism | | | | | | | | | | |
| F287 | PPAR signaling | 3 | −0.0606 | −0.3960 | −0.2476 | 0.0038 | 0.2760 | −0.1343 | −0.3393 | −0.1143 | −0.1297 |
| | pathway | | | | | | | | | | |
| F288 | 1,1,,-Trichloro-2,2- | 3 | 0.0267 | 0.2012 | 0.0827 | −0.0216 | 0.2066 | 0.0981 | 0.1456 | 0.0083 | 0.0043 |
| | bis(4- | | | | | | | | | | |
| | chlorophenyl)ethane | | | | | | | | | | |
| | (DDT) degradation | | | | | | | | | | |
| F289 | Two-component | 3 | −0.0588 | −0.3631 | 0.0382 | −0.0352 | 0.1277 | −0.0635 | −0.2064 | −0.1025 | −0.0881 |
| | system | | | | | | | | | | |
| F290 | Electron tramsfer | 3 | −0.0104 | −0.1053 | 0.0160 | 0.0567 | 0.2326 | 0.2082 | −0.0426 | −0.0797 | −0.1156 |
| | carriers | | | | | | | | | | |
| F291 | Protein export | 3 | −0.0689 | −0.4836 | −0.0086 | −0.0769 | 0.1999 | −0.1930 | −0.3558 | −0.1162 | −0.1328 |
| F292 | Isoquinoline alkaloid | 3 | −0.0072 | −0.2268 | −0.0083 | −0.0756 | 0.2823 | −0.0673 | −0.2614 | −0.0910 | −0.0721 |
| | biosynthesis | | | | | | | | | | |
| F293 | Protein kinases | 3 | −0.0733 | −0.4424 | 0.0507 | −0.0556 | 0.0165 | −0.1103 | −0.1903 | −0.0940 | −0.0774 |
| F294 | Function unknown | 3 | −0.0621 | −0.3253 | 0.0169 | −0.0199 | 0.1203 | −0.0357 | −0.2133 | −0.1273 | −0.0889 |
| F297 | Viral myocarditis | 3 | 0.0221 | 0.1799 | 0.0204 | −0.0247 | −0.1277 | 0.1296 | 0.2684 | 0.0131 | 0.0665 |
| F298 | DNA replication | 3 | −0.0658 | −0.4576 | 0.0271 | −0.0790 | 0.1863 | −0.1790 | −0.3470 | −0.1143 | −0.1323 |
| F299 | Primary | 3 | −0.0251 | −0.2894 | 0.0839 | −0.1409 | 0.0930 | −0.0904 | −0.1595 | −0.1323 | −0.0294 |
| | immunodeficiency | | | | | | | | | | |
| F300 | Galactose | 3 | −0.0385 | −0.4871 | −0.2070 | −0.0783 | 0.0360 | −0.0939 | −0.1769 | −0.0878 | −0.0762 |
| | metabolism | | | | | | | | | | |
| F301 | Metabolism of | 3 | −0.0615 | −0.0351 | −0.1166 | 0.1558 | 0.0943 | 0.0268 | −0.1979 | −0.0689 | −0.1048 |
| | xenobiotics by | | | | | | | | | | |
| | cytochrome P450 | | | | | | | | | | |
| F303 | Systemic lupus | 3 | 0.0665 | 0.2418 | 0.1024 | 0.0834 | −0.0616 | 0.0736 | 0.1703 | 0.0800 | 0.0554 |
| | erythematosus | | | | | | | | | | |
| F305 | Protein processing in | 3 | −0.0215 | −0.2801 | −0.2585 | 0.0568 | 0.2603 | −0.0872 | −0.3652 | −0.0518 | −0.1420 |
| | endoplasmic | | | | | | | | | | |
| | reticulum | | | | | | | | | | |
| F306 | Cyanoamino acid | 3 | −0.0346 | −0.4344 | −0.1901 | −0.0792 | 0.1002 | −0.1421 | −0.2359 | −0.0902 | −0.0590 |
| | metabolism | | | | | | | | | | |
| F309 | Bacterial motility | 3 | −0.0239 | −0.2280 | 0.1658 | −0.0194 | −0.0361 | −0.1133 | −0.2065 | −0.0442 | −0.0684 |
| | proteins | | | | | | | | | | |
| F310 | Nucleotide | 3 | 0.0034 | −0.0533 | 0.1501 | −0.0029 | 0.0078 | 0.1056 | −0.0495 | −0.1131 | −0.0536 |
| | metabolism | | | | | | | | | | |
| F311 | Flagellar assembly | 3 | −0.0250 | −0.1840 | 0.1722 | −0.0370 | −0.0410 | −0.1081 | −0.1966 | −0.0378 | −0.0473 |
| F312 | NOD-like receptor | 3 | −0.0394 | −0.4559 | −0.1504 | −0.0539 | 0.2196 | −0.1813 | −0.3726 | −0.0909 | −0.1294 |
| | signaling pathway | | | | | | | | | | |
| F313 | Amyotrophic lateral | 3 | −0.0192 | 0.1234 | −0.1224 | 0.0736 | 0.1827 | 0.2294 | 0.0035 | −0.0960 | 0.0072 |
| | sclerosis (ALS) | | | | | | | | | | |
| F314 | Base excision repair | 3 | −0.0727 | −0.5084 | 0.0068 | −0.0492 | 0.1337 | −0.1784 | −0.3158 | −0.1041 | −0.1279 |
| F315 | Photosynthesis- | 3 | −0.0007 | 0.0927 | 0.1005 | 0.0127 | −0.0722 | −0.0335 | 0.0134 | 0.0088 | 0.0236 |
| | antenna proteins | | | | | | | | | | |
| F316 | Pyrimidine | 3 | −0.0708 | −0.4824 | 0.0596 | −0.1037 | 0.1859 | −0.2046 | −0.3430 | −0.1106 | −0.1215 |
| | metabolism | | | | | | | | | | |
| F317 | Tuberculosis | 3 | −0.0713 | −0.5229 | −0.0077 | −0.0555 | 0.1819 | −0.2101 | −0.3597 | −0.1059 | −0.1373 |
| F318 | Taurine and | 3 | −0.0582 | −0.3659 | −0.1361 | −0.0033 | 0.1867 | −0.0736 | −0.2879 | −0.1332 | −0.1254 |
| | hypotaurine | | | | | | | | | | |
| | metabolism | | | | | | | | | | |
| F319 | Small cell lung | 3 | 0.0221 | 0.1799 | 0.0204 | −0.0247 | −0.1277 | 0.1296 | 0.2684 | 0.0131 | 0.0665 |
| | cancer | | | | | | | | | | |
| F320 | Nitrogen metabolism | 3 | −0.0381 | −0.4365 | −0.0804 | −0.0837 | 0.2132 | −0.0770 | −0.2571 | −0.1165 | −0.1008 |
| F322 | Drug metabolism- | 3 | −0.0624 | −0.4471 | 0.0366 | −0.0957 | 0.1771 | −0.1893 | −0.3320 | −0.1191 | −0.1093 |
| | other enzymes | | | | | | | | | | |
| F323 | Restriction enzyme | 3 | −0.0230 | −0.4000 | −0.2520 | −0.0135 | 0.1504 | −0.1148 | −0.2847 | −0.0834 | −0.1333 |
| F324 | Biosynthesis of type | 3 | 0.1182 | 0.1552 | −0.0026 | −0.0381 | −0.0501 | 0.0046 | 0.0160 | 0.0965 | 0.0035 |
| | II polyketide | | | | | | | | | | |
| | products | | | | | | | | | | |
| F325 | Homologous | 3 | −0.0686 | −0.4978 | 0.0262 | −0.0893 | 0.1886 | −0.2137 | −0.3577 | −0.1111 | −0.1310 |
| | recombination | | | | | | | | | | |
| F326 | Tyrosine metabolism | 3 | −0.0698 | −0.4635 | 0.0318 | −0.0269 | 0.1211 | −0.1354 | −0.3057 | −0.1143 | −0.1243 |
| F327 | Pantothenate and | 3 | −0.0518 | −0.5906 | −0.0314 | −0.0708 | 0.0901 | −0.2063 | −0.3095 | −0.0826 | −0.1190 |
| | CoA biosynthesis | | | | | | | | | | |
| F328 | Prion diseases | 3 | −0.0027 | −0.0285 | 0.0330 | −0.0570 | 0.2286 | 0.1979 | 0.0686 | −0.0530 | 0.0386 |
| F329 | Glycophingolipid | 3 | 0.0352 | 0.0851 | −0.4502 | 0.0952 | 0.1854 | 0.1002 | −0.0985 | −0.0518 | −0.0184 |
| | biosynthesis-ganglio | | | | | | | | | | |
| | series | | | | | | | | | | |
| F330 | Toluene degradation | 3 | −0.0187 | 0.0260 | −0.0764 | 0.0267 | 0.6240 | 0.0671 | −0.1574 | −0.0996 | −0.0373 |
| F331 | Nicotinate and | 3 | −0.0732 | −0.4826 | 0.0636 | −0.1150 | 0.1606 | −0.2091 | −0.3296 | −0.1151 | −0.1104 |
| | nicotinamide | | | | | | | | | | |
| | metabolism | | | | | | | | | | |
| F332 | DNA repair and | 3 | −0.0704 | −0.5009 | 0.0406 | −0.0836 | 0.1778 | −0.1976 | −0.3413 | −0.1100 | −0.1272 |
| | recombination | | | | | | | | | | |
| | proteins | | | | | | | | | | |
| F333 | Glycerolipid | 3 | −0.0422 | −0.5085 | 0.0130 | −0.0572 | 0.0059 | −0.1099 | −0.1968 | −0.0854 | −0.1156 |
| | metabolism | | | | | | | | | | |
| F334 | Biosynthesis of | 3 | −0.0373 | −0.5687 | −0.0291 | −0.0887 | 0.0377 | −0.1607 | −0.2382 | −0.0767 | −0.1284 |
| | ansamycins | | | | | | | | | | |
| F335 | Glycolysis/ | 3 | −0.0703 | −0.5472 | −0.0323 | −0.0578 | 0.0858 | −0.1626 | −0.2896 | −0.1078 | −0.1318 |
| | Gluconeogenesis | | | | | | | | | | |
| F336 | Styrene degradation | 3 | −0.0372 | −0.2673 | 0.0232 | 0.0394 | −0.0138 | 0.0553 | −0.0234 | −0.0515 | −0.0529 |
| F337 | Retinol metabolism | 3 | −0.0599 | −0.0354 | −0.0305 | 0.0848 | 0.1842 | −0.0186 | −0.2441 | −0.0547 | −0.0933 |
| F338 | Amoebiasis | 3 | −0.0344 | −0.2298 | −0.3104 | 0.1472 | −0.0770 | 0.0173 | −0.1025 | −0.0343 | −0.1022 |
| F339 | Biosynthesis of | 3 | −0.0370 | −0.4325 | −0.1558 | −0.0342 | 0.1896 | −0.1436 | −0.3221 | −0.0774 | −0.0986 |
| | vancomycin group | | | | | | | | | | |
| | antibiotics | | | | | | | | | | |
| F340 | Toxoplasmosis | 3 | 0.0221 | 0.1799 | 0.0204 | −0.0247 | −0.1277 | 0.1296 | 0.2684 | 0.0131 | 0.0665 |
| F342 | Hepatitis C | 3 | 0.0042 | 0.1957 | −0.0251 | −0.0790 | −0.0990 | 0.0282 | 0.3080 | 0.1800 | 0.1665 |
| F344 | Chagas disease | 3 | −0.0493 | 0.2415 | 0.1853 | 0.1642 | −0.1122 | 0.0242 | 0.0291 | −0.0412 | 0.0073 |
| | (American | | | | | | | | | | |
| | trypanosomiasis) | | | | | | | | | | |
| F345 | Glycosphingolipid | 3 | −0.0144 | 0.3487 | 0.1360 | −0.0380 | −0.1738 | 0.2779 | 0.2730 | 0.0090 | 0.0886 |
| | biosynthesis-lacto | | | | | | | | | | |
| | and neolacto series | | | | | | | | | | |
| F346 | Phenylalanine, | 3 | −0.0397 | −0.5891 | −0.0355 | −0.0805 | 0.0762 | −0.2013 | −0.2960 | −0.0771 | −0.1179 |
| | tyrosine and | | | | | | | | | | |
| | tryptophan | | | | | | | | | | |
| | biosynthesis | | | | | | | | | | |
| F347 | Photosynthesis | 3 | −0.0643 | −0.5596 | 0.0353 | −0.1412 | 0.1206 | −0.2406 | −0.2695 | −0.0768 | −0.0515 |
| | proteins | | | | | | | | | | |
| F348 | Progesterone- | 3 | −0.0509 | −0.4782 | −0.0345 | −0.0573 | 0.2017 | −0.1975 | −0.4005 | −0.0749 | −0.1323 |
| | mediated oocyte | | | | | | | | | | |
| | maturation | | | | | | | | | | |
| F350 | Ubiquinone and other | 3 | −0.0131 | 0.1723 | −0.0279 | 0.0788 | 0.2920 | 0.0958 | −0.1376 | −0.1044 | −0.0074 |
| | terpenoid-quinone | | | | | | | | | | |
| | biosynthesis | | | | | | | | | | |
| F351 | Chaperones and | 3 | −0.0614 | −0.4576 | −0.0395 | −0.0751 | 0.2418 | −0.1881 | −0.3599 | −0.1092 | −0.1162 |
| | folding catalysts | | | | | | | | | | |
| F353 | Proteasome | 3 | −0.0559 | −0.4657 | 0.0047 | −0.0598 | 0.1921 | −0.1995 | −0.3841 | −0.0831 | −0.1282 |
| F356 | Non-homologous | 3 | −0.0136 | −0.1180 | −0.1232 | 0.2055 | −0.0605 | 0.0342 | −0.1445 | −0.0098 | −0.1445 |
| | end-joining | | | | | | | | | | |
| F357 | Fluorobenzoate | 3 | 0.0398 | 0.2484 | 0.1244 | 0.1380 | −0.0781 | 0.0338 | 0.0201 | 0.0066 | 0.0244 |
| | degradation | | | | | | | | | | |
| F358 | Replication, | 3 | −0.0684 | −0.4609 | 0.0979 | −0.0369 | 0.1010 | −0.1220 | −0.2624 | −0.1207 | −0.1171 |
| | recombination and | | | | | | | | | | |
| | repair proteins | | | | | | | | | | |
| F359 | alpha-Linolenic acid | 3 | −0.0372 | 0.1194 | −0.0836 | 0.0977 | 0.0747 | 0.1937 | −0.0358 | −0.0947 | −0.0304 |
| | metabolism | | | | | | | | | | |
| F360 | Nitrotoluene | 3 | −0.0413 | −0.4270 | 0.0729 | −0.0840 | 0.0265 | −0.0818 | −0.1212 | −0.0480 | −0.0550 |
| | degradation | | | | | | | | | | |
| F361 | ABC transporters | 3 | −0.0604 | −0.4673 | 0.1147 | −0.0958 | 0.0292 | −0.1175 | −0.1649 | −0.0866 | −0.0852 |
| F363 | Plant-pathogen | 3 | −0.0403 | −0.5557 | 0.0305 | −0.1005 | 0.0883 | −0.2179 | −0.3082 | −0.0863 | −0.1283 |
| | enteraction | | | | | | | | | | |
| F365 | Mineral absorption | 3 | −0.0815 | −0.0666 | 0.2065 | 0.0358 | 0.1426 | −0.0182 | −0.2224 | −0.0709 | −0.1038 |
| F366 | Parkinson's disease | 3 | 0.0294 | 0.1872 | 0.0802 | −0.0380 | −0.0908 | −0.0830 | 0.0396 | 0.0124 | 0.0148 |
| F367 | Ubiquitin system | 3 | −0.0063 | 0.2026 | −0.3933 | 0.1529 | 0.0409 | 0.1775 | 0.0864 | −0.0871 | 0.0298 |
| F368 | Chromosome | 3 | −0.0662 | −0.5275 | 0.0490 | −0.1061 | 0.1825 | −0.2164 | −0.3512 | −0.0991 | −0.1204 |

TABLE 22

| Microbiome Sub- | |
|---|---|
| System | Importance |
| 0 | 0.122 |
| 1 | 0.111 |
| 2 | 0.141 |
| 3 | 0.113 |
| 4 | 0.095 |
| 5 | 0.206 |
| 6 | 0.135 |
| 7 | 0.052 |
| 8 | 0.020 |

TABLE 23

| Taxa ID | Taxonomy name |
|---|---|
| 544 | Citrobacter |
| 817 | Bacteroides fragilis |
| 872 | Desulfovibrio |
| 901 | Desulfovibrio piger |
| 28118 | Odoribacter splanchnicus |
| 28221 | Deltaproteobacteria |
| 35832 | Bilophila |
| 35833 | Bilophila wadsworthia |
| 47678 | Bacteroides caccae |
| 100883 | Coprobacillus |
| 194924 | Desulfovibrionaceae |
| 213115 | Desulfovibrionales |
| 216572 | Oscillospiraceae |
| 283168 | Odoribacter |
| 292800 | Flavonifractor plautii |
| 310298 | Bacteroides coprocola |
| 408103 | Citrobacter sp. BW4 |
| 447027 | Alistipes sp. EBA6-25cl2 |
| 556262 | Coprobacillus sp. D6 |
| 650643 | Alistipes sp. RMA 9912 |
| 693988 | Bilophila sp. 4_1_30 |
| 946234 | Flavonifractor |

TABLE 24

| id_fun | name_fun |
|---|---|
| F4 | Cell Growth and Death_KEGG_Pathways_Level_2.spf |
| F9 | Immune System_KEGG_Pathways_Level_2.spf |
| F20 | Translation_KEGG_Pathways_Level_2.spf |
| F29 | Replication and Repair_KEGG_Pathways_Level_2.spf |
| F36 | Environmental Adaptation_KEGG_Pathways_Level_2.spf |
| F44 | Cell cycle - Caulobacter_KEGG_Pathways_Level_3.spf |
| F58 | Nucleotide excision repair_KEGG_Pathways_Level_3.spf |
| F62 | One carbon pool by folate_KEGG_Pathways_Level_3.spf |
| F63 | Peptidoglycan biosynthesis_KEGG_Pathways_Level_3.spf |
| F65 | Aminoacyl-tRNA biosynthesis_KEGG_Pathways_Level_3.spf |
| F100 | Ribosome Biogenesis_KEGG_Pathways_Level_3.spf |
| F111 | Photosynthesis_KEGG_Pathways_Level_3.spf |
| F183 | Terpenoid backbone biosynthesis_KEGG_Pathways_Level_3.spf |
| F189 | Polycyclic aromatic hydrocarbon |
| | degradation_KEGG_Pathways_Level_3.spf |
| F190 | Ribosome_KEGG_Pathways_Level_3.spf |
| F214 | Cytoskeleton proteins_KEGG_Pathways_Level_3.spf |
| F224 | Ribosome biogenesis in |
| | eukaryotes_KEGG_Pathways_Level_ 3.spf |
| F225 | Epithelial cell signaling in Helicobacter pylori |
| | infection_KEGG_Pathways_Level_3.spf |
| F243 | Translation factors_KEGG_Pathways_Level_3.spf |
| F271 | Pertussis_KEGG_Pathways_Level_3.spf |
| F272 | Transcription related proteins_KEGG_Pathways_Level_3.spf |
| F281 | Amino acid related enzymes_KEGG_Pathways_Level_3.spf |
| F290 | Electron transfer carriers_KEGG_Pathways_Level_3.spf |
| F313 | Amyotrophic lateral sclerosis |
| | (ALS)_KEGG_Pathways_Level_3.spf |
| F316 | Pyrimidine metabolism_KEGG_Pathways_Level_3.spf |
| F317 | Tuberculosis_KEGG_Pathways_Level_3.spf |
| F325 | Homologous recombination_KEGG_Pathways_ Level_3.spf |
| F327 | Pantothenate and CoA |
| | biosynthesis_KEGG_Pathways_Level_3.spf |
| F331 | Nicotinate and nicotinamide |
| | metabolism_KEGG_Pathways_Level_3.spf |
| F345 | Glycosphingolipid biosynthesis - lacto and neolacto |
| | series_KEGG_Pathways_Level_3.spf |
| F346 | Phenylalanine, tyrosine and tryptophan |
| | biosynthesis_KEGG_Pathways_Level_3.spf |
| F347 | Photosynthesis proteins_KEGG_Pathways_Level_3.spf |
| F363 | Plant-pathogen interaction_KEGG_Pathways_Level_3.spf |
| F368 | Chromosome_KEGG_Pathways_Level_3.spf |

TABLE 25

| Taxa ID | Taxonomy name | Molecule |
|---|---|---|
| 817 | Bacteroides fragilis | Dimetridazole |
| | | (DMZ) |
| | | Norfloxacin |
| 28,118 | Odoribacter | Resveratrol |
| | splanchnicus | Pterostilbene |
| | | Sulfonolipids |
| | | Acarbose |
| 35,833 | Bilophila | Taurine |
| | wadsworthia | Taurocholic acid |
| 47,678 | Bacteroides caccae | Imipenem, ampicillin, and ampicillin/ |
| | | sulbactam |
| 292,800 | Flavonifractor plautii | Flavonoids |
| 310,298 | Bacteroides coprocola | Dihydrothymine |
| | | 3,5-hydroxybenzoate |

TABLE 26

| | Observed Scale | | | |
|---|---|---|---|---|
| | | | Percentile | |
| Condition | Mean | Std | 32 | 68 |
| Bloating | 0.377 | 0.111 | 0.325 | 0.425 |
| Bloody stool | 0.393 | 0.087 | 0.349 | 0.434 |
| Celiac disease | 0.347 | 0.080 | 0.306 | 0.379 |
| Constipation | 0.305 | 0.077 | 0.267 | 0.337 |
| Crohn's disease | 0.466 | 0.076 | 0.433 | 0.502 |
| Dairy allergy | 0.315 | 0.086 | 0.272 | 0.353 |
| Diarrhea | 0.329 | 0.075 | 0.291 | 0.363 |
| Egg allergy | 0.283 | 0.078 | 0.243 | 0.316 |
| Gluten intolerance | 0.293 | 0.072 | 0.257 | 0.326 |
| Grave's disease | 0.405 | 0.102 | 0.354 | 0.453 |
| Hashimoto's thyroiditis | 0.498 | 0.088 | 0.461 | 0.542 |
| Hemorrhoids diseases | 0.208 | 0.065 | 0.174 | 0.233 |
| Irritable bowel disease | 0.377 | 0.093 | 0.333 | 0.419 |
| Irritable bowel syndrome | 0.221 | 0.058 | 0.191 | 0.244 |
| Multiple sclerosis | 0.115 | 0.089 | 0.060 | 0.144 |
| Osteoarthritis | 0.270 | 0.074 | 0.233 | 0.305 |
| Peanut allergy | 0.362 | 0.080 | 0.323 | 0.399 |
| Photosensitivity | 0.109 | 0.041 | 0.087 | 0.121 |
| Psoriasis | 0.385 | 0.097 | 0.340 | 0.431 |
| Reflux disease | 0.197 | 0.063 | 0.163 | 0.219 |
| Rheumatoid arthritis | 0.174 | 0.116 | 0.098 | 0.220 |
| Rosacea | 0.330 | 0.102 | 0.280 | 0.379 |
| Soy allergy | 0.432 | 0.086 | 0.395 | 0.472 |
| Tree nut allergy | 0.266 | 0.081 | 0.223 | 0.301 |
| Ulcerative colitis | 0.632 | 0.070 | 0.599 | 0.667 |
| Wheat allergy | 0.354 | 0.086 | 0.311 | 0.393 |

TABLE 27

| | | Significantly | | | |
|---|---|---|---|---|---|
| Cluster | Conditions | associated pairs | mean | std | p-value |
| Cluster 1 | Rosacea | Celiac disease - Wheat | 0.323 | 0.071 | 2.5 × 10⁻⁶ |
| | Celiac disease | allergy | | | |
| | Photosensibility | Celiac disease - Gluten | 0.385 | 0.073 | 7.17 × 10⁻⁸ |
| | Wheat allergy | intolerance | | | |
| | Gluten | Celiac disease - | 0.251 | 0.059 | 1.12 × 10⁻⁵ |
| | intolerance | Photosensibility | | | |
| | | Rosacea - Photosensibility | 0.236 | 0.062 | 7.49 × 10⁻⁵ |
| | | Gluten intolerance - | 0.359 | 0.073 | 4.36 × 10⁻⁷ |
| | | Wheat allergy | | | |
| | | Gluten intolerance - | 0.297 | 0.071 | 1.47 × 10⁻⁵ |
| | | Rosacea | | | |
| | | Gluten intolerance - | 0.253 | 0.063 | 2.68 × 10⁻⁵ |
| | | Photosensibility | | | |
| Cluster | Dairy allergy | — | | | |
| II | Bloating^{†} | | | | |
| | Rheumatoid | | | | |
| | arthritis | | | | |
| Cluster | Inflammatory | Constipation - | | | |
| III | bowel syndrome | Inflammatory Bowel | 0.279 | 0.070 | 3.20 × 10⁻⁵ |
| | (IBS) | Syndrome | | | |
| | Hemorrhoidal | Hemorrhoidal disease - | 0.214 | 0.056 | 7.3 × 10⁻⁵ |
| | disease | Reflux | | | |
| | Constipation^{†} | Hemorrhoidal disease - | 0.224 | 0.059 | 7.6 × 10⁻⁵ |
| | Reflux | Inflammatory Bowel | | | |
| | | Syndrome | | | |
| | | Inflammatory Bowel | 0.249 | 0.064 | 5.2 × 10⁻⁵ |
| | | Syndrome - Reflux | | | |
| Cluster | Multiple sclerosis | — | | | |
| IV | Osteoarthritis | | | | |
| Cluster | Ulcerative colitis | Ulcerative colitis - | 0.601 | 0.069 | 1.57 × 10⁻¹⁸ |
| V | Crohn's disease | Crohn's disease | | | |
| | Diarrheat | Ulcerative colitis - | 0.366 | 0.092 | 3.45 × 10⁻⁵ |
| | | Diarrhea | | | |
| | | Crohn's disease - Diarrhea | 0.434 | 0.076 | 5.72 × 10⁻⁹ |
| Cluster | Soy allergy | Bloody stool - | 0.457 | 0.083 | 1.72 × 10⁻⁸ |
| VI | Peanut allergy | Inflammatory Bowel | | | |
| | Treenut allergy | Disease | | | |
| | Egg allergy | Bloody stool- Peanut | 0.346 | 0.086 | 2.72 × 10⁻⁵ |
| | Psoriasis | allergy | | | |
| | Hashimoto's | Bloody stool - Treenut | 0.358 | 0.09:7 | 7.67 × 10⁻⁵ |
| | thyroiditis | allergy | | | |
| | Graves disease | Bloody stool - Egg allergy | 0.345 | 0.090 | 6.83 × 10⁻⁵ |
| | Inflammatory | Egg allergy - Soy allergy | 0.359 | 0.083 | 8.38 × 10⁻⁶ |
| | Bowel Disease | Egg allergy - Treenut | 0.349 | 0.084 | 1.55 × 10⁻⁵ |
| | Bloody stool^{†} | allergy | | | |
| | | Graves disease - Psoriasis | 0.373 | 0.098 | 7.35 × 10⁻⁵ |
| | | Hashimoto's thyroiditis - | 0.423 | 0.097 | 6.81 × 10⁻⁶ |
| | | Inflammatory Bowel | | | |
| | | Disease | | | |
| | | Hashimoto's thyroiditis - | 0.295 | 0.078 | 7.76 × 10⁻⁵ |
| | | Treenut allergy | | | |

^{†}Symptoms

TABLE 28

| | | Gluten | |
|---|---|---|---|
| | Celiac disease | intolerance | Wheat allergy |
| Celiac disease | 1827 | | |
| Gluten | 1796 | 1818 | |
| intolerance | | | |
| Wheat allergy | 751 | 740 | 905 |

TABLE 29

| | | Lactose |
|---|---|---|
| | Dairy Allergy | Intolerance |
| Dairy Allergy | 415 | |
| Lactose | | |
| Intolerance | | |

TABLE 30

| | | | Crohn's | Ulcerative |
|---|---|---|---|---|
| | IBS | IBD | Disease | Colitis |
| IBS | 1701 | | | |
| IBD | 111 | 502 | | |
| Crohn's | 60 | 238 | 238 | |
| Disease | | | | |
| Ulcerative | 61 | 293 | 32 | 293 |
| Colitis | | | | |

TABLE 31

| Number of Conditions | Female % | Male % |
|---|---|---|
| 0 | xx | Xx |
| 1 | 27.3 | 24.7 |
| 2 | 22.3 | 16.8 |
| 3 | 17.2 | 9.9 |
| 4 | 11.9 | 5.0 |
| 5 | 7.9 | 3.6 |
| 6 | 4.6 | 1.5 |
| 7 | 3.4 | 1.2 |
| 8+ | 4.3 | 1.2 |

We claim:

1. A system for characterization of a microorganism-related condition, the system comprising:
a sample handling system comprising a sequencing system operable to determine microorganism genetic sequences based on samples associated with a set of subjects, wherein the samples comprise microorganism nucleic acids associated with the microorganism-related condition;
a set of microbiome characterization modules operable to apply a set of analytical techniques comprising at least two of a statistical test, a dimensionality reduction technique, and an artificial intelligence approach, and wherein the set of microbiome characterization modules comprises:
a first microbiome characterization module operable to apply a first analytical technique, of the set of analytical techniques, to determine a set of microbiome features based on the microorganism genetic sequences, wherein the set of microbiome features is associated with the microorganism-related condition; and
a second microbiome characterization module operable to apply a second analytical technique, of the set of analytical techniques, to determine a processed microbiome feature set based on the set of microbiome features, wherein the processed microbiome feature set is adapted to improve the characterization of the microorganism-related condition; and
a microorganism-related condition model generated based on the processed microbiome feature set, wherein the microorganism-related condition model is operable to determine a characterization of the microorganism-related condition for a user.

2. The system of claim 1, wherein the first analytical technique comprises a statistical test comprising at least one of a t-test, a Kolmogorov-Smirnov test, and a regression model, and wherein the first microbiome characterization module is operable to apply the statistical test to determine the set of microbiome features based on the microorganism genetic sequences.

3. The system of claim 1,
wherein the samples comprise site-diverse samples collected from a plurality of collection sites comprising at least two of gut, genitals, mouth, skin, and nose, and
wherein the first microbiome characterization module is operable to apply the statistical test to determine first subsets of microbiome features of the set microbiome features based on the site-diverse samples, wherein each subset of microbiome features from the first subsets of microbiome features corresponds to a different collection site from the plurality of collection sites.

4. The system of claim 3,
wherein the second microbiome characterization module is operable to apply an additional statistical test to determine second subsets of microbiome features of the set of microbiome features based on the site-diverse samples, and
wherein the microorganism-related condition model is generated based on the first subsets and the second subsets of microbiome features.

5. The system of claim 1, wherein the second microbiome characterization module is operable to apply the second analytical technique to perform at least one of feature selection, feature weighting, and warm start, for processing the set of microbiome features into the processed microbiome feature set.

6. The system of claim 1, wherein the microorganism-related condition model comprises a skin-related characterization model generated based on the processed microbiome feature set, wherein the skin-related characterization model is operable to determine the characterization of a photosensitivity-associated condition for the user, and wherein the set of microbiome features comprises features associated with at least one of: Alloprevotella (genus), Prevotella sp. WAL 2039G (species), Corynebacterium mastitidis (species), Bacteroidaceae (family), Blautia (genus), Bacteroides (genus), Desulfovibrio (genus), Clostridium (genus), Bacteroides vulgatus (species), Faecalibacterium prausnitzii (species), Blautia faecis (species), Alistipes putredinis (species), Bacteroides sp. AR20 (species), Bacteroides sp. AR29 (species), Bacteroides acidifaciens (species), Dielma (genus), Slackia (genus), Eggerthella (genus), Adlercreutzia (genus), Paraprevotella (genus), Alistipes (genus), Holdemania (genus), Eisenbergiella (genus), Enterorhabdus (genus), Adlercreutzia equolifaciens (species), Phascolarctobacterium succinatutens (species), Roseburia inulinivorans (species), Phascolarctobacterium sp. 377 (species), Desulfovibrio piger (species), Eggerthella sp. HGA1 (species), Lactonifactor longoviformis (species), Alistipes sp. HGB5 (species), Holdemania filiformis (species), Collinsella intestinalis (species), Neisseria macacae (species), Clostridiaceae (family), Gemella sanguinis (species), Bacteroides fragilis (species), Enterobacteriaceae (family), Lachnospiraceae (family), Pasteurellaceae (family), Pasteurellales (order), Enterobacteriales (order), Sphingobacteriales (order), Haemophilus (genus), Leuconostoc (genus), Brevundimonas (genus), Prevotella oris (species), Odoribacter (genus), Capnocytophaga (genus), Flavobacterium (genus), Pseudomonas brenneri (species), Flavobacterium ceti (species), Brevundimonas sp. FXJ8.0080 (species), Ruminococcaceae (family), Vibrionaceae (family), Flavobacteriaceae (family), Fusobacteriaceae (family), Porphyromonadaceae (family), Brevibacteriaceae (family), Rhodobacteraceae (family), Intrasporangiaceae (family), Bifidobacteriaceae (family), Sphingobacteriaceae (family), Caulobacteraceae (family), Campylobacteraceae (family), Bacteroidia (class), Fusobacteriia (class), Flavobacteriia (class), Bifidobacteriales (order), Neisseriales (order), Bacteroidales (order), Rhodobacterales (order), Flavobacteriales (order), Vibrionales (order), Fusobacteriales (order), Caulobacterales (order), Fusobacteria (phylum), Actinobaculum (genus), Varibaculum (genus), Fusicatenibacter (genus), Brevibacterium (genus), Faecalibacterium (genus), Campylobacter (genus), Actinobacillus (genus), Porphyromonas (genus), Fusobacterium (genus), Chryseobacterium (genus), Megasphaera (genus), Rothia (genus), Neisseria (genus), Lactobacillus sp. BL302 (species), Bacteroides plebeius (species), Corynebacterium ulcerans (species), Varibaculum cambriense (species), Blautia wexlerae (species), Staphylococcus sp. WB18-16 (species), Streptococcus sp. oral taxon G63 (species), Propionibacterium acnes (species), Anaerococcus sp. 9401487 (species), Haemophilus parainfluenzae (species), Staphiococcus epidermidis (species), Campylobacter ureolyticus (species), Janibacter sp. M3-5 (species), Prevotella timonensis (species), Peptoniphilus sp. DNF00840 (species), Finegoldia sp. S8 F7 (species), Prevotella disiens (species), Porphromonas catoniae (species), Fusobacterium periodonticum (species), Infectious Diseases (KEGG2), Poorly Characterized (KEGG2), Metabolic Diseases (KEGG2), Immune System Diseases (KEGG2), Cellular Processes and Signaling (KEGG2), Restriction enzyme (KEGG3), Nucleotide excision repair (KEGG3).

7. The system of claim 1, wtierein the microorganism-related condition model comprises a skin-related characterization model generated based on the processed microbiome feature set, wherein the skin-related characterization model is operable to determine the characterization of a dry skin-associated condition for the user, and wherein the set of microbiome features comprises features associated with at least one of: Corynebacteriaceae (family), Bacilli (class), Lactobacillales (order), Actinomycetales (order), Firmicutes (phylum), Corynebacterium (genus), Dermabacteraceae (family), Lactobacillaceae (family), Propionibacteriaceae (family), Actinobacteria (class), Dermabacter (genus), Dialister (genus), Facklamia (genus), Lactobacillus (genus), Propionibacterium (genus), Corynebacterium ulcerans (species), Facklamia hominis (species), Corynebacterium sp. (species), Propionibacterium sp. MSP09A (species), Facklamia sp. 1440-97 (species), Staphylococcus sp. C912 (species), Anaerococcus sp. 9402080 (species), Corynebacterium glucuronolyticum (species), Dermabacter hominis (species), Enterobacteriaceae (family), Pseudomonadaceae (family), Staphylococcaceae (family), Gammaproteobacteria (class), Bacillales (order), Enterobacteriales (order), Bifidobacterium (genus), Pseudomonas (genus), Anaeroglobus (genus), Kluyvera (genus), Atopobium (genus), Staphylococcus (genus), Lactobacillus sp. BL302 (species), Corynebacterium mastitidis (species), Bifidobacterium longum (species), Anaeroglobus geminatus (species), Anaerococcus sp. S9 PR-16 (species), Prevotella timonensis (species), Kluyvera georgiana (species), Actinobaculum (genus), Finegoldia (genus), Cronobacter (genus), Acinetobacter sp. WB22-23 (species), Anaerococcus octavius (species), Finegoldia sp. 59 AA1-5 (species), Staphylococcus sp. C-D-MA2 (species), Peptoniphilus sp. 7-2 (species), Cronobacter sakazakii (species), Pasteurellaceae (family), Acidobacteriia (class), Sphingobacteriia (class), Sphingobacteriales (order), Acidobacteria (phylum), Porphyromonas (genus), Haemophilus (genus), Acinetobacter (genus), Anaerococcus sp. 8405254 (species), Sphingomonadaceae (family), Sphingomonadales (order), Kocuria (genus), Gemella (genus), Veillonella sp. CM60 (species), Lactobacillus sp. 7_1_47FAA (species), Gemella sp. 933-88 (species), Porphyromonas catoniae (species), Haemophilus parainfluenzae (species), Bacteroides sp. AR20 (species), Bacteroides vulgatus (species), Bacteroides sp. D22 (species), Dorea longicatena (species), Parabacteroides merdae (species), Bacteroides sp. AR29 (species), Dorea (genus), Collinsella (genus), Bacteroides (genus), Oscillospiraceae (family), Ruminococcaceae (family), Bacteroidaceae (family), Verrucomicrobiaceae (family), Coriobacteriaceae (family), Clostridiales (order), Bacteroidales (order), Verrucomicrobiales (order), Coriobacteriales (order), Thermoanaerobacterales (order), Clostridia (class), Bacteroidia (class), Verrucomicrobiae (class), Verrucomicrobia (phylum), Bacteroidetes (phylum), Translation (KEGG2), Cellular Processes and Signaling (KEGG2), Amino Acid Metabolism (KEGG2), Cell Growth and Death (KEGG2), Replication and Repair (KEGG2), Metabolism of Other Amino Acids (KEGG2), Neurodegenerative Diseases (KEGG2), Metabolism of Cofactors and Vitamins (KEGG2), Transport and Catabolism (KEGG2), Endocrine System (KEGG2), Immune System Diseases (KEGG2), Excretory System (KEGG2), Enzyme Families (KEGG2), Membrane Transport (KEGG2), Carbohydrate Metabolism (KEGG2), Biosynthesis of Other Secondary Metabolites (KEGG2), Metabolism of Terpenoids and Polyketides (KEGG2), Infectious Diseases (KEGG2), Genetic Information Processing (KEGG2), Nervous System (KEGG2), Environmental Adaptation (KEGG2), Nucleotide Metabolism (KEGG2), Signaling Molecules and Interaction (KEGG2), Signal Transduction (KEGG2), Inorganic ion transport and metabolism (KEGG3), Chromosome (KEGG3), Cell cycle—Caulobacter (KEGG3), Ribosome Biogenesis (KEGG3), DNA replication proteins (KEGG3), Translation factors (KEGG3), Glycine, serine and threonine metabolism (KEGG3), Sulfur metabolism (KEGG3), Other ion-coupled transporters (KEGG3), Valine, leucine and isoleucine biosynthesis (KEGG3), Nitrogen metabolism (KEGG3), Peptidoglycan biosynthesis (KEGG3), Homologous recombination (KEGG3), Peroxisome (KEGG3), Sulfur relay system (KEGG3), Peptidases (KEGG3), Protein kinases (KEGG3), Mismatch repair (KEGG3), Xylene degradation (KEGG3), Ribosome (KEGG3), RNA polymerase (KEGG3), Tryptophan metabolism (KEGG3), Histidine metabolism (KEGG3), Vitamin metabolism (KEGG3), Cell motility and secretion (KEGG3), Pyrimidine metabolism (KEGG3), Cytoskeleton proteins (KEGG3), DNA replication (KEGG3), Amino sugar and nucleotide sugar metabolism (KEGG3), Folate biosynthesis (KEGG3), Carbon fixation in photosynthetic organisms (KEGG3), Phosphatidylinositol signaling system (KEGG3), Lysine degradation (KEGG3), Selenocompound metabolism (KEGG3), Fructose and mannose metabolism (KEGG3), Inositol phosphate metabolism (KEGG3), Protein folding and associated processing (KEGG3), PPAR signaling pathway (KEGG3), Lipid metabolism (KEGG3), Valine, leucine and isoleucine degradation (KEGG3), Glyoxylate and dicarboxylate metabolism (KEGG3), Arginine and proline metabolism (KEGG3), Limonene and pinene degradation (KEGG3), D-Alanine metabolism (KEGG3), Porphyrin and chlorophyll metabolism (KEGG3), C5-Branched dibasic acid metabolism (KEGG3), Chaperones and folding catalysts (KEGG3), Fatty acid metabolism (KEGG3), Glutathione metabolism (KEGG3), Pentose phosphate pathway (KEGG3), Phosphotransferase system (PTS) (KEGG3), Pantothenate and CoA biosynthesis (KEGG3), Proximal tubule bicarbonate reclamation (KEGG3), Galactose metabolism (KEGG3), Starch and sucrose metabolism (KEGG3), Primary immmunodeficiency (KEGG3), Cysteine and methionine metabolism (KEGG3), Ubiquinone and other terpenoid-quinone biosynthesis (KEGG3), DNA repair and recombination proteins (KEGG3), Tyrosine metabolism (KEGG3), Phenylalanine, tyrosine and tryptophan biosynthesis (KEGG3), Aminoacyl-tRNA biosynthesis (KEGG3), Alanine, aspartate and glutamate metabolism (KEGG3), Photosynthesis (KEGG3), Other transporters (KEGG3), Butanoate metabolism (KEGG3), Bacterial secretion system (KEGG3), Glycerophospholipid metabolism (KEGG3), Oxidative phosphorylation (KEGG3), Type I diabetes mellitus (KEGG3), Glycolysis/Gluconeogenesis (KEGG3), Photosynthesis proteins (KEGG3), Transporters (KEGG3), Terpenoid backbone biosynthesis (KEGG3), Biosynthesis of unsaturated fatty acids (KEGG3), Signal transduction mechanisms (KEGG3), Synthesis and degradation of ketone bodies (KEGG3), Nucleotide excision repair (KEGG3), Secretion system (KEGG3), Alzheimer's disease (KEGG3), Zeatin biosynthesis (KEGG3), Type II diabetes mellitus (KEGG3), D-Glutamine and D-glutamate metabolism (KEGG3), Taurine and hypotaurine metabolism (KEGG3), Glutamatergic synapse (KEGG3), Plant-pathogen interaction (KEGG3), Vitamin B6 metabolism (KEGG3), Citrate cycle (TCA cycle) (KEGG3), Ethylbenzene degradation (KEGG3), Base excision repair (KEGG3), Replication, recombination and repair proteins (KEGG3), Ribosome biogenesis in eukaryotes (KEGG3), Aminobenzoate degradation (KEGG3), Bacterial motility proteins (KEGG3), Biosynthesis of ansamycins (KEGG3), Ion channels (KEGG3), Metabolism (KEGG2), Poorly Characterized (KEGG2), Biosynthesis and biodegradation of secondary metabolites (KEGG3), Lipoic acid metabolism (KEGG3), Amino acid related enzymes (KEGG3), Translation proteins (KEGG3), Ascorbate and aldarate metabolism (KEGG3), Thiamine metabolism (KEGG3), Function unknown (KEGG3), Glycosaminoglycan degradation (KEGG3), Others (KEGG3), Pentose and glucuronate interconversions (KEGG3), Biotin metabolism (KEGG3), Phenylalanine metabolism (KEGG3), Glycosphingolipid biosynthesis—ganglio series (KEGG3), Pores ion channels (KEGG3), Membrane and intracellular structural molecules (KEGG3), Purine metabolism (KEGG3), One carbon pool by folate (KEGG3), Phosphonate and phosphinate metabolism (KEGG3), Lysosome (KEGG3), Drug metabolism—other enzymes (KEGG3), Penicillin and cephalosporin biosynthesis (KEGG3), Huntington's disease (KEGG3), Nicotinate and nicotinamide metabolism (KEGG3), Drug metabolism—cytochrome P450 (KEGG3), Lipopolysaccharide biosynthesis proteins (KEGG3), Metabolism of xenobiotics by cytochrome P450 (KEGG3), Tuberculosis (KEGG3), and Polycyclic aromatic hydrocarbon degradation (KEGG3).

8. The system of claim 1, wherein the microorganism-related condition model comprises a skin-related characterization model generated based on the processed microbiome feature set, wherein the skin-related characterization model is operable to determine the characterization of a scalp-related condition for the user, and wherein the set of microbiome features comprises features associated with at least one of: Actinobacteria (class), Lactobacillales (order), Actinomycetales (order), Firmicutes (phylum), Dermabacteraceae (family), Lactobacillaceae (family), Propionibacteriaceae (family), Corynebacteriaceae (family), Lactobacillus (genus), Corynebacterium (genus), Propionibacterium (genus), Dermabacter (genus), Eremococcus (genus), Corynebacterium freiburgense (species), Eremoc(KEGG3)occus coleocola (species), Corynebacterium sp. (species), Staphylococcus sp. C912 (species), Anaerococcus sp. 8405254 (species), Corynebacterium glucuronolyticum (species), Dermabacter hominis (species), Coriobacteriaceae (family), Enterobacteriaceae (family), Staphylococcaceae (family), Enterobacteriales (order), Bacillales (order), Bifidobacterium (genus), Staphylococcus (genus), Atopobium (genus), Megasphaera (genus), Corynebacterium mastitidis (species), Streptococcus sp. BS35a (species), Finegoldia magna (species), Staphylococcus aureus (species), Haemophilus influenzae (species), Corynebacterium sp. NML 97-0186 (species), Streptococcus sp. oral taxon G59 (species), Dorea (genus), Roseburia sp. 11SE39 (species), Dorea longicatena (species), Prevotellaceae (family), Veillonellaceae (family), Oscillospiraceae (family), Negativicutes class, Selenomonadales (order), Finegoldia (genus), Oscillospira (genus), Intestinimonas (genus), Flavonifractor (genus), Prevotella (genus), Moryella (genus), Catenibacterium mitsuokai (species), Collinsella aerofaciens (species), Peptoniphilus sp. 2002-2300004 (species), Corynebacterium canis (species), Finegoldia sp. S9 AA1-5 (species), Prevotella buccalis (species), Dialister invisus (species), Moraxella (genus), Neisseria (genus), Neisseria mucosa (species), Rikenellaceae (family), Metabolism of Cofactors and Vitamins (KEGG2), Enzyme Families (KEGG2), Lipid Metabolism (KEGG2), Immune System Diseases (KEGG2), Glycolysis/Gluconeogenesis (KEGG3), Primary immunodeficiency (KEGG3), Pyruvate metabolism (KEGG3), Transport and Catabolism (KEGG2), Neurodegenerative Diseases (KEGG2), Endocrine System (KEGG2), Amino Acid Metabolism (KEGG2), Cellular Processes and Signaling (KEGG2), Signaling Molecules and Interaction (KEGG2), Metabolism of Other Amino Acids (KEGG2), Replication and Repair (KEGG2), Translation (KEGG2), Cell Growth and Death (KEGG2), Membrane Transport (KEGG2), Biosynthesis of Other Secondary Metabolites (KEGG2), Metabolism of Terpenoids and Polyketides (KEGG2), Inorganic ion transport and metabolism (KEGG3), Vitamin metabolism (KEGG3), Valine, leucine and isoleucine biosynthesis (KEGG3), Peroxisome (KEGG3), Ribosome Biogenesis (KEGG3), Selenocompound metabolism (KEGG3), Histidine metabolism (KEGG3), Chromosome (KEGG3), Sulfur metabolism (KEGG3), PPAR signaling pathway (KEGG3), Porphyrin and chlorophyll metabolism (KEGG3), Phosphatidylinositol signaling system (KEGG3), Inositol phosphate metabolism (KEGG3), Sulfur relay system (KEGG3), Glycine, serine and threonine metabolism (KEGG3), DNA replication proteins (KEGG3), Pantothenate and CoA biosynthesis (KEGG3), Translation factors (KEGG3), Protein folding and associated processing (KEGG3), Type 11 diabetes mellitus (KEGG3), Protein kinases (KEGG3), Folate biosynthesis (KEGG3), Lysine degradation (KEGG3), RNA polymerase (KEGG3), D-Alanine metabolism (KEGG3), Carbon fixation in photosynthetic organisms (KEGG3), Nitrogen metabolism (KEGG3), Glycerophospholipid metabolism (KEGG3), Biosynthesis of ansamycins (KEGG3), Valine, leucine and isoleucine degradation (KEGG3), Cytoskeleton proteins (KEGG3), Peptidases (KEGG3), Fatty acid metabolism (KEGG3), Cell cycle—Caulobacter (KEGG3), Phosphotransferase system (FTS) (KEGG3), Pyrimidine metabolism (KEGG3), Alzheimer's disease (KEGG3), Butanoate metabolism (KEGG3), Tryptophan metabolism (KEGG3), Signal transduction mechanisms (KEGG3), Pentose phosphate pathway (KEGG3), Other ion-coupled transporters (KEGG3), Homologous recombination (KEGG3), Replication, recombination and repair proteins (KEGG3), Xylene degradation (KEGG3), Mismatch repair (KEGG3), Glyoxylate and dicarboxylate metabolism (KEGG3), Arginine and proline metabolism (KEGG3), Peptidoglycan biosynthesis (KEGG3), Chaperones and folding catalysts (KEGG3), Type I diabetes mellitus (KEGG3), DNA replication (KEGG3), Bacterial secretion system (KEGG3), Tyrosine metabolism (KEGG3), Citrate cycle (TCA cycle) (KEGG3), Amino sugar and nucleotide sugar metabolism (KEGG3), Ribosome (KEGG3), Limonene and pinene degradation (KEGG3), Cell motility and secretion (KEGG3), Taurine and hypotaurine metabolism (KEGG3), Oxidative phosphorylation (KEGG3), Fructose and mannose metabolism (KEGG3), Vitamin B6 metabolism (KEGG3), Ion channels (KEGG3), Synthesis and degradation of ketone bodies (KEGG3), Other transporters (KEGG3), Galactose metabolism (KEGG3), Polycyclic aromatic hydrocarbon degradation (KEGG3), Transporters (KEGG3), DNA repair and recombination proteins (KEGG3), Starch and sucrose metabolism (KEGG3), Alanine, aspartate and glutamate metabolism (KEGG3), Ribosome biogenesis in eukaryotes (KEGG3), Secretion system (KEGG3), Biosynthesis of unsaturated fatty acids (KEGG3), Cysteine and methionine metabolism (KEGG3), Base excision repair (KEGG3), Aminobenzoate degradation (KEGG3), Photosynthesis (KEGG3), Photosynthesis proteins (KEGG3), Pores ion channels (KEGG3), Lipid biosynthesis proteins (KEGG3), and D-Glutamine and D-glutamate metabolism (KEGG3).

9. A method for characterizing a microorganism-related condition, the method comprising:
determining a microorganism sequence dataset for a user based on microorganism nucleic acids from a sample associated with the user; and
determining a characterization of the microorganism-related condition for the user based on the microorganism sequence dataset and a microorganism-related condition model generated based on the application, with a set of microbiome characterization modules, of a set of analytical techniques to determine a set of microbiome features,
wherein the set of analytical techniques comprises at least one of a statistical test, a dimensionality reduction technique, and an artificial intelligence approach,
wherein the set of microbiome characterization modules comprises:
a first microbiome characterization module operable to apply a first analytical technique of the set of analytical techniques, and
a second microbiome characterization module operable to apply a second analytical technique of the set of analytical techniques.

10. The method of claim 9, wherein the application of the set of analytical techniques to determine the set of microbiome features comprises:
determination of an initial set of microbiome features based on the microorganism sequence dataset; and
application, with the first microbiome characterization module of the set of microbiome characterization modules, of the dimensionality reduction technique on the initial set of microbiome features to determine the set of microbiome features, wherein the dimensionality reduction technique comprises at least one of missing values ratio, principal component analysis, probabilistic principal component analysis, matrix factorization techniques, compositional mixture models, and feature embedding techniques.

11. The method of claim 10, wherein the determination of the initial set of microbiome features comprises application, with the second microbiome characterization module of the set of microbiome characterization modules, of the statistical test with the microorganism sequence dataset to determine the initial set of microbiome features, wherein the statistical test comprises at least one of a t-test, a Kolmogorov-Smirnov test, and a regression model.

12. The method of claim 10, wherein the application of the set of analytical techniques comprises, with the second microbiome characterization module of the set of microbiome characterization modules, application of a machine learning approach to determine relevance scores for the set of microbiome features, wherein the microorganism-related condition is generated based on the set of microbiome features and the relevance scores.

13. The method of claim 10, wherein determining the characterization comprises determining a drug metabolism characterization associated with the microorganism-related condition based on the microorganism-related condition model, the sample from the user, and known associations between the set of microbiome features and drug metabolization.

14. The method of claim 9, wherein determining the characterization of the microorganism-related condition for the user comprises:
collecting, from the user, a set of site-diverse samples corresponding to a plurality of collection sites comprising at least two of gut, genitals, mouth, skin, and nose, wherein the set of site-diverse samples comprises the sample from the user;
determining a set of site-wise disease propensity metrics based on the set of site-diverse samples and the microorganism-related condition model, wherein each site-wise disease propensity metric, of the set of site-wise disease propensity metrics, corresponds to a different collection site of the plurality of collection sites and is associated with the microorganism-related condition;
determining an overall disease propensity metric for the user based on the set of site-wise disease propensity metrics, wherein the overall disease propensity metric is associated with the microorganism-related condition.

15. The method of claim 14, further comprising determining a microorganism dataset associated with the plurality of collection sites based on the set of site-diverse samples, wherein determining the overall disease propensity metric comprises:
determining at least one of a covariance metric and a correlation metric, based on the microorganism dataset, wherein the at least one of the covariance metric and the correlation metric is associated with the plurality of collection sites; and
determining the overall disease propensity metric for the user based on the set of site-wise disease propensity metrics and the at least one of the covariance metric and the correlation metric.

16. The method of claim 9, wherein the microorganism-related condition model is generated based on, prior to the application of the set of analytical techniques to determine the microbiome features, filtering of the microorganism sequence dataset by at least one of: a) removing first sample data corresponding to first sample outliers of the set of samples, wherein the first sample outliers are determined by at least one of principal component analysis, a dimensionality reduction technique, and a multivariate methodology; b) removing second sample data corresponding to second sample outliers of the set of samples, wherein the second sample outliers are determined based on corresponding data quality for the set of microbiome features; and c) removing a microbiome feature from the set of microbiome features based on a sample number for the microbiome feature failing to satisfy a threshold sample number condition, wherein the sample number corresponds to a number of samples associated with high quality data for the microbiome feature.

17. The method of claim 9, wherein determining the characterization of the microorganism-related condition for the user comprises determining a skin-related characterization of a photosensitivity-associated condition for the user based on a set of user microbiome features and the microorganism-related condition model, wherein the set of user microbiome features comprises features associated with at least one of: Alloprevotella (genus), Prevotella sp. WAL 2039G (species), Corynebacterium mastitidis (species), Bacteroidaceae (family), Blautia (genus), Bacteroides (genus), Desulfovibrio (genus), Clostridium (genus), Bacteroides vulgatus (species), Faecalibacterium prausnitzii (species), Blautia faecis (species), Alistipes putredinis (species), Bacteroides sp. AR20 (species), Bacteroides sp. AR29 (species), Bacteroides acidifaciens (species), Dielma (genus), Slackia (genus), Eggerthella (genus), Adlercreutzia (genus), Paraprevotella (genus), Alistipes (genus), Holdemania (genus), Eisenbergiella (genus), Enterorhabdus (genus), Adlercreutzia equolifaciens (species), Phascolarctobacterium succinatutens (species), Roseburia inulinivorans (species), Phascolarctobacterium sp. 377 (species), Desulfovibrio piger (species), Eggerthella sp. HGA1 (species), Lactonifactor longoviformis (species), Alistipes sp. HGB5 (species), Holdemania filiformis (species), Collinsella intestinalis (species), Neisseria macacae (species), Clostridiaceae (family), Gemella sanguinis (species), Bacteroides fragilis (species), Enterobacteriaceae (family), Lachnospiraceae (family), Pasteurellaceae (family), Pasteurellales (order), Enterobacteriales (order), Sphingobacteriales (order), Haemophilus (genus), Leuconostoc (genus), Brevundimonas (genus), Prevotella oris (species), Odoribacter (genus), Capnocytophaga (genus), Flavobacterium (genus), Pseudomonas brenneri (species), Flavobacterium ceti (species), Brevundimonas sp. FXJ8.080 (species), Ruminococcaceae (family), Vibrionaceae (family), Flavobacteriaceae (family), Fusobacteriaceae (family), Porphyromonadaceae (family), Brevibacteriaceae (family), Rhodobacteraceae (family), Intrasporangiaceae (family), Bifidobacteriaceae (family), Sphingobacteriaceae (family), Caulobacteraceae (family), Campylobacteraceae (family), Bacteroidia (class), Fusobacteriia (class), Flavobacteriia (class), Bifidobacteriales (order), Neisseriales (order), Bacteroidales (order), Rhodobacterales (order), Flavobacteriales (order), Vibrionales (order), Fusobacteriales (order), Caulobacterales (order), Fusobacteria (phylum), Actinobaculum (genus), Varibaculum (genus), Fusicatenibacter (genus), Brevibacterium (genus), Faecalibacterium (genus), Campylobacter (genus), Actinobacillus (genus), Porphyromonas (genus), Fusobacterium (genus), Chryseobacterium (genus), Megasphaera (genus), Rothia (genus), Neisseria (genus), Lactobacillus sp. BL302 (species), Bacteroides plebeius (species), Corynebacterium ulcerans (species), Varibaculum cambriense (species), Blautia wexlerae (species), Staphylococcus sp. WB18-16 (species), Streptococcus sp. oral taxon G63 (species), Propionibacterium acnes (species), Anaerococcus sp. 9401487 (species), Haemophilus parainfluenzae (species), Staphylococcus epidermidis (species), Campylobacter ureolyticus (species), Janibacter sp. M3-5 (species), Prevotella timonensis (species), Peptoniphilus sp. DNF00840 (species), Finegoldia sp. S8 F7 (species), Prevotella disiens (species), Porphyromonas catoniae (species), Fusobacterium periodonticum (species), Infectious Diseases (KEGG2), Poorly Characterized (KEGG2), Metabolic Diseases (KEGG2), Immune System Diseases (KEGG2), Cellular Processes and Signaling (KEGG2), Restriction enzyme (KEGG3), Nucleotide excision repair (KEGG3).

18. The method of claim 9, wherein determining the characterization of the microorganism-related condition for the user comprises determining a skin-related characterization of a dry skin-associated condition for the user based on a set of user microbiome features and the microorganism-related condition model, wherein the set of user microbiome features comprises features associated with at least one of: Corynebacteriaceae (family), Bacilli (class), Lactobacillales (order), Actinomycetales (order), Firmicutes (phylum), Corynebacterium (genus), Dermabacteraceae (family), Lactobacillaceae (family), Propionibacteriaceae (family), Actinobacteria (class), Dermabacter (genus), Dialister (genus), Facklamia (genus), Lactobacillus (genus), Propionibacterium (genus), Corynebacterium ulcerans (species), Facklamia hominis (species), Corynebacterium sp. (species), Propionibacterium sp. MSP09A (species), Facklamia sp. 1440-97 (species), Staphylococcus sp. C912 (species), Anaerococcus sp. 9402080 (species), Corynebacterium glucuronolyticum (species), Dermabacter hominis (species), Enterobacteriaceae (family), Pseudomonadaceae (family), Staphylococcaceae (family), Gammaproteobacteria (class), Bacillales (order), Enterobacteriales (order), Bifidobacterium (genus), Pseudomonas (genus), Anaeroglobus (genus), Kluyvera (genus), Atopobium (genus), Staphylococcus (genus), Lactobacillus sp. BL302 (species), Corynebacterium mastitidis (species), Bifidobacterium longum (species), Anaeroglobus geminatus (species), Anaerococcus sp. 89 PR-16 (species), Prevotella timonensis (species), Kluyvera georgiana (species), Actinobaculum (genus), Finegoldia (genus), Cronobacter (genus), Acinetobacter sp. WB22-23 (species), Anaerococcus octavius (species), Finegoldia sp. S9 AA1-5 (species), Staphylococcus sp. C-D-MA2 (species), Peptoniphilus sp. 7-2 (species), Cronobacter sakazakii (species), Pasteurellaceae (family), Acidobacteriia (class), Sphingobacteriia (class), Sphingobacteriales (order), Acidobacteria (phylum), Porphyromonas (genus), Haemophilus (genus), Acinetobacter (genus), Anaerococcus sp. 8405254 (species), Sphingomonadaceae (family), Sphingomonadales (order), Kocuria (genus), Gemella (genus), Veillonella sp. CM60 (species), Lactobacillus sp. 7_1_47FAA (species), Gemella sp. 933-88 (species), Porphyromonas catoniae (species), Haemophilus parainfluenzae (species), Bacteroides sp. AR20 (species), Bacteroides vulgatus (species), Bacteroides sp. D22 (species), Dorea longicatena (species), Parabacteroides merdae (species), Bacteroides sp. AR29 (species), Dorea (genus), Collinsella (genus), Bacteroides (genus), Oscillospiraceae (family), Ruminococcaceae (family), Bacteroidaceae (family), Verrucomicrobiaceae (family), Coriobacteriaceae (family), Clostridiales (order), Bacteroidales (order), Verrucomicrobiales (order), Coriobacteriales (order), Thermoanaerobacterales (order), Clostridia (class), Bacteroidia (class), Verrucomicrobiae (class), Verrucomicrobia (phylum), Bacteroidetes (phylum), Translation (KEGG2), Cellular Processes and Signaling (KEGG2), Amino Acid Metabolism (KEGG2), Cell Growth and Death (KEGG2), Replication and Repair (KEGG2), Metabolism of Other Amino Acids (KEGG2), Neurodegenerative Diseases (KEGG2), Metabolism of Cofactors and Vitamins (KEGG2), Transport and Catabolism (KEGG2), Endocrine System (KEGG2), Immune System Diseases (KEGG2), Excretory System (KEGG2), Enzyme Families (KEGG2), Membrane Transport (KEGG2), Carbohydrate Metabolism (KEGG2), Biosynthesis of Other Secondary Metabolites (KEGG2), Metabolism of Terpenoids and Polyketides (KEGG2), Infectious Diseases (KEGG2), Genetic Information Processing (KEGG2), Nervous System (KEGG2), Environmental Adaptation (KEGG2), Nucleotide Metabolism (KEGG2), Signaling Molecules and Interaction (KEGG2), Signal Transduction (KEGG2), Inorganic ion transport and metabolism (KEGG3), Chromosome (KEGG3), Cell cycle—Caulobacter (KEGG3), Ribosome Biogenesis (KEGG3), DNA replication proteins (KEGG3), Translation factors (KEGG3), Glycine, serine and threonine metabolism (KEGG3), Sulfur metabolism (KEGG3), Other ion-coupled transporters (KEGG3), Valine, leucine and isoleucine biosynthesis (KEGG3), Nitrogen metabolism (KEGG3), Peptidoglycan biosynthesis (KEGG3), Homologous recombination (KEGG3), Peroxisome (KEGG3), Sulfur relay system (KEGG3), Peptidases (KEGG3), Protein kinases (KEGG3), Mismatch repair (KEGG3), Xylene degradation (KEGG3), Ribosome (KEGG3), RNA polymerase (KEGG3), Tryptophan metabolism (KEGG3), Histidine metabolism (KEGG3), Vitamin metabolism (KEGG3), Cell motility and secretion (KEGG3), Pyrimidine metabolism (KEGG3), Cytoskeleton proteins (KEGG3), DNA replication (KEGG3), Amino sugar and nucleotide sugar metabolism (KEGG3), Folate biosynthesis (KEGG3), Carbon fixation in photosynthetic organisms (KEGG3), Phosphatidylinositol signaling system (KEGG3), Lysine degradation (KEGG3), Selenocompound metabolism (KEGG3), Fructose and mannose metabolism (KEGG3), Inositol phosphate metabolism (KEGG3), Protein folding and associated processing (KEGG3), PPAR signaling pathway (KEGG3), Lipid metabolism (KEGG3), Valine, leucine and isoleucine degradation (KEGG3), Glyoxylate and dicarboxylate metabolism (KEGG3), Arginine and proline metabolism (KEGG3), Limonene and pinene degradation (KEGG3), D-Alanine metabolism (KEGG3), Porphyrin and chlorophyll metabolism (KEGG3), C5-Branched dibasic acid metabolism (KEGG3), Chaperones and folding catalysts (KEGG3), Fatty acid metabolism (KEGG3), Glutathione metabolism (KEGG3), Pentose phosphate pathway (KEGG3), Phosphotransferase system (PTS) (KEGG3), Pantothenate and CoA biosynthesis (KEGG3), Proximal tubule bicarbonate reclamation (KEGG3), Galactose metabolism (KEGG3), Starch and sucrose metabolism (KEGG3), Primary immunodeficiency (KEGG3), Cysteine and methionine metabolism (KEGG3), Ubiquinone and other terpenoid-quinone biosynthesis (KEGG3), DNA repair and recombination proteins (KEGG3), Tyrosine metabolism (KEGG3), Phenylalanine, tyrosine and tryptophan biosynthesis (KEGG3), Aminoacyl-tRNA biosynthesis (KEGG3), Alanine, aspartate and glutamate metabolism (KEGG3), Photosynthesis (KEGG3), Other transporters (KEGG3), Butanoate metabolism (KEGG3), Bacterial secretion system (KEGG3), Glycerophospholipid metabolism (KEGG3), Oxidative phosphorylation (KEGG3), Type I diabetes mellitus (KEGG3), Glycolysis/Gluconeogenesis (KEGG3), Photosynthesis proteins (KEGG3), Transporters (KEGG3), Terpenoid backbone biosynthesis (KEGG3), Biosynthesis of unsaturated fatty acids (KEGG3), Signal transduction mechanisms (KEGG3), Synthesis and degradation of ketone bodies (KEGG3), Nucleotide excision repair (KEGG3), Secretion system (KEGG3), Alzheimer's disease (KEGG3), Zeatin biosynthesis (KEGG3), Type II diabetes mellitus (KEGG3), D-Glutamine and D-glutamate metabolism (KEGG3), Taurine and hypotaurine metabolism (KEGG3), Glutamatergic synapse (KEGG3), Plant-pathogen interaction (KEGG3), Vitamin B6 metabolism (KEGG3), Citrate cycle (TCA cycle) (KEGG3), Ethylbenzene degradation (KEGG3), Base excision repair (KEGG3), Replication, recombination and repair proteins (KEGG3), Ribosome biogenesis in eukaryotes (KEGG3), Aminobenzoate degradation (KEGG3), Bacterial motility proteins (KEGG3), Biosynthesis of ansamycins (KEGG3), Ion channels (KEGG3), Metabolism (KEGG2), Poorly Characterized (KEGG2), Biosynthesis and biodegradation of secondary metabolites (KEGG3), Lipoic acid metabolism (KEGG3), Amino acid related enzymes (KEGG3), Translation proteins (KEGG3), Ascorbate and aldarate metabolism (KEGG3), Thiamine metabolism (KEGG3), Function unknown (KEGG3), Glycosaminoglycan degradation (KEGG3), Others (KEGG3), Pentose and glucuronate interconversions (KEGG3), Biotin metabolism (KEGG3), Phenylalanine metabolism (KEGG3), Glycosphingolipid biosynthesis—ganglio series (KEGG3), Pores ion channels (KEGG3), Membrane and intracellular structural molecules (KEGG3), Purine metabolism (KEGG3), One carbon pool by folate (KEGG3), Phosphonate and phosphinate metabolism (KEGG3), Lysosome (KEGG3), Drug metabolism—other enzymes (KEGG3), Penicillin and cephalosporin biosynthesis (KEGG3), Huntington's disease (KEGG3), Nicotinate and nicotinamide metabolism (KEGG3), Drug metabolism—cytochrome P450 (KEGG3), Lipopolysaccharide biosynthesis proteins (KEGG3), Metabolism of xenobiotics by cytochrome P450 (KEGG3), Tuberculosis (KEGG3), and Polycyclic aromatic hydrocarbon degradation (KEGG3).

19. The method of claim 9, wherein determining the characterization of the microorganism-related condition for the user comprises determining a skin-related characterization of a scalp-related condition for the user based on a set of user microbiome features and the microorganism-related condition model, wherein the set of user microbiome features comprises features associated with at least one of: Actinobacteria (class), Lactobacillales (order), Actinomycetales (order), Firmicutes (phylum), Dermabacteraceae (family), Lactobacillaceae (family), Propionibacteriaceae (family), Cornebacteriaceae (family), Lactobacillus (genus), Corynebacterium (genus), Propionibacterium (genus), Dermabacter (genus), Eremococcus (genus), Corynebacterium freiburgense (species), Eremoc(KEGG3)occus coleocola (species), Corynebacterium sp. (species), Staphylococcus sp. C912 (species), Anaerococcus sp. 8405254 (species), Corynebacterium glucuronolyticum (species), Dermabacter hominis (species), Coriobacteriaceae (family), Enterobacteriaceae (family), Staphylococcaceae (family), Enterobacteriales (order), Bacillales (order), Bifidobacterium (genus), Staphylococcus (genus), Atopobium (genus), Megasphaera (genus), Corynebacterium mastitidis (species), Streptococcus sp. BS35a (species), Finegoldia magna (species), Staphylococcus aureus (species), Haemophilus influenzae (species), Corynebacterium sp. NML 97-0186 (species), Streptococcus sp. oral taxon G59 (species), Dorea (genus), Roseburia sp. 11SE39 (species), Dorea longicatena (species), Prevotellaceae (family), Veillonellaceae (family), Oscillospiraceae (family), Negativicutes class, Selenomonadales (order), Finegoldia (genus), Oscillospira (genus), Intestinimonas (genus), Flavonifractor (genus), Prevotella (genus), Moryella (genus), Catenibacterium mitsuokai (species), Collinsella aerofaciens (species), Peptoniphilus sp. 2002-2300004 (species), Cornebacterium canis (species), Finegoldia sp. S9 AA1-5 (species), Prevotella buccalis (species), Dialister invisus (species), Moraxella (genus), Neisseria (genus), Neisseria mucosa (species), Rikenellaceae (family), Metabolism of Cofactors and Vitamins (KEGG2), Enzyme Families (KEGG2), Lipid Metabolism (KEGG2), Immune System Diseases (KEGG2), Glycolysis/Gluconeogenesis (KEGG3), Primary immunodeficiency (KEGG3), Pyruvate metabolism (KEGG3), Transport and Catabolism (KEGG2), Neurodegenerative Diseases (KEGG2), Endocrine System (KEGG2), Amino Acid Metabolism (KEGG2), Cellular Processes and Signaling (KEGG2), Signaling Molecules and Interaction (KEGG2), Metabolism of Other Amino Acids (KEGG2), Replication and Repair (KEGG2), Translation (KEGG2), Cell Growth and Death (KEGG2), Membrane Transport (KEGG2), Biosynthesis of Other Secondary Metabolites (KEGG2), Metabolism of Terpenoids and Polyketides (KEGG2), Inorganic ion transport and metabolism (KEGG3), Vitamin metabolism (KEGG3), Valine, leucine and isoleucine biosynthesis (KEGG3), Peroxisome (KEGG3), Ribosome Biogenesis (KEGG3), Selenocompound metabolism (KEGG3), Histidine metabolism (KEGG3), Chromosome (KEGG3), Sulfur metabolism (KEGG3), PPAR signaling pathway (KEGG3), Porphyrin and chlorophyll metabolism (KEGG3), Phosphatidylinositol signaling system (KEGG3), Inositol phosphate metabolism (KEGG3), Sulfur relay system (KEGG3), Glycine, serine and threonine metabolism (KEGG3), DNA replication proteins (KEGG3), Pantothenate and CoA biosynthesis (KEGG3), Translation factors (KEGG3), Protein folding and associated processing (KEGG3), Type II diabetes mellitus (KEGG3), Protein kinases (KEGG3), Folate biosynthesis (KEGG3), Lysine degradation (KEGG3), RNA polymerase (KEGG3), D-Alanine metabolism (KEGG3), Carbon fixation in photosynthetic organisms (KEGG3), Nitrogen metabolism (KEGG3), Glycerophospholipid metabolism (KEGG3), Biosynthesis of ansamycins (KEGG3), Valine, leucine and isoleucine degradation (KEGG3), Cytoskeleton proteins (KEGG3), Peptidases (KEGG3), Fatty acid metabolism (KEGG3), Cell cycle—Caulobacter (KEGG3), Phosphotransferase system (PITS) (KEGG3), Pyrimidine metabolism (KEGG3), Alzheimer's disease (KEGG3), Butanoate metabolism (KEGG3), Tryptophan metabolism (KEGG3), Signal transduction mechanisms (KEGG3), Pentose phosphate pathway (KEGG3), Other ion-coupled transporters (KEGG3), Homologous recombination (KEGG3), Replication, recombination and repair proteins (KEGG3), Xylene degradation (KEGG3), Mismatch repair (KEGG3), Glyoxylate and dicarboxylate metabolism (KEGG3), Arginine and proline metabolism (KEGG3), Peptidoglycan biosynthesis (KEGG3), Chaperones and folding catalysts (KEGG3), Type I diabetes mellitus (KEGG3), DNA replication (KEGG3), Bacterial secretion system (KEGG3), Tyrosine metabolism (KEGG3), Citrate cycle (TCA cycle) (KEGG3), Amino sugar and nucleotide sugar metabolism (KEGG3), Ribosome (KEGG3), Limonene and pinene degradation (KEGG3), Cell motility and secretion (KEGG3), Taurine and hypotaurine metabolism (KEGG3), Oxidative phosphorylation (KEGG3), Fructose and mannose metabolism (KEGG3), Vitamin B6 metabolism (KEGG3), Ion channels (KEGG3), Synthesis and degradation of ketone bodies (KEGG3), Other transporters (KEGG3), Galactose metabolism (KEGG3), Polycyclic aromatic hydrocarbon degradation (KEGG3), Transporters (KEGG3), DNA repair and recombination proteins (KEGG3), Starch and sucrose metabolism (KEGG3), Alanine, aspartate and glutamate metabolism (KEGG3), Ribosome biogenesis in eukaryotes (KEGG3), Secretion system (KEGG3), Biosynthesis of unsaturated fatty acids (KEGG3), Cysteine and methionine metabolism (KEGG3), Base excision repair (KEGG3), Aminobenzoate degradation (KEGG3), Photosynthesis (KEGG3), Photosynthesis proteins (KEGG3), Pores ion channels (KEGG3), Lipid biosynthesis proteins (KEGG3), and D-Glutamine and D-glutamate metabolism (KEGG3).

20. The method of claim 9, wherein the microorganism-related condition comprises a skin-related condition, wherein the method further comprises promoting a probiotic therapy to the user for the skin-related condition based on the characterization, and wherein the probiotic therapy is associated with microorganisms associated with any one of the following: Corynebacterium ulcerans, Facklamia hominis, Corynebacterium sp., Propionibacterium sp. MSP09A, Facklamia sp. 1440-97, Staphylococcus sp. C912, Anaerococcus sp. 9402080, Corynebacterium glucuronolyticum, Dermabacter hominis, Lactobacillus sp. BL302, Corynebacterium mastitidis, Bifidobacterium longum, Anaeroglobus geminatus, Anaerococcus sp. S9 PR-16, Prevotella timonensis, Kluyvera georgiana, Acinetobacter sp. WB22-23, Anaerococcus octavius, Finegoldia sp. S9 AA1-5, Staphylococcus sp. C-D-MA2, Peptoniphilus sp. 7-2, Cronobacter sakazakii, Anaerococcus sp. 8405254, Veillonella sp. CM60, Lactobacillus sp. 7_1_47FAA, Gemella sp. 933-88, Porphyromonas catoniae, Haemophilus parainfluenzae, Bacteroides sp. AR20, Bacteroides vulgatus, Bacteroides sp. D22, Dorea longicatena, Parabacteroides merdae, Bacteroides sp. AR29, Prevotella sp. WAL 2039G, Faecalibacterium prausnitzii, Blautia faecis, Alistipes putredinis, Bacteroides acidifaciens, Adlercreutzia equolifaciens, Phascolarctobacterium succinatutens, Roseburia inulinivorans, Phascolarctobacterium sp. 377, Desulfovibrio piger, Eggerthella sp. HGA1, Lactonifactor longoviformis, Alistipes sp. HGB5, Holdemania filiformis, Collinsella intestinalis, Neisseria macacae, Gemella sanguinis, Bacteroides fragilis, Prevotella oris, Pseudomonas brenneri, Flavobacterium ceti, Brevundimonas sp. FXJ8.080, Bacteroides plebeius, Varibaculum cambriense, Blautia wexlerae, Staphylococcus sp. WB18-16, Streptococcus sp. oral taxon G63, Propionibacterium acnes, Anaerococcus sp. 9401487, Staphylococcus epidermidis, Campylobacter ureolyticus, Janibacter sp. M3-5, Peptoniphilus sp. DNF00840, Finegoldia sp. S8 F7, Prevotella disiens, Fusobacterium periodonticum, Corynebacterium freiburgense, Eremococcus coleocola, Streptococcus sp. BS35a, Finegoldia magna, Staphylococcus aureus, Haemophilus influenzae, Corynebacterium sp. NML97-0186, Streptococcus sp. oral taxon G59, Roseburia sp. 11SE39, Catenibacterium mitsuokai, Collinsella aerofaciens, Peptoniphilus sp. 2002-2300004, Corynebacterium canis, Prevotella buccalis, Dialister invisus, and Neisseria mucosa.

21. A method for characterization of a plurality of microorganism-related conditions, the method comprising:
determining a microorganism sequence dataset associated with the set of subjects, based on microorganism nucleic acids from samples associated with the set of subjects, wherein the microorganism nucleic acids are associated with the plurality of microorganism-related conditions;
with a set of microbiome characterization modules, determining a set of multi-condition microbiome features based on the microorganism sequence dataset, wherein each multi-condition microbiome feature of the set of multi-condition microbiome features is associated with at least two microorganism-related conditions of the plurality of microorganism-related conditions;
determining, for a user, a multi-condition characterization of microorganism-related conditions of the plurality of microorganism-related conditions based on the set of multi-condition microbiome features and a sample from the user; and
facilitating therapeutic intervention for the microorganism-related conditions of the plurality of microorganism-related conditions based on the multi-condition characterization.

22. The method of claim 21,
wherein determining the set of multi-condition microbiome features comprises applying, with a first microbiome characterization module of the set of microbiome characterization modules, a dimensionality reduction technique to an initial set of microbiome features determined based on the microorganism sequence dataset,
wherein the method further comprises determining, with a second microbiome characterization module of the set of microbiome characterization modules, a cross-condition correlation analysis between different conditions of the plurality of microorganism-related conditions, and
wherein determining the multi-condition characterization comprises determining the multi-condition characterization based on the cross-condition correlation metric, the set of multi-condition microbiome features, and the sample from the user.

23. The method of claim 22, wherein determining the multi-condition characterization for the user comprises determining a characterization of an additional condition analysis of the plurality of microorganism-related conditions based on a current user condition of the plurality of microorganism-related conditions, the set of multi-condition microbiome features, the sample from the user, and the cross-condition correlation metric.

24. The method of claim 22, wherein performing the cross-condition correlation analysis with the second microbiome characterization module comprises applying at least one of a multivariate model, a canonical correlation model, and a multi-label artificial intelligence approach, for the different conditions of the plurality of microorganism-related conditions.

25. The method of claim 21, further comprising determining a set of microorganism-related condition groups from the plurality of microorganism-related conditions based on the multi-condition microbiome features, wherein facilitating therapeutic intervention comprises facilitating therapeutic intervention for the microorganism-related conditions based on the set of microorganism-related condition groups and the multi-condition characterization.

26. The method of claim 25, wherein facilitating therapeutic intervention comprises at least one of: a) promoting a first therapy for the user based on an assignment of the user to at least one microorganism-related condition group of the set of microorganism-related condition groups; b) promoting a second therapy for the user based on associations between microorganism-related conditions belonging to a same microorganism-related condition group of the set of microorganism-related condition groups; and c) discouraging a third therapy for the user based on associations between microorganism-related conditions belonging to different microorganism-related condition groups of the set of microorganism-related condition groups.

27. The method of claim 25, wherein the set of microorganism-related condition groups comprises at least one of a first group comprising an allergy-related condition, a second group comprising a locomotor-related condition, and a third group comprising a gastrointestinal-related condition, and wherein facilitating therapeutic intervention comprises facilitating therapeutic intervention for the microorganism-related conditions based on the multi-condition characterization and the at least one of the first, the second, and the third groups.
